(19) United States
(12) Patent Application Publication
(10) Pub. No.: US 2009/0270508 A1
(43) Pub. Date: Oct. 29, 2009

(54) GluR2 receptor modulators

(76) Inventors: James Eric Gouaux, Portland, OR (US); Langzhou Song, Freehold, NJ (US); Yuli Xie, Shanghai (CN); Donald W. Landry, New York, NY (US)

Correspondence Address: Kevin C. Hooper, Bryan Cave, 1290 Avenue of the Americas, New York, NY, 10104, US

(21) Appl. No.: 12/322,274

(22) Filed: Jan. 30, 2009

Related U.S. Application Data
(63) Continuation-in-part of application No. 11/678,396, filed on Feb. 23, 2007, now abandoned.

(51) Int. Cl. A61K 31/18; C07C 311/01; A61P 25/24

(52) U.S. Cl. 514/605; 564/99

(57) ABSTRACT

The present invention provides for compositions and methods for modulating the GluR2 receptor. It is based, at least in part, on the discovery, by X-ray crystallography, that a known GluR2 agonist binds to the receptor in two different orientations, thereby diminishing its potency. The present invention provides for structural alternatives in which alternative binding possibilities are substantially eliminated.

### PRIORITY CLAIM

This application claims priority to U.S. patent application Ser. No. 11/678,396, filed Feb. 23, 2007, and U.S. Provisional Application No. 60/776,708, filed Feb. 24, 2006, each of which is incorporated herein by reference in its entirety.

### GRANT INFORMATION

The subject matter of this application was developed, at least in part, using National Institutes of Health Grant No. 5RO1NS38631-04, so that the United States Government has certain rights herein.

### INTRODUCTION

The present invention provides for compositions and methods for modulating the GluR2 receptor. It is based, at least in part, on the discovery, by X-ray crystallography, that LY451646, a known GluR2 modulator, binds to the receptor in two different orientations, thereby diminishing its potency. The present invention provides for structural alternatives in which alternative binding possibilities are substantially eliminated.

### BACKGROUND OF THE INVENTION

Communication between cells of the mammalian central nervous system (CNS) is controlled by the interactions between neurotransmitters, produced by a sending (presynaptic) cell, and their receptors in the membrane of a receiving (postsynaptic) cell. These interactions can have excitatory or inhibitory affects on the postsynaptic cell. The major excitatory pathways in the mammalian CNS are mediated by the amino acid neurotransmitter glutamate (Watkins and Evans, 1981, Annu Rev Pharmacol Toxicol. 21: 165-204; Monaghan et al., 1989, Annu Rev Pharmacol Toxicol. 29: 365-402; Watkins et al., 1990, Trends Pharmacol Sci. 11: 25-33).

The excitatory amino acid receptors that bind glutamate are classified into two different receptor types, “ionotropic” and “metabotropic.” Metabotropic glutamate receptors are slower acting than the ionotropic receptors, but are active for a longer time period upon neurotransmitter binding. The metabotropic glutamate receptors are coupled to G-protein second messenger pathways. Activation of these receptors results in changes in cytoplasmic cAMP levels, which in turn alters the functionality of membrane bound ion channels. The metabotropic glutamate receptors are composed of subunits encoded for by the genes mGlu1-8 (Schoepp and Conn, 1993, Trends Pharmacol-Sci. 14: 13-20).

Ionotropic receptors are directly coupled to ion channels in the plasma membranes of postsynaptic cells. Ionotropic glutamate receptors are fast acting. Binding of a neurotransmitter to an inactive receptor induces the activation of the receptor and opening of the ion channel, allowing the passage of ions across the cell membrane. Three different types of ionotropic glutamate receptors have been identified which are defined by the excitatory actions of their major activators: N-methyl-D-aspartate (NM DA), α-amino-3-hydroxy-5-methylisoxazole-4-propionic acid (AMPA), and kainic acid (Kainate); although AMPA and Kainate can activate each others' receptor types (Bleakman et al., 1996, Neuropharmacology 35: 1689-702).

Functional AMPA receptors are composed of subunits encoded by the genes GluR1-4. Functional Kainate receptors may be composed of the high affinity subunits encoded by the genes KA1 and KA2, and the low affinity subunits encoded by the genes GluR5-7. Each Kainate and AMPA receptor is composed of a heteromeric or homomeric cluster of four receptor subunits. The subunits that compose functional NMDA receptors are encoded by the genes NR1 and NR2. All of the genes that encode the various subunits for the three ionotropic glutamate receptors can undergo alternative splicing, generating a large diversity of potential subunit compositions.

The biophysical and pharmacological properties of the ionotropic glutamate receptors are dependent on their subunit composition (Hollmann and Heinemann, 1994, Annu Rev Neurosci 17:31-108). The GluR2 subunit has been shown to play a major role in modulating ionic currents, susceptibility to excitotoxicity, and ion permeability. Specifically, the presence of the GluR2 subunit in AMPA receptors decreases receptor permeability to Ca²⁺ (Hume et al., 1991, Science 253:1028-1031; Burnashev et al., 1992, Neuron 8:189-198). Furthermore, the C-terminal cytoplasmic tail of GluR2 is important for mediating interactions with various cytoplasmic proteins that may influence membrane receptor turnover (Man et al., 2000, Neuron 25:649-662), synaptic transmission, efficacy, and plasticity (Jia et al., 1996, Neuron 17:945-956), and receptor clustering (Matsuda et al., 2000, EMBO J 19:2765-2774).

Current models propose that the ionotropic receptor subunits, including GluR2, consist of three transmembrane spanning domains and a reentrant loop (Paas, 1998, Trends Neurosci. 21: 117-125; Wo and Oswald, 1995, Trends Neurosci. 18: 161-168). The ligand binding domain is composed of a clamshell-like dimer formed by the S1 region located before the first transmembrane domain, and the S2 region located between the second and third transmembrane domains (Armstrong and Gouaux, 2000, Neuron 28: 165-181; Armstrong et al., 1998, Nature 395: 913-917). The dimer is formed by two structural domains, one of which (“D1”) is composed mostly of amino acid residues from S1, and the other (“D2”), which is composed mostly of amino acid residues from S2 (as discussed in Valentine and Palmer, 2005, Biochem. 44:3410-3417). When the receptor is activated (see FIG. 1) by binding of an activator, the cleft between the two domains closes (hence the term, “clamshell”) and the ion channel opens. If the receptor is desensitized, the “clamshell” is held in a closed position, the activator is bound tightly, yet the ion channel is closed (FIG. 1).

While the ionotropic glutamate receptors are tetrameric complexes comprised of “dimers-of-dimers” (Sun et al., 2002, Nature 417:245-253), study of the conformational effects of binding of various modulators has been facilitated by the creation of a soluble ligand-binding core of GluR2, referred to as GluR2-S1S2J (Armstrong and Gouaux, 2000, Neuron 28: 165-181). GluR2-S1S2J comprises the two domains, S1 and S2, which, rather than being bound to transmembrane domains, are joined by a single linker (see FIG. 2). GluR2-S1S2J can be crystallized, as can be complexes between it and various modulators. High resolution X-ray structures of GluR2-S1S2J with various activators and at least one inactivator have revealed different degrees of cleft closure between D1 and D2 (Lunn et al., 2003, J. Med. Chem. 2003, 46:872-875; Jin et al., 2005, J. Neurosci. 25(39):9027-9036; Valentine and Palmer III, 2005, Biochemistry 44:3410-3417; Kubo and Ito, 2004, Proteins 56:411-419; Jin and Gouaux, 2003, Biochemistry 42:5201-5213; Hogner et al., 2003, J. Med. Chem. 46:214-221; Armstrong et al., 2003, Proc. Natl. Acad. Sci. U.S.A. 100(10):5736-5741; Carcache et al., 2003, Bioorganic & Med. Chem. 11:551-559; Jin et al., 2002, Biochem. 41:15635-15643; Hogner et al., 2002, J. Mol. Biol. 322: 93-109).

Inappropriate stimulation of the glutamate receptors can lead to neurological disorders and neurodegeneration. For example, excessive activation of the ionotropic receptors can result in a condition known as excitotoxicity in which overactivation results in the death of the glutaminergic cells. Additional conditions such as stroke, cerebral ischemia, Alzheimer's Disease, amyotrophic lateral sclerosis, Huntington's disease, AIDS-induced dementia, depression, and anxiety related disorders can result from the inappropriate activation of glutamate receptors (see U.S. Pat. Nos. 5,446,051 and 5,670,516).

The development of compounds that can modulate the activity of the glutamate receptors when they bind neurotransmitter is essential to alleviating these neurological conditions (Weiser, 2005, Curr Drug Targets CNS Neurol Disord. 4:153-9). Currently, many candidate anti-depressant and anti-anxiety drugs are tested for their ability to reduce “despair” in mice and rats during forced swim tests (FST) and tail suspension tests (TST). 87%-94% of the compounds that successfully reduce despair in mice and rats are clinically effective at reducing depression and anxiety in humans (Porsolt and Lenerge, 1992, Behavioral models of depression. In: Elliott, J. M., Heal, D. J., and Marsden, C. A. editors. Experimental approaches to anxiety and depression. London. pp. 73-85; Borsini and Meli, 1988, Psychopharmacology 94: 147-60).

LY451646, an active isomer of LY404187 (R,S—N-2-(4-(4-Cyanophenyl)phenyl)propyl 2-propanesulfonamide) and an allosteric AMPA receptor potentiator (Miu et al., 2001, Neuropharmacology 40: 976-83), has been observed to reduce “despair” in the FST and TST (Bai et al., 2001, Pharmacology, Biochemistry, and Behavior 70: 187-192), and may therefore have potential as a candidate anti-depressant or anti-anxiety drug. It has also been found to increase hippocampal cell proliferation in rats (Bai et al., 2003, Neuropharmacol. 44(8):1013-1021). Additionally, its isomer LY404187 increases glutamate dependent AMPA stimulation of the rat brain as evident by increased glucose utilization and c-fos expression in the hippocampus, neocortex, dorsal raphe nucleus, lateral habenula, and locus coeruleus; areas associated with memory processing and cognition (Fowler et al., 2004, J Cereb Blood Flow Metab. 24: 1098-109). LY404187 interacts functionally with GluR2's Flip/Flop splice domain which is adjacent to the S1S2 dimer. Interacting with the Flip splice variant, LY404187 can potentiate the receptor's response to glutamate by decreasing the receptor's desensitization to prolonged glutamate exposure (Quirk and Nisenbaum, 2003, Journal of Neuroscience. 23: 10953-10962). LY404187 has also been found to exhibit a neurotrophic effect in a rat model of Parkinson's disease (O'Neil et al., 2004, Eur. J. Pharmacol. 486(2):163-174). Understanding glutamate receptor structure and allosteric binding sites can facilitate the development of candidate compounds that may bind the receptor and modulate its response to the neurotransmitter glutamate.

### SUMMARY OF THE INVENTION

The present invention provides for compounds, pharmaceutical compositions and for methods of modulating the activity of the GluR2 receptor. It is based, in part, on the discovery that the GluR2 agonist, LY451646, bound to GluR2-S1S2J, equally occupies two different orientations, thereby diminishing its potency.

The present invention provides for new compounds that are designed to bind the GluR2 receptor in essentially one structural orientation, and for methods of identifying further modulator compounds. In particular, non-limiting embodiments, the present invention provides for molecules which exhibit 2-fold molecular symmetry and which may comprise a napthalene core or a bi-aryl core.

In various embodiments, the present invention encompasses agonists as well as antagonists of GluR2 activity, where an agonist may enhance, and an antagonist may inhibit, the effect of an endogenous activator of the GluR2 receptor, such as the effect of glutamate on the GluR2 receptor. Agonists may be used for the treatment of depression, anxiety, and cognitive disorders, and to facilitate learning. Antagonists may be used for the treatment of ischemic, infectious, and/or degenerative neurological diseases.

In further embodiments, the present invention provides for a crystal structure of GluR2-S1S2J bound to ligand LY451646, as set forth herein as atomic coordinates in Appendix A. In related embodiments, the present invention provides for a Fo-Fc electron density map of LY451646, as bound to GluR2(S1S2J), and for a virtual form of said map as embodied in a computer simulation of said map.

### BRIEF DESCRIPTION OF THE FIGURES

FIG. 1. Schematic drawing showing the mechanisms of ionotropic glutamine receptor activation and desensitization.

FIG. 2. Schematic drawing showing the design of GluR2-S1S2J, wherein the connection between S1 and S2 and transmembrane domains 1 and 2 is severed and replaced by a linker molecule, and S2 is separated from transmembrane domain S3.

FIG. 3. LY451646 potentiates glutamate currents from Delta ATD GluR2 as measured by TEVC in oocytes. The top tracing represents application of glutamate alone, the bottom tracing glutamate plus LY451646.

FIG. 4A-B. LY451646 promotes dimerization of GluR2 S1S2. (A) GluR2 alone; (B) GluR2S1S2 in the presence of LY451646.

FIG. 5. Data collection and refinement statistics for LY451646 bound to GluR2-S1S2J.

FIG. 6. Schematic drawing, showing ribbon structures of GluR2-S1S2J, depicting one molecule of LY451646 binding to the dimer interface.

FIG. 7. Schematic drawing, showing ribbon structures of GluR2-S1S2J, depicting LY451646 binding near the “hinge” of the GluR2-S1S2J “clamshell.”

FIG. 8. Schematic drawing, showing a close-up of the LY451646 at its binding site in GluR2-S1S2J.

FIG. 9A-C. (A) Schematic drawing of LY451646 bound to GluR2-S1S2J in a first orientation. (B) Schematic drawing of LY451646 bound to GluR2-S1S2J bound to GluR2-S1S2J in a second orientation. (C) Schematic drawing of LY404187.

FIG. 10A-B. Fo-Fc electron density for LY451646, as bound to GluR2-S1S2J. (A) View parallel to the 2-fold axis of symmetry. (B) View perpendicular to the 2-fold axis of symmetry.

FIG. 11. Fo-Fc electron density resulting from the two binding orientations of LY451646, with their associated molecular structures superimposed.

FIG. 12. Structure and Fo-Fc electron density of SYM1, having formula 3.

FIG. 13. SYM1 fits within the Fo-Fc electron density of LY451646, in this view parallel to the 2-fold axis.

FIG. 14A-C. Depiction of SYM1 in the binding cleft of GluR2-S1S2J. (A) Two-fold disordered molecules; (B) two-fold symmetric SYM1; (C) Schematic drawing of SYM1 bound to GluR2-S1S2J.

FIG. 15. Depiction of LY404187 and its interactions with the GluR2 S1S2 binding site.

FIG. 16A-E. Electrophysiologic studies comparing N,N′-(2R,2′R)-2,2′-(biphenyl-4,4′-diyl)bis(propane-2,1-diyl)dipropane-2-sulfonamide) with LY451646. A. Microamperes of current over various periods of time in a GluR2 expressing oocyte (Xenopus laevis oocyte with microinjected rat GluR2 receptor-encoding mRNA), exposed to various concentrations (0.001 μM, 0.01 μM, 0.1 μM and 1 μM) of N,N′-(2R,2′R)-2,2′-(biphenyl-4,4′-diyl)bis(propane-2,1-diyl)dipropane-2-sulfonamide). B. Microamperes of current over various periods of time in a GluR2 expressing oocyte exposed to various concentrations (0.001 μM, 0.01 μM, 0.1 μM, 1 μM and 10 μM) of LY451646. C. In an oocyte expressing GluR2, ratio of current/maximal current (I/Iₘₐₓ) versus concentration of N,N′-(2R,2′R)-2,2′-(biphenyl-4,4′-diyl)bis(propane-2,1-diyl)dipropane-2-sulfonamide). D. In an oocyte expressing GluR2, ratio of current/maximal current (I/Iₘₐₓ) versus concentration of LY451646. E. N,N′-(2R,2′R)-2,2′-(biphenyl-4,4′-diyl)bis(propane-2,1-diyl)dipropane-2-sulfonamide).

FIG. 17. Effect of N,N′-(2R,2′R)-2,2′-(biphenyl-4,4′-diyl)bis(propane-2,1-diyl)dipropane-2-sulfonamide) on locomotor activity in the context of various doses of amphetamine.

FIG. 18. (A) Potentiation of steady-state glutamate-induced currents for GluR2; receptors and (B) % current potentiation bar graph.

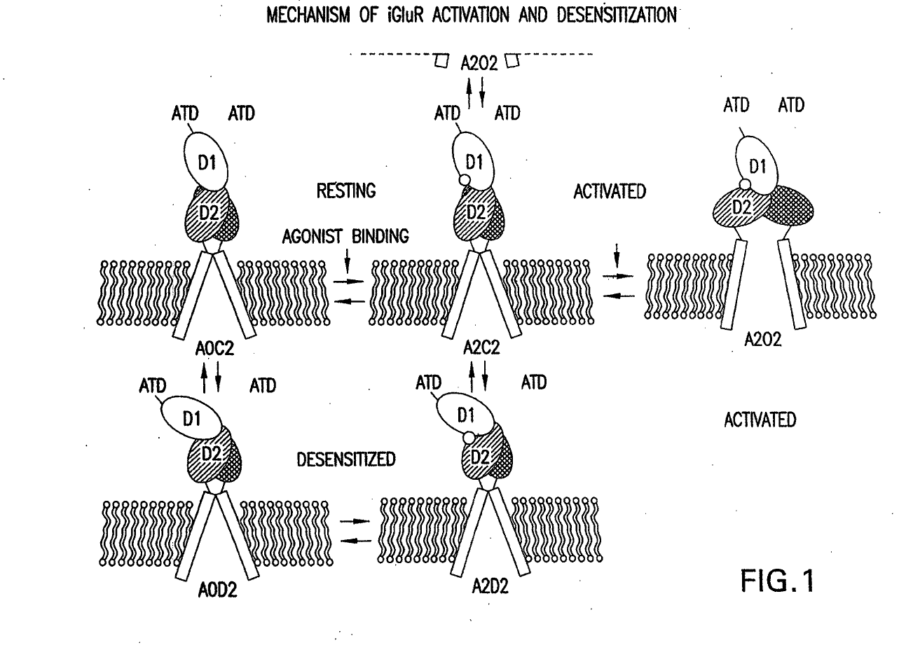

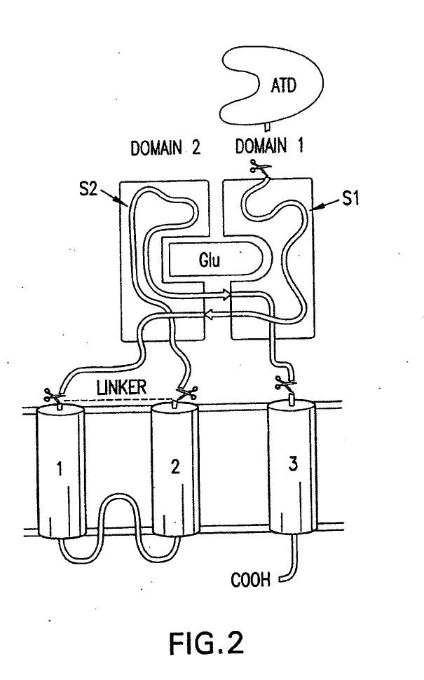

### DETAILED DESCRIPTION OF THE INVENTION

For clarity, and not by way of limitation, the detailed description of the invention is divided into the following subsections:
GluR2 Modulators;
Methods For Characterizing GluR2 Modulators; and
Uses Of GluR2 Modulators.

### GluR2 Modulators and Other Ion Channel Receptor Modulators

The present invention provides modulators with binding properties similar to LY451646, but which bind essentially in one orientation to the receptor. In the case of GluR2, for example, the modulators of the invention bind essentially in one orientation to GluR2-S1S2J. As used herein, “bind essentially in one orientation” means that desirably the modulator binds in only one structural orientation, but in the event that more than one binding orientation is observed (e.g., by X-ray crystallography), there exists one structural orientation in which the modulator is found bound to the receptor, e.g., GluR2, in at least about 75 percent of occurrences.

In particular non-limiting embodiments, the modulator exhibits 2-fold (C₂) symmetry, which means that it looks the same after being rotated by 180 degrees. Because of this symmetry, even if a modulator at a particular position were labeled and it was found that the labeled molecule occurred sometimes at a first position, and sometimes at a second position in which the modulator is rotated 180 degrees, said modulator would still be said to bind in only one structural orientation, even where several detectable orientations exist.

It will be apparent that compounds of the invention may have one or more chiral atoms. When such compounds have 2-fold symmetry, the symmetric/opposing chiral atoms have the same absolute configuration relative to one another. In the simplest case where the compound has two-fold symmetry and two opposing chiral atoms, each chiral atom will be “R” or “S” and the compound will be designated “RR” or “SS.” Compounds having identical chiral atoms but lacking two-fold symmetry yield meso compounds. In the case where such a compound lacks two-fold symmetry, the compound is designated “RS” or “SR.”

In particular non-limiting embodiments, the modulator, which may or may not exhibit 2-fold symmetry, may exhibit, when bound to GluR2, a Fo-Fc electron density which conforms to the Fo-Fc electron density of LY451646 bound to GluR2(S1S2J). “Conforms” means that the Fo-Fc electron density determined for a modulator bound to GluR2(S1S2J) overlaps with the Fo-Fc electron density of LY451646, determined by the same methods (for example, at a resolution of approximately 1.0 Angstroms or less), by at least about 60 percent, at least about 70 percent, at least about 80 percent, at least about 90 percent, or at least about 95 percent. The electron density of LY451646 may be determined using the coordinates set forth in Appendix A.

In particular non-limiting examples of species of any of the above embodiments, a modulator of the invention may be a molecule comprising a linear conformation, in which both ends of the linear conformation show increased electron density relative to the mid-portion of the linear conformation and optionally may exhibit a net negative charge. In further non-limiting examples, the areas of increased electron density may, when bound to GluR2, such as GluR2(S1S2J), form a polar or non-polar attracting interaction with one or more of the following residues of GluR2: Ile 481, Lys 493, Pro 494, Phe 495, Met 496, Ser 497, Leu 498, Ser 729, Lys 730, Gly 731, Leu 751, Ser 754, Leu 759.

In particular non-limiting examples of species of any of the above embodiments, a modulator of the invention may, in GluR2, e.g. GluR2-S1S2J, form an attracting interaction, which may be polar or non-polar, with one or more of the following GluR2 amino acid residues: Ile 481, Lys 493, Pro 494, Phe 495, Met 496, Ser 497, Leu 498, Ser 729, Lys 730, Gly 731, Leu 751, Ser 754, Leu 759.

In particular non-limiting examples of species of any of the above embodiments, a modulator of the invention may, in GluR2, e.g. GluR2-S1S2J, form an attracting interaction, which may be polar or non-polar, with one or more of the following GluR2 amino acid residues, where subscripts A and B denote the different protomers (see Example 1, below, and Appendix A: Ile_{B} 481, Pro_{A} 494, Ser_{B} 497, Ser_{A} 729, Ser_{B} 729, Lys_{A} 730, Gly_{B} 731, Leu_{A} 751, and Ser_{A} 754.

In particular non-limiting embodiments of the invention, the modulator of the invention may be represented as formula 1:

R¹—R²R³R⁴—R⁵

where R¹ and R⁵ are preferably, but not necessarily, the same, and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl;
where R² and R⁴ are preferably, but not necessarily, the same, and are ring structures, optionally substituted, which may be aryl, cycloalkyl, or heterocyclyl; and
where R³ may be a bond between R² and R⁴ which either is shared by or joins the two rings without being shared in the rings themselves.

In non-limiting embodiments, R², R³, R⁴ may be a naphthyl group, a piperazine group, or a biaryl group.

In further non-limiting embodiments, R², R³, R⁴ may be a naphthyl group, a piperazine group, or a biaryl group, and R¹ and R⁵ may be the same, and may each be an alkylaminosulfonyl or an alkylaminosulfonylalkyl group.

In a particular set of embodiments, the present invention provides for a compound having formula 2:

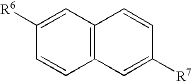

where R⁶ and R⁷ are preferably, but not necessarily, the same, and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl. In one specific non-limiting embodiment, the present invention provides for a compound having formula 3A:

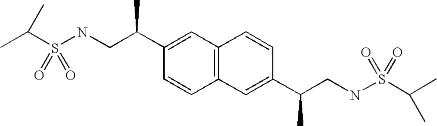

Another non-limiting embodiment, the present invention provides for a compound having formula 3B (also referred to as “SYM1”):

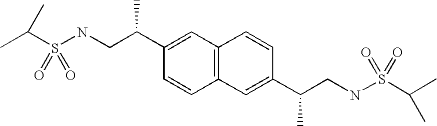

In another particular set of embodiments, the present invention provides for a compound having formula 4:

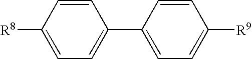

where R⁸ and R⁹ are preferably, but not necessarily, the same, and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl.

In one specific non-limiting embodiment, the present invention provides for a compound having formula 5:

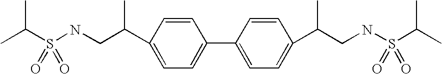

The compound above may exist in three absolute configurations, viz., “R,R” or “R,S”; or S,S.”

In a preferred embodiment, the present invention provides for N,N′-(2R,2′R)-2,2′-(biphenyl-4,4′-diyl)bis(propane-2,1-diyl)dipropane-2-sulfonamide), a compound having 2-fold symmetry as shown in formula 6:

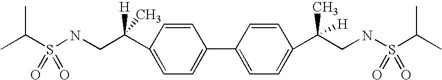

In another set of non-limiting embodiments, the present invention provides for a compound of formula 7:

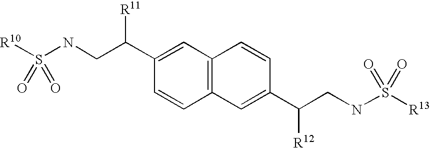

where R¹⁰ and R¹³ are the same and/or R¹¹ and R¹² are the same, and preferably R¹⁰ and R¹³ are the same and R¹¹ and R¹² are the same, and each of R¹⁰, R¹¹, R¹², and R¹³ individually may be H or alkyl, including but not limited to methyl, ethyl, propyl, isopropyl or butyl, and preferably where R¹⁰ and/or R¹³ is propyl where the linkage to the sulfur is via the middle carbon.

In another set of non-limiting embodiments, the present invention provides for a compound of formula 8:

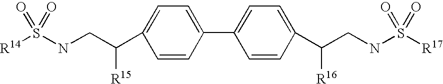

where R¹⁴ and R¹⁷ are the same, or, if they are not, then R¹⁵ and R¹⁶ are the same, and each of R¹⁴, R¹⁵, R¹⁶, and R¹⁷ individually may be H or alkyl, including but not limited to methyl, ethyl, propyl, isopropyl or butyl, and preferably where R¹⁴ and/or R¹⁷ is propyl where the linkage to the sulfur is via the middle carbon.

In another set of non limiting embodiments, the present invention provides for a compound of formula 9:

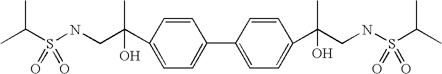

In a preferred embodiment, the present invention provides for N,N′-(2S,2′S)-2,2′-(biphenyl-4,4′-diyl)bis(2-hydroxypropane-2,1-diyl)dipropane-2-sulfonamide), a compound having a 2-fold symmetry of formula 10:

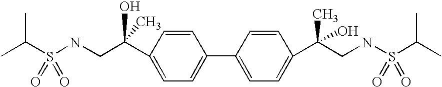

In another set of non limiting embodiments, the present invention provides for a compound of formula 11:

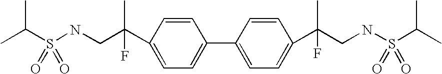

In a preferred embodiment, the present invention provides for N,N′-(2S,2′S)-2,2′-(biphenyl-4,4′-diyl)bis(2-fluoropropane-2,1-diyl)dipropane-2-sulfonamide), a compound having a 2-fold symmetry of formula 12:

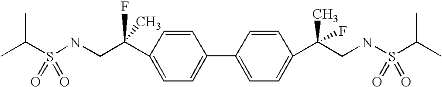

The receptor-modulating compounds of the invention have structures, preferably with 2-fold symmetry, that closely conform to the dimensions of the three dimensional pockets forming their respective receptors. Receptor dimensions may be measured or estimated via any method familiar to those skilled in the art, including without limitation x-ray crystallography, (Ilari, A. and Savino, C. Methods Mol. Biol. 452, 63-87 (2008)); and nuclear magnetic resonance spectroscopy, (Altieri, A. S. and Byrd, R. A. Curr. Opin. Struct. Biol. 14, 547-553 (2004)).

Synthesis of the receptor-modulating compounds of the invention may be carried out as convenient, employing any methods familiar to those skilled in the art. General synthetic methods for compounds having a biaryl core are described in Magnus et al., Organic Process Research & Development, 2005, 9, 621-628.

The compounds of the invention are preferably purified and isolated in crystalline form. For example, compounds of the invention are preferably at least 95% pure, more preferably at least 98% pure.

The foregoing compounds and classes of compounds, in non-limiting embodiments of the invention, (i) may exhibit, when bound to GluR2, a Fo-Fc electron density which conforms to the Fo-Fc electron density of LY451646 bound to GluR2(S1S2J); and/or (ii) may, in GluR2, e.g. GluR2(S1S2J), form an attracting interaction, which may be polar or non-polar, with one or more of the following GluR2 amino acid residues: Ile 481, Lys 493, Pro 494, Phe 495, Met 496, Ser 497, Leu 498, Ser 729, Lys 730, Gly 731, Leu 751, Ser 754, Leu 759; and/or (iii) may exhibit GluR2 agonist activity; and/or (iv) may exhibit GluR2 antagonist activity.

### Methods For Characterizing GluR2 Modulators

The present invention further provides for methods of identifying agonists (including partial agonists) as well as antagonists of GluR2.

One skilled in the art may select and employ various modalities to identify and/or design compounds that modulate GluR2 in accordance with the invention. Such include, without limitation, rational drug design, molecular replacement, homology modeling and in silico screening. Identification of additional compounds that modulate receptors in accordance with the invention may also be achieved by performing binding assays which compare the relative binding of reference and test compounds to the receptor. In this manner, compounds having increasingly higher potency for modulating the receptor may be identified.

Methods of identifying GluR2 modulators include using the solved crystal structure of GluR2-S1S2 bound to LY451646 to identify additional compounds that bind substantially to the same site as LY451646 but which show advantageous properties, such as increased potency, less side effects, and/or slower deactivation of GluR2 (see below). For example, a virtual model of LY451646 as bound in GluR2 may be used to develop additional compounds which fit, with appropriate electrostatic interactions, into the binding site.

A modulator of the invention may be identified as an agonist by any method known in the art. For example, upon binding to GluR2(S1S2J), an agonist tends to result in a closure of the cleft between S1 and S2 by approximately 20°, as measured, for example, by determining the crystal structure. A partial agonist, in contrast, would result in closure of the cleft by approximately 10-18°. Binding of an antagonist is not associated with substantial cleft closure. An agonist of the present invention stabilizes the receptor in a closed-cleft state, which resembles the conformation of the receptor when it is bound to an activator, such as glutamate. The modulators of the invention may be used in the presence or absence of an activator.

In particular embodiments, a modulator of the invention may be identified as an agonist (full or partial agonist) by determining that the modulator slows deactivation of GluR2. For example, a slowing of deactivation may be determined using studies as described in Jin et al., 2005, J. Neurosci. 25(39): 9027-9036, in which electrophysiological responses of GluR2(flop), expressed on outside-out patches of transiently transfected HEK 293 cells, were measured. A modulator of the invention may be considered an agonist if it slows deactivation more than 100 μM cyclothiazide, and/or slows deactivation at least as much as 5 mM antiracetam, and/or slows deactivation at least as much as 100 μM CX614. An antagonist, on the other hand, would promote deactivation (whereby the ion-conducting pore of the receptor closes).

### Uses of GluR2 Modulators

Therapeutic methods of the invention may be carried out by administering an effective amount of receptor-modulating compounds of the invention. Conditions to be treated may be selected based upon the receptor and the nature of the modulation desired.

By way of example, an agonist of the invention (either a full agonist or a partial agonist) may be used to treat (alleviate) depression, to improve cognition/learning, and to treat (alleviate) disorders such as mild cognitive impairment, Parkinson's disease, Alzheimer's disease, or schizophrenia. An agonist of the invention may be used to promote neurotrophic effects.

An inhibitor of the invention may be used to treat conditions, diseases and disorders associated with glutamate toxicity, including, but not limited to, ischemia, amyotrophic lateral sclerosis, and, notwithstanding the cognitive benefits which may be associated with agonists, Alzheimer's disease, Parkinson's Disease, HIV-associated dementia, and Huntington's chorea.

The present invention provides for the use of any of the compounds set forth herein, in effective amounts, for treating a disorder which, in non-limiting embodiments, is selected from the group consisting of anxiety, depression, and a combination thereof. In specific, non-limiting embodiments, an effective amount, which may be administered daily as a single or divided dose, may be between about 1-25 mg, in a single or divided dose of between 0.01 and 100 mg/kg, or between about 0.1 and 10 mg/kg, or between about 0.1 and 5 mg/kg, or between about 0.5 and 5 mg/kg.

Compounds of the invention may be administered by any route known in the art, including, but not limited to, oral, nasal or pulmonary inhalation, subcutaneous, intravenous, transcutaneous, intrathecal, rectal, etc.

Compounds of the invention may be comprised in a pharmaceutical composition with a suitable pharmaceutical carrier, including, but not limited to, water, saline, etc.

Compounds of the invention may be administered in pharmaceutical compositions in solid (e.g., tablets, capsule, suppositories) or liquid form.

Compounds of the invention may be administered together with one or more bioactive agent, including other anti-depressant and/or anti-anxiety compounds.

Typically, in the present invention, an effective amount of a receptor modulating compound is about 1-25 mg per day or 0.02-0.50 mg/kg/day. In the present invention, the foregoing amounts may be provided to a patient for the desired treatment course.

In the present invention, when a range is stated for a particular parameter, e.g., an effective amount, all values within that range, including the endpoints, are intended to be included. In addition to the foregoing, effective dosage forms, modes of administration, and dosage amounts of the compounds may be determined empirically, and making such determinations is within the skill of the art in view of the disclosure herein.

It is understood by those skilled in the art that the dosage amount will vary with the route of administration, the rate of excretion, the duration of the treatment, the identity of any other drugs being administered, the age, size, and species of mammal, and like factors well known in the arts of medicine and veterinary medicine. In general, a suitable dose of a compound according to the invention will be that amount of the VMAT2 antagonist, which is the lowest dose effective to produce the desired effect. The effective dose of a compound may be administered as one, two, three, four, five, six or more sub-doses, administered separately at appropriate intervals throughout the day.

A compound of the present invention may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. In the present invention, a preferred route of administration is intravenous. Further, a compound of the present invention may be administered in conjunction with other treatments. A compound or composition containing such an antagonist may be encapsulated or otherwise protected against gastric or other secretions, if desired.

While it is possible for a compound of the invention to be administered alone, it is preferable to administer the compound as a pharmaceutical formulation (composition). Pharmaceutically acceptable compositions of the invention comprise one receptor-modulating compounds as an active ingredient in admixture with one or more pharmaceutically-acceptable carriers and, optionally, one or more other compounds, drugs, ingredients and/or materials. Regardless of the route of administration selected, the compounds of the present invention are formulated into pharmaceutically-acceptable dosage forms by conventional methods known to those of skill in the art. See, e.g., Remington's Pharmaceutical Sciences (Mack Publishing Co., Easton, Pa.).

Pharmaceutically acceptable carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose; mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active VMAT2 antagonist. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise one or more compound in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

In a preferred embodiment of the invention, there is provided a pharmaceutical composition in a unit dosage form for oral administration comprising 1 to 25 mg of for N,N′-(2R,2′R)-2,2′-(biphenyl-4,4′-diyl)bis(propane-2,1-diyl)dipropane-2-sulfonamide) or a pharmaceutically acceptable salt thereof and a pharmaceutical acceptable carrier.

In a preferred embodiment of the invention, there is provided a pharmaceutical composition in a unit dosage form for oral administration comprising 1 to 25 mg of for N,N′-(2S,2′S)-2,2′-(biphenyl-4,4′-diyl)bis(2-hydroxypropane-2,1-diyl)dipropane-2-sulfonamide) or a pharmaceutically acceptable salt thereof and a pharmaceutical acceptable carrier.

In a preferred embodiment of the invention, there is provided a pharmaceutical composition in a unit dosage form for oral administration comprising 1 to 25 mg of for N,N′-(2S,2′S)-2,2′-(biphenyl-4,4′-diyl)bis(2-fluoropropane-2,1-diyl)dipropane-2-sulfonamide) or a pharmaceutically acceptable salt thereof and a pharmaceutical acceptable carrier.

### EXAMPLES

The following examples are provided to further illustrate the compounds, pharmaceutical compositions and methods of the present invention. These examples are illustrative only and are not intended to limit the scope of the invention in any way.

### Example 1

N,N′-(2R,2′R)-2,2′-(biphenyl-4,4′-diyl)bis(propane-2,1-diyl)dipropane-2-sulfonamide) is synthesized as follows:

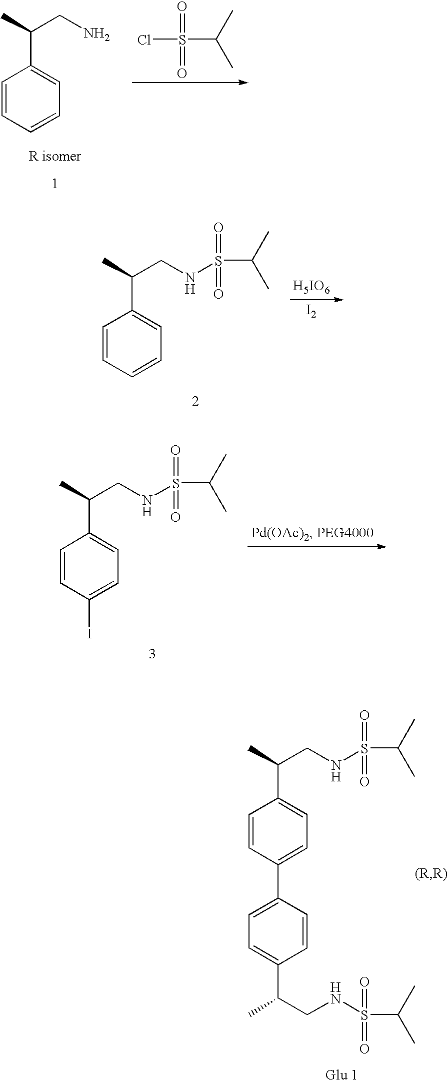

1. Synthesis of (R)—N-(2-phenylpropyl) propane-2-sulfonamide (compound 2)

1 g of (R)-2-phenylpropan-1-amine, 1.2 ml of triethylamine and a catalytic amount of DMAP were dissolved in 20 ml of CH₂Cl₂. The solution was cooled to 0° C. and to the solution, 1.1 g of propane-2-sulfonyl chloride was added slowly. The reaction was stirred at 0° C. for 0.5 h and then warmed to room temperature for 3 h. The solution was washed by 1NHCl, saturated NaHCO₃ and brine. After removing solvent, 1.5 g of light yellow oil was obtained. The product was used for next step without purification.

2. Synthesis of (R)—N-(2-(4-iodopheyl)propyl) propane-2-sulfonamide (compound 3)

0.9 g of compound 2 was dissolved in 4.5 ml of HAc and 0.4 g of concentrated H₂SO₄ was added slowly, rinsed by 1 ml of water. To this solution, 0.2 g of and 0.45 g of 12 was added. The reaction was heated at 60 0° C. for 3 h. The reaction was quenched by saturated Na₂SO₃, extracted with CH₂Cl₂ three times. The product was obtained as brown solid and was used for next step without purification.

3. Synthesis of N,N′-(2R,2′R)-2,2′-(biphenyl-4,4′-diyl)bis(propane-2,1-diyl)diropane-2-sulfonamide

190 mg of compound 3 was mixed with 15 mg of Pd(OAc)₂, 71 mg of K₂CO₃, 0.5 g of PEG4000. The mixture was heated at 120° C. for 5 h. after cooled down, the black solid was extracted with ether 10 times. After removing solvent, the residue was purified by chemotography to give 75 mg of pure product (Yield: 60.3%). ¹HNMR (CD₃Cl) δ: 1.27-1.36 (18H, m, CH₃); 2.97-3.15 (4H, m, CH₂); 3.21-3.31 (2H, m, CH); 3.35-3.42 (2H, m, CH); 3.95 (2H, broad, NH); 7.29 (4H, dd, Ph-H); 7.53 (4H, dd, Ph-H). ¹³CNMR (CD₃Cl) δ: 17.04, 40.88, 50.67, 53.75, 127.71, 127.95, 139.74, 142.30. MS (M⁺): 480.95

### Example 2

N,N′-(2S,2′S)-2,2′-(biphenyl-4,4′-diyl)bis(2-fluoropropane-2,1-diyl)dipropane-2,1-diyl)dipropane-2-sulfonamide) is synthesized as follows:

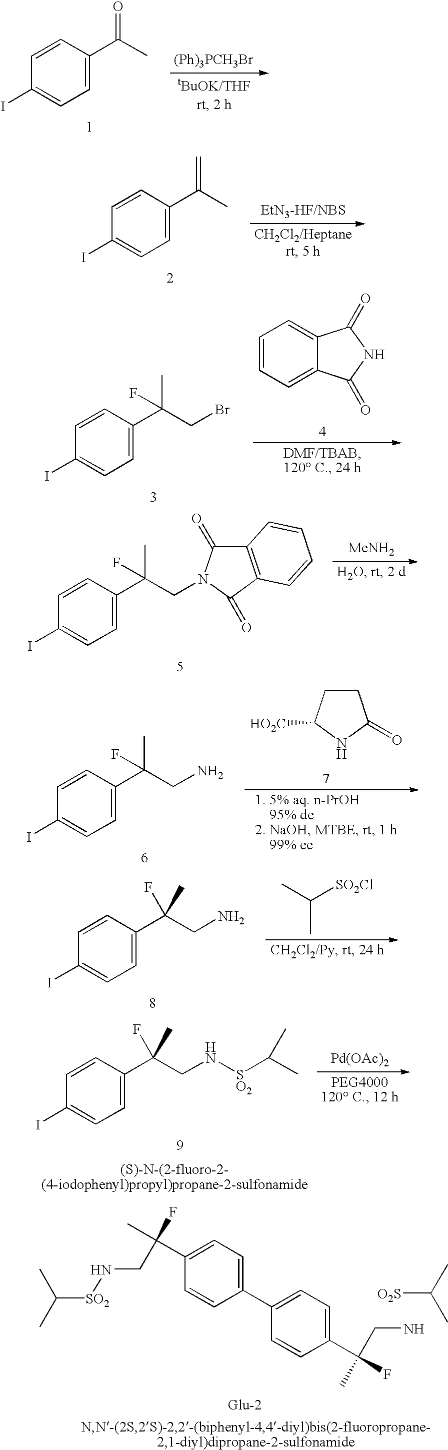

(S)—N-(2-fluoro-2-(4-iodophenyl)propyl)propane-2-sulfonamide (compound 9) was synthesized according to similar procedure described in literature (Organic Process Research & Development 2005, 9, 621-628)

385 mg of compound 9 was mixed with 5 mg of Pd(OAc)₂, 70 mg of K₂CO₃, 0.5 g of PEG4000. The mixture was heated at 120° C. for 12 h. After cooled down, the black residue was loaded to a silicon gel packed column and the residue was purified by chemotography (elute: CH₂Cl₂) to give 333 mg of the final product Glu-2 (Yield: 80%). ¹HNMR (CD₃Cl) δ: 1.25-1.31 (12H, m, CH₃); 1.72-1.79 (6H, d, CH₃); 3.02-3.07 (2H, m, CH); 3.54-3.63 (4H, m, CH₂); 4.44 (2H, t, NH); 7.41-7.43 (4H, d, Ph-H); 7.58-7.61 (4H, d, Ph-H). ¹³CNMR (CD₃Cl) δ: 16.88, 16.97, (24.99; 25.31), (52.81; 53.12), 54.25, (95.97; 98.29), 124.99, 125.11, 127.50, 140.37, (140.79; 141.09). ESI-MS (M⁺+H): 517; ESI-MS (M⁺+Na): 539.

### Example 3

N,N′-(2S,2′S)-2,2′-(biphenyl-4,4′-diyl)bis(2-hydroxypropane-2,1-diyl)dipropane-2-sulfonamide is synthesized as follows:

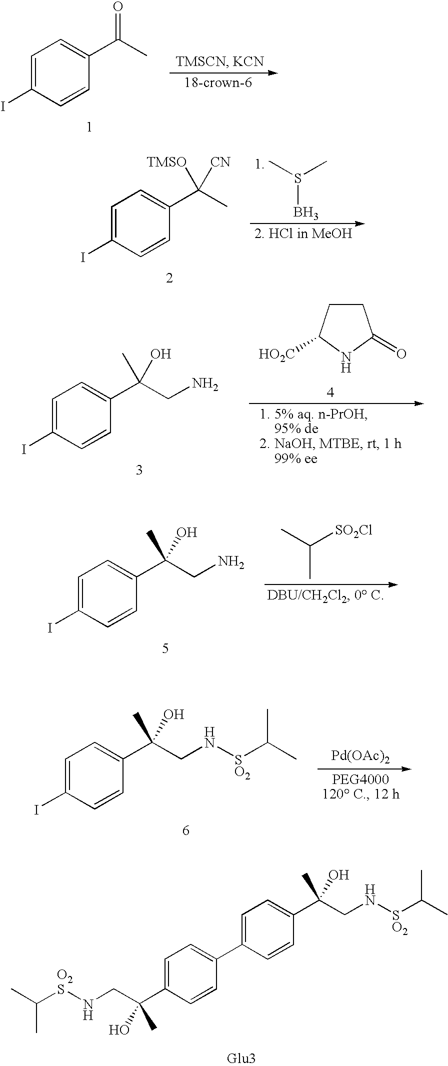

1. 2-(4-iodophenyl)-2-(trimethylsilyloxy)propanenitrile (2) is prepared in situ following generally the method disclosed in Tetrahedron Lett., 24(42), 4559 (1983).

2. 1-amino-2-(4-iodophenyl)propan-2-ol (3)

A 10M solution of borane in dimethyl sulfide (5 mL, 0.05 mol) is added rapidly to the solution of 13.8 g of 2 (0.04 mol) in 25 mL of THF and the resulting mixture is heated at reflux for 16 h. The mixture is cooled to room temperature and anhydrous 10% HCl in methanol was added slowly. The solution is allowed to stir for an additional hour, and is concentrated under reduced pressure to give the crude product (3) as white solid and as hydrochloride salt.

3. (S)-1-amino-2-(4-iodophenyl)propan-2-ol (5)

5 is prepared according the similar method as the compound 8 in the synthesis of Glu2

4. (S)—N-(2-hydroxy-2-(4-iodophenyl)propyl)propane-2-sulfonamide (6)

In a 100 mL-3 nack flask fitted with a stirrer and thermometer, 713 mg of propanesulfonyl chloride (5 mmol) in 5 mL of CH₂Cl₂ is added dropwise to 1.10 g of 5 (4 mmol) and 760 mg of DBU (5 mmol) in CH₂Cl₂ (50 mL) while stirring at 0° C. under argon. The reaction is allowed to warm to rt and stirred overnight. Then the reaction mixture is washed twice with water, dried over anhydrous Na₂SO₄, filtered and concentrated under reduced vacuum to yield a viscous oil which is purified via chromatography, eluting with a solvent of CH₂Cl₂/MeOH to yield the product 6 (461 mg, 30%) as a viscous oil.

5. N,N′-(2S,2′S)-2,2′-(biphenyl-4,4′-diyl)bis(2-hydroxypropane-2,1-diyl)dipropane-2-sulfonamide (Glu3) is prepared as the similar method of Glu2. ¹HNMR (CD₃Cl) δ 1.30-1.36 (m, 18H), 2.95-3.04 (m, 4H), 3.21 (m, 2H), 3.34 (m, 2H), 4.95 (broad, 2H), 5.50 (board, 2H), 7.35 (dd, 4H), 7.47 (dd, 4H). ¹³CNMR (CD₃Cl) δ 16.1, 26.5, 50.4, 56.4, 73.9, 126.6, 127.9, 138.7, 146.8. ESI-MS (M⁺+H): 513.

### Example 4

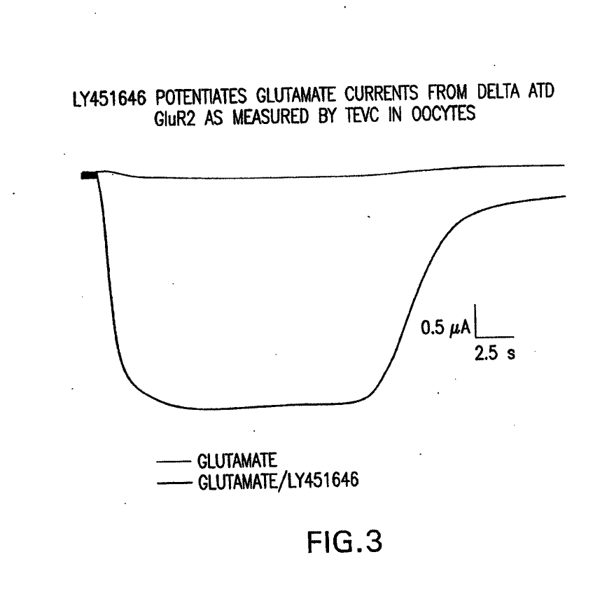

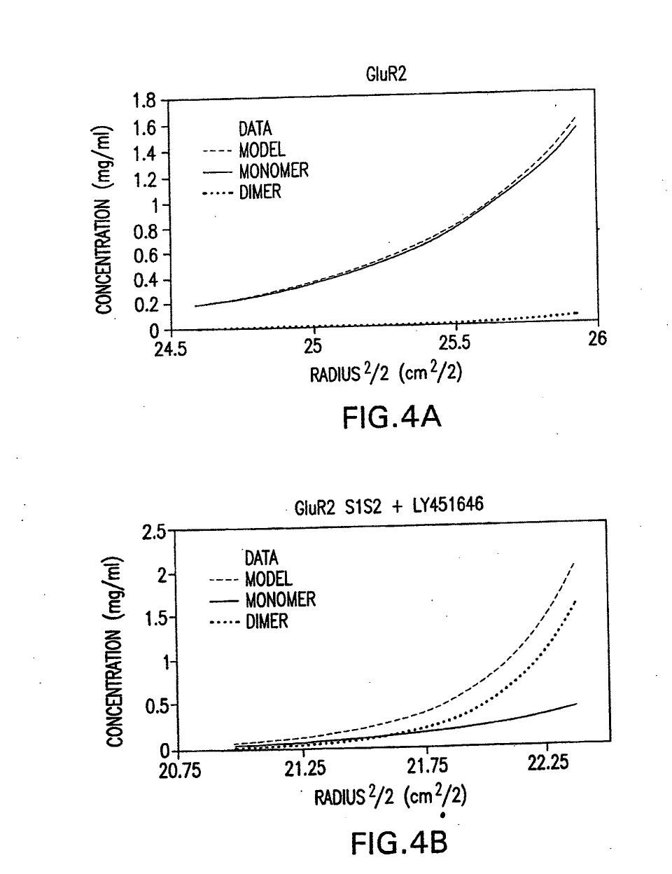

LY451646 is an agonist of GluR2. For example, in experiments using two electrode voltage clamp (“TEVC”) recording, LY451646 has been shown to potentiate glutamate currents from Delta ATD GluR2 in oocytes, as shown in FIG. 3. By showing that the modulator acts on the Delta ATD GluR2 construct, we demonstrate that the binding site of the compound is not within the ATD but rather is within the S1S2 portion of the receptor, as determined by X-ray crystallography. The top tracing represents application of glutamate alone, the bottom tracing glutamate plus LY451646. Further, as shown in FIG. 4A-B, LY451646 promotes dimerization of GluR2 S1S2. In the absence of LY451646, the amount of dimerized GluR2 barely rises above baseline (FIG. 4A). With the addition of LY451646, dimers overtake monomers (FIG. 4B).

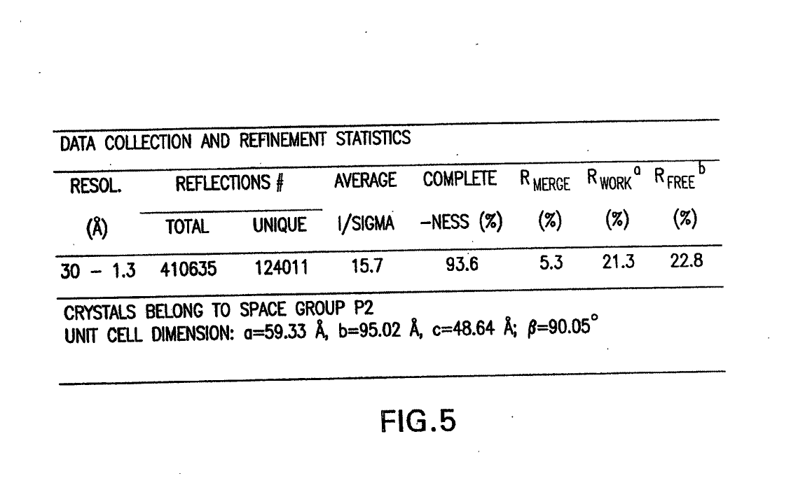

Crystals of LY451646 bound to GluR2-S1S2J were prepared in a manner analogous to the methods used in Jin et al., 2005, J. Neurosci. 25(39):9027-9036), LY451646 was used at a saturating concentration. Briefly, the crystals were grown at 4° C. by vapor diffusion and each drop contained a 1:1 ratio of reservoir solution, which for LY451646, contained 10 mM kainate, 8%-15% PEG1450, and 0.1 M NaOAc at pH 5.0. The resulting crystals were soaked in the crystallization buffer, supplemented with LY451646, and 12-16% glycerol before flash cooling in liquid nitrogen. The data set was collected at 110 K at the NSLS X4A beam line, and was processed with the HKL suite of programs. The obtained coordinates for the LY451646/GluR2-S1S2J structure are attached hereto as Appendix A. The LY451646/GluR2-S1S2J crystals belonged to the P2₁ space group. Data collection and refinement statistics for the crystallographic study are presented in FIG. 5.

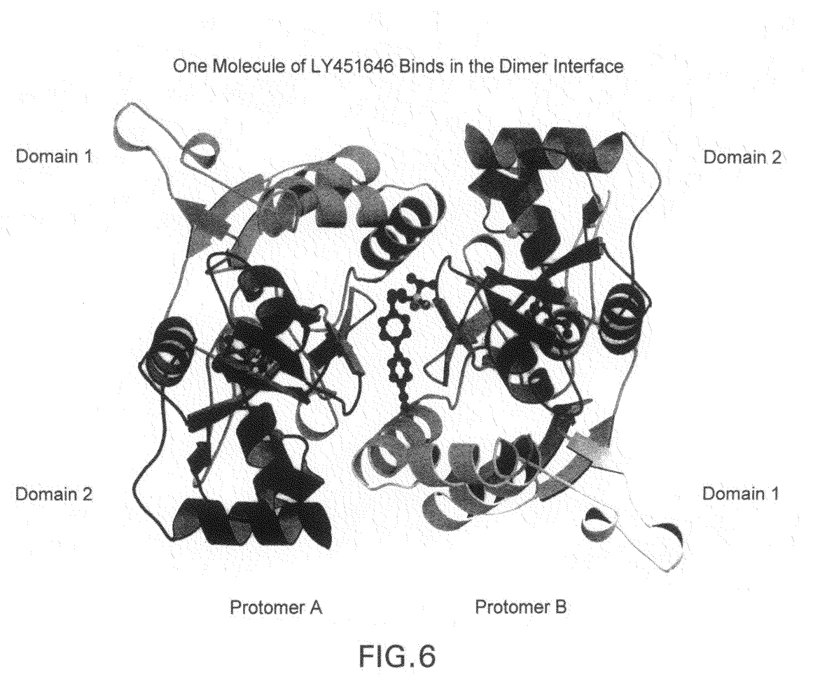

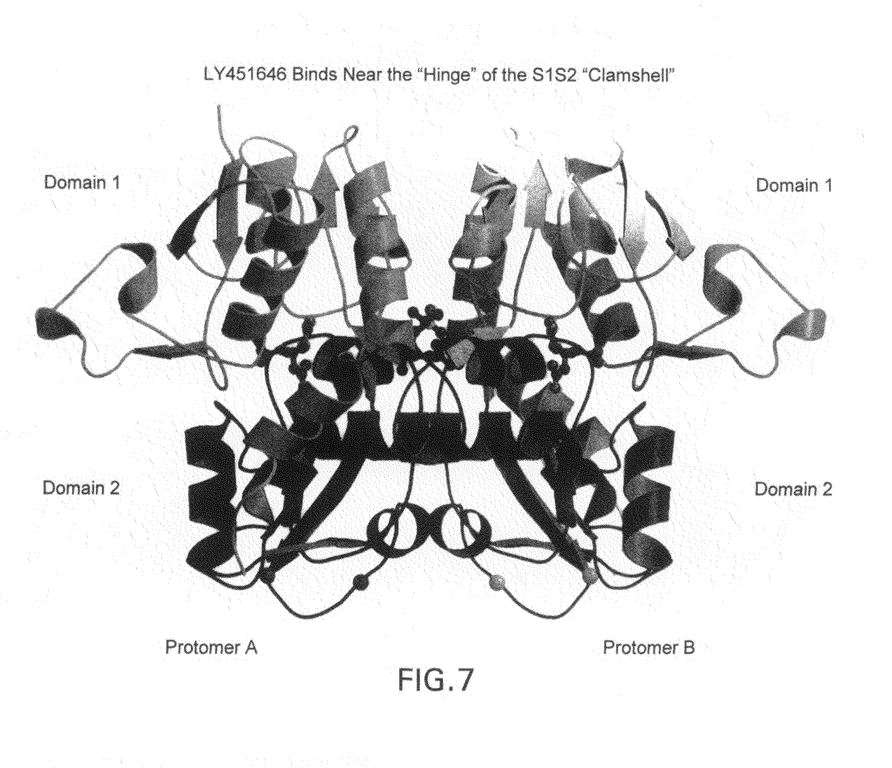

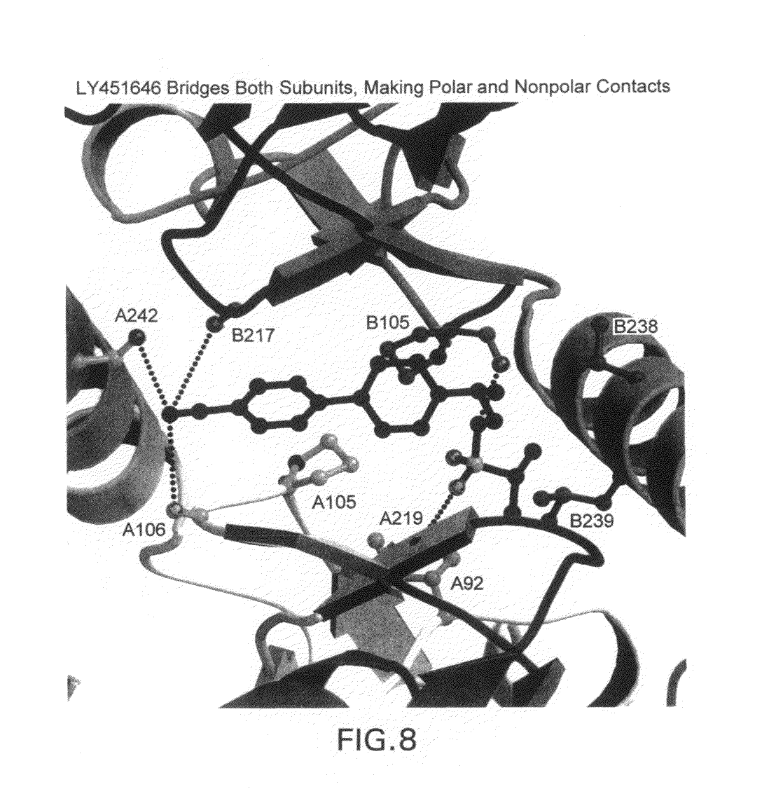

The structural data indicated that LY451646 binds at the interface between domains 1 and 2 (FIG. 6) near the “hinge” of the S1S2 “clamshell” (FIG. 7). The ligand bridges both subunits, binding via polar as well as non-polar interactions (FIG. 8). In particular, The nitrogen of the terminal nitrile group interacts with Phe_{A}106, Ser_{A}242, and Ser_{B}217, wherein the “A” designates protomer A, and “B” designates protomer B, and each residue corresponds to the identifiers and residue numbers provided in the X-ray structure provided in Appendix A. It is noted that the X-ray structure provided in Appendix A is a fragment of the full length receptor, and accordingly the residue numbers do not necessarily correspond to the full length receptor. LY451646 also interacts with Pro_{B}105, Pro_{A}105, Gly_{A}219, Ile_{A}92, and Leu_{B}239.

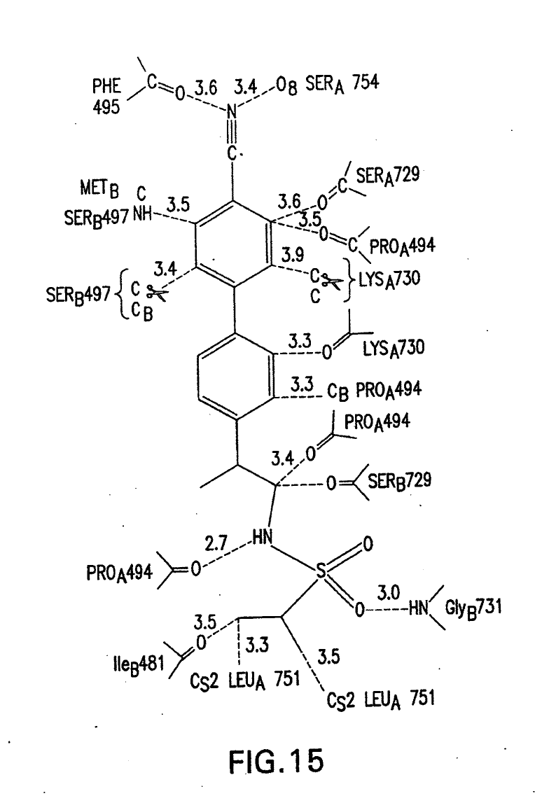

For comparison, LY404187 also interacts at the interface between domains 1 and 2 near the “hinge” of the S1S2 “clamshell” (FIG. 15). In particular, the nitrogen of the terminal nitrile group interacts with the O_{δ} atom of Ser_{A}754 and the carbonyl group of Phe495, the nitrogen in the sulfonamide bond interacts with the carbonyl group of Pro_{A}494, and the oxygen of the sulfonyl group interacts with the nitrogen of Gly_{B}731. The carbons of the isopropyl group adjacent to the sulfonyl group interact with the carbonyl of Ile_{B}481 and/or the C_{δ}2 of Leu_{A}751. The carbon atom adjacent to the nitrogen in the sulfonamide bond interacts with the carbonyl groups of Pro_{A}494 and/or Ser_{B}729. The carbon atoms in the phenyl group adjacent to the nitrile group interact with the carbonyl groups of Ser_{A}729 and Pro_{A}494, the C_{α} and/or C atom of Lys_{A}730, C_{α} and/or C atom of Ser_{B}497, or the nitrogen in Ser_{B}497 and the C atom of the Met_{B}. The carbon atoms of the remaining phenyl group interact with the carbonyl group of Lys_{A}730 or the C_{β} atom of Pro_{A}494. These interactions can be seen in the schematic depicted in FIG. 15.

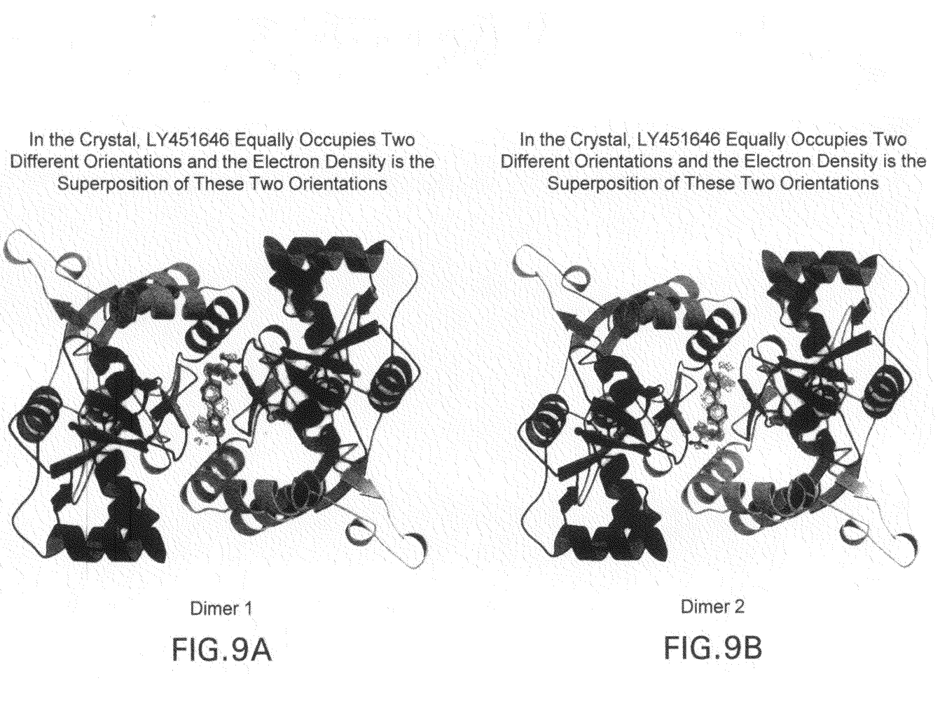

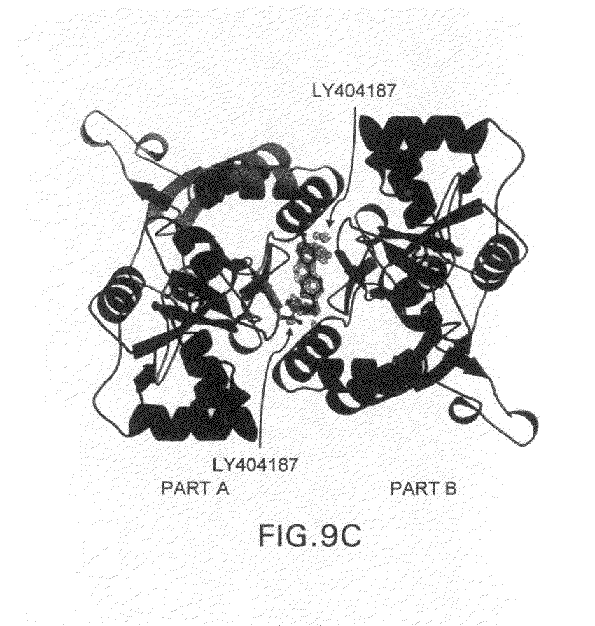

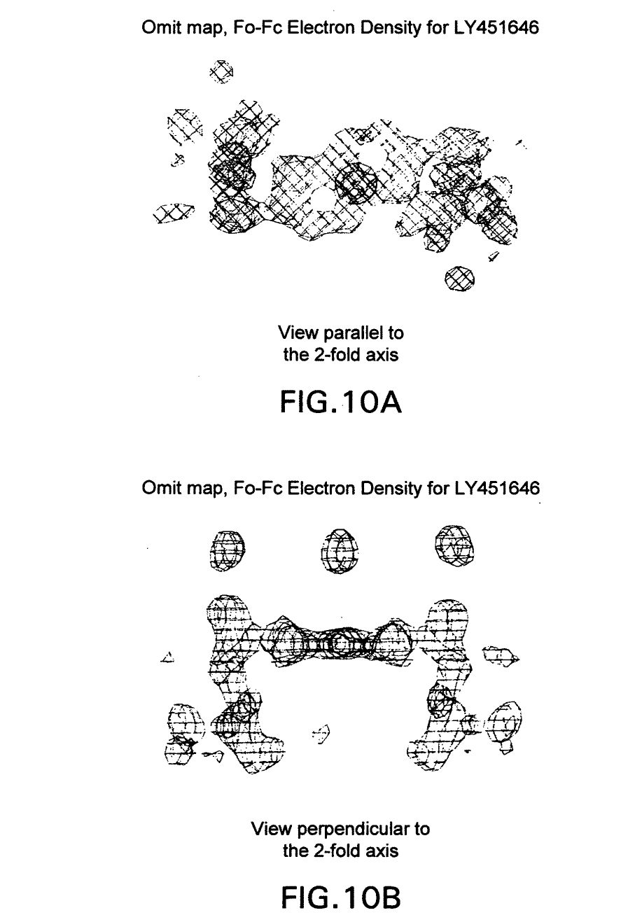

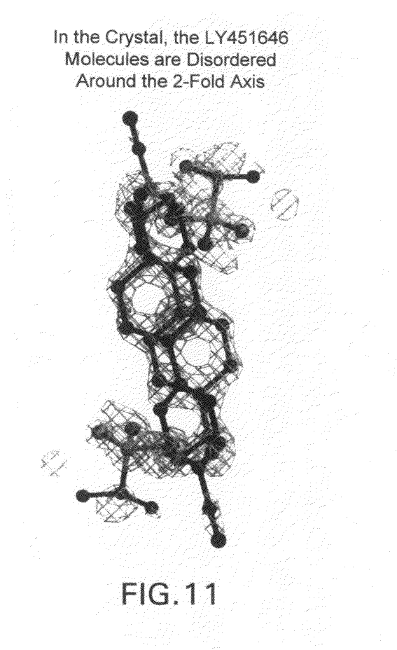

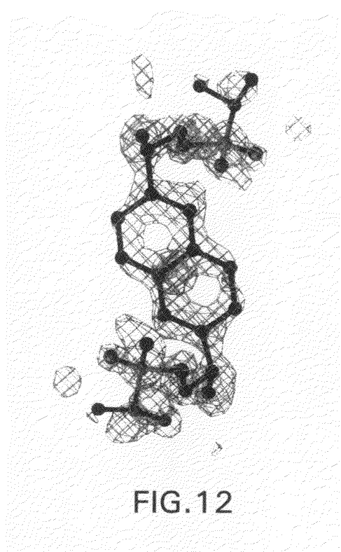

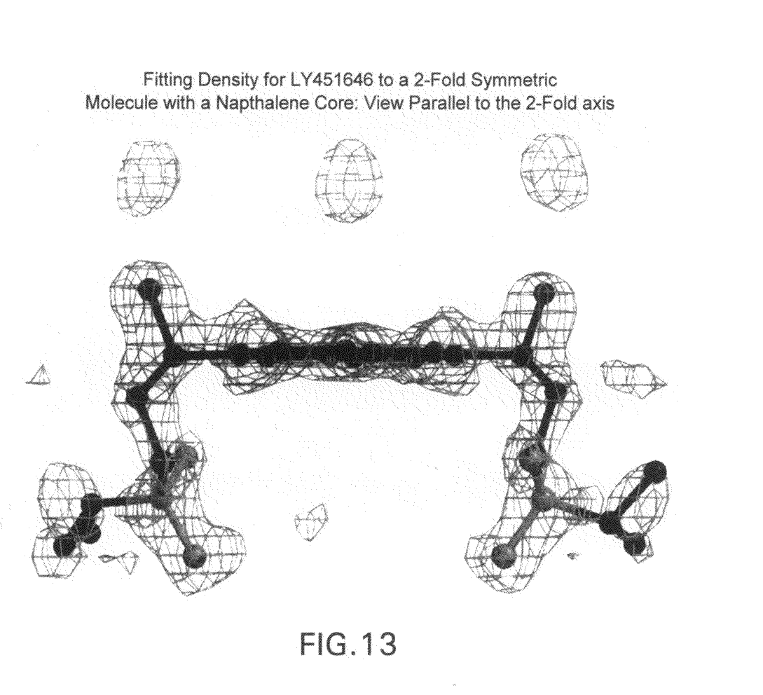

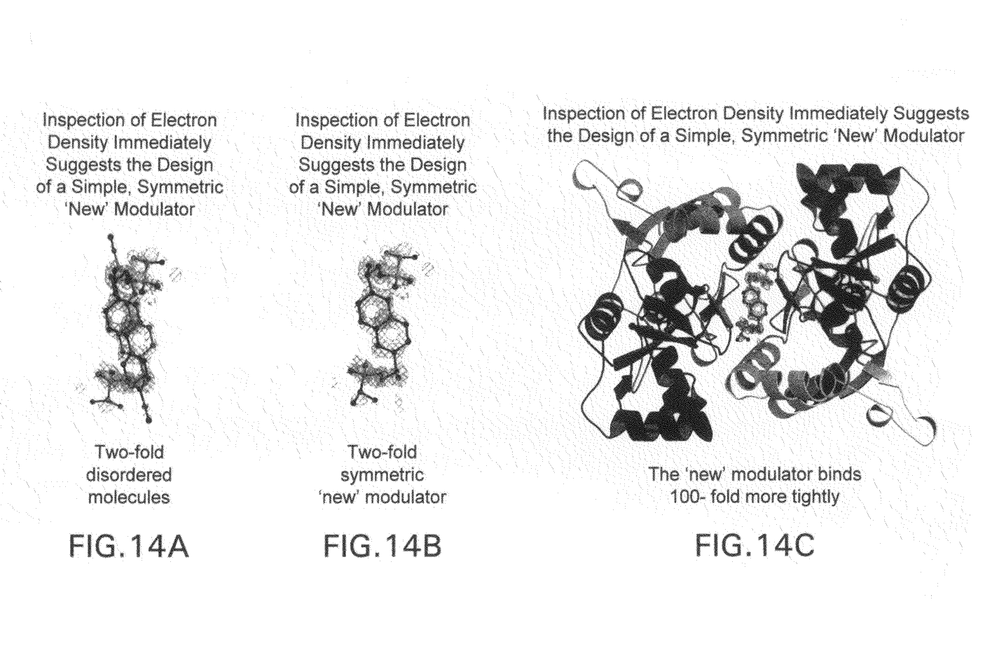

Interestingly, it has been found that the electron density of LY451646, as bound to GluR2(S1S2J), corresponds to a superposition of predicted electron densities for LY451646 bound in two overlapping orientations; the alternative orientations are depicted in FIG. 9A-B. FIG. 10A-B shows the electron densities of the ligand in a view parallel to the 2-fold axis (FIG. 10A) or perpendicular to the 2-fold axis (FIG. 10B). FIG. 11 depicts the electron density observed with structural drawings of LY451646 in the alternative orientations.

### Example 5

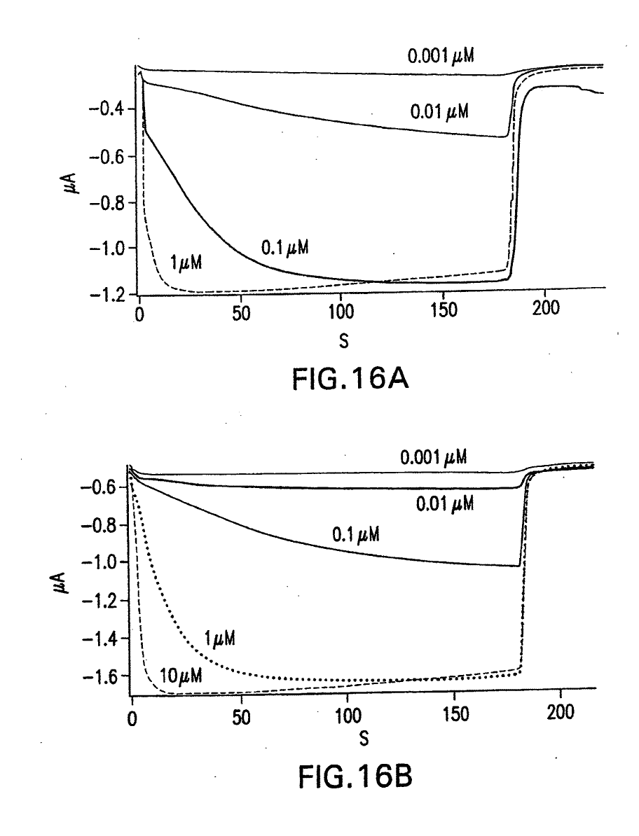

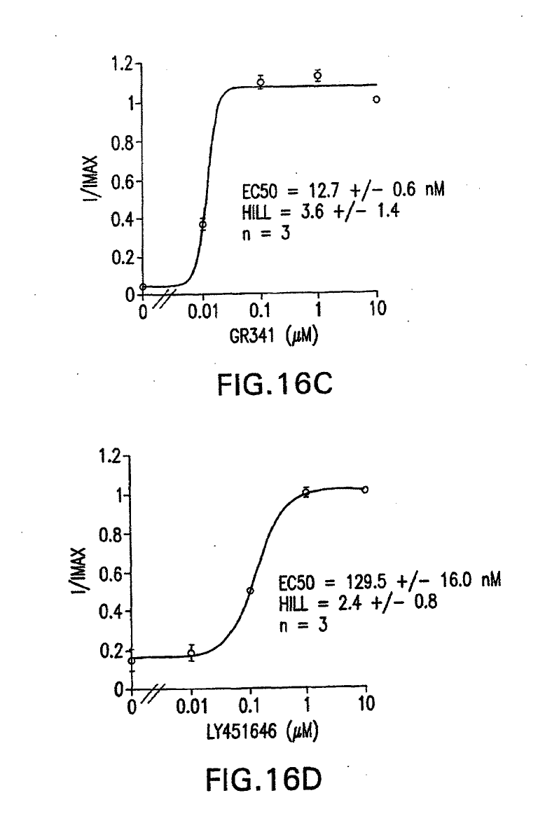

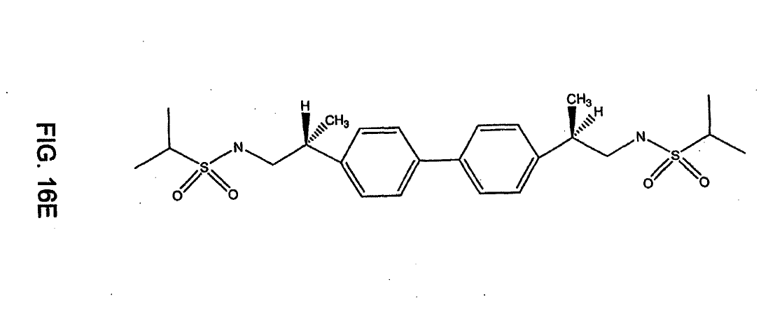

Electrophysiologic studies were performed comparing N,N′-(2R,2′R)-2,2′-(biphenyl-4,4′-diyl)bis(propane-2,1-diyl)dipropane-2-sulfonamide) (GR341) with LY451646 in cells expressing the GluR2 receptor, with results shown in FIG. 16A-D. GR341 was observed to have an EC₅₀ of 12.7±0.6 nM, and therefore was more potent than LY451646, which was observed to have an EC₅₀ of 129.5±16.0 nM.

### Example 6

Mice (male, 8 weeks of age; 129S6/SvEv from Taconic) were housed in groups of five for one week. Animals were injected (i.p.) with either vehicle (diluent:saline at a ratio of 3:50 v/v), or one of two doses of GR341 (1 mg/kg and 3 mg/kg). After 15 minutes, animals were injected (i.p.) either with saline or Amphetamine at 3 mg/kg. Mice were held individually without food or water for an additional 15 minutes and then placed in Plexiglas activity chambers (model ENV-520; Med Associates, St. Albans, Vt.) (43.2 cm long×43.2 cm wide×30.5 cm high) to assess locomotor activity. Mice were placed into the center of the open field, and activity was recorded for 30 minutes with subtotals for 5-minute increments. Testing was performed in darkness to minimize anxiety.

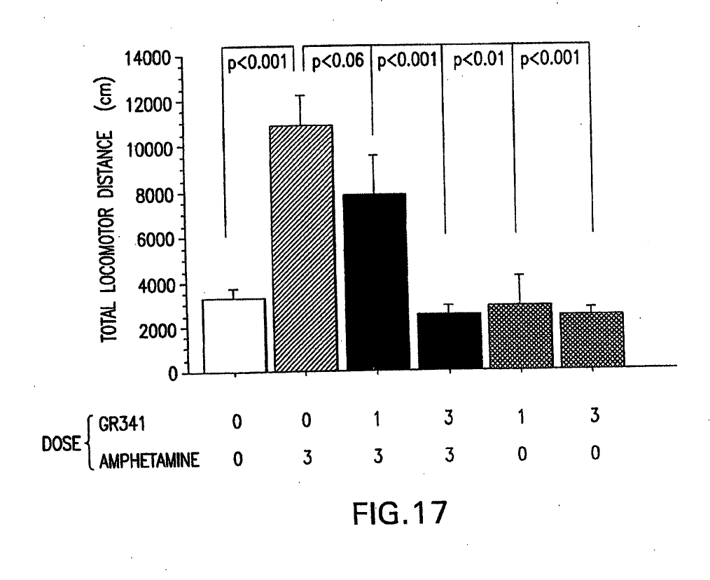

As shown in FIG. 17, each dose of GR341 inhibited amphetamine-induced locomotor activity. There was no significant effect of either dose of GR341 alone on locomotor activity.

### Example 7

Potentiation of steady-state glutamate-induced currents for GluR21 receptors by N,N′-(2S,2′S)-2,2′-(biphenyl-4,4′-diyl)bis(2-fluoropropane-2,1-diyl)dipropane-2-sulfonamide).

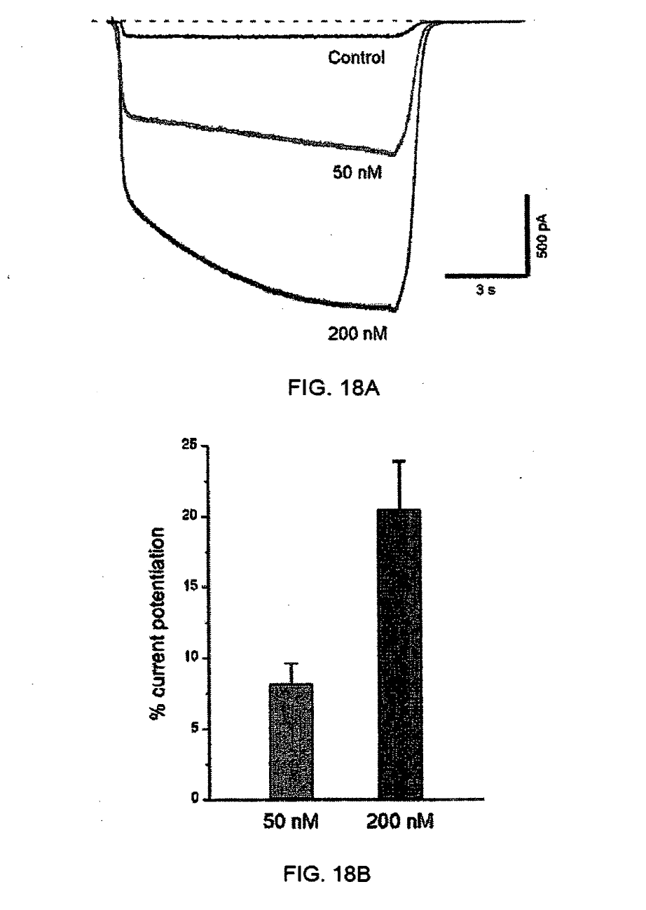

As shown in FIG. 18, (a) the compound N,N′-(2S,2′S)-2,2′-(biphenyl-4,4′-diyl)bis(2-fluoropropane-2,1-diyl)dipropane-2-sulfonamide) is active on ionotropic glutamate receptors, (b) the compound N,N′-(2S,2′S)-2,2′-(biphenyl-4,4′-diyl)bis(2-fluoropropane-2,1-diyl)dipropane-2-sulfonamide) increases, i.e. potentiates glutamate receptor currents, as desired.

The recordings are in whole-cell mode (−60 mV) and show inward currents elicited by 1 mM glutamate in the presence of various concentrations of N,N′-(2S,2′S)-2,2′-(biphenyl-4,4′-diyl)bis(2-fluoropropane-2,1-diyl)dipropane-2-sulfonamide). The concentration of 200 nM is saturating and the concentration of 50 nM is approximately half saturating, thus demonstrating that the EC50 value of N,N′-(2S,2′S)-2,2′-(biphenyl-4,4′-diyl)bis(2-fluoropropane-2,1-diyl)dipropane-2-sulfonamide) modulator is about 50 nM (FIG. 18B).

Various patents, patent applications, and other publications are cited herein, the contents of which are hereby incorporated by reference in their entireties.

APPENDIX A

| REMARK | coordinates from minimization refinement | | | |
|---|---|---|---|---|
| REMARK | refinement resolution: 10.0-1.3 A | | | |
| REMARK | starting | r = 0.2169 free_r = 0.2298 | | |
| REMARK | final | r = 0.2141 free_r = 0.2277 | | |
| REMARK | rmsd bonds = 0.005543 rmsd angles = 1.38837 | | | |
| REMARK | wa = 0.261266 | | | |
| REMARK | target = mlf cycles = 1 steps = 100 | | | |
| REMARK | sg = P2(1) a = 59.330 b = 95.018 c = 48.635 alpha = 90.0 beta = 90.004 | | | |
| | gamma = 90.0 | | | |
| REMARK | parameter file 1 : CNS_TOPPAR: protein_rep.param | | | |
| REMARK | parameter file 2 : kai.par | | | |
| REMARK | parameter file 3 : ly_1.par | | | |
| REMARK | parameter file 4 : CNS_TOPPAR: water_rep.param | | | |
| REMARK | molecular structure file: nska1_ly12.mtf | | | |
| REMARK | input coordinates: nska1_ly12.pdb | | | |
| REMARK | reflection file = nska1_p21freeR.hkl | | | |
| REMARK | ncs = none | | | |
| REMARK | B-correction resolution: 6.0-1.3 | | | |
| REMARK | initial B-factor correction applied to fobs: | | | |
| REMARK | B11 = | 2.006 B22 = | −1.594 B33 = | −0.413 |
| REMARK | B12 = | 0.000 B13 = | 0.000 B23 = | 0.000 |

| REMARK | B-factor correction applied to coordinate array B: 0.032 | |
|---|---|---|
| REMARK | bulk solvent: density level = 0.497227 e/A\hat{ }3, B-factor = 61.6121 A\hat{ }2 | |
| REMARK | reflections with \|Fobs\|/sigma_F < 0.0 rejected | |
| REMARK | reflections with \|Fobs\| > 10000 * rms(Fobs) rejected | |
| REMARK | theoretical total number of refl. in resol. range: 131701(100.0%) | |
| REMARK | number of unobserved reflections (no entry or \|F\| = 0): | 12652 |
| | (9.6%) | |
| REMARK | number of reflections rejected: | 0 |
| | (0.0%) | |
| REMARK | total number of reflections used: | 119049 |
| | (90.4%) | |
| REMARK | number of reflections in working set: | 107100 |
| | (81.3%) | |
| REMARK | number of reflections in test set: | 11949 |
| | (9.1%) | |

CRYST1 59.330 95.018 48.635 90.00 90.00 90.00 P 21

| | |
|---|---|
| REMARK | FILENAME = “min_ly12.pdb” |
| REMARK | DATE: 01-Aug-02 13:52:05 created by user: lsong |
| REMARK | VERSION: 1.0 |

| | | | | | | | | | | | |
|---|---|---|---|---|---|---|---|---|---|---|---|
| ATOM | 1 | C | GLY | A | 4 | 22.575 | 72.784 | 45.568 | 1.00 | 26.51 | A |
| ATOM | 2 | O | GLY | A | 4 | 23.484 | 72.192 | 44.987 | 1.00 | 27.75 | A |
| ATOM | 3 | N | GLY | A | 4 | 23.280 | 71.627 | 47.677 | 1.00 | 29.13 | A |
| ATOM | 4 | CA | GLY | A | 4 | 22.412 | 72.676 | 47.072 | 1.00 | 27.67 | A |
| ATOM | 5 | N | THR | A | 5 | 21.688 | 73.543 | 44.934 | 1.00 | 23.94 | A |
| ATOM | 6 | CA | THR | A | 5 | 21.739 | 73.732 | 43.492 | 1.00 | 20.81 | A |
| ATOM | 7 | CB | THR | A | 5 | 20.488 | 74.481 | 42.991 | 1.00 | 20.59 | A |
| ATOM | 8 | OG1 | THR | A | 5 | 19.325 | 73.683 | 43.246 | 1.00 | 21.84 | A |
| ATOM | 9 | CG2 | THR | A | 5 | 20.587 | 74.760 | 41.497 | 1.00 | 20.68 | A |
| ATOM | 10 | C | THR | A | 5 | 22.989 | 74.515 | 43.098 | 1.00 | 18.01 | A |
| ATOM | 11 | O | THR | A | 5 | 23.277 | 75.571 | 43.659 | 1.00 | 19.75 | A |
| ATOM | 12 | N | VAL | A | 6 | 23.730 | 73.978 | 42.136 | 1.00 | 15.06 | A |
| ATOM | 13 | CA | VAL | A | 6 | 24.950 | 74.610 | 41.655 | 1.00 | 14.03 | A |
| ATOM | 14 | CB | VAL | A | 6 | 25.810 | 73.591 | 40.876 | 1.00 | 12.06 | A |
| ATOM | 15 | CG1 | VAL | A | 6 | 26.974 | 74.292 | 40.183 | 1.00 | 13.80 | A |
| ATOM | 16 | CG2 | VAL | A | 6 | 26.324 | 72.522 | 41.831 | 1.00 | 13.53 | A |
| ATOM | 17 | C | VAL | A | 6 | 24.621 | 75.794 | 40.757 | 1.00 | 12.40 | A |
| ATOM | 18 | O | VAL | A | 6 | 23.814 | 75.683 | 39.836 | 1.00 | 13.80 | A |
| ATOM | 19 | N | VAL | A | 7 | 25.239 | 76.935 | 41.044 | 1.00 | 10.97 | A |
| ATOM | 20 | CA | VAL | A | 7 | 25.020 | 78.131 | 40.248 | 1.00 | 11.86 | A |
| ATOM | 21 | CB | VAL | A | 7 | 25.117 | 79.406 | 41.104 | 1.00 | 12.22 | A |
| ATOM | 22 | CG1 | VAL | A | 7 | 24.985 | 80.637 | 40.215 | 1.00 | 13.54 | A |
| ATOM | 23 | CG2 | VAL | A | 7 | 24.022 | 79.395 | 42.163 | 1.00 | 14.32 | A |
| ATOM | 24 | C | VAL | A | 7 | 26.053 | 78.201 | 39.132 | 1.00 | 9.54 | A |
| ATOM | 25 | O | VAL | A | 7 | 27.246 | 78.376 | 39.379 | 1.00 | 10.52 | A |
| ATOM | 26 | N | VAL | A | 8 | 25.580 | 78.054 | 37.902 | 1.00 | 10.17 | A |
| ATOM | 27 | CA | VAL | A | 8 | 26.455 | 78.107 | 36.746 | 1.00 | 10.75 | A |
| ATOM | 28 | CB | VAL | A | 8 | 26.038 | 77.067 | 35.678 | 1.00 | 11.39 | A |
| ATOM | 29 | CG1 | VAL | A | 8 | 26.953 | 77.164 | 34.452 | 1.00 | 12.01 | A |
| ATOM | 30 | CG2 | VAL | A | 8 | 26.080 | 75.670 | 36.274 | 1.00 | 10.32 | A |
| ATOM | 31 | C | VAL | A | 8 | 26.357 | 79.482 | 36.120 | 1.00 | 10.46 | A |
| ATOM | 32 | O | VAL | A | 8 | 25.255 | 79.992 | 35.911 | 1.00 | 10.77 | A |
| ATOM | 33 | N | THR | A | 9 | 27.500 | 80.105 | 35.860 | 1.00 | 9.23 | A |
| ATOM | 34 | CA | THR | A | 9 | 27.478 | 81.390 | 35.189 | 1.00 | 8.97 | A |
| ATOM | 35 | CB | THR | A | 9 | 28.420 | 82.436 | 35.838 | 1.00 | 10.14 | A |
| ATOM | 36 | OG1 | THR | A | 9 | 28.332 | 83.665 | 35.099 | 1.00 | 10.93 | A |
| ATOM | 37 | CG2 | THR | A | 9 | 29.867 | 81.960 | 35.831 | 1.00 | 11.82 | A |
| ATOM | 38 | C | THR | A | 9 | 27.926 | 81.117 | 33.760 | 1.00 | 8.83 | A |
| ATOM | 39 | O | THR | A | 9 | 28.849 | 80.326 | 33.511 | 1.00 | 9.36 | A |
| ATOM | 40 | N | THR | A | 10 | 27.229 | 81.731 | 32.816 | 1.00 | 8.91 | A |
| ATOM | 41 | CA | THR | A | 10 | 27.562 | 81.572 | 31.417 | 1.00 | 8.91 | A |
| ATOM | 42 | CB | THR | A | 10 | 26.830 | 80.351 | 30.792 | 1.00 | 9.80 | A |
| ATOM | 43 | OG1 | THR | A | 10 | 27.387 | 80.067 | 29.502 | 1.00 | 9.14 | A |
| ATOM | 44 | CG2 | THR | A | 10 | 25.342 | 80.602 | 30.668 | 1.00 | 10.14 | A |
| ATOM | 45 | C | THR | A | 10 | 27.218 | 82.883 | 30.724 | 1.00 | 9.78 | A |
| ATOM | 46 | O | THR | A | 10 | 26.866 | 83.864 | 31.384 | 1.00 | 8.65 | A |
| ATOM | 47 | N | ILE | A | 11 | 27.309 | 82.912 | 29.404 | 1.00 | 9.26 | A |
| ATOM | 48 | CA | ILE | A | 11 | 27.068 | 84.147 | 28.675 | 1.00 | 9.83 | A |
| ATOM | 49 | CB | ILE | A | 11 | 28.415 | 84.918 | 28.539 | 1.00 | 9.13 | A |
| ATOM | 50 | CG2 | ILE | A | 11 | 29.381 | 84.119 | 27.675 | 1.00 | 10.83 | A |
| ATOM | 51 | CG1 | ILE | A | 11 | 28.196 | 86.325 | 27.985 | 1.00 | 10.63 | A |
| ATOM | 52 | CD1 | ILE | A | 11 | 29.425 | 87.221 | 28.126 | 1.00 | 12.27 | A |
| ATOM | 53 | C | ILE | A | 11 | 26.476 | 83.819 | 27.312 | 1.00 | 9.66 | A |
| ATOM | 54 | O | ILE | A | 11 | 26.716 | 82.737 | 26.771 | 1.00 | 9.86 | A |
| ATOM | 55 | N | LEU | A | 12 | 25.690 | 84.741 | 26.762 | 1.00 | 10.35 | A |
| ATOM | 56 | CA | LEU | A | 12 | 25.083 | 84.521 | 25.460 | 1.00 | 12.08 | A |
| ATOM | 57 | CB | LEU | A | 12 | 23.825 | 85.391 | 25.334 | 1.00 | 11.83 | A |
| ATOM | 58 | CG | LEU | A | 12 | 22.744 | 85.141 | 26.394 | 1.00 | 11.57 | A |
| ATOM | 59 | CD1 | LEU | A | 12 | 21.572 | 86.085 | 26.158 | 1.00 | 14.26 | A |
| ATOM | 60 | CD2 | LEU | A | 12 | 22.279 | 83.691 | 26.351 | 1.00 | 13.48 | A |
| ATOM | 61 | C | LEU | A | 12 | 26.116 | 84.775 | 24.367 | 1.00 | 12.55 | A |
| ATOM | 62 | O | LEU | A | 12 | 26.608 | 85.894 | 24.206 | 1.00 | 15.65 | A |
| ATOM | 63 | N | GLU | A | 13 | 26.453 | 83.719 | 23.634 | 1.00 | 11.29 | A |
| ATOM | 64 | CA | GLU | A | 13 | 27.416 | 83.777 | 22.534 | 1.00 | 12.00 | A |
| ATOM | 65 | CB | GLU | A | 13 | 28.849 | 83.782 | 23.093 | 1.00 | 11.61 | A |
| ATOM | 66 | CG | GLU | A | 13 | 29.952 | 83.756 | 22.018 | 1.00 | 12.91 | A |
| ATOM | 67 | CD | GLU | A | 13 | 30.165 | 85.099 | 21.328 | 1.00 | 14.45 | A |
| ATOM | 68 | OE1 | GLU | A | 13 | 30.740 | 85.111 | 20.218 | 1.00 | 14.76 | A |
| ATOM | 69 | OE2 | GLU | A | 13 | 29.776 | 86.143 | 21.892 | 1.00 | 14.04 | A |
| ATOM | 70 | C | GLU | A | 13 | 27.114 | 82.557 | 21.668 | 1.00 | 11.99 | A |
| ATOM | 71 | O | GLU | A | 13 | 27.210 | 81.426 | 22.137 | 1.00 | 12.31 | A |
| ATOM | 72 | N | SER | A | 14 | 26.743 | 82.784 | 20.412 | 1.00 | 11.74 | A |
| ATOM | 73 | CA | SER | A | 14 | 26.419 | 81.690 | 19.499 | 1.00 | 10.92 | A |
| ATOM | 74 | CB | SER | A | 14 | 25.658 | 82.255 | 18.300 | 1.00 | 12.79 | A |
| ATOM | 75 | OG | SER | A | 14 | 24.496 | 82.939 | 18.710 | 1.00 | 15.59 | A |
| ATOM | 76 | C | SER | A | 14 | 27.682 | 80.950 | 19.082 | 1.00 | 11.02 | A |
| ATOM | 77 | O | SER | A | 14 | 28.713 | 81.570 | 18.832 | 1.00 | 13.24 | A |
| ATOM | 78 | N | PRO | A | 15 | 27.618 | 79.615 | 18.969 | 1.00 | 10.96 | A |
| ATOM | 79 | CD | PRO | A | 15 | 28.696 | 78.928 | 18.235 | 1.00 | 10.29 | A |
| ATOM | 80 | CA | PRO | A | 15 | 26.493 | 78.705 | 19.193 | 1.00 | 11.07 | A |
| ATOM | 81 | CB | PRO | A | 15 | 26.640 | 77.742 | 18.022 | 1.00 | 10.06 | A |
| ATOM | 82 | CG | PRO | A | 15 | 28.133 | 77.463 | 18.043 | 1.00 | 11.18 | A |
| ATOM | 83 | C | PRO | A | 15 | 26.597 | 78.055 | 20.571 | 1.00 | 10.83 | A |
| ATOM | 84 | O | PRO | A | 15 | 26.046 | 76.981 | 20.794 | 1.00 | 10.59 | A |
| ATOM | 85 | N | TYR | A | 16 | 27.305 | 78.709 | 21.489 | 1.00 | 10.45 | A |
| ATOM | 86 | CA | TYR | A | 16 | 27.517 | 78.184 | 22.840 | 1.00 | 9.96 | A |
| ATOM | 87 | CB | TYR | A | 16 | 28.710 | 78.909 | 23.464 | 1.00 | 11.23 | A |
| ATOM | 88 | CG | TYR | A | 16 | 29.952 | 78.896 | 22.606 | 1.00 | 9.43 | A |
| ATOM | 89 | CD1 | TYR | A | 16 | 30.820 | 77.801 | 22.598 | 1.00 | 9.41 | A |
| ATOM | 90 | CE1 | TYR | A | 16 | 31.967 | 77.799 | 21.787 | 1.00 | 8.96 | A |
| ATOM | 91 | CD2 | TYR | A | 16 | 30.253 | 79.980 | 21.789 | 1.00 | 10.09 | A |
| ATOM | 92 | CE2 | TYR | A | 16 | 31.387 | 79.986 | 20.980 | 1.00 | 10.47 | A |
| ATOM | 93 | CZ | TYR | A | 16 | 32.237 | 78.899 | 20.984 | 1.00 | 10.17 | A |
| ATOM | 94 | OH | TYR | A | 16 | 33.363 | 78.920 | 20.190 | 1.00 | 11.52 | A |
| ATOM | 95 | C | TYR | A | 16 | 26.275 | 78.266 | 23.712 | 1.00 | 10.43 | A |
| ATOM | 96 | O | TYR | A | 16 | 25.892 | 77.283 | 24.342 | 1.00 | 10.31 | A |
| ATOM | 97 | N | VAL | A | 17 | 25.674 | 79.450 | 23.773 | 1.00 | 10.32 | A |
| ATOM | 98 | CA | VAL | A | 17 | 24.465 | 79.671 | 24.557 | 1.00 | 10.55 | A |
| ATOM | 99 | CB | VAL | A | 17 | 24.780 | 80.166 | 25.990 | 1.00 | 9.76 | A |
| ATOM | 100 | CG1 | VAL | A | 17 | 23.489 | 80.287 | 26.792 | 1.00 | 11.30 | A |
| ATOM | 101 | CG2 | VAL | A | 17 | 25.739 | 79.210 | 26.680 | 1.00 | 11.77 | A |
| ATOM | 102 | C | VAL | A | 17 | 23.634 | 80.728 | 23.850 | 1.00 | 10.95 | A |
| ATOM | 103 | O | VAL | A | 17 | 24.127 | 81.814 | 23.543 | 1.00 | 11.05 | A |
| ATOM | 104 | N | MET | A | 18 | 22.377 | 80.390 | 23.586 | 1.00 | 10.77 | A |
| ATOM | 105 | CA | MET | A | 18 | 21.441 | 81.279 | 22.913 | 1.00 | 12.48 | A |
| ATOM | 106 | CB | MET | A | 18 | 21.409 | 80.927 | 21.417 | 1.00 | 15.54 | A |
| ATOM | 107 | CG | MET | A | 18 | 22.783 | 80.824 | 20.761 | 1.00 | 18.73 | A |
| ATOM | 108 | SD | MET | A | 18 | 22.773 | 79.981 | 19.154 | 1.00 | 22.12 | A |
| ATOM | 109 | CE | MET | A | 18 | 22.727 | 78.246 | 19.684 | 1.00 | 15.92 | A |
| ATOM | 110 | C | MET | A | 18 | 20.082 | 81.091 | 23.579 | 1.00 | 12.27 | A |
| ATOM | 111 | O | MET | A | 18 | 19.805 | 80.030 | 24.140 | 1.00 | 12.26 | A |
| ATOM | 112 | N | MET | A | 19 | 19.239 | 82.117 | 23.541 | 1.00 | 12.48 | A |
| ATOM | 113 | CA | MET | A | 19 | 17.904 | 81.974 | 24.101 | 1.00 | 12.14 | A |
| ATOM | 114 | CB | MET | A | 19 | 17.347 | 83.338 | 24.535 | 1.00 | 15.09 | A |
| ATOM | 115 | CG | MET | A | 19 | 17.994 | 83.934 | 25.770 | 1.00 | 13.78 | A |
| ATOM | 116 | SD | MET | A | 19 | 16.874 | 85.087 | 26.613 | 1.00 | 15.38 | A |
| ATOM | 117 | CE | MET | A | 19 | 16.000 | 83.961 | 27.704 | 1.00 | 16.72 | A |
| ATOM | 118 | C | MET | A | 19 | 17.069 | 81.322 | 23.006 | 1.00 | 13.23 | A |
| ATOM | 119 | O | MET | A | 19 | 17.135 | 81.735 | 21.848 | 1.00 | 14.65 | A |
| ATOM | 120 | N | LYS | A | 20 | 16.296 | 80.301 | 23.360 | 1.00 | 13.52 | A |
| ATOM | 121 | CA | LYS | A | 20 | 15.447 | 79.636 | 22.379 | 1.00 | 14.94 | A |
| ATOM | 122 | CB | LYS | A | 20 | 14.725 | 78.453 | 23.029 | 1.00 | 13.74 | A |
| ATOM | 123 | CG | LYS | A | 20 | 15.604 | 77.280 | 23.438 | 1.00 | 15.44 | A |
| ATOM | 124 | CD | LYS | A | 20 | 14.750 | 76.205 | 24.096 | 1.00 | 16.23 | A |
| ATOM | 125 | CE | LYS | A | 20 | 15.567 | 75.003 | 24.533 | 1.00 | 14.95 | A |
| ATOM | 126 | NZ | LYS | A | 20 | 14.725 | 74.058 | 25.322 | 1.00 | 16.86 | A |
| ATOM | 127 | C | LYS | A | 20 | 14.474 | 80.678 | 21.842 | 1.00 | 16.34 | A |
| ATOM | 128 | O | LYS | A | 20 | 14.138 | 81.639 | 22.535 | 1.00 | 15.24 | A |
| ATOM | 129 | N | ALA | A | 21 | 14.012 | 80.483 | 20.613 | 1.00 | 17.51 | A |
| ATOM | 130 | CA | ALA | A | 21 | 13.072 | 81.412 | 20.004 | 1.00 | 18.87 | A |
| ATOM | 131 | CB | ALA | A | 21 | 12.758 | 80.972 | 18.578 | 1.00 | 20.19 | A |
| ATOM | 132 | C | ALA | A | 21 | 11.790 | 81.484 | 20.832 | 1.00 | 18.60 | A |
| ATOM | 133 | O | ALA | A | 21 | 11.182 | 82.548 | 20.963 | 1.00 | 19.20 | A |
| ATOM | 134 | N | ASN | A | 22 | 11.401 | 80.350 | 21.405 | 1.00 | 17.71 | A |
| ATOM | 135 | CA | ASN | A | 22 | 10.187 | 80.263 | 22.213 | 1.00 | 18.07 | A |
| ATOM | 136 | CB | ASN | A | 22 | 9.431 | 78.992 | 21.812 | 1.00 | 18.69 | A |
| ATOM | 137 | CG | ASN | A | 22 | 10.150 | 77.726 | 22.242 | 1.00 | 20.55 | A |
| ATOM | 138 | OD1 | ASN | A | 22 | 11.365 | 77.601 | 22.079 | 1.00 | 20.26 | A |
| ATOM | 139 | ND2 | ASN | A | 22 | 9.397 | 76.773 | 22.786 | 1.00 | 22.20 | A |
| ATOM | 140 | C | ASN | A | 22 | 10.500 | 80.310 | 23.707 | 1.00 | 17.10 | A |
| ATOM | 141 | O | ASN | A | 22 | 9.757 | 79.753 | 24.518 | 1.00 | 19.00 | A |
| ATOM | 142 | N | HIS | A | 23 | 11.588 | 80.986 | 24.072 | 1.00 | 15.85 | A |
| ATOM | 143 | CA | HIS | A | 23 | 11.997 | 81.068 | 25.470 | 1.00 | 15.45 | A |
| ATOM | 144 | CB | HIS | A | 23 | 13.315 | 81.857 | 25.562 | 1.00 | 15.21 | A |
| ATOM | 145 | CG | HIS | A | 23 | 13.186 | 83.314 | 25.234 | 1.00 | 15.86 | A |
| ATOM | 146 | CD2 | HIS | A | 23 | 13.486 | 84.002 | 24.107 | 1.00 | 16.43 | A |
| ATOM | 147 | ND1 | HIS | A | 23 | 12.720 | 84.244 | 26.138 | 1.00 | 15.55 | A |
| ATOM | 148 | CE1 | HIS | A | 23 | 12.742 | 85.443 | 25.584 | 1.00 | 16.33 | A |
| ATOM | 149 | NE2 | HIS | A | 23 | 13.204 | 85.324 | 24.351 | 1.00 | 16.65 | A |
| ATOM | 150 | C | HIS | A | 23 | 10.897 | 81.598 | 26.391 | 1.00 | 15.46 | A |
| ATOM | 151 | O | HIS | A | 23 | 10.841 | 81.232 | 27.561 | 1.00 | 15.24 | A |
| ATOM | 152 | N | ALA | A | 24 | 10.014 | 82.441 | 25.863 | 1.00 | 16.95 | A |
| ATOM | 153 | CA | ALA | A | 24 | 8.932 | 83.001 | 26.670 | 1.00 | 17.83 | A |
| ATOM | 154 | CB | ALA | A | 24 | 8.183 | 84.064 | 25.872 | 1.00 | 19.08 | A |
| ATOM | 155 | C | ALA | A | 24 | 7.952 | 81.943 | 27.178 | 1.00 | 18.59 | A |
| ATOM | 156 | O | ALA | A | 24 | 7.300 | 82.136 | 28.202 | 1.00 | 18.94 | A |
| ATOM | 157 | N | MET | A | 25 | 7.848 | 80.826 | 26.465 | 1.00 | 18.33 | A |
| ATOM | 158 | CA | MET | A | 25 | 6.936 | 79.753 | 26.859 | 1.00 | 18.03 | A |
| ATOM | 159 | CB | MET | A | 25 | 6.331 | 79.113 | 25.608 | 1.00 | 20.26 | A |
| ATOM | 160 | CG | MET | A | 25 | 5.647 | 80.067 | 24.662 | 1.00 | 20.74 | A |
| ATOM | 161 | SD | MET | A | 25 | 4.819 | 79.150 | 23.355 | 1.00 | 24.57 | A |
| ATOM | 162 | CE | MET | A | 25 | 6.205 | 78.477 | 22.466 | 1.00 | 23.59 | A |
| ATOM | 163 | C | MET | A | 25 | 7.653 | 78.723 | 27.718 | 1.00 | 18.31 | A |
| ATOM | 164 | O | MET | A | 25 | 7.079 | 77.686 | 28.068 | 1.00 | 17.96 | A |
| ATOM | 165 | N | LEU | A | 26 | 8.906 | 79.009 | 28.053 | 1.00 | 17.37 | A |
| ATOM | 166 | CA | LEU | A | 26 | 9.717 | 78.091 | 28.834 | 1.00 | 16.23 | A |
| ATOM | 167 | CB | LEU | A | 26 | 10.892 | 77.641 | 27.966 | 1.00 | 15.94 | A |
| ATOM | 168 | CG | LEU | A | 26 | 10.507 | 77.102 | 26.585 | 1.00 | 18.54 | A |
| ATOM | 169 | CD1 | LEU | A | 26 | 11.757 | 76.868 | 25.756 | 1.00 | 18.19 | A |
| ATOM | 170 | CD2 | LEU | A | 26 | 9.719 | 75.812 | 26.738 | 1.00 | 17.91 | A |
| ATOM | 171 | C | LEU | A | 26 | 10.158 | 78.703 | 30.155 | 1.00 | 14.96 | A |
| ATOM | 172 | O | LEU | A | 26 | 9.945 | 79.889 | 30.414 | 1.00 | 15.22 | A |
| ATOM | 173 | N | ALA | A | 27 | 10.786 | 77.883 | 30.987 | 1.00 | 14.45 | A |
| ATOM | 174 | CA | ALA | A | 27 | 11.251 | 78.335 | 32.289 | 1.00 | 14.51 | A |
| ATOM | 175 | CB | ALA | A | 27 | 10.211 | 77.988 | 33.356 | 1.00 | 17.17 | A |
| ATOM | 176 | C | ALA | A | 27 | 12.588 | 77.705 | 32.644 | 1.00 | 14.03 | A |
| ATOM | 177 | O | ALA | A | 27 | 12.923 | 76.619 | 32.176 | 1.00 | 14.44 | A |
| ATOM | 178 | N | GLY | A | 28 | 13.357 | 78.404 | 33.469 | 1.00 | 12.71 | A |
| ATOM | 179 | CA | GLY | A | 28 | 14.641 | 77.885 | 33.907 | 1.00 | 12.56 | A |
| ATOM | 180 | C | GLY | A | 28 | 15.641 | 77.561 | 32.817 | 1.00 | 10.82 | A |
| ATOM | 181 | O | GLY | A | 28 | 15.749 | 78.276 | 31.818 | 1.00 | 10.69 | A |
| ATOM | 182 | N | ASN | A | 29 | 16.373 | 76.468 | 33.009 | 1.00 | 10.83 | A |
| ATOM | 183 | CA | ASN | A | 29 | 17.394 | 76.046 | 32.056 | 1.00 | 10.02 | A |
| ATOM | 184 | CB | ASN | A | 29 | 18.108 | 74.797 | 32.591 | 1.00 | 12.03 | A |
| ATOM | 185 | CG | ASN | A | 29 | 18.886 | 75.050 | 33.875 | 1.00 | 12.74 | A |
| ATOM | 186 | OD1 | ASN | A | 29 | 19.350 | 74.104 | 34.519 | 1.00 | 14.94 | A |
| ATOM | 187 | ND2 | ASN | A | 29 | 19.048 | 76.314 | 34.245 | 1.00 | 10.03 | A |
| ATOM | 188 | C | ASN | A | 29 | 16.823 | 75.817 | 30.659 | 1.00 | 10.58 | A |
| ATOM | 189 | O | ASN | A | 29 | 17.539 | 75.941 | 29.670 | 1.00 | 11.93 | A |
| ATOM | 190 | N | GLU | A | 30 | 15.540 | 75.468 | 30.585 | 1.00 | 10.30 | A |
| ATOM | 191 | CA | GLU | A | 30 | 14.872 | 75.199 | 29.310 | 1.00 | 11.21 | A |
| ATOM | 192 | CB | GLU | A | 30 | 13.458 | 74.677 | 29.598 | 1.00 | 13.90 | A |
| ATOM | 193 | CG | GLU | A | 30 | 13.410 | 73.267 | 30.163 | 1.00 | 18.50 | A |
| ATOM | 194 | CD | GLU | A | 30 | 13.463 | 72.200 | 29.086 | 1.00 | 21.23 | A |
| ATOM | 195 | OE1 | GLU | A | 30 | 13.680 | 72.550 | 27.907 | 1.00 | 23.76 | A |
| ATOM | 196 | OE2 | GLU | A | 30 | 13.290 | 71.007 | 29.420 | 1.00 | 22.98 | A |
| ATOM | 197 | C | GLU | A | 30 | 14.860 | 76.380 | 28.345 | 1.00 | 11.64 | A |
| ATOM | 198 | O | GLU | A | 30 | 14.645 | 76.200 | 27.148 | 1.00 | 12.13 | A |
| ATOM | 199 | N | ARG | A | 31 | 15.082 | 77.585 | 28.860 | 1.00 | 11.14 | A |
| ATOM | 200 | CA | ARG | A | 31 | 15.078 | 78.782 | 28.023 | 1.00 | 11.20 | A |
| ATOM | 201 | CB | ARG | A | 31 | 15.056 | 80.026 | 28.910 | 1.00 | 11.11 | A |
| ATOM | 202 | CG | ARG | A | 31 | 13.806 | 80.249 | 29.730 | 1.00 | 12.33 | A |
| ATOM | 203 | CD | ARG | A | 31 | 14.066 | 81.376 | 30.713 | 1.00 | 11.17 | A |
| ATOM | 204 | NE | ARG | A | 31 | 15.069 | 80.984 | 31.699 | 1.00 | 11.12 | A |
| ATOM | 205 | CZ | ARG | A | 31 | 15.636 | 81.817 | 32.564 | 1.00 | 9.72 | A |
| ATOM | 206 | NH1 | ARG | A | 31 | 15.305 | 83.100 | 32.564 | 1.00 | 11.45 | A |
| ATOM | 207 | NH2 | ARG | A | 31 | 16.520 | 81.367 | 33.447 | 1.00 | 9.95 | A |
| ATOM | 208 | C | ARG | A | 31 | 16.255 | 78.855 | 27.064 | 1.00 | 10.81 | A |
| ATOM | 209 | O | ARG | A | 31 | 16.183 | 79.557 | 26.060 | 1.00 | 11.62 | A |
| ATOM | 210 | N | TYR | A | 32 | 17.327 | 78.133 | 27.380 | 1.00 | 10.66 | A |
| ATOM | 211 | CA | TYR | A | 32 | 18.569 | 78.178 | 26.609 | 1.00 | 9.14 | A |
| ATOM | 212 | CB | TYR | A | 32 | 19.703 | 78.518 | 27.581 | 1.00 | 10.45 | A |
| ATOM | 213 | CG | TYR | A | 32 | 19.446 | 79.736 | 28.438 | 1.00 | 9.84 | A |
| ATOM | 214 | CD1 | TYR | A | 32 | 19.592 | 81.020 | 27.918 | 1.00 | 10.05 | A |
| ATOM | 215 | CE1 | TYR | A | 32 | 19.334 | 82.148 | 28.697 | 1.00 | 11.55 | A |
| ATOM | 216 | CD2 | TYR | A | 32 | 19.032 | 79.604 | 29.764 | 1.00 | 11.10 | A |
| ATOM | 217 | CE2 | TYR | A | 32 | 18.768 | 80.726 | 30.554 | 1.00 | 10.48 | A |
| ATOM | 218 | CZ | TYR | A | 32 | 18.922 | 81.994 | 30.012 | 1.00 | 10.66 | A |
| ATOM | 219 | OH | TYR | A | 32 | 18.653 | 83.107 | 30.779 | 1.00 | 12.60 | A |
| ATOM | 220 | C | TYR | A | 32 | 18.898 | 76.927 | 25.806 | 1.00 | 10.03 | A |
| ATOM | 221 | O | TYR | A | 32 | 18.448 | 75.831 | 26.123 | 1.00 | 10.26 | A |
| ATOM | 222 | N | GLU | A | 33 | 19.714 | 77.111 | 24.771 | 1.00 | 10.24 | A |
| ATOM | 223 | CA | GLU | A | 33 | 20.175 | 76.011 | 23.930 | 1.00 | 9.97 | A |
| ATOM | 224 | CB | GLU | A | 33 | 19.195 | 75.773 | 22.777 | 1.00 | 12.37 | A |
| ATOM | 225 | CG | GLU | A | 33 | 19.079 | 76.948 | 21.824 | 1.00 | 15.72 | A |
| ATOM | 226 | CD | GLU | A | 33 | 18.127 | 76.690 | 20.669 | 1.00 | 18.27 | A |
| ATOM | 227 | OE1 | GLU | A | 33 | 17.767 | 77.664 | 19.973 | 1.00 | 20.76 | A |
| ATOM | 228 | OE2 | GLU | A | 33 | 17.743 | 75.520 | 20.455 | 1.00 | 20.90 | A |
| ATOM | 229 | C | GLU | A | 33 | 21.572 | 76.360 | 23.444 | 1.00 | 10.37 | A |
| ATOM | 230 | O | GLU | A | 33 | 21.979 | 77.527 | 23.468 | 1.00 | 10.72 | A |
| ATOM | 231 | N | GLY | A | 34 | 22.311 | 75.349 | 23.002 | 1.00 | 9.68 | A |
| ATOM | 232 | CA | GLY | A | 34 | 23.652 | 75.600 | 22.508 | 1.00 | 9.45 | A |
| ATOM | 233 | C | GLY | A | 34 | 24.671 | 74.603 | 23.019 | 1.00 | 9.63 | A |
| ATOM | 234 | O | GLY | A | 34 | 24.384 | 73.791 | 23.902 | 1.00 | 10.51 | A |
| ATOM | 235 | N | TYR | A | 35 | 25.875 | 74.682 | 22.465 | 1.00 | 8.89 | A |
| ATOM | 236 | CA | TYR | A | 35 | 26.957 | 73.785 | 22.837 | 1.00 | 9.99 | A |
| ATOM | 237 | CB | TYR | A | 35 | 28.211 | 74.162 | 22.034 | 1.00 | 10.00 | A |
| ATOM | 238 | CG | TYR | A | 35 | 29.438 | 73.363 | 22.414 | 1.00 | 9.08 | A |
| ATOM | 239 | CD1 | TYR | A | 35 | 30.141 | 73.643 | 23.587 | 1.00 | 8.42 | A |
| ATOM | 240 | CE1 | TYR | A | 35 | 31.260 | 72.904 | 23.950 | 1.00 | 9.42 | A |
| ATOM | 241 | CD2 | TYR | A | 35 | 29.889 | 72.315 | 21.611 | 1.00 | 10.27 | A |
| ATOM | 242 | CE2 | TYR | A | 35 | 31.009 | 71.567 | 21.965 | 1.00 | 10.07 | A |
| ATOM | 243 | CZ | TYR | A | 35 | 31.688 | 71.870 | 23.137 | 1.00 | 9.20 | A |
| ATOM | 244 | OH | TYR | A | 35 | 32.794 | 71.145 | 23.506 | 1.00 | 13.71 | A |
| ATOM | 245 | C | TYR | A | 35 | 27.214 | 73.745 | 24.337 | 1.00 | 9.46 | A |
| ATOM | 246 | O | TYR | A | 35 | 27.307 | 72.665 | 24.922 | 1.00 | 9.83 | A |
| ATOM | 247 | N | CYS | A | 36 | 27.338 | 74.912 | 24.963 | 1.00 | 8.94 | A |
| ATOM | 248 | CA | CYS | A | 36 | 27.617 | 74.954 | 26.393 | 1.00 | 9.59 | A |
| ATOM | 249 | CB | CYS | A | 36 | 28.170 | 76.336 | 26.763 | 1.00 | 10.02 | A |
| ATOM | 250 | SG | CYS | A | 36 | 29.894 | 76.573 | 26.253 | 1.00 | 10.91 | A |
| ATOM | 251 | C | CYS | A | 36 | 26.428 | 74.527 | 27.236 | 1.00 | 9.87 | A |
| ATOM | 252 | O | CYS | A | 36 | 26.602 | 74.108 | 28.377 | 1.00 | 9.72 | A |
| ATOM | 253 | N | VAL | A | 37 | 25.224 | 74.649 | 26.688 | 1.00 | 9.91 | A |
| ATOM | 254 | CA | VAL | A | 37 | 24.034 | 74.223 | 27.413 | 1.00 | 10.54 | A |
| ATOM | 255 | CB | VAL | A | 37 | 22.749 | 74.708 | 26.710 | 1.00 | 10.90 | A |
| ATOM | 256 | CG1 | VAL | A | 37 | 21.521 | 74.155 | 27.424 | 1.00 | 12.65 | A |
| ATOM | 257 | CG2 | VAL | A | 37 | 22.709 | 76.232 | 26.715 | 1.00 | 12.51 | A |
| ATOM | 258 | C | VAL | A | 37 | 24.075 | 72.695 | 27.458 | 1.00 | 9.89 | A |
| ATOM | 259 | O | VAL | A | 37 | 23.850 | 72.086 | 28.502 | 1.00 | 11.28 | A |
| ATOM | 260 | N | ASP | A | 38 | 24.393 | 72.082 | 26.320 | 1.00 | 10.26 | A |
| ATOM | 261 | CA | ASP | A | 38 | 24.497 | 70.630 | 26.231 | 1.00 | 10.64 | A |
| ATOM | 262 | CB | ASP | A | 38 | 24.680 | 70.215 | 24.765 | 1.00 | 11.34 | A |
| ATOM | 263 | CG | ASP | A | 38 | 23.425 | 70.391 | 23.931 | 1.00 | 11.41 | A |
| ATOM | 264 | OD1 | ASP | A | 38 | 23.522 | 70.223 | 22.699 | 1.00 | 13.04 | A |
| ATOM | 265 | OD2 | ASP | A | 38 | 22.347 | 70.685 | 24.492 | 1.00 | 14.15 | A |
| ATOM | 266 | C | ASP | A | 38 | 25.652 | 70.157 | 27.108 | 1.00 | 10.10 | A |
| ATOM | 267 | O | ASP | A | 38 | 25.539 | 69.145 | 27.802 | 1.00 | 10.88 | A |
| ATOM | 268 | N | LEU | A | 39 | 26.770 | 70.880 | 27.065 | 1.00 | 10.24 | A |
| ATOM | 269 | CA | LEU | A | 39 | 27.931 | 70.512 | 27.860 | 1.00 | 10.57 | A |
| ATOM | 270 | CB | LEU | A | 39 | 29.109 | 71.423 | 27.496 | 1.00 | 10.29 | A |
| ATOM | 271 | CG | LEU | A | 39 | 30.386 | 71.177 | 28.307 | 1.00 | 10.48 | A |
| ATOM | 272 | CD1 | LEU | A | 39 | 30.832 | 69.725 | 28.174 | 1.00 | 11.52 | A |
| ATOM | 273 | CD2 | LEU | A | 39 | 31.474 | 72.133 | 27.830 | 1.00 | 11.16 | A |
| ATOM | 274 | C | LEU | A | 39 | 27.591 | 70.558 | 29.339 | 1.00 | 9.75 | A |
| ATOM | 275 | O | LEU | A | 39 | 27.937 | 69.646 | 30.094 | 1.00 | 10.20 | A |
| ATOM | 276 | N | ALA | A | 40 | 26.921 | 71.626 | 29.756 | 1.00 | 10.00 | A |
| ATOM | 277 | CA | ALA | A | 40 | 26.543 | 71.776 | 31.157 | 1.00 | 10.04 | A |
| ATOM | 278 | CB | ALA | A | 40 | 25.773 | 73.082 | 31.359 | 1.00 | 11.46 | A |
| ATOM | 279 | C | ALA | A | 40 | 25.707 | 70.584 | 31.625 | 1.00 | 11.75 | A |
| ATOM | 280 | O | ALA | A | 40 | 25.927 | 70.049 | 32.714 | 1.00 | 11.90 | A |
| ATOM | 281 | N | ALA | A | 41 | 24.759 | 70.160 | 30.795 | 1.00 | 11.48 | A |
| ATOM | 282 | CA | ALA | A | 41 | 23.904 | 69.030 | 31.139 | 1.00 | 11.88 | A |
| ATOM | 283 | CB | ALA | A | 41 | 22.835 | 68.837 | 30.068 | 1.00 | 13.22 | A |
| ATOM | 284 | C | ALA | A | 41 | 24.725 | 67.754 | 31.303 | 1.00 | 12.71 | A |
| ATOM | 285 | O | ALA | A | 41 | 24.481 | 66.969 | 32.219 | 1.00 | 13.49 | A |
| ATOM | 286 | N | GLU | A | 42 | 25.696 | 67.545 | 30.417 | 1.00 | 12.51 | A |
| ATOM | 287 | CA | GLU | A | 42 | 26.545 | 66.360 | 30.493 | 1.00 | 13.27 | A |
| ATOM | 288 | CB | GLU | A | 42 | 27.338 | 66.219 | 29.188 | 1.00 | 15.77 | A |
| ATOM | 289 | CG | GLU | A | 42 | 26.575 | 65.510 | 28.082 | 1.00 | 18.63 | A |
| ATOM | 290 | CD | GLU | A | 42 | 26.451 | 64.013 | 28.327 | 1.00 | 19.37 | A |
| ATOM | 291 | OE1 | GLU | A | 42 | 26.787 | 63.556 | 29.440 | 1.00 | 18.40 | A |
| ATOM | 292 | OE2 | GLU | A | 42 | 26.015 | 63.292 | 27.407 | 1.00 | 20.92 | A |
| ATOM | 293 | C | GLU | A | 42 | 27.435 | 66.389 | 31.728 | 1.00 | 13.94 | A |
| ATOM | 294 | O | GLU | A | 42 | 27.607 | 65.375 | 32.399 | 1.00 | 14.45 | A |
| ATOM | 295 | N | ILE | A | 43 | 28.018 | 67.545 | 32.023 | 1.00 | 12.51 | A |
| ATOM | 296 | CA | ILE | A | 43 | 28.878 | 67.665 | 33.193 | 1.00 | 13.29 | A |
| ATOM | 297 | CB | ILE | A | 43 | 29.491 | 69.085 | 33.300 | 1.00 | 13.12 | A |
| ATOM | 298 | CG2 | ILE | A | 43 | 30.204 | 69.245 | 34.634 | 1.00 | 12.80 | A |
| ATOM | 299 | CG1 | ILE | A | 43 | 30.456 | 69.333 | 32.137 | 1.00 | 14.92 | A |
| ATOM | 300 | CD1 | ILE | A | 43 | 31.665 | 68.428 | 32.127 | 1.00 | 14.91 | A |
| ATOM | 301 | C | ILE | A | 43 | 28.059 | 67.376 | 34.452 | 1.00 | 13.51 | A |
| ATOM | 302 | O | ILE | A | 43 | 28.484 | 66.616 | 35.325 | 1.00 | 14.79 | A |
| ATOM | 303 | N | ALA | A | 44 | 26.875 | 67.977 | 34.532 | 1.00 | 12.91 | A |
| ATOM | 304 | CA | ALA | A | 44 | 25.996 | 67.792 | 35.681 | 1.00 | 13.94 | A |
| ATOM | 305 | CB | ALA | A | 44 | 24.763 | 68.661 | 35.534 | 1.00 | 13.58 | A |
| ATOM | 306 | C | ALA | A | 44 | 25.592 | 66.335 | 35.867 | 1.00 | 14.33 | A |
| ATOM | 307 | O | ALA | A | 44 | 25.549 | 65.840 | 36.993 | 1.00 | 15.33 | A |
| ATOM | 308 | N | LYS | A | 45 | 25.296 | 65.651 | 34.766 | 1.00 | 15.00 | A |
| ATOM | 309 | CA | LYS | A | 45 | 24.908 | 64.246 | 34.828 | 1.00 | 17.52 | A |
| ATOM | 310 | CB | LYS | A | 45 | 24.467 | 63.776 | 33.440 | 1.00 | 19.45 | A |
| ATOM | 311 | CG | LYS | A | 45 | 23.146 | 64.350 | 32.965 | 1.00 | 23.23 | A |
| ATOM | 312 | CD | LYS | A | 45 | 22.709 | 63.696 | 31.662 | 1.00 | 26.44 | A |
| ATOM | 313 | CE | LYS | A | 45 | 22.539 | 62.192 | 31.836 | 1.00 | 28.68 | A |
| ATOM | 314 | NZ | LYS | A | 45 | 22.151 | 61.510 | 30.575 | 1.00 | 29.68 | A |
| ATOM | 315 | C | LYS | A | 45 | 26.041 | 63.402 | 35.395 | 1.00 | 17.69 | A |
| ATOM | 316 | O | LYS | A | 45 | 25.828 | 62.585 | 36.289 | 1.00 | 18.84 | A |
| ATOM | 317 | N | HIS | A | 46 | 27.248 | 63.597 | 34.876 | 1.00 | 16.11 | A |
| ATOM | 318 | CA | HIS | A | 46 | 28.396 | 62.828 | 35.340 | 1.00 | 16.88 | A |
| ATOM | 319 | CB | HIS | A | 46 | 29.540 | 62.966 | 34.328 | 1.00 | 15.26 | A |
| ATOM | 320 | CG | HIS | A | 46 | 29.312 | 62.206 | 33.060 | 1.00 | 16.43 | A |
| ATOM | 321 | CD2 | HIS | A | 46 | 29.739 | 60.982 | 32.668 | 1.00 | 18.25 | A |
| ATOM | 322 | ND1 | HIS | A | 46 | 28.511 | 62.675 | 32.042 | 1.00 | 18.48 | A |
| ATOM | 323 | CE1 | HIS | A | 46 | 28.454 | 61.774 | 31.078 | 1.00 | 18.78 | A |
| ATOM | 324 | NE2 | HIS | A | 46 | 29.190 | 60.737 | 31.433 | 1.00 | 21.02 | A |
| ATOM | 325 | C | HIS | A | 46 | 28.823 | 63.157 | 36.766 | 1.00 | 17.40 | A |
| ATOM | 326 | O | HIS | A | 46 | 29.336 | 62.291 | 37.474 | 1.00 | 19.71 | A |
| ATOM | 327 | N | CYS | A | 47 | 28.609 | 64.397 | 37.193 | 1.00 | 17.64 | A |
| ATOM | 328 | CA | CYS | A | 47 | 28.990 | 64.817 | 38.538 | 1.00 | 17.02 | A |
| ATOM | 329 | CB | CYS | A | 47 | 29.478 | 66.265 | 38.489 | 1.00 | 16.75 | A |
| ATOM | 330 | SG | CYS | A | 47 | 31.003 | 66.476 | 37.551 | 1.00 | 16.17 | A |
| ATOM | 331 | C | CYS | A | 47 | 27.862 | 64.610 | 39.534 | 1.00 | 17.55 | A |
| ATOM | 332 | O | CYS | A | 47 | 28.084 | 64.628 | 40.743 | 1.00 | 19.42 | A |
| ATOM | 333 | N | GLY | A | 48 | 26.652 | 64.426 | 39.022 | 1.00 | 16.92 | A |
| ATOM | 334 | CA | GLY | A | 48 | 25.513 | 64.207 | 39.890 | 1.00 | 18.16 | A |
| ATOM | 335 | C | GLY | A | 48 | 25.034 | 65.421 | 40.664 | 1.00 | 17.73 | A |
| ATOM | 336 | O | GLY | A | 48 | 24.782 | 65.329 | 41.865 | 1.00 | 20.39 | A |
| ATOM | 337 | N | PHE | A | 49 | 24.916 | 66.565 | 39.996 | 1.00 | 16.58 | A |
| ATOM | 338 | CA | PHE | A | 49 | 24.429 | 67.758 | 40.673 | 1.00 | 14.80 | A |
| ATOM | 339 | CB | PHE | A | 49 | 25.606 | 68.696 | 41.026 | 1.00 | 15.66 | A |
| ATOM | 340 | CG | PHE | A | 49 | 26.337 | 69.285 | 39.832 | 1.00 | 14.59 | A |
| ATOM | 341 | CD1 | PHE | A | 49 | 25.757 | 70.155 | 39.064 | 1.00 | 13.66 | A |
| ATOM | 342 | CD2 | PHE | A | 49 | 27.572 | 68.955 | 39.615 | 1.00 | 13.77 | A |
| ATOM | 343 | CE1 | PHE | A | 49 | 26.426 | 70.770 | 37.992 | 1.00 | 13.65 | A |
| ATOM | 344 | CE2 | PHE | A | 49 | 28.327 | 69.513 | 38.571 | 1.00 | 14.71 | A |
| ATOM | 345 | CZ | PHE | A | 49 | 27.739 | 70.438 | 37.748 | 1.00 | 14.34 | A |
| ATOM | 346 | C | PHE | A | 49 | 23.333 | 68.436 | 39.868 | 1.00 | 14.31 | A |
| ATOM | 347 | O | PHE | A | 49 | 23.230 | 68.252 | 38.654 | 1.00 | 14.47 | A |
| ATOM | 348 | N | ALA | A | 50 | 22.486 | 69.184 | 40.569 | 1.00 | 13.89 | A |
| ATOM | 349 | CA | ALA | A | 50 | 21.418 | 69.937 | 39.928 | 1.00 | 12.51 | A |
| ATOM | 350 | CB | ALA | A | 50 | 20.184 | 69.989 | 40.824 | 1.00 | 15.91 | A |
| ATOM | 351 | C | ALA | A | 50 | 22.019 | 71.327 | 39.762 | 1.00 | 12.02 | A |
| ATOM | 352 | O | ALA | A | 50 | 22.841 | 71.750 | 40.578 | 1.00 | 12.80 | A |
| ATOM | 353 | N | TYR | A | 51 | 21.621 | 72.040 | 38.717 | 1.00 | 12.82 | A |
| ATOM | 354 | CA | TYR | A | 51 | 22.193 | 73.360 | 38.481 | 1.00 | 11.50 | A |
| ATOM | 355 | CB | TYR | A | 51 | 23.423 | 73.214 | 37.582 | 1.00 | 10.79 | A |
| ATOM | 356 | CG | TYR | A | 51 | 23.074 | 72.687 | 36.211 | 1.00 | 10.07 | A |
| ATOM | 357 | CD1 | TYR | A | 51 | 22.770 | 71.340 | 36.023 | 1.00 | 11.63 | A |
| ATOM | 358 | CE1 | TYR | A | 51 | 22.394 | 70.858 | 34.775 | 1.00 | 11.65 | A |
| ATOM | 359 | CD2 | TYR | A | 51 | 22.996 | 73.544 | 35.111 | 1.00 | 11.90 | A |
| ATOM | 360 | CE2 | TYR | A | 51 | 22.621 | 73.071 | 33.862 | 1.00 | 11.04 | A |
| ATOM | 361 | CZ | TYR | A | 51 | 22.321 | 71.728 | 33.701 | 1.00 | 11.39 | A |
| ATOM | 362 | OH | TYR | A | 51 | 21.941 | 71.251 | 32.475 | 1.00 | 13.99 | A |
| ATOM | 363 | C | TYR | A | 51 | 21.209 | 74.344 | 37.891 | 1.00 | 11.97 | A |
| ATOM | 364 | O | TYR | A | 51 | 20.161 | 73.973 | 37.366 | 1.00 | 11.86 | A |
| ATOM | 365 | N | ALA | A | 52 | 21.594 | 75.612 | 37.961 | 1.00 | 11.76 | A |
| ATOM | 366 | CA | ALA | A | 52 | 20.802 | 76.702 | 37.423 | 1.00 | 11.93 | A |
| ATOM | 367 | CB | ALA | A | 52 | 20.249 | 77.552 | 38.555 | 1.00 | 15.06 | A |
| ATOM | 368 | C | ALA | A | 52 | 21.706 | 77.543 | 36.532 | 1.00 | 11.65 | A |
| ATOM | 369 | O | ALA | A | 52 | 22.748 | 78.021 | 36.975 | 1.00 | 11.80 | A |
| ATOM | 370 | N | LEU | A | 53 | 21.314 | 77.702 | 35.273 | 1.00 | 10.72 | A |
| ATOM | 371 | CA | LEU | A | 53 | 22.081 | 78.517 | 34.336 | 1.00 | 10.99 | A |
| ATOM | 372 | CB | LEU | A | 53 | 21.746 | 78.108 | 32.898 | 1.00 | 11.40 | A |
| ATOM | 373 | CG | LEU | A | 53 | 22.095 | 76.694 | 32.431 | 1.00 | 11.91 | A |
| ATOM | 374 | CD1 | LEU | A | 53 | 21.540 | 76.471 | 31.032 | 1.00 | 13.32 | A |
| ATOM | 375 | CD2 | LEU | A | 53 | 23.601 | 76.503 | 32.442 | 1.00 | 14.04 | A |
| ATOM | 376 | C | LEU | A | 53 | 21.755 | 79.980 | 34.597 | 1.00 | 11.07 | A |
| ATOM | 377 | O | LEU | A | 53 | 20.587 | 80.366 | 34.589 | 1.00 | 11.97 | A |
| ATOM | 378 | N | THR | A | 54 | 22.784 | 80.786 | 34.839 | 1.00 | 10.70 | A |
| ATOM | 379 | CA | THR | A | 54 | 22.601 | 82.213 | 35.073 | 1.00 | 9.61 | A |
| ATOM | 380 | CB | THR | A | 54 | 22.956 | 82.625 | 36.522 | 1.00 | 11.92 | A |
| ATOM | 381 | OG1 | THR | A | 54 | 24.356 | 82.428 | 36.757 | 1.00 | 13.30 | A |
| ATOM | 382 | CG2 | THR | A | 54 | 22.150 | 81.806 | 37.519 | 1.00 | 12.21 | A |
| ATOM | 383 | C | THR | A | 54 | 23.518 | 82.940 | 34.097 | 1.00 | 10.63 | A |
| ATOM | 384 | O | THR | A | 54 | 24.583 | 82.433 | 33.742 | 1.00 | 10.64 | A |
| ATOM | 385 | N | ILE | A | 55 | 23.098 | 84.119 | 33.658 | 1.00 | 10.43 | A |
| ATOM | 386 | CA | ILE | A | 55 | 23.869 | 84.893 | 32.699 | 1.00 | 9.86 | A |
| ATOM | 387 | CB | ILE | A | 55 | 22.929 | 85.569 | 31.682 | 1.00 | 9.45 | A |
| ATOM | 388 | CG2 | ILE | A | 55 | 23.743 | 86.278 | 30.612 | 1.00 | 10.05 | A |
| ATOM | 389 | CG1 | ILE | A | 55 | 21.994 | 84.523 | 31.061 | 1.00 | 12.39 | A |
| ATOM | 390 | CD1 | ILE | A | 55 | 22.700 | 83.370 | 30.354 | 1.00 | 14.56 | A |
| ATOM | 391 | C | ILE | A | 55 | 24.705 | 85.939 | 33.418 | 1.00 | 11.14 | A |
| ATOM | 392 | O | ILE | A | 55 | 24.183 | 86.741 | 34.191 | 1.00 | 11.26 | A |
| ATOM | 393 | N | VAL | A | 56 | 26.009 | 85.926 | 33.160 | 1.00 | 10.84 | A |
| ATOM | 394 | CA | VAL | A | 56 | 26.931 | 86.853 | 33.805 | 1.00 | 10.67 | A |
| ATOM | 395 | CB | VAL | A | 56 | 28.374 | 86.689 | 33.232 | 1.00 | 9.37 | A |
| ATOM | 396 | CG1 | VAL | A | 56 | 28.408 | 86.996 | 31.748 | 1.00 | 8.67 | A |
| ATOM | 397 | CG2 | VAL | A | 56 | 29.335 | 87.577 | 33.996 | 1.00 | 11.63 | A |
| ATOM | 398 | C | VAL | A | 56 | 26.454 | 88.304 | 33.705 | 1.00 | 11.20 | A |
| ATOM | 399 | O | VAL | A | 56 | 26.191 | 88.824 | 32.617 | 1.00 | 12.28 | A |
| ATOM | 400 | N | GLY | A | 57 | 26.354 | 88.944 | 34.866 | 1.00 | 12.47 | A |
| ATOM | 401 | CA | GLY | A | 57 | 25.864 | 90.309 | 34.948 | 1.00 | 13.12 | A |
| ATOM | 402 | C | GLY | A | 57 | 26.506 | 91.389 | 34.102 | 1.00 | 14.20 | A |
| ATOM | 403 | O | GLY | A | 57 | 25.799 | 92.153 | 33.442 | 1.00 | 15.02 | A |
| ATOM | 404 | N | ASP | A | 58 | 27.833 | 91.466 | 34.111 | 1.00 | 14.42 | A |
| ATOM | 405 | CA | ASP | A | 58 | 28.524 | 92.506 | 33.356 | 1.00 | 14.36 | A |
| ATOM | 406 | CB | ASP | A | 58 | 29.749 | 92.969 | 34.156 | 1.00 | 14.26 | A |
| ATOM | 407 | CG | ASP | A | 58 | 30.813 | 91.898 | 34.273 | 1.00 | 13.53 | A |
| ATOM | 408 | OD1 | ASP | A | 58 | 31.796 | 92.125 | 35.008 | 1.00 | 15.20 | A |
| ATOM | 409 | OD2 | ASP | A | 58 | 30.672 | 90.834 | 33.629 | 1.00 | 13.56 | A |
| ATOM | 410 | C | ASP | A | 58 | 28.858 | 92.131 | 31.918 | 1.00 | 14.32 | A |
| ATOM | 411 | O | ASP | A | 58 | 29.520 | 92.898 | 31.218 | 1.00 | 15.28 | A |
| ATOM | 412 | N | GLY | A | 59 | 28.414 | 90.956 | 31.481 | 1.00 | 14.37 | A |
| ATOM | 413 | CA | GLY | A | 59 | 28.666 | 90.525 | 30.116 | 1.00 | 12.51 | A |
| ATOM | 414 | C | GLY | A | 59 | 30.126 | 90.394 | 29.717 | 1.00 | 12.13 | A |
| ATOM | 415 | O | GLY | A | 59 | 30.459 | 90.527 | 28.539 | 1.00 | 13.27 | A |
| ATOM | 416 | N | LYS | A | 60 | 30.994 | 90.123 | 30.688 | 1.00 | 10.36 | A |
| ATOM | 417 | CA | LYS | A | 60 | 32.429 | 89.986 | 30.435 | 1.00 | 11.62 | A |
| ATOM | 418 | CB | LYS | A | 60 | 33.190 | 91.006 | 31.291 | 1.00 | 12.17 | A |
| ATOM | 419 | CG | LYS | A | 60 | 32.771 | 92.459 | 31.118 | 1.00 | 15.61 | A |
| ATOM | 420 | CD | LYS | A | 60 | 33.202 | 93.020 | 29.780 | 1.00 | 19.19 | A |
| ATOM | 421 | CE | LYS | A | 60 | 32.861 | 94.502 | 29.692 | 1.00 | 19.93 | A |
| ATOM | 422 | NZ | LYS | A | 60 | 33.267 | 95.095 | 28.390 | 1.00 | 22.12 | A |
| ATOM | 423 | C | LYS | A | 60 | 32.891 | 88.562 | 30.710 | 1.00 | 10.40 | A |
| ATOM | 424 | O | LYS | A | 60 | 32.187 | 87.781 | 31.354 | 1.00 | 10.69 | A |
| ATOM | 425 | N | TYR | A | 61 | 34.086 | 88.231 | 30.227 | 1.00 | 9.97 | A |
| ATOM | 426 | CA | TYR | A | 61 | 34.647 | 86.904 | 30.440 | 1.00 | 9.43 | A |
| ATOM | 427 | CB | TYR | A | 61 | 35.378 | 86.445 | 29.173 | 1.00 | 9.37 | A |
| ATOM | 428 | CG | TYR | A | 61 | 34.427 | 85.969 | 28.109 | 1.00 | 9.04 | A |
| ATOM | 429 | CD1 | TYR | A | 61 | 33.629 | 86.872 | 27.405 | 1.00 | 9.32 | A |
| ATOM | 430 | CE1 | TYR | A | 61 | 32.698 | 86.432 | 26.471 | 1.00 | 10.34 | A |
| ATOM | 431 | CD2 | TYR | A | 61 | 34.272 | 84.609 | 27.846 | 1.00 | 10.11 | A |
| ATOM | 432 | CE2 | TYR | A | 61 | 33.343 | 84.158 | 26.911 | 1.00 | 9.38 | A |
| ATOM | 433 | CZ | TYR | A | 61 | 32.560 | 85.075 | 26.230 | 1.00 | 11.16 | A |
| ATOM | 434 | OH | TYR | A | 61 | 31.646 | 84.628 | 25.306 | 1.00 | 11.81 | A |
| ATOM | 435 | C | TYR | A | 61 | 35.512 | 86.878 | 31.689 | 1.00 | 9.35 | A |
| ATOM | 436 | O | TYR | A | 61 | 35.276 | 86.075 | 32.588 | 1.00 | 10.72 | A |
| ATOM | 437 | N | GLY | A | 62 | 36.514 | 87.750 | 31.753 | 1.00 | 9.91 | A |
| ATOM | 438 | CA | GLY | A | 62 | 37.359 | 87.782 | 32.931 | 1.00 | 10.68 | A |
| ATOM | 439 | C | GLY | A | 62 | 38.661 | 88.539 | 32.767 | 1.00 | 10.33 | A |
| ATOM | 440 | O | GLY | A | 62 | 39.585 | 88.061 | 32.106 | 1.00 | 10.43 | A |
| ATOM | 441 | N | ALA | A | 63 | 38.735 | 89.718 | 33.380 | 1.00 | 11.66 | A |
| ATOM | 442 | CA | ALA | A | 63 | 39.930 | 90.554 | 33.334 | 1.00 | 11.95 | A |
| ATOM | 443 | CB | ALA | A | 63 | 39.865 | 91.513 | 32.152 | 1.00 | 13.77 | A |
| ATOM | 444 | C | ALA | A | 63 | 39.992 | 91.336 | 34.639 | 1.00 | 13.84 | A |
| ATOM | 445 | O | ALA | A | 63 | 38.968 | 91.556 | 35.286 | 1.00 | 13.50 | A |
| ATOM | 446 | N | ARG | A | 64 | 41.192 | 91.754 | 35.025 | 1.00 | 14.65 | A |
| ATOM | 447 | CA | ARG | A | 64 | 41.366 | 92.509 | 36.257 | 1.00 | 17.59 | A |
| ATOM | 448 | CB | ARG | A | 64 | 42.431 | 91.832 | 37.122 | 1.00 | 19.09 | A |
| ATOM | 449 | CG | ARG | A | 64 | 42.557 | 92.447 | 38.509 | 1.00 | 22.61 | A |
| ATOM | 450 | CD | ARG | A | 64 | 43.792 | 91.954 | 39.238 | 1.00 | 25.53 | A |
| ATOM | 451 | NE | ARG | A | 64 | 45.020 | 92.426 | 38.605 | 1.00 | 28.82 | A |
| ATOM | 452 | CZ | ARG | A | 64 | 46.236 | 92.250 | 39.111 | 1.00 | 31.37 | A |
| ATOM | 453 | NH1 | ARG | A | 64 | 46.390 | 91.608 | 40.262 | 1.00 | 33.09 | A |
| ATOM | 454 | NH2 | ARG | A | 64 | 47.297 | 92.719 | 38.469 | 1.00 | 32.81 | A |
| ATOM | 455 | C | ARG | A | 64 | 41.710 | 93.958 | 35.946 | 1.00 | 18.49 | A |
| ATOM | 456 | O | ARG | A | 64 | 42.621 | 94.233 | 35.163 | 1.00 | 18.63 | A |
| ATOM | 457 | N | ASP | A | 65 | 40.980 | 94.883 | 36.558 | 1.00 | 20.04 | A |
| ATOM | 458 | CA | ASP | A | 65 | 41.235 | 96.303 | 36.351 | 1.00 | 22.22 | A |
| ATOM | 459 | CB | ASP | A | 65 | 40.117 | 97.120 | 37.003 | 1.00 | 22.81 | A |
| ATOM | 460 | CG | ASP | A | 65 | 40.205 | 98.598 | 36.667 | 1.00 | 24.22 | A |
| ATOM | 461 | OD1 | ASP | A | 65 | 39.885 | 98.967 | 35.518 | 1.00 | 26.48 | A |
| ATOM | 462 | OD2 | ASP | A | 65 | 40.603 | 99.387 | 37.548 | 1.00 | 26.15 | A |
| ATOM | 463 | C | ASP | A | 65 | 42.611 | 96.581 | 36.947 | 1.00 | 23.72 | A |
| ATOM | 464 | O | ASP | A | 65 | 42.884 | 96.210 | 38.087 | 1.00 | 23.99 | A |
| ATOM | 465 | N | ALA | A | 66 | 43.477 | 97.229 | 36.176 | 1.00 | 26.04 | A |
| ATOM | 466 | CA | ALA | A | 66 | 44.828 | 97.531 | 36.636 | 1.00 | 28.03 | A |
| ATOM | 467 | CB | ALA | A | 66 | 45.652 | 98.090 | 35.483 | 1.00 | 28.75 | A |
| ATOM | 468 | C | ALA | A | 66 | 44.857 | 98.499 | 37.814 | 1.00 | 28.79 | A |
| ATOM | 469 | O | ALA | A | 66 | 45.809 | 98.504 | 38.595 | 1.00 | 29.26 | A |
| ATOM | 470 | N | ASN | A | 67 | 43.812 | 99.310 | 37.946 | 1.00 | 29.06 | A |
| ATOM | 471 | CA | ASN | A | 67 | 43.740 | 100.288 | 39.025 | 1.00 | 29.85 | A |
| ATOM | 472 | CB | ASN | A | 67 | 43.019 | 101.537 | 38.509 | 1.00 | 30.58 | A |
| ATOM | 473 | CG | ASN | A | 67 | 43.700 | 102.145 | 37.298 | 1.00 | 32.46 | A |
| ATOM | 474 | OD1 | ASN | A | 67 | 44.886 | 102.473 | 37.339 | 1.00 | 32.86 | A |
| ATOM | 475 | ND2 | ASN | A | 67 | 42.952 | 102.300 | 36.212 | 1.00 | 33.03 | A |
| ATOM | 476 | C | ASN | A | 67 | 43.105 | 99.717 | 40.288 | 1.00 | 29.38 | A |
| ATOM | 477 | O | ASN | A | 67 | 43.744 | 99.655 | 41.339 | 1.00 | 29.66 | A |
| ATOM | 478 | N | THR | A | 68 | 41.847 | 99.301 | 40.179 | 1.00 | 27.82 | A |
| ATOM | 479 | CA | THR | A | 68 | 41.109 | 98.755 | 41.314 | 1.00 | 26.63 | A |
| ATOM | 480 | CB | THR | A | 68 | 39.595 | 98.807 | 41.060 | 1.00 | 25.72 | A |
| ATOM | 481 | OG1 | THR | A | 68 | 39.265 | 97.930 | 39.975 | 1.00 | 24.07 | A |
| ATOM | 482 | CG2 | THR | A | 68 | 39.166 | 100.222 | 40.710 | 1.00 | 25.34 | A |
| ATOM | 483 | C | THR | A | 68 | 41.479 | 97.313 | 41.648 | 1.00 | 26.22 | A |
| ATOM | 484 | O | THR | A | 68 | 41.233 | 96.848 | 42.761 | 1.00 | 26.49 | A |
| ATOM | 485 | N | LYS | A | 69 | 42.059 | 96.611 | 40.681 | 1.00 | 25.60 | A |
| ATOM | 486 | CA | LYS | A | 69 | 42.461 | 95.221 | 40.867 | 1.00 | 25.08 | A |
| ATOM | 487 | CB | LYS | A | 69 | 43.465 | 95.142 | 42.022 | 1.00 | 28.90 | A |
| ATOM | 488 | CG | LYS | A | 69 | 44.710 | 95.990 | 41.814 | 1.00 | 33.71 | A |
| ATOM | 489 | CD | LYS | A | 69 | 45.662 | 95.872 | 42.991 | 1.00 | 38.30 | A |
| ATOM | 490 | CE | LYS | A | 69 | 46.885 | 96.758 | 42.808 | 1.00 | 40.62 | A |
| ATOM | 491 | NZ | LYS | A | 69 | 47.821 | 96.646 | 43.961 | 1.00 | 42.64 | A |
| ATOM | 492 | C | LYS | A | 69 | 41.249 | 94.314 | 41.063 | 1.00 | 23.09 | A |
| ATOM | 493 | O | LYS | A | 69 | 41.370 | 93.209 | 41.591 | 1.00 | 22.36 | A |
| ATOM | 494 | N | ILE | A | 70 | 40.082 | 94.785 | 40.636 | 1.00 | 18.73 | A |
| ATOM | 495 | CA | ILE | A | 70 | 38.851 | 94.007 | 40.752 | 1.00 | 17.41 | A |
| ATOM | 496 | CB | ILE | A | 70 | 37.619 | 94.926 | 40.921 | 1.00 | 18.61 | A |
| ATOM | 497 | CG2 | ILE | A | 70 | 36.343 | 94.096 | 40.924 | 1.00 | 19.24 | A |
| ATOM | 498 | CG1 | ILE | A | 70 | 37.743 | 95.733 | 42.215 | 1.00 | 19.23 | A |
| ATOM | 499 | CD1 | ILE | A | 70 | 36.620 | 96.736 | 42.423 | 1.00 | 20.47 | A |
| ATOM | 500 | C | ILE | A | 70 | 38.656 | 93.173 | 39.490 | 1.00 | 15.83 | A |
| ATOM | 501 | O | ILE | A | 70 | 38.887 | 93.656 | 38.383 | 1.00 | 15.20 | A |
| ATOM | 502 | N | TRP | A | 71 | 38.240 | 91.921 | 39.663 | 1.00 | 13.08 | A |
| ATOM | 503 | CA | TRP | A | 71 | 38.003 | 91.026 | 38.535 | 1.00 | 12.55 | A |
| ATOM | 504 | CB | TRP | A | 71 | 38.318 | 89.583 | 38.942 | 1.00 | 12.03 | A |
| ATOM | 505 | CG | TRP | A | 71 | 39.772 | 89.255 | 38.964 | 1.00 | 13.66 | A |
| ATOM | 506 | CD2 | TRP | A | 71 | 40.549 | 88.766 | 37.868 | 1.00 | 13.45 | A |
| ATOM | 507 | CE2 | TRP | A | 71 | 41.874 | 88.607 | 38.330 | 1.00 | 15.39 | A |
| ATOM | 508 | CE3 | TRP | A | 71 | 40.254 | 88.450 | 36.536 | 1.00 | 13.75 | A |
| ATOM | 509 | CD1 | TRP | A | 71 | 40.630 | 89.371 | 40.022 | 1.00 | 13.77 | A |
| ATOM | 510 | NE1 | TRP | A | 71 | 41.896 | 88.981 | 39.649 | 1.00 | 15.77 | A |
| ATOM | 511 | CZ2 | TRP | A | 71 | 42.905 | 88.142 | 37.505 | 1.00 | 15.44 | A |
| ATOM | 512 | CZ3 | TRP | A | 71 | 41.280 | 87.987 | 35.715 | 1.00 | 14.18 | A |
| ATOM | 513 | CH2 | TRP | A | 71 | 42.588 | 87.839 | 36.206 | 1.00 | 15.37 | A |
| ATOM | 514 | C | TRP | A | 71 | 36.576 | 91.164 | 38.036 | 1.00 | 12.49 | A |
| ATOM | 515 | O | TRP | A | 71 | 35.641 | 91.188 | 38.831 | 1.00 | 12.74 | A |
| ATOM | 516 | N | ASN | A | 72 | 36.407 | 91.244 | 36.718 | 1.00 | 11.07 | A |
| ATOM | 517 | CA | ASN | A | 72 | 35.075 | 91.350 | 36.133 | 1.00 | 11.34 | A |
| ATOM | 518 | CB | ASN | A | 72 | 35.022 | 92.534 | 35.155 | 1.00 | 10.91 | A |
| ATOM | 519 | CG | ASN | A | 72 | 35.893 | 92.322 | 33.928 | 1.00 | 12.43 | A |
| ATOM | 520 | OD1 | ASN | A | 72 | 36.262 | 91.195 | 33.599 | 1.00 | 12.97 | A |
| ATOM | 521 | ND2 | ASN | A | 72 | 36.209 | 93.410 | 33.233 | 1.00 | 14.86 | A |
| ATOM | 522 | C | ASN | A | 72 | 34.692 | 90.034 | 35.463 | 1.00 | 10.96 | A |
| ATOM | 523 | O | ASN | A | 72 | 35.432 | 89.049 | 35.524 | 1.00 | 11.26 | A |
| ATOM | 524 | N | GLY | A | 73 | 33.521 | 90.034 | 34.836 | 1.00 | 11.00 | A |
| ATOM | 525 | CA | GLY | A | 73 | 33.038 | 88.871 | 34.112 | 1.00 | 10.39 | A |
| ATOM | 526 | C | GLY | A | 73 | 32.798 | 87.602 | 34.904 | 1.00 | 9.77 | A |
| ATOM | 527 | O | GLY | A | 73 | 32.636 | 87.625 | 36.126 | 1.00 | 9.76 | A |
| ATOM | 528 | N | MET | A | 74 | 32.783 | 86.480 | 34.189 | 1.00 | 8.85 | A |
| ATOM | 529 | CA | MET | A | 74 | 32.551 | 85.179 | 34.798 | 1.00 | 9.58 | A |
| ATOM | 530 | CB | MET | A | 74 | 32.388 | 84.130 | 33.696 | 1.00 | 9.64 | A |
| ATOM | 531 | CG | MET | A | 74 | 31.126 | 84.314 | 32.874 | 1.00 | 10.68 | A |
| ATOM | 532 | SD | MET | A | 74 | 30.972 | 83.136 | 31.533 | 1.00 | 9.47 | A |
| ATOM | 533 | CE | MET | A | 74 | 32.107 | 83.851 | 30.331 | 1.00 | 10.20 | A |
| ATOM | 534 | C | MET | A | 74 | 33.644 | 84.841 | 35.792 | 1.00 | 9.16 | A |
| ATOM | 535 | O | MET | A | 74 | 33.374 | 84.250 | 36.835 | 1.00 | 9.49 | A |
| ATOM | 536 | N | VAL | A | 75 | 34.882 | 85.203 | 35.469 | 1.00 | 9.50 | A |
| ATOM | 537 | CA | VAL | A | 75 | 35.979 | 84.938 | 36.383 | 1.00 | 10.09 | A |
| ATOM | 538 | CB | VAL | A | 75 | 37.334 | 85.382 | 35.784 | 1.00 | 9.77 | A |
| ATOM | 539 | CG1 | VAL | A | 75 | 38.440 | 85.254 | 36.831 | 1.00 | 11.29 | A |
| ATOM | 540 | CG2 | VAL | A | 75 | 37.663 | 84.512 | 34.568 | 1.00 | 11.69 | A |
| ATOM | 541 | C | VAL | A | 75 | 35.690 | 85.687 | 37.683 | 1.00 | 8.71 | A |
| ATOM | 542 | O | VAL | A | 75 | 35.847 | 85.131 | 38.771 | 1.00 | 9.77 | A |
| ATOM | 543 | N | GLY | A | 76 | 35.243 | 86.936 | 37.566 | 1.00 | 9.90 | A |
| ATOM | 544 | CA | GLY | A | 76 | 34.919 | 87.713 | 38.753 | 1.00 | 10.44 | A |
| ATOM | 545 | C | GLY | A | 76 | 33.828 | 87.064 | 39.590 | 1.00 | 9.56 | A |
| ATOM | 546 | O | GLY | A | 76 | 33.922 | 87.026 | 40.815 | 1.00 | 9.93 | A |
| ATOM | 547 | N | GLU | A | 77 | 32.789 | 86.547 | 38.943 | 1.00 | 9.57 | A |
| ATOM | 548 | CA | GLU | A | 77 | 31.707 | 85.905 | 39.680 | 1.00 | 10.67 | A |
| ATOM | 549 | CB | GLU | A | 77 | 30.583 | 85.522 | 38.715 | 1.00 | 12.21 | A |
| ATOM | 550 | CG | GLU | A | 77 | 29.957 | 86.719 | 38.025 | 1.00 | 14.32 | A |
| ATOM | 551 | CD | GLU | A | 77 | 28.570 | 86.433 | 37.489 | 1.00 | 16.51 | A |
| ATOM | 552 | OE1 | GLU | A | 77 | 28.226 | 85.246 | 37.321 | 1.00 | 18.52 | A |
| ATOM | 553 | OE2 | GLU | A | 77 | 27.825 | 87.397 | 37.228 | 1.00 | 16.53 | A |
| ATOM | 554 | C | GLU | A | 77 | 32.241 | 84.732 | 40.490 | 1.00 | 10.91 | A |
| ATOM | 555 | O | GLU | A | 77 | 31.785 | 84.483 | 41.604 | 1.00 | 11.18 | A |
| ATOM | 556 | N | LEU | A | 78 | 33.209 | 84.014 | 39.932 | 1.00 | 9.49 | A |
| ATOM | 557 | CA | LEU | A | 78 | 33.815 | 82.880 | 40.619 | 1.00 | 10.07 | A |
| ATOM | 558 | CB | LEU | A | 78 | 34.630 | 82.074 | 39.607 | 1.00 | 10.23 | A |
| ATOM | 559 | CG | LEU | A | 78 | 34.212 | 80.693 | 39.093 | 1.00 | 16.68 | A |
| ATOM | 560 | CD1 | LEU | A | 78 | 32.707 | 80.498 | 39.069 | 1.00 | 11.70 | A |
| ATOM | 561 | CD2 | LEU | A | 78 | 34.814 | 80.533 | 37.707 | 1.00 | 12.46 | A |
| ATOM | 562 | C | LEU | A | 78 | 34.669 | 83.375 | 41.782 | 1.00 | 10.32 | A |
| ATOM | 563 | O | LEU | A | 78 | 34.561 | 82.883 | 42.902 | 1.00 | 10.65 | A |
| ATOM | 564 | N | VAL | A | 79 | 35.530 | 84.344 | 41.495 | 1.00 | 9.96 | A |
| ATOM | 565 | CA | VAL | A | 79 | 36.433 | 84.906 | 42.490 | 1.00 | 10.88 | A |
| ATOM | 566 | CB | VAL | A | 79 | 37.312 | 86.008 | 41.842 | 1.00 | 10.55 | A |
| ATOM | 567 | CG1 | VAL | A | 79 | 38.072 | 86.795 | 42.909 | 1.00 | 12.96 | A |
| ATOM | 568 | CG2 | VAL | A | 79 | 38.292 | 85.361 | 40.867 | 1.00 | 12.38 | A |
| ATOM | 569 | C | VAL | A | 79 | 35.702 | 85.474 | 43.703 | 1.00 | 11.38 | A |
| ATOM | 570 | O | VAL | A | 79 | 36.130 | 85.276 | 44.843 | 1.00 | 11.63 | A |
| ATOM | 571 | N | TYR | A | 80 | 34.594 | 86.165 | 43.459 | 1.00 | 11.08 | A |
| ATOM | 572 | CA | TYR | A | 80 | 33.828 | 86.777 | 44.542 | 1.00 | 11.80 | A |
| ATOM | 573 | CB | TYR | A | 80 | 33.337 | 88.151 | 44.075 | 1.00 | 11.15 | A |
| ATOM | 574 | CG | TYR | A | 80 | 34.484 | 89.044 | 43.675 | 1.00 | 11.18 | A |
| ATOM | 575 | CD1 | TYR | A | 80 | 35.504 | 89.342 | 44.580 | 1.00 | 12.28 | A |
| ATOM | 576 | CE1 | TYR | A | 80 | 36.612 | 90.094 | 44.197 | 1.00 | 13.38 | A |
| ATOM | 577 | CD2 | TYR | A | 80 | 34.593 | 89.531 | 42.374 | 1.00 | 11.57 | A |
| ATOM | 578 | CE2 | TYR | A | 80 | 35.696 | 90.284 | 41.981 | 1.00 | 12.40 | A |
| ATOM | 579 | CZ | TYR | A | 80 | 36.703 | 90.557 | 42.894 | 1.00 | 11.80 | A |
| ATOM | 580 | OH | TYR | A | 80 | 37.815 | 91.267 | 42.500 | 1.00 | 14.10 | A |
| ATOM | 581 | C | TYR | A | 80 | 32.724 | 85.889 | 45.105 | 1.00 | 12.27 | A |
| ATOM | 582 | O | TYR | A | 80 | 31.949 | 86.330 | 45.959 | 1.00 | 12.74 | A |
| ATOM | 583 | N | GLY | A | 81 | 32.650 | 84.650 | 44.625 | 1.00 | 12.12 | A |
| ATOM | 584 | CA | GLY | A | 81 | 31.662 | 83.703 | 45.121 | 1.00 | 13.73 | A |
| ATOM | 585 | C | GLY | A | 81 | 30.223 | 83.897 | 44.685 | 1.00 | 13.22 | A |
| ATOM | 586 | O | GLY | A | 81 | 29.306 | 83.441 | 45.373 | 1.00 | 15.91 | A |
| ATOM | 587 | N | LYS | A | 82 | 30.016 | 84.551 | 43.548 | 1.00 | 12.33 | A |
| ATOM | 588 | CA | LYS | A | 82 | 28.670 | 84.796 | 43.038 | 1.00 | 13.83 | A |
| ATOM | 589 | CB | LYS | A | 82 | 28.671 | 86.099 | 42.232 | 1.00 | 14.46 | A |
| ATOM | 590 | CG | LYS | A | 82 | 29.157 | 87.319 | 43.004 | 1.00 | 18.53 | A |
| ATOM | 591 | CD | LYS | A | 82 | 28.213 | 87.684 | 44.134 | 1.00 | 21.87 | A |
| ATOM | 592 | CE | LYS | A | 82 | 28.698 | 88.929 | 44.864 | 1.00 | 24.28 | A |
| ATOM | 593 | NZ | LYS | A | 82 | 27.763 | 89.348 | 45.947 | 1.00 | 25.39 | A |
| ATOM | 594 | C | LYS | A | 82 | 28.168 | 83.602 | 42.232 | 1.00 | 12.74 | A |
| ATOM | 595 | O | LYS | A | 82 | 26.965 | 83.452 | 42.013 | 1.00 | 14.81 | A |
| ATOM | 596 | N | ALA | A | 83 | 29.096 | 82.763 | 41.780 | 1.00 | 12.25 | A |
| ATOM | 597 | CA | ALA | A | 83 | 28.753 | 81.571 | 41.011 | 1.00 | 11.32 | A |
| ATOM | 598 | CB | ALA | A | 83 | 28.886 | 81.847 | 39.516 | 1.00 | 12.14 | A |
| ATOM | 599 | C | ALA | A | 83 | 29.681 | 80.434 | 41.424 | 1.00 | 10.84 | A |
| ATOM | 600 | O | ALA | A | 83 | 30.773 | 80.679 | 41.941 | 1.00 | 10.45 | A |
| ATOM | 601 | N | ASP | A | 84 | 29.244 | 79.197 | 41.195 | 1.00 | 10.99 | A |
| ATOM | 602 | CA | ASP | A | 84 | 30.026 | 78.021 | 41.562 | 1.00 | 10.42 | A |
| ATOM | 603 | CB | ASP | A | 84 | 29.091 | 76.929 | 42.093 | 1.00 | 12.81 | A |
| ATOM | 604 | CG | ASP | A | 84 | 28.252 | 77.389 | 43.264 | 1.00 | 14.10 | A |
| ATOM | 605 | OD1 | ASP | A | 84 | 28.829 | 77.871 | 44.261 | 1.00 | 18.48 | A |
| ATOM | 606 | OD2 | ASP | A | 84 | 27.014 | 77.258 | 43.186 | 1.00 | 15.34 | A |
| ATOM | 607 | C | ASP | A | 84 | 30.873 | 77.493 | 40.420 | 1.00 | 10.51 | A |
| ATOM | 608 | O | ASP | A | 84 | 31.906 | 76.864 | 40.648 | 1.00 | 10.54 | A |
| ATOM | 609 | N | ILE | A | 85 | 30.433 | 77.735 | 39.192 | 1.00 | 9.58 | A |
| ATOM | 610 | CA | ILE | A | 85 | 31.148 | 77.224 | 38.031 | 1.00 | 10.06 | A |
| ATOM | 611 | CB | ILE | A | 85 | 30.740 | 75.740 | 37.802 | 1.00 | 10.69 | A |
| ATOM | 612 | CG2 | ILE | A | 85 | 29.253 | 75.664 | 37.530 | 1.00 | 12.85 | A |
| ATOM | 613 | CG1 | ILE | A | 85 | 31.559 | 75.099 | 36.679 | 1.00 | 10.70 | A |
| ATOM | 614 | CD1 | ILE | A | 85 | 31.458 | 73.580 | 36.685 | 1.00 | 14.57 | A |
| ATOM | 615 | C | ILE | A | 85 | 30.812 | 78.062 | 36.802 | 1.00 | 9.88 | A |
| ATOM | 616 | O | ILE | A | 85 | 29.707 | 78.594 | 36.694 | 1.00 | 10.58 | A |
| ATOM | 617 | N | ALA | A | 86 | 31.776 | 78.201 | 35.895 | 1.00 | 8.99 | A |
| ATOM | 618 | CA | ALA | A | 86 | 31.569 | 78.949 | 34.659 | 1.00 | 8.68 | A |
| ATOM | 619 | CB | ALA | A | 86 | 32.602 | 80.066 | 34.521 | 1.00 | 8.41 | A |
| ATOM | 620 | C | ALA | A | 86 | 31.713 | 77.961 | 33.512 | 1.00 | 8.34 | A |
| ATOM | 621 | O | ALA | A | 86 | 32.723 | 77.261 | 33.414 | 1.00 | 8.88 | A |
| ATOM | 622 | N | ILE | A | 87 | 30.691 | 77.885 | 32.667 | 1.00 | 8.85 | A |
| ATOM | 623 | CA | ILE | A | 87 | 30.692 | 76.986 | 31.518 | 1.00 | 8.53 | A |
| ATOM | 624 | CB | ILE | A | 87 | 29.648 | 75.857 | 31.693 | 1.00 | 10.55 | A |
| ATOM | 625 | CG2 | ILE | A | 87 | 29.684 | 74.918 | 30.490 | 1.00 | 10.47 | A |
| ATOM | 626 | CG1 | ILE | A | 87 | 29.954 | 75.079 | 32.978 | 1.00 | 9.16 | A |
| ATOM | 627 | CD1 | ILE | A | 87 | 28.994 | 73.948 | 33.269 | 1.00 | 11.00 | A |
| ATOM | 628 | C | ILE | A | 87 | 30.371 | 77.856 | 30.311 | 1.00 | 9.03 | A |
| ATOM | 629 | O | ILE | A | 87 | 29.214 | 78.208 | 30.060 | 1.00 | 9.01 | A |
| ATOM | 630 | N | ALA | A | 88 | 31.421 | 78.206 | 29.576 | 1.00 | 8.95 | A |
| ATOM | 631 | CA | ALA | A | 88 | 31.309 | 79.098 | 28.433 | 1.00 | 8.86 | A |
| ATOM | 632 | CB | ALA | A | 88 | 31.149 | 80.523 | 28.948 | 1.00 | 11.38 | A |
| ATOM | 633 | C | ALA | A | 88 | 32.589 | 78.994 | 27.609 | 1.00 | 8.27 | A |
| ATOM | 634 | O | ALA | A | 88 | 33.492 | 78.240 | 27.960 | 1.00 | 9.26 | A |
| ATOM | 635 | N | PRO | A | 89 | 32.668 | 79.730 | 26.485 | 1.00 | 9.01 | A |
| ATOM | 636 | CD | PRO | A | 89 | 31.561 | 80.392 | 25.770 | 1.00 | 9.13 | A |
| ATOM | 637 | CA | PRO | A | 89 | 33.883 | 79.688 | 25.659 | 1.00 | 8.61 | A |
| ATOM | 638 | CB | PRO | A | 89 | 33.378 | 80.165 | 24.294 | 1.00 | 8.98 | A |
| ATOM | 639 | CG | PRO | A | 89 | 32.312 | 81.179 | 24.635 | 1.00 | 9.79 | A |
| ATOM | 640 | C | PRO | A | 89 | 34.921 | 80.580 | 26.345 | 1.00 | 7.75 | A |
| ATOM | 641 | O | PRO | A | 89 | 35.333 | 81.614 | 25.812 | 1.00 | 9.37 | A |
| ATOM | 642 | N | LEU | A | 90 | 35.324 | 80.169 | 27.544 | 1.00 | 8.01 | A |
| ATOM | 643 | CA | LEU | A | 90 | 36.275 | 80.911 | 28.362 | 1.00 | 8.41 | A |
| ATOM | 644 | CB | LEU | A | 90 | 35.867 | 80.769 | 29.833 | 1.00 | 9.56 | A |
| ATOM | 645 | CG | LEU | A | 90 | 36.698 | 81.544 | 30.860 | 1.00 | 7.48 | A |
| ATOM | 646 | CD1 | LEU | A | 90 | 36.513 | 83.048 | 30.672 | 1.00 | 8.55 | A |
| ATOM | 647 | CD2 | LEU | A | 90 | 36.265 | 81.138 | 32.258 | 1.00 | 9.99 | A |
| ATOM | 648 | C | LEU | A | 90 | 37.679 | 80.408 | 28.084 | 1.00 | 8.10 | A |
| ATOM | 649 | O | LEU | A | 90 | 38.040 | 79.293 | 28.460 | 1.00 | 8.62 | A |
| ATOM | 650 | N | THR | A | 91 | 38.483 | 81.250 | 27.452 | 1.00 | 7.27 | A |
| ATOM | 651 | CA | THR | A | 91 | 39.836 | 80.876 | 27.092 | 1.00 | 8.12 | A |
| ATOM | 652 | CB | THR | A | 91 | 40.400 | 81.907 | 26.114 | 1.00 | 9.08 | A |
| ATOM | 653 | OG1 | THR | A | 91 | 39.507 | 82.005 | 24.999 | 1.00 | 9.56 | A |
| ATOM | 654 | CG2 | THR | A | 91 | 41.785 | 81.497 | 25.622 | 1.00 | 10.14 | A |
| ATOM | 655 | C | THR | A | 91 | 40.745 | 80.732 | 28.300 | 1.00 | 7.70 | A |
| ATOM | 656 | O | THR | A | 91 | 40.746 | 81.572 | 29.199 | 1.00 | 8.49 | A |
| ATOM | 657 | N | ILE | A | 92 | 41.505 | 79.645 | 28.324 | 1.00 | 8.89 | A |
| ATOM | 658 | CA | ILE | A | 92 | 42.436 | 79.391 | 29.409 | 1.00 | 9.54 | A |
| ATOM | 659 | CB | ILE | A | 92 | 42.883 | 77.904 | 29.418 | 1.00 | 8.47 | A |
| ATOM | 660 | CG2 | ILE | A | 92 | 43.970 | 77.690 | 30.460 | 1.00 | 9.72 | A |
| ATOM | 661 | CG1 | ILE | A | 92 | 41.681 | 77.000 | 29.699 | 1.00 | 8.90 | A |
| ATOM | 662 | CD1 | ILE | A | 92 | 41.977 | 75.524 | 29.544 | 1.00 | 11.06 | A |
| ATOM | 663 | C | ILE | A | 92 | 43.652 | 80.290 | 29.200 | 1.00 | 10.70 | A |
| ATOM | 664 | O | ILE | A | 92 | 44.369 | 80.160 | 28.203 | 1.00 | 11.28 | A |
| ATOM | 665 | N | THR | A | 93 | 43.860 | 81.221 | 30.126 | 1.00 | 9.30 | A |
| ATOM | 666 | CA | THR | A | 93 | 44.995 | 82.135 | 30.053 | 1.00 | 10.20 | A |
| ATOM | 667 | CB | THR | A | 93 | 44.561 | 83.577 | 29.717 | 1.00 | 10.42 | A |
| ATOM | 668 | OG1 | THR | A | 93 | 43.804 | 84.120 | 30.807 | 1.00 | 11.85 | A |
| ATOM | 669 | CG2 | THR | A | 93 | 43.720 | 83.602 | 28.448 | 1.00 | 10.24 | A |
| ATOM | 670 | C | THR | A | 93 | 45.709 | 82.156 | 31.394 | 1.00 | 10.29 | A |
| ATOM | 671 | O | THR | A | 93 | 45.131 | 81.803 | 32.423 | 1.00 | 11.44 | A |
| ATOM | 672 | N | LEU | A | 94 | 46.965 | 82.586 | 31.376 | 1.00 | 10.68 | A |
| ATOM | 673 | CA | LEU | A | 94 | 47.771 | 82.644 | 32.583 | 1.00 | 10.99 | A |
| ATOM | 674 | CB | LEU | A | 94 | 49.185 | 83.111 | 32.223 | 1.00 | 11.64 | A |
| ATOM | 675 | CG | LEU | A | 94 | 50.083 | 83.424 | 33.423 | 1.00 | 13.09 | A |
| ATOM | 676 | CD1 | LEU | A | 94 | 50.364 | 82.151 | 34.203 | 1.00 | 14.45 | A |
| ATOM | 677 | CD2 | LEU | A | 94 | 51.380 | 84.051 | 32.935 | 1.00 | 16.24 | A |
| ATOM | 678 | C | LEU | A | 94 | 47.148 | 83.501 | 33.677 | 1.00 | 10.53 | A |
| ATOM | 679 | O | LEU | A | 94 | 47.025 | 83.055 | 34.812 | 1.00 | 10.79 | A |
| ATOM | 680 | N | VAL | A | 95 | 46.768 | 84.730 | 33.345 | 1.00 | 11.04 | A |
| ATOM | 681 | CA | VAL | A | 95 | 46.202 | 85.610 | 34.360 | 1.00 | 11.98 | A |
| ATOM | 682 | CB | VAL | A | 95 | 45.950 | 87.038 | 33.807 | 1.00 | 12.84 | A |
| ATOM | 683 | CG1 | VAL | A | 95 | 47.273 | 87.682 | 33.419 | 1.00 | 17.48 | A |
| ATOM | 684 | CG2 | VAL | A | 95 | 45.006 | 86.993 | 32.625 | 1.00 | 13.38 | A |
| ATOM | 685 | C | VAL | A | 95 | 44.927 | 85.059 | 34.991 | 1.00 | 10.75 | A |
| ATOM | 686 | O | VAL | A | 95 | 44.688 | 85.262 | 36.181 | 1.00 | 12.45 | A |
| ATOM | 687 | N | ARG | A | 96 | 44.119 | 84.350 | 34.206 | 1.00 | 10.25 | A |
| ATOM | 688 | CA | ARG | A | 96 | 42.890 | 83.764 | 34.734 | 1.00 | 10.47 | A |
| ATOM | 689 | CB | ARG | A | 96 | 41.944 | 83.429 | 33.575 | 1.00 | 9.65 | A |
| ATOM | 690 | CG | ARG | A | 96 | 41.356 | 84.636 | 32.883 | 1.00 | 10.28 | A |
| ATOM | 691 | CD | ARG | A | 96 | 40.559 | 84.214 | 31.668 | 1.00 | 9.89 | A |
| ATOM | 692 | NE | ARG | A | 96 | 39.948 | 85.350 | 30.993 | 1.00 | 8.57 | A |
| ATOM | 693 | CZ | ARG | A | 96 | 39.434 | 85.296 | 29.772 | 1.00 | 8.23 | A |
| ATOM | 694 | NH1 | ARG | A | 96 | 39.458 | 84.160 | 29.087 | 1.00 | 9.93 | A |
| ATOM | 695 | NH2 | ARG | A | 96 | 38.897 | 86.380 | 29.234 | 1.00 | 8.91 | A |
| ATOM | 696 | C | ARG | A | 96 | 43.231 | 82.551 | 35.596 | 1.00 | 9.78 | A |
| ATOM | 697 | O | ARG | A | 96 | 42.658 | 82.357 | 36.668 | 1.00 | 11.31 | A |
| ATOM | 698 | N | GLU | A | 97 | 44.172 | 81.740 | 35.122 | 1.00 | 10.06 | A |
| ATOM | 699 | CA | GLU | A | 97 | 44.589 | 80.539 | 35.841 | 1.00 | 9.63 | A |
| ATOM | 700 | CB | GLU | A | 97 | 45.602 | 79.771 | 34.984 | 1.00 | 10.82 | A |
| ATOM | 701 | CG | GLU | A | 97 | 46.124 | 78.475 | 35.602 | 1.00 | 12.19 | A |
| ATOM | 702 | CD | GLU | A | 97 | 45.075 | 77.380 | 35.668 | 1.00 | 13.87 | A |
| ATOM | 703 | OE1 | GLU | A | 97 | 44.169 | 77.468 | 36.522 | 1.00 | 13.02 | A |
| ATOM | 704 | OE2 | GLU | A | 97 | 45.156 | 76.429 | 34.859 | 1.00 | 13.68 | A |
| ATOM | 705 | C | GLU | A | 97 | 45.140 | 80.882 | 37.217 | 1.00 | 10.77 | A |
| ATOM | 706 | O | GLU | A | 97 | 45.077 | 80.073 | 38.137 | 1.00 | 11.69 | A |
| ATOM | 707 | N | GLU | A | 98 | 45.691 | 82.084 | 37.349 | 1.00 | 11.36 | A |
| ATOM | 708 | CA | GLU | A | 98 | 46.266 | 82.522 | 38.612 | 1.00 | 11.91 | A |
| ATOM | 709 | CB | GLU | A | 98 | 47.142 | 83.755 | 38.352 | 1.00 | 13.62 | A |
| ATOM | 710 | CG | GLU | A | 98 | 48.450 | 83.437 | 37.640 | 1.00 | 15.61 | A |
| ATOM | 711 | CD | GLU | A | 98 | 49.211 | 84.675 | 37.211 | 1.00 | 17.68 | A |
| ATOM | 712 | OE1 | GLU | A | 98 | 50.372 | 84.531 | 36.771 | 1.00 | 19.08 | A |
| ATOM | 713 | OE2 | GLU | A | 98 | 48.655 | 85.790 | 37.302 | 1.00 | 18.26 | A |
| ATOM | 714 | C | GLU | A | 98 | 45.223 | 82.763 | 39.697 | 1.00 | 12.62 | A |
| ATOM | 715 | O | GLU | A | 98 | 45.542 | 82.687 | 40.887 | 1.00 | 12.91 | A |
| ATOM | 716 | N | VAL | A | 99 | 43.980 | 83.034 | 39.303 | 1.00 | 11.67 | A |
| ATOM | 717 | CA | VAL | A | 99 | 42.941 | 83.321 | 40.287 | 1.00 | 10.90 | A |
| ATOM | 718 | CB | VAL | A | 99 | 42.364 | 84.740 | 40.071 | 1.00 | 11.56 | A |
| ATOM | 719 | CG1 | VAL | A | 99 | 43.480 | 85.766 | 40.189 | 1.00 | 13.71 | A |
| ATOM | 720 | CG2 | VAL | A | 99 | 41.682 | 84.836 | 38.714 | 1.00 | 11.03 | A |
| ATOM | 721 | C | VAL | A | 99 | 41.795 | 82.318 | 40.405 | 1.00 | 11.51 | A |
| ATOM | 722 | O | VAL | A | 99 | 41.082 | 82.320 | 41.405 | 1.00 | 11.13 | A |
| ATOM | 723 | N | ILE | A | 100 | 41.595 | 81.481 | 39.393 | 1.00 | 10.46 | A |
| ATOM | 724 | CA | ILE | A | 100 | 40.552 | 80.457 | 39.464 | 1.00 | 9.90 | A |
| ATOM | 725 | CB | ILE | A | 100 | 39.286 | 80.834 | 38.631 | 1.00 | 9.71 | A |
| ATOM | 726 | CG2 | ILE | A | 100 | 38.515 | 81.951 | 39.338 | 1.00 | 10.50 | A |
| ATOM | 727 | CG1 | ILE | A | 100 | 39.667 | 81.254 | 37.204 | 1.00 | 11.01 | A |
| ATOM | 728 | CD1 | ILE | A | 100 | 40.105 | 80.117 | 36.296 | 1.00 | 12.75 | A |
| ATOM | 729 | C | ILE | A | 100 | 41.116 | 79.136 | 38.959 | 1.00 | 10.00 | A |
| ATOM | 730 | O | ILE | A | 100 | 42.239 | 79.101 | 38.456 | 1.00 | 10.35 | A |
| ATOM | 731 | N | ASP | A | 101 | 40.359 | 78.053 | 39.131 | 1.00 | 10.22 | A |
| ATOM | 732 | CA | ASP | A | 101 | 40.775 | 76.727 | 38.661 | 1.00 | 11.33 | A |
| ATOM | 733 | CB | ASP | A | 101 | 40.368 | 75.639 | 39.672 | 1.00 | 12.26 | A |
| ATOM | 734 | CG | ASP | A | 101 | 41.200 | 75.651 | 40.937 | 1.00 | 12.57 | A |
| ATOM | 735 | OD1 | ASP | A | 101 | 42.299 | 76.244 | 40.944 | 1.00 | 15.62 | A |
| ATOM | 736 | OD2 | ASP | A | 101 | 40.747 | 75.036 | 41.927 | 1.00 | 16.24 | A |
| ATOM | 737 | C | ASP | A | 101 | 40.144 | 76.460 | 37.294 | 1.00 | 11.64 | A |
| ATOM | 738 | O | ASP | A | 101 | 38.968 | 76.761 | 37.083 | 1.00 | 13.13 | A |
| ATOM | 739 | N | PHE | A | 102 | 40.927 | 75.894 | 36.377 | 1.00 | 11.49 | A |
| ATOM | 740 | CA | PHE | A | 102 | 40.457 | 75.544 | 35.035 | 1.00 | 11.37 | A |
| ATOM | 741 | CB | PHE | A | 102 | 41.356 | 76.177 | 33.965 | 1.00 | 10.36 | A |
| ATOM | 742 | CG | PHE | A | 102 | 40.941 | 77.549 | 33.515 | 1.00 | 11.81 | A |
| ATOM | 743 | CD1 | PHE | A | 102 | 39.781 | 77.742 | 32.964 | 1.00 | 9.78 | A |
| ATOM | 744 | CD2 | PHE | A | 102 | 41.785 | 78.523 | 33.604 | 1.00 | 11.62 | A |
| ATOM | 745 | CE1 | PHE | A | 102 | 39.408 | 78.988 | 32.442 | 1.00 | 9.57 | A |
| ATOM | 746 | CE2 | PHE | A | 102 | 41.505 | 79.800 | 33.114 | 1.00 | 11.74 | A |
| ATOM | 747 | CZ | PHE | A | 102 | 40.292 | 80.033 | 32.520 | 1.00 | 10.93 | A |
| ATOM | 748 | C | PHE | A | 102 | 40.489 | 74.029 | 34.880 | 1.00 | 11.55 | A |
| ATOM | 749 | O | PHE | A | 102 | 41.405 | 73.366 | 35.369 | 1.00 | 12.64 | A |
| ATOM | 750 | N | SER | A | 103 | 39.497 | 73.487 | 34.188 | 1.00 | 10.67 | A |
| ATOM | 751 | CA | SER | A | 103 | 39.470 | 72.064 | 33.895 | 1.00 | 11.23 | A |
| ATOM | 752 | CB | SER | A | 103 | 38.057 | 71.657 | 33.485 | 1.00 | 11.53 | A |
| ATOM | 753 | OG | SER | A | 103 | 37.750 | 72.193 | 32.198 | 1.00 | 11.77 | A |
| ATOM | 754 | C | SER | A | 103 | 40.421 | 71.950 | 32.712 | 1.00 | 11.41 | A |
| ATOM | 755 | O | SER | A | 103 | 40.866 | 72.960 | 32.167 | 1.00 | 12.01 | A |
| ATOM | 756 | N | LYS | A | 104 | 40.737 | 70.723 | 32.314 | 1.00 | 12.11 | A |
| ATOM | 757 | CA | LYS | A | 104 | 41.548 | 70.528 | 31.127 | 1.00 | 13.10 | A |
| ATOM | 758 | CB | LYS | A | 104 | 41.866 | 69.037 | 30.969 | 1.00 | 14.27 | A |
| ATOM | 759 | CG | LYS | A | 104 | 42.804 | 68.479 | 32.033 | 1.00 | 16.73 | A |
| ATOM | 760 | CD | LYS | A | 104 | 44.135 | 69.216 | 32.026 | 1.00 | 16.89 | A |
| ATOM | 761 | CE | LYS | A | 104 | 45.140 | 68.607 | 33.008 | 1.00 | 16.32 | A |
| ATOM | 762 | NZ | LYS | A | 104 | 44.760 | 68.757 | 34.448 | 1.00 | 15.33 | A |
| ATOM | 763 | C | LYS | A | 104 | 40.632 | 71.099 | 30.037 | 1.00 | 11.89 | A |
| ATOM | 764 | O | LYS | A | 104 | 39.415 | 71.156 | 30.212 | 1.00 | 11.44 | A |
| ATOM | 765 | N | PRO | A | 105 | 41.191 | 71.519 | 28.899 | 1.00 | 11.77 | A |
| ATOM | 766 | CD | PRO | A | 105 | 42.611 | 71.548 | 28.522 | 1.00 | 13.43 | A |
| ATOM | 767 | CA | PRO | A | 105 | 40.350 | 72.085 | 27.839 | 1.00 | 12.57 | A |
| ATOM | 768 | CB | PRO | A | 105 | 41.374 | 72.559 | 26.806 | 1.00 | 14.53 | A |
| ATOM | 769 | CG | PRO | A | 105 | 42.655 | 72.794 | 27.577 | 1.00 | 16.88 | A |
| ATOM | 770 | C | PRO | A | 105 | 39.288 | 71.146 | 27.281 | 1.00 | 9.98 | A |
| ATOM | 771 | O | PRO | A | 105 | 39.530 | 69.954 | 27.108 | 1.00 | 10.01 | A |
| ATOM | 772 | N | PHE | A | 106 | 38.111 | 71.696 | 26.993 | 1.00 | 9.32 | A |
| ATOM | 773 | CA | PHE | A | 106 | 37.035 | 70.899 | 26.423 | 1.00 | 9.75 | A |
| ATOM | 774 | CB | PHE | A | 106 | 35.718 | 71.162 | 27.177 | 1.00 | 8.88 | A |
| ATOM | 775 | CG | PHE | A | 106 | 35.210 | 72.583 | 27.087 | 1.00 | 8.44 | A |
| ATOM | 776 | CD1 | PHE | A | 106 | 34.605 | 73.001 | 26.013 | 1.00 | 8.87 | A |
| ATOM | 777 | CD2 | PHE | A | 106 | 35.328 | 73.370 | 28.111 | 1.00 | 9.33 | A |
| ATOM | 778 | CE1 | PHE | A | 106 | 34.055 | 74.295 | 25.921 | 1.00 | 8.35 | A |
| ATOM | 779 | CE2 | PHE | A | 106 | 34.813 | 74.677 | 28.115 | 1.00 | 9.88 | A |
| ATOM | 780 | CZ | PHE | A | 106 | 34.165 | 75.142 | 26.999 | 1.00 | 8.63 | A |
| ATOM | 781 | C | PHE | A | 106 | 36.935 | 71.147 | 24.926 | 1.00 | 8.06 | A |
| ATOM | 782 | O | PHE | A | 106 | 36.241 | 70.422 | 24.222 | 1.00 | 9.73 | A |
| ATOM | 783 | N | MET | A | 107 | 37.652 | 72.159 | 24.439 | 1.00 | 8.72 | A |
| ATOM | 784 | CA | MET | A | 107 | 37.636 | 72.506 | 23.021 | 1.00 | 9.16 | A |
| ATOM | 785 | CB | MET | A | 107 | 36.377 | 73.331 | 22.711 | 1.00 | 12.85 | A |
| ATOM | 786 | CG | MET | A | 107 | 36.224 | 73.735 | 21.245 | 1.00 | 12.90 | A |
| ATOM | 787 | SD | MET | A | 107 | 34.755 | 74.745 | 20.904 | 1.00 | 15.47 | A |
| ATOM | 788 | CE | MET | A | 107 | 33.499 | 73.506 | 20.828 | 1.00 | 15.97 | A |
| ATOM | 789 | C | MET | A | 107 | 38.916 | 73.255 | 22.683 | 1.00 | 9.96 | A |
| ATOM | 790 | O | MET | A | 107 | 39.394 | 74.071 | 23.474 | 1.00 | 8.85 | A |
| ATOM | 791 | N | SER | A | 108 | 39.463 | 72.978 | 21.506 | 1.00 | 8.50 | A |
| ATOM | 792 | CA | SER | A | 108 | 40.679 | 73.639 | 21.056 | 1.00 | 9.44 | A |
| ATOM | 793 | CB | SER | A | 108 | 41.524 | 72.639 | 20.273 | 1.00 | 11.24 | A |
| ATOM | 794 | OG | SER | A | 108 | 40.862 | 72.290 | 19.072 | 1.00 | 14.99 | A |
| ATOM | 795 | C | SER | A | 108 | 40.332 | 74.858 | 20.212 | 1.00 | 10.05 | A |
| ATOM | 796 | O | SER | A | 108 | 39.270 | 74.929 | 19.593 | 1.00 | 10.23 | A |
| ATOM | 797 | N | LEU | A | 109 | 41.244 | 75.819 | 20.189 | 1.00 | 11.63 | A |
| ATOM | 798 | CA | LEU | A | 109 | 41.057 | 77.008 | 19.376 | 1.00 | 9.32 | A |
| ATOM | 799 | CB | LEU | A | 109 | 39.908 | 77.859 | 19.924 | 1.00 | 11.66 | A |
| ATOM | 800 | CG | LEU | A | 109 | 40.249 | 78.706 | 21.148 | 1.00 | 13.88 | A |
| ATOM | 801 | CD1 | LEU | A | 109 | 39.330 | 79.886 | 21.191 | 1.00 | 19.27 | A |
| ATOM | 802 | CD2 | LEU | A | 109 | 40.156 | 77.885 | 22.415 | 1.00 | 14.66 | A |
| ATOM | 803 | C | LEU | A | 109 | 42.353 | 77.786 | 19.313 | 1.00 | 9.33 | A |
| ATOM | 804 | O | LEU | A | 109 | 43.278 | 77.553 | 20.087 | 1.00 | 9.96 | A |
| ATOM | 805 | N | GLY | A | 110 | 42.396 | 78.715 | 18.370 | 1.00 | 8.83 | A |
| ATOM | 806 | CA | GLY | A | 110 | 43.541 | 79.582 | 18.207 | 1.00 | 8.86 | A |
| ATOM | 807 | C | GLY | A | 110 | 43.043 | 80.860 | 17.570 | 1.00 | 9.39 | A |
| ATOM | 808 | O | GLY | A | 110 | 42.017 | 80.852 | 16.890 | 1.00 | 9.15 | A |
| ATOM | 809 | N | ILE | A | 111 | 43.740 | 81.969 | 17.802 | 1.00 | 9.81 | A |
| ATOM | 810 | CA | ILE | A | 111 | 43.344 | 83.226 | 17.186 | 1.00 | 10.07 | A |
| ATOM | 811 | CB | ILE | A | 111 | 44.243 | 84.383 | 17.669 | 1.00 | 9.38 | A |
| ATOM | 812 | CG2 | ILE | A | 111 | 43.991 | 85.640 | 16.840 | 1.00 | 11.77 | A |
| ATOM | 813 | CG1 | ILE | A | 111 | 43.964 | 84.639 | 19.151 | 1.00 | 10.08 | A |
| ATOM | 814 | CD1 | ILE | A | 111 | 44.944 | 85.579 | 19.802 | 1.00 | 11.40 | A |
| ATOM | 815 | C | ILE | A | 111 | 43.483 | 82.993 | 15.684 | 1.00 | 8.75 | A |
| ATOM | 816 | O | ILE | A | 111 | 44.429 | 82.340 | 15.231 | 1.00 | 9.13 | A |
| ATOM | 817 | N | SER | A | 112 | 42.531 | 83.512 | 14.918 | 1.00 | 8.66 | A |
| ATOM | 818 | CA | SER | A | 112 | 42.510 | 83.305 | 13.479 | 1.00 | 9.16 | A |
| ATOM | 819 | CB | SER | A | 112 | 41.622 | 82.096 | 13.175 | 1.00 | 8.16 | A |
| ATOM | 820 | OG | SER | A | 112 | 40.264 | 82.396 | 13.473 | 1.00 | 10.60 | A |
| ATOM | 821 | C | SER | A | 112 | 42.036 | 84.546 | 12.750 | 1.00 | 9.12 | A |
| ATOM | 822 | O | SER | A | 112 | 41.534 | 85.492 | 13.356 | 1.00 | 9.91 | A |
| ATOM | 823 | N | ILE | A | 113 | 42.165 | 84.510 | 11.430 | 1.00 | 10.69 | A |
| ATOM | 824 | CA | ILE | A | 113 | 41.763 | 85.622 | 10.583 | 1.00 | 11.17 | A |
| ATOM | 825 | CB | ILE | A | 113 | 42.876 | 85.965 | 9.568 | 1.00 | 11.13 | A |
| ATOM | 826 | CG2 | ILE | A | 113 | 42.430 | 87.120 | 8.676 | 1.00 | 12.54 | A |
| ATOM | 827 | CG1 | ILE | A | 113 | 44.168 | 86.320 | 10.304 | 1.00 | 10.82 | A |
| ATOM | 828 | CD1 | ILE | A | 113 | 45.405 | 86.307 | 9.420 | 1.00 | 10.07 | A |
| ATOM | 829 | C | ILE | A | 113 | 40.495 | 85.307 | 9.791 | 1.00 | 10.13 | A |
| ATOM | 830 | O | ILE | A | 113 | 40.408 | 84.267 | 9.136 | 1.00 | 10.81 | A |
| ATOM | 831 | N | MET | A | 114 | 39.512 | 86.200 | 9.872 | 1.00 | 10.04 | A |
| ATOM | 832 | CA | MET | A | 114 | 38.286 | 86.053 | 9.099 | 1.00 | 10.61 | A |
| ATOM | 833 | CB | MET | A | 114 | 37.045 | 86.178 | 9.988 | 1.00 | 10.16 | A |
| ATOM | 834 | CG | MET | A | 114 | 35.760 | 86.220 | 9.157 | 1.00 | 10.60 | A |
| ATOM | 835 | SD | MET | A | 114 | 34.283 | 86.713 | 10.057 | 1.00 | 12.18 | A |
| ATOM | 836 | CE | MET | A | 114 | 34.047 | 85.278 | 11.121 | 1.00 | 13.11 | A |
| ATOM | 837 | C | MET | A | 114 | 38.310 | 87.142 | 8.038 | 1.00 | 11.63 | A |
| ATOM | 838 | O | MET | A | 114 | 38.488 | 88.320 | 8.356 | 1.00 | 10.82 | A |
| ATOM | 839 | N | ILE | A | 115 | 38.133 | 86.749 | 6.782 | 1.00 | 11.48 | A |
| ATOM | 840 | CA | ILE | A | 115 | 38.105 | 87.705 | 5.687 | 1.00 | 11.96 | A |
| ATOM | 841 | CB | ILE | A | 115 | 39.272 | 87.499 | 4.685 | 1.00 | 11.71 | A |
| ATOM | 842 | CG2 | ILE | A | 115 | 40.595 | 87.827 | 5.340 | 1.00 | 13.51 | A |
| ATOM | 843 | CG1 | ILE | A | 115 | 39.241 | 86.067 | 4.140 | 1.00 | 12.50 | A |
| ATOM | 844 | CD1 | ILE | A | 115 | 40.170 | 85.830 | 2.954 | 1.00 | 16.93 | A |
| ATOM | 845 | C | ILE | A | 115 | 36.825 | 87.521 | 4.894 | 1.00 | 11.78 | A |
| ATOM | 846 | O | ILE | A | 115 | 36.151 | 86.495 | 5.001 | 1.00 | 11.27 | A |
| ATOM | 847 | N | LYS | A | 116 | 36.494 | 88.541 | 4.112 | 1.00 | 13.14 | A |
| ATOM | 848 | CA | LYS | A | 116 | 35.351 | 88.500 | 3.220 | 1.00 | 12.56 | A |
| ATOM | 849 | CB | LYS | A | 116 | 34.941 | 89.936 | 2.880 | 1.00 | 13.86 | A |
| ATOM | 850 | CG | LYS | A | 116 | 33.922 | 90.095 | 1.758 | 1.00 | 18.08 | A |
| ATOM | 851 | CD | LYS | A | 116 | 32.539 | 89.615 | 2.142 | 1.00 | 20.52 | A |
| ATOM | 852 | CE | LYS | A | 116 | 31.476 | 90.375 | 1.351 | 1.00 | 21.38 | A |
| ATOM | 853 | NZ | LYS | A | 116 | 31.798 | 90.486 | −0.103 | 1.00 | 22.41 | A |
| ATOM | 854 | C | LYS | A | 116 | 35.954 | 87.735 | 2.046 | 1.00 | 12.06 | A |
| ATOM | 855 | O | LYS | A | 116 | 37.093 | 88.000 | 1.662 | 1.00 | 12.42 | A |
| ATOM | 856 | N | LYS | A | 117 | 35.221 | 86.780 | 1.488 | 1.00 | 12.31 | A |
| ATOM | 857 | CA | LYS | A | 117 | 35.751 | 86.009 | 0.366 | 1.00 | 12.17 | A |
| ATOM | 858 | CB | LYS | A | 117 | 34.659 | 85.090 | −0.188 | 1.00 | 13.23 | A |
| ATOM | 859 | CG | LYS | A | 117 | 34.338 | 83.905 | 0.701 | 1.00 | 13.86 | A |
| ATOM | 860 | CD | LYS | A | 117 | 33.367 | 82.955 | 0.020 | 1.00 | 13.48 | A |
| ATOM | 861 | CE | LYS | A | 117 | 33.108 | 81.727 | 0.879 | 1.00 | 15.63 | A |
| ATOM | 862 | NZ | LYS | A | 117 | 32.169 | 80.780 | 0.215 | 1.00 | 16.49 | A |
| ATOM | 863 | C | LYS | A | 117 | 36.341 | 86.924 | −0.697 | 1.00 | 11.97 | A |
| ATOM | 864 | O | LYS | A | 117 | 35.742 | 87.938 | −1.063 | 1.00 | 12.59 | A |
| ATOM | 865 | N | GLY | A | 118 | 37.525 | 86.565 | −1.183 | 1.00 | 13.45 | A |
| ATOM | 866 | CA | GLY | A | 118 | 38.167 | 87.359 | −2.216 | 1.00 | 13.64 | A |
| ATOM | 867 | C | GLY | A | 118 | 39.234 | 88.317 | −1.721 | 1.00 | 14.42 | A |
| ATOM | 868 | O | GLY | A | 118 | 40.003 | 88.860 | −2.515 | 1.00 | 14.72 | A |
| ATOM | 869 | N | THR | A | 119 | 39.287 | 88.532 | −0.410 | 1.00 | 13.34 | A |
| ATOM | 870 | CA | THR | A | 119 | 40.283 | 89.429 | 0.171 | 1.00 | 13.21 | A |
| ATOM | 871 | CB | THR | A | 119 | 40.025 | 89.633 | 1.676 | 1.00 | 13.24 | A |
| ATOM | 872 | OG1 | THR | A | 119 | 38.732 | 90.220 | 1.858 | 1.00 | 14.61 | A |
| ATOM | 873 | CG2 | THR | A | 119 | 41.075 | 90.551 | 2.279 | 1.00 | 14.14 | A |
| ATOM | 874 | C | THR | A | 119 | 41.684 | 88.845 | −0.032 | 1.00 | 13.53 | A |
| ATOM | 875 | O | THR | A | 119 | 41.934 | 87.685 | 0.292 | 1.00 | 13.93 | A |
| ATOM | 876 | N | PRO | A | 120 | 42.619 | 89.649 | −0.569 | 1.00 | 14.13 | A |
| ATOM | 877 | CD | PRO | A | 120 | 42.403 | 90.986 | −1.148 | 1.00 | 14.29 | A |
| ATOM | 878 | CA | PRO | A | 120 | 43.991 | 89.195 | −0.813 | 1.00 | 15.21 | A |
| ATOM | 879 | CB | PRO | A | 120 | 44.466 | 90.166 | −1.893 | 1.00 | 16.18 | A |
| ATOM | 880 | CG | PRO | A | 120 | 43.871 | 91.469 | −1.451 | 1.00 | 15.91 | A |
| ATOM | 881 | C | PRO | A | 120 | 44.856 | 89.133 | 0.442 | 1.00 | 14.83 | A |
| ATOM | 882 | O | PRO | A | 120 | 45.859 | 89.842 | 0.557 | 1.00 | 16.28 | A |
| ATOM | 883 | N | ILE | A | 121 | 44.461 | 88.269 | 1.373 | 1.00 | 15.05 | A |
| ATOM | 884 | CA | ILE | A | 121 | 45.173 | 88.072 | 2.634 | 1.00 | 14.62 | A |
| ATOM | 885 | CB | ILE | A | 121 | 44.468 | 88.816 | 3.801 | 1.00 | 14.17 | A |
| ATOM | 886 | CG2 | ILE | A | 121 | 45.026 | 88.343 | 5.135 | 1.00 | 13.82 | A |
| ATOM | 887 | CG1 | ILE | A | 121 | 44.628 | 90.330 | 3.643 | 1.00 | 14.82 | A |
| ATOM | 888 | CD1 | ILE | A | 121 | 46.058 | 90.828 | 3.780 | 1.00 | 14.64 | A |
| ATOM | 889 | C | ILE | A | 121 | 45.193 | 86.576 | 2.945 | 1.00 | 15.30 | A |
| ATOM | 890 | O | ILE | A | 121 | 44.154 | 85.917 | 2.902 | 1.00 | 15.51 | A |
| ATOM | 891 | N | GLU | A | 122 | 46.370 | 86.044 | 3.263 | 1.00 | 18.03 | A |
| ATOM | 892 | CA | GLU | A | 122 | 46.507 | 84.622 | 3.563 | 1.00 | 19.65 | A |
| ATOM | 893 | CB | GLU | A | 122 | 47.231 | 83.936 | 2.398 | 1.00 | 23.87 | A |
| ATOM | 894 | CG | GLU | A | 122 | 46.538 | 84.052 | 1.051 | 1.00 | 29.93 | A |
| ATOM | 895 | CD | GLU | A | 122 | 47.314 | 83.362 | −0.055 | 1.00 | 34.09 | A |
| ATOM | 896 | OE1 | GLU | A | 122 | 48.492 | 83.720 | −0.268 | 1.00 | 36.67 | A |
| ATOM | 897 | OE2 | GLU | A | 122 | 46.749 | 82.462 | −0.712 | 1.00 | 36.93 | A |
| ATOM | 898 | C | GLU | A | 122 | 47.200 | 84.348 | 4.894 | 1.00 | 18.90 | A |
| ATOM | 899 | O | GLU | A | 122 | 47.339 | 83.189 | 5.290 | 1.00 | 20.61 | A |
| ATOM | 900 | N | SER | A | 123 | 47.633 | 85.396 | 5.590 | 1.00 | 16.61 | A |
| ATOM | 901 | CA | SER | A | 123 | 48.331 | 85.205 | 6.858 | 1.00 | 15.22 | A |
| ATOM | 902 | CB | SER | A | 123 | 49.737 | 84.683 | 6.569 | 1.00 | 15.40 | A |
| ATOM | 903 | OG | SER | A | 123 | 50.528 | 85.721 | 6.012 | 1.00 | 17.88 | A |
| ATOM | 904 | C | SER | A | 123 | 48.380 | 86.481 | 7.681 | 1.00 | 13.61 | A |
| ATOM | 905 | O | SER | A | 123 | 48.069 | 87.567 | 7.190 | 1.00 | 13.29 | A |
| ATOM | 906 | N | ALA | A | 124 | 48.798 | 86.335 | 8.936 | 1.00 | 14.71 | A |
| ATOM | 907 | CA | ALA | A | 124 | 48.931 | 87.475 | 9.833 | 1.00 | 15.10 | A |
| ATOM | 908 | CB | ALA | A | 124 | 49.289 | 87.000 | 11.235 | 1.00 | 15.67 | A |
| ATOM | 909 | C | ALA | A | 124 | 50.037 | 88.371 | 9.281 | 1.00 | 14.20 | A |
| ATOM | 910 | O | ALA | A | 124 | 49.943 | 89.595 | 9.337 | 1.00 | 14.14 | A |
| ATOM | 911 | N | ALA | A | 125 | 51.089 | 87.751 | 8.752 | 1.00 | 14.72 | A |
| ATOM | 912 | CA | ALA | A | 125 | 52.196 | 88.505 | 8.172 | 1.00 | 14.98 | A |
| ATOM | 913 | CB | ALA | A | 125 | 53.245 | 87.552 | 7.613 | 1.00 | 17.46 | A |
| ATOM | 914 | C | ALA | A | 125 | 51.654 | 89.412 | 7.068 | 1.00 | 14.92 | A |
| ATOM | 915 | O | ALA | A | 125 | 52.038 | 90.578 | 6.970 | 1.00 | 15.73 | A |
| ATOM | 916 | N | ASP | A | 126 | 50.758 | 88.876 | 6.240 | 1.00 | 15.00 | A |
| ATOM | 917 | CA | ASP | A | 126 | 50.151 | 89.654 | 5.160 | 1.00 | 13.86 | A |
| ATOM | 918 | CB | ASP | A | 126 | 49.142 | 88.792 | 4.389 | 1.00 | 16.41 | A |
| ATOM | 919 | CG | ASP | A | 126 | 49.799 | 87.817 | 3.430 | 1.00 | 16.11 | A |
| ATOM | 920 | OD1 | ASP | A | 126 | 51.019 | 87.929 | 3.176 | 1.00 | 17.69 | A |
| ATOM | 921 | OD2 | ASP | A | 126 | 49.074 | 86.940 | 2.915 | 1.00 | 18.28 | A |
| ATOM | 922 | C | ASP | A | 126 | 49.494 | 90.914 | 5.721 | 1.00 | 14.64 | A |
| ATOM | 923 | O | ASP | A | 126 | 49.682 | 92.011 | 5.198 | 1.00 | 16.49 | A |
| ATOM | 924 | N | LEU | A | 127 | 48.702 | 90.749 | 6.776 | 1.00 | 14.07 | A |
| ATOM | 925 | CA | LEU | A | 127 | 48.017 | 91.878 | 7.397 | 1.00 | 13.65 | A |
| ATOM | 926 | CB | LEU | A | 127 | 47.061 | 91.367 | 8.480 | 1.00 | 14.34 | A |
| ATOM | 927 | CG | LEU | A | 127 | 45.763 | 90.664 | 8.087 | 1.00 | 13.53 | A |
| ATOM | 928 | CD1 | LEU | A | 127 | 45.078 | 90.165 | 9.349 | 1.00 | 13.86 | A |
| ATOM | 929 | CD2 | LEU | A | 127 | 44.857 | 91.617 | 7.323 | 1.00 | 12.53 | A |
| ATOM | 930 | C | LEU | A | 127 | 48.997 | 92.901 | 7.955 | 1.00 | 14.30 | A |
| ATOM | 931 | O | LEU | A | 127 | 48.853 | 94.098 | 7.724 | 1.00 | 14.36 | A |
| ATOM | 932 | N | SER | A | 128 | 49.986 | 92.416 | 8.696 | 1.00 | 15.05 | A |
| ATOM | 933 | CA | SER | A | 128 | 50.973 | 93.282 | 9.331 | 1.00 | 15.56 | A |
| ATOM | 934 | CB | SER | A | 128 | 51.931 | 92.419 | 10.159 | 1.00 | 15.97 | A |
| ATOM | 935 | OG | SER | A | 128 | 52.837 | 91.719 | 9.321 | 1.00 | 17.93 | A |
| ATOM | 936 | C | SER | A | 128 | 51.722 | 94.188 | 8.362 | 1.00 | 17.20 | A |
| ATOM | 937 | O | SER | A | 128 | 52.102 | 95.301 | 8.724 | 1.00 | 17.49 | A |
| ATOM | 938 | N | LYS | A | 129 | 51.928 | 93.712 | 7.138 | 1.00 | 17.72 | A |
| ATOM | 939 | CA | LYS | A | 129 | 52.672 | 94.464 | 6.129 | 1.00 | 20.04 | A |
| ATOM | 940 | CB | LYS | A | 129 | 53.389 | 93.469 | 5.210 | 1.00 | 20.87 | A |
| ATOM | 941 | CG | LYS | A | 129 | 54.392 | 92.568 | 5.902 | 1.00 | 23.87 | A |
| ATOM | 942 | CD | LYS | A | 129 | 55.000 | 91.592 | 4.907 | 1.00 | 27.53 | A |
| ATOM | 943 | CE | LYS | A | 129 | 56.036 | 90.695 | 5.561 | 1.00 | 29.36 | A |
| ATOM | 944 | NZ | LYS | A | 129 | 56.599 | 89.713 | 4.592 | 1.00 | 30.61 | A |
| ATOM | 945 | C | LYS | A | 129 | 51.860 | 95.474 | 5.329 | 1.00 | 21.23 | A |
| ATOM | 946 | O | LYS | A | 129 | 52.348 | 95.992 | 4.324 | 1.00 | 23.30 | A |
| ATOM | 947 | N | GLN | A | 130 | 50.636 | 95.771 | 5.753 | 1.00 | 19.95 | A |
| ATOM | 948 | CA | GLN | A | 130 | 49.824 | 96.721 | 5.003 | 1.00 | 18.30 | A |
| ATOM | 949 | CB | GLN | A | 130 | 49.225 | 95.991 | 3.795 | 1.00 | 18.41 | A |
| ATOM | 950 | CG | GLN | A | 130 | 48.329 | 94.814 | 4.160 | 1.00 | 16.67 | A |
| ATOM | 951 | CD | GLN | A | 130 | 46.934 | 95.248 | 4.557 | 1.00 | 16.07 | A |
| ATOM | 952 | OE1 | GLN | A | 130 | 46.181 | 95.769 | 3.736 | 1.00 | 16.30 | A |
| ATOM | 953 | NE2 | GLN | A | 130 | 46.583 | 95.039 | 5.822 | 1.00 | 14.90 | A |
| ATOM | 954 | C | GLN | A | 130 | 48.781 | 97.420 | 5.865 | 1.00 | 17.82 | A |
| ATOM | 955 | O | GLN | A | 130 | 48.595 | 97.083 | 7.037 | 1.00 | 18.78 | A |
| ATOM | 956 | N | THR | A | 131 | 48.108 | 98.404 | 5.274 | 1.00 | 18.29 | A |
| ATOM | 957 | CA | THR | A | 131 | 47.083 | 99.166 | 5.975 | 1.00 | 18.33 | A |
| ATOM | 958 | CB | THR | A | 131 | 47.611 | 100.552 | 6.404 | 1.00 | 19.51 | A |
| ATOM | 959 | OG1 | THR | A | 131 | 48.105 | 101.250 | 5.255 | 1.00 | 19.09 | A |
| ATOM | 960 | CG2 | THR | A | 131 | 48.731 | 100.404 | 7.426 | 1.00 | 19.39 | A |
| ATOM | 961 | C | THR | A | 131 | 45.824 | 99.367 | 5.133 | 1.00 | 18.32 | A |
| ATOM | 962 | O | THR | A | 131 | 44.800 | 99.819 | 5.645 | 1.00 | 18.96 | A |
| ATOM | 963 | N | GLU | A | 132 | 45.904 | 99.029 | 3.848 | 1.00 | 18.96 | A |
| ATOM | 964 | CA | GLU | A | 132 | 44.767 | 99.165 | 2.936 | 1.00 | 18.71 | A |
| ATOM | 965 | CB | GLU | A | 132 | 45.192 | 98.712 | 1.535 | 1.00 | 21.48 | A |
| ATOM | 966 | CG | GLU | A | 132 | 44.076 | 98.733 | 0.498 | 1.00 | 27.82 | A |
| ATOM | 967 | CD | GLU | A | 132 | 43.467 | 100.110 | 0.317 | 1.00 | 31.71 | A |
| ATOM | 968 | OE1 | GLU | A | 132 | 44.223 | 101.062 | 0.031 | 1.00 | 34.52 | A |
| ATOM | 969 | OE2 | GLU | A | 132 | 42.232 | 100.240 | 0.458 | 1.00 | 34.23 | A |
| ATOM | 970 | C | GLU | A | 132 | 43.583 | 98.375 | 3.483 | 1.00 | 16.80 | A |
| ATOM | 971 | O | GLU | A | 132 | 42.447 | 98.854 | 3.501 | 1.00 | 16.77 | A |
| ATOM | 972 | N | ILE | A | 133 | 43.858 | 97.150 | 3.912 | 1.00 | 14.67 | A |
| ATOM | 973 | CA | ILE | A | 133 | 42.834 | 96.297 | 4.488 | 1.00 | 13.77 | A |
| ATOM | 974 | CB | ILE | A | 133 | 43.047 | 94.823 | 4.089 | 1.00 | 13.06 | A |
| ATOM | 975 | CG2 | ILE | A | 133 | 41.967 | 93.948 | 4.716 | 1.00 | 13.29 | A |
| ATOM | 976 | CG1 | ILE | A | 133 | 43.007 | 94.691 | 2.564 | 1.00 | 15.61 | A |
| ATOM | 977 | CD1 | ILE | A | 133 | 43.390 | 93.322 | 2.050 | 1.00 | 16.33 | A |
| ATOM | 978 | C | ILE | A | 133 | 42.974 | 96.453 | 5.997 | 1.00 | 12.06 | A |
| ATOM | 979 | O | ILE | A | 133 | 43.949 | 95.998 | 6.595 | 1.00 | 13.27 | A |
| ATOM | 980 | N | ALA | A | 134 | 42.010 | 97.131 | 6.606 | 1.00 | 12.33 | A |
| ATOM | 981 | CA | ALA | A | 134 | 42.040 | 97.350 | 8.044 | 1.00 | 12.13 | A |
| ATOM | 982 | CB | ALA | A | 134 | 41.011 | 98.398 | 8.429 | 1.00 | 12.49 | A |
| ATOM | 983 | C | ALA | A | 134 | 41.749 | 96.035 | 8.754 | 1.00 | 11.32 | A |
| ATOM | 984 | O | ALA | A | 134 | 41.132 | 95.136 | 8.179 | 1.00 | 13.18 | A |
| ATOM | 985 | N | TYR | A | 135 | 42.200 | 95.916 | 9.998 | 1.00 | 10.47 | A |
| ATOM | 986 | CA | TYR | A | 135 | 41.964 | 94.694 | 10.754 | 1.00 | 9.42 | A |
| ATOM | 987 | CB | TYR | A | 135 | 43.006 | 93.637 | 10.351 | 1.00 | 9.29 | A |
| ATOM | 988 | CG | TYR | A | 135 | 44.446 | 94.040 | 10.576 | 1.00 | 9.02 | A |
| ATOM | 989 | CD1 | TYR | A | 135 | 45.090 | 93.755 | 11.779 | 1.00 | 10.76 | A |
| ATOM | 990 | CE1 | TYR | A | 135 | 46.421 | 94.111 | 11.987 | 1.00 | 11.52 | A |
| ATOM | 991 | CD2 | TYR | A | 135 | 45.171 | 94.699 | 9.582 | 1.00 | 11.38 | A |
| ATOM | 992 | CE2 | TYR | A | 135 | 46.504 | 95.061 | 9.781 | 1.00 | 10.76 | A |
| ATOM | 993 | CZ | TYR | A | 135 | 47.122 | 94.763 | 10.982 | 1.00 | 11.29 | A |
| ATOM | 994 | OH | TYR | A | 135 | 48.437 | 95.127 | 11.183 | 1.00 | 11.63 | A |
| ATOM | 995 | C | TYR | A | 135 | 41.924 | 94.991 | 12.244 | 1.00 | 10.12 | A |
| ATOM | 996 | O | TYR | A | 135 | 42.615 | 95.882 | 12.730 | 1.00 | 10.23 | A |
| ATOM | 997 | N | GLY | A | 136 | 41.104 | 94.238 | 12.969 | 1.00 | 10.76 | A |
| ATOM | 998 | CA | GLY | A | 136 | 40.988 | 94.464 | 14.394 | 1.00 | 9.01 | A |
| ATOM | 999 | C | GLY | A | 136 | 40.458 | 93.275 | 15.167 | 1.00 | 10.02 | A |
| ATOM | 1000 | O | GLY | A | 136 | 40.264 | 92.186 | 14.617 | 1.00 | 10.81 | A |
| ATOM | 1001 | N | THR | A | 137 | 40.212 | 93.508 | 16.453 | 1.00 | 10.56 | A |
| ATOM | 1002 | CA | THR | A | 137 | 39.721 | 92.486 | 17.372 | 1.00 | 11.45 | A |
| ATOM | 1003 | CB | THR | A | 137 | 40.820 | 92.075 | 18.379 | 1.00 | 13.34 | A |
| ATOM | 1004 | OG1 | THR | A | 137 | 41.070 | 93.168 | 19.276 | 1.00 | 15.72 | A |
| ATOM | 1005 | CG2 | THR | A | 137 | 42.115 | 91.724 | 17.663 | 1.00 | 13.60 | A |
| ATOM | 1006 | C | THR | A | 137 | 38.570 | 93.052 | 18.200 | 1.00 | 10.89 | A |
| ATOM | 1007 | O | THR | A | 137 | 38.135 | 94.192 | 18.000 | 1.00 | 11.43 | A |
| ATOM | 1008 | N | LEU | A | 138 | 38.077 | 92.241 | 19.130 | 1.00 | 11.39 | A |
| ATOM | 1009 | CA | LEU | A | 138 | 37.026 | 92.684 | 20.032 | 1.00 | 11.58 | A |
| ATOM | 1010 | CB | LEU | A | 138 | 36.451 | 91.476 | 20.774 | 1.00 | 12.41 | A |
| ATOM | 1011 | CG | LEU | A | 138 | 35.496 | 90.568 | 20.003 | 1.00 | 14.07 | A |
| ATOM | 1012 | CD1 | LEU | A | 138 | 35.299 | 89.276 | 20.784 | 1.00 | 14.31 | A |
| ATOM | 1013 | CD2 | LEU | A | 138 | 34.174 | 91.283 | 19.771 | 1.00 | 15.53 | A |
| ATOM | 1014 | C | LEU | A | 138 | 37.703 | 93.691 | 20.955 | 1.00 | 14.74 | A |
| ATOM | 1015 | O | LEU | A | 138 | 38.923 | 93.641 | 21.151 | 1.00 | 15.25 | A |
| ATOM | 1016 | N | ASP | A | 139 | 36.922 | 94.603 | 21.525 | 1.00 | 15.55 | A |
| ATOM | 1017 | CA | ASP | A | 139 | 37.483 | 95.618 | 22.411 | 1.00 | 19.69 | A |
| ATOM | 1018 | CB | ASP | A | 139 | 36.539 | 96.823 | 22.455 | 1.00 | 24.00 | A |
| ATOM | 1019 | CG | ASP | A | 139 | 35.192 | 96.487 | 23.050 | 1.00 | 29.20 | A |
| ATOM | 1020 | OD1 | ASP | A | 139 | 34.547 | 95.536 | 22.560 | 1.00 | 32.68 | A |
| ATOM | 1021 | OD2 | ASP | A | 139 | 34.776 | 97.178 | 24.005 | 1.00 | 32.36 | A |
| ATOM | 1022 | C | ASP | A | 139 | 37.815 | 95.061 | 23.792 | 1.00 | 19.51 | A |
| ATOM | 1023 | O | ASP | A | 139 | 38.333 | 95.777 | 24.648 | 1.00 | 20.23 | A |
| ATOM | 1024 | N | SER | A | 140 | 37.512 | 93.787 | 24.009 | 1.00 | 17.95 | A |
| ATOM | 1025 | CA | SER | A | 140 | 37.802 | 93.135 | 25.280 | 1.00 | 17.27 | A |
| ATOM | 1026 | CB | SER | A | 140 | 36.722 | 93.499 | 26.305 | 1.00 | 19.12 | A |
| ATOM | 1027 | OG | SER | A | 140 | 35.477 | 92.929 | 25.958 | 1.00 | 24.29 | A |
| ATOM | 1028 | C | SER | A | 140 | 37.908 | 91.636 | 25.025 | 1.00 | 15.40 | A |
| ATOM | 1029 | O | SER | A | 140 | 37.276 | 91.110 | 24.109 | 1.00 | 16.77 | A |
| ATOM | 1030 | N | GLY | A | 141 | 38.707 | 90.951 | 25.835 | 1.00 | 14.00 | A |
| ATOM | 1031 | CA | GLY | A | 141 | 38.863 | 89.520 | 25.658 | 1.00 | 11.82 | A |
| ATOM | 1032 | C | GLY | A | 141 | 40.307 | 89.091 | 25.482 | 1.00 | 11.66 | A |
| ATOM | 1033 | O | GLY | A | 141 | 41.208 | 89.922 | 25.354 | 1.00 | 12.35 | A |
| ATOM | 1034 | N | SER | A | 142 | 40.521 | 87.780 | 25.445 | 1.00 | 10.38 | A |
| ATOM | 1035 | CA | SER | A | 142 | 41.860 | 87.215 | 25.323 | 1.00 | 10.71 | A |
| ATOM | 1036 | CB | SER | A | 142 | 41.788 | 85.707 | 25.573 | 1.00 | 12.33 | A |
| ATOM | 1037 | OG | SER | A | 142 | 41.281 | 85.427 | 26.861 | 1.00 | 20.39 | A |
| ATOM | 1038 | C | SER | A | 142 | 42.582 | 87.540 | 24.025 | 1.00 | 10.09 | A |
| ATOM | 1039 | O | SER | A | 142 | 43.809 | 87.576 | 23.995 | 1.00 | 11.57 | A |
| ATOM | 1040 | N | THR | A | 143 | 41.840 | 87.758 | 22.947 | 1.00 | 8.66 | A |
| ATOM | 1041 | CA | THR | A | 143 | 42.491 | 88.067 | 21.683 | 1.00 | 10.00 | A |
| ATOM | 1042 | CB | THR | A | 143 | 41.506 | 87.996 | 20.517 | 1.00 | 9.34 | A |
| ATOM | 1043 | OG1 | THR | A | 143 | 40.996 | 86.660 | 20.424 | 1.00 | 10.10 | A |
| ATOM | 1044 | CG2 | THR | A | 143 | 42.199 | 88.361 | 19.216 | 1.00 | 11.12 | A |
| ATOM | 1045 | C | THR | A | 143 | 43.128 | 89.447 | 21.738 | 1.00 | 9.95 | A |
| ATOM | 1046 | O | THR | A | 143 | 44.276 | 89.617 | 21.333 | 1.00 | 11.74 | A |
| ATOM | 1047 | N | LYS | A | 144 | 42.394 | 90.432 | 22.248 | 1.00 | 10.61 | A |
| ATOM | 1048 | CA | LYS | A | 144 | 42.940 | 91.779 | 22.361 | 1.00 | 12.70 | A |
| ATOM | 1049 | CB | LYS | A | 144 | 41.858 | 92.730 | 22.881 | 1.00 | 14.41 | A |
| ATOM | 1050 | CG | LYS | A | 144 | 42.307 | 94.186 | 22.949 | 1.00 | 16.73 | A |
| ATOM | 1051 | CD | LYS | A | 144 | 41.216 | 95.069 | 23.544 | 1.00 | 21.27 | A |
| ATOM | 1052 | CE | LYS | A | 144 | 41.672 | 96.512 | 23.677 | 1.00 | 23.82 | A |
| ATOM | 1053 | NZ | LYS | A | 144 | 40.633 | 97.363 | 24.322 | 1.00 | 25.58 | A |
| ATOM | 1054 | C | LYS | A | 144 | 44.162 | 91.715 | 23.271 | 1.00 | 12.70 | A |
| ATOM | 1055 | O | LYS | A | 144 | 45.197 | 92.314 | 22.980 | 1.00 | 13.06 | A |
| ATOM | 1056 | N | GLU | A | 145 | 44.040 | 90.976 | 24.369 | 1.00 | 13.84 | A |
| ATOM | 1057 | CA | GLU | A | 145 | 45.141 | 90.840 | 25.317 | 1.00 | 14.37 | A |
| ATOM | 1058 | CB | GLU | A | 145 | 44.646 | 90.108 | 26.567 | 1.00 | 18.35 | A |
| ATOM | 1059 | CG | GLU | A | 145 | 43.661 | 90.914 | 27.394 | 1.00 | 23.95 | A |
| ATOM | 1060 | CD | GLU | A | 145 | 44.154 | 92.323 | 27.666 | 1.00 | 27.85 | A |
| ATOM | 1061 | OE1 | GLU | A | 145 | 45.300 | 92.474 | 28.140 | 1.00 | 29.32 | A |
| ATOM | 1062 | OE2 | GLU | A | 145 | 43.393 | 93.279 | 27.408 | 1.00 | 31.79 | A |
| ATOM | 1063 | C | GLU | A | 145 | 46.350 | 90.172 | 24.677 | 1.00 | 13.58 | A |
| ATOM | 1064 | O | GLU | A | 145 | 47.489 | 90.542 | 24.963 | 1.00 | 13.47 | A |
| ATOM | 1065 | N | PHE | A | 146 | 46.105 | 89.189 | 23.814 | 1.00 | 11.98 | A |
| ATOM | 1066 | CA | PHE | A | 146 | 47.188 | 88.488 | 23.135 | 1.00 | 12.54 | A |
| ATOM | 1067 | CB | PHE | A | 146 | 46.609 | 87.440 | 22.178 | 1.00 | 11.57 | A |
| ATOM | 1068 | CG | PHE | A | 146 | 47.635 | 86.830 | 21.255 | 1.00 | 12.50 | A |
| ATOM | 1069 | CD1 | PHE | A | 146 | 48.313 | 85.795 | 21.634 | 1.00 | 12.88 | A |
| ATOM | 1070 | CD2 | PHE | A | 146 | 47.850 | 87.354 | 20.089 | 1.00 | 12.84 | A |
| ATOM | 1071 | CE1 | PHE | A | 146 | 49.290 | 85.202 | 20.820 | 1.00 | 13.90 | A |
| ATOM | 1072 | CE2 | PHE | A | 146 | 48.807 | 86.837 | 19.201 | 1.00 | 13.55 | A |
| ATOM | 1073 | CZ | PHE | A | 146 | 49.539 | 85.737 | 19.581 | 1.00 | 14.05 | A |
| ATOM | 1074 | C | PHE | A | 146 | 48.049 | 89.509 | 22.410 | 1.00 | 11.89 | A |
| ATOM | 1075 | O | PHE | A | 146 | 49.269 | 89.521 | 22.553 | 1.00 | 12.44 | A |
| ATOM | 1076 | N | PHE | A | 147 | 47.401 | 90.363 | 21.626 | 1.00 | 11.06 | A |
| ATOM | 1077 | CA | PHE | A | 147 | 48.110 | 91.377 | 20.861 | 1.00 | 12.11 | A |
| ATOM | 1078 | CB | PHE | A | 147 | 47.148 | 91.980 | 19.831 | 1.00 | 10.46 | A |
| ATOM | 1079 | CG | PHE | A | 147 | 46.901 | 91.088 | 18.640 | 1.00 | 11.19 | A |
| ATOM | 1080 | CD1 | PHE | A | 147 | 47.825 | 90.940 | 17.745 | 1.00 | 10.73 | A |
| ATOM | 1081 | CD2 | PHE | A | 147 | 45.774 | 90.457 | 18.522 | 1.00 | 10.42 | A |
| ATOM | 1082 | CE1 | PHE | A | 147 | 47.658 | 90.111 | 16.634 | 1.00 | 10.56 | A |
| ATOM | 1083 | CE2 | PHE | A | 147 | 45.509 | 89.603 | 17.446 | 1.00 | 11.65 | A |
| ATOM | 1084 | CZ | PHE | A | 147 | 46.472 | 89.428 | 16.486 | 1.00 | 10.21 | A |
| ATOM | 1085 | C | PHE | A | 147 | 48.776 | 92.416 | 21.752 | 1.00 | 13.23 | A |
| ATOM | 1086 | O | PHE | A | 147 | 49.932 | 92.769 | 21.531 | 1.00 | 12.92 | A |
| ATOM | 1087 | N | ARG | A | 148 | 48.054 | 92.901 | 22.755 | 1.00 | 12.51 | A |
| ATOM | 1088 | CA | ARG | A | 148 | 48.594 | 93.909 | 23.660 | 1.00 | 13.44 | A |
| ATOM | 1089 | CB | ARG | A | 148 | 47.506 | 94.320 | 24.660 | 1.00 | 15.03 | A |
| ATOM | 1090 | CG | ARG | A | 148 | 47.954 | 95.387 | 25.653 | 1.00 | 17.55 | A |
| ATOM | 1091 | CD | ARG | A | 148 | 47.032 | 95.439 | 26.863 | 1.00 | 23.81 | A |
| ATOM | 1092 | NE | ARG | A | 148 | 45.666 | 95.820 | 26.517 | 1.00 | 27.85 | A |
| ATOM | 1093 | CZ | ARG | A | 148 | 45.297 | 97.046 | 26.162 | 1.00 | 29.59 | A |
| ATOM | 1094 | NH1 | ARG | A | 148 | 46.195 | 98.020 | 26.105 | 1.00 | 31.38 | A |
| ATOM | 1095 | NH2 | ARG | A | 148 | 44.028 | 97.302 | 25.870 | 1.00 | 30.78 | A |
| ATOM | 1096 | C | ARG | A | 148 | 49.868 | 93.442 | 24.355 | 1.00 | 14.63 | A |
| ATOM | 1097 | O | ARG | A | 148 | 50.803 | 94.224 | 24.529 | 1.00 | 15.99 | A |
| ATOM | 1098 | N | ARG | A | 149 | 49.912 | 92.169 | 24.739 | 1.00 | 14.85 | A |
| ATOM | 1099 | CA | ARG | A | 149 | 51.060 | 91.622 | 25.461 | 1.00 | 16.63 | A |
| ATOM | 1100 | CB | ARG | A | 149 | 50.530 | 90.711 | 26.575 | 1.00 | 18.90 | A |
| ATOM | 1101 | CG | ARG | A | 149 | 49.718 | 91.426 | 27.642 | 1.00 | 23.96 | A |
| ATOM | 1102 | CD | ARG | A | 149 | 49.027 | 90.436 | 28.571 | 1.00 | 28.31 | A |
| ATOM | 1103 | NE | ARG | A | 149 | 49.958 | 89.477 | 29.163 | 1.00 | 32.87 | A |
| ATOM | 1104 | CZ | ARG | A | 149 | 49.601 | 88.510 | 30.004 | 1.00 | 34.71 | A |
| ATOM | 1105 | NH1 | ARG | A | 149 | 48.330 | 88.370 | 30.356 | 1.00 | 35.27 | A |
| ATOM | 1106 | NH2 | ARG | A | 149 | 50.512 | 87.678 | 30.491 | 1.00 | 35.30 | A |
| ATOM | 1107 | C | ARG | A | 149 | 52.119 | 90.918 | 24.617 | 1.00 | 14.67 | A |
| ATOM | 1108 | O | ARG | A | 149 | 53.164 | 90.531 | 25.141 | 1.00 | 16.69 | A |
| ATOM | 1109 | N | SER | A | 150 | 51.877 | 90.748 | 23.323 | 1.00 | 14.80 | A |
| ATOM | 1110 | CA | SER | A | 150 | 52.843 | 90.038 | 22.494 | 1.00 | 15.33 | A |
| ATOM | 1111 | CB | SER | A | 150 | 52.209 | 89.706 | 21.142 | 1.00 | 16.25 | A |
| ATOM | 1112 | OG | SER | A | 150 | 53.102 | 88.924 | 20.362 | 1.00 | 16.71 | A |
| ATOM | 1113 | C | SER | A | 150 | 54.187 | 90.738 | 22.335 | 1.00 | 16.91 | A |
| ATOM | 1114 | O | SER | A | 150 | 54.266 | 91.967 | 22.254 | 1.00 | 18.19 | A |
| ATOM | 1115 | N | ALA | A | 151 | 55.240 | 89.926 | 22.288 | 1.00 | 17.17 | A |
| ATOM | 1116 | CA | ALA | A | 151 | 56.605 | 90.401 | 22.100 | 1.00 | 17.74 | A |
| ATOM | 1117 | CB | ALA | A | 151 | 57.504 | 89.905 | 23.233 | 1.00 | 17.92 | A |
| ATOM | 1118 | C | ALA | A | 151 | 57.085 | 89.849 | 20.757 | 1.00 | 18.11 | A |
| ATOM | 1119 | O | ALA | A | 151 | 58.243 | 90.018 | 20.376 | 1.00 | 19.71 | A |
| ATOM | 1120 | N | ILE | A | 152 | 56.179 | 89.171 | 20.053 | 1.00 | 16.75 | A |
| ATOM | 1121 | CA | ILE | A | 152 | 56.469 | 88.599 | 18.739 | 1.00 | 16.86 | A |
| ATOM | 1122 | CB | ILE | A | 152 | 55.448 | 87.494 | 18.380 | 1.00 | 16.79 | A |
| ATOM | 1123 | CG2 | ILE | A | 152 | 55.760 | 86.920 | 17.008 | 1.00 | 15.71 | A |
| ATOM | 1124 | CG1 | ILE | A | 152 | 55.458 | 86.396 | 19.451 | 1.00 | 19.51 | A |
| ATOM | 1125 | CD1 | ILE | A | 152 | 56.784 | 85.681 | 19.602 | 1.00 | 22.08 | A |
| ATOM | 1126 | C | ILE | A | 152 | 56.367 | 89.727 | 17.709 | 1.00 | 15.48 | A |
| ATOM | 1127 | O | ILE | A | 152 | 55.329 | 90.373 | 17.596 | 1.00 | 15.67 | A |
| ATOM | 1128 | N | ALA | A | 153 | 57.438 | 89.950 | 16.954 | 1.00 | 15.61 | A |
| ATOM | 1129 | CA | ALA | A | 153 | 57.485 | 91.028 | 15.962 | 1.00 | 14.18 | A |
| ATOM | 1130 | CB | ALA | A | 153 | 58.690 | 90.836 | 15.052 | 1.00 | 14.47 | A |
| ATOM | 1131 | C | ALA | A | 153 | 56.231 | 91.241 | 15.114 | 1.00 | 12.53 | A |
| ATOM | 1132 | O | ALA | A | 153 | 55.697 | 92.353 | 15.051 | 1.00 | 13.88 | A |
| ATOM | 1133 | N | VAL | A | 154 | 55.762 | 90.193 | 14.448 | 1.00 | 13.33 | A |
| ATOM | 1134 | CA | VAL | A | 154 | 54.576 | 90.330 | 13.613 | 1.00 | 12.94 | A |
| ATOM | 1135 | CB | VAL | A | 154 | 54.255 | 89.008 | 12.870 | 1.00 | 14.20 | A |
| ATOM | 1136 | CG1 | VAL | A | 154 | 52.905 | 89.108 | 12.179 | 1.00 | 14.95 | A |
| ATOM | 1137 | CG2 | VAL | A | 154 | 55.342 | 88.715 | 11.849 | 1.00 | 16.93 | A |
| ATOM | 1138 | C | VAL | A | 154 | 53.352 | 90.758 | 14.420 | 1.00 | 11.60 | A |
| ATOM | 1139 | O | VAL | A | 154 | 52.606 | 91.649 | 14.006 | 1.00 | 12.26 | A |
| ATOM | 1140 | N | PHE | A | 155 | 53.161 | 90.141 | 15.582 | 1.00 | 11.98 | A |
| ATOM | 1141 | CA | PHE | A | 155 | 52.004 | 90.448 | 16.413 | 1.00 | 12.64 | A |
| ATOM | 1142 | CB | PHE | A | 155 | 51.753 | 89.287 | 17.387 | 1.00 | 12.89 | A |
| ATOM | 1143 | CG | PHE | A | 155 | 51.641 | 87.936 | 16.718 | 1.00 | 12.14 | A |
| ATOM | 1144 | CD1 | PHE | A | 155 | 51.079 | 87.824 | 15.556 | 1.00 | 13.16 | A |
| ATOM | 1145 | CD2 | PHE | A | 155 | 52.103 | 86.888 | 17.317 | 1.00 | 13.67 | A |
| ATOM | 1146 | CE1 | PHE | A | 155 | 50.950 | 86.587 | 14.903 | 1.00 | 13.25 | A |
| ATOM | 1147 | CE2 | PHE | A | 155 | 52.024 | 85.609 | 16.756 | 1.00 | 14.31 | A |
| ATOM | 1148 | CZ | PHE | A | 155 | 51.436 | 85.459 | 15.523 | 1.00 | 13.23 | A |
| ATOM | 1149 | C | PHE | A | 155 | 52.088 | 91.801 | 17.110 | 1.00 | 12.95 | A |
| ATOM | 1150 | O | PHE | A | 155 | 51.076 | 92.498 | 17.223 | 1.00 | 12.80 | A |
| ATOM | 1151 | N | ASP | A | 156 | 53.268 | 92.182 | 17.597 | 1.00 | 14.04 | A |
| ATOM | 1152 | CA | ASP | A | 156 | 53.364 | 93.473 | 18.262 | 1.00 | 14.31 | A |
| ATOM | 1153 | CB | ASP | A | 156 | 54.637 | 93.553 | 19.130 | 1.00 | 17.42 | A |
| ATOM | 1154 | CG | ASP | A | 156 | 55.910 | 93.340 | 18.347 | 1.00 | 20.36 | A |
| ATOM | 1155 | OD1 | ASP | A | 156 | 56.114 | 94.029 | 17.330 | 1.00 | 21.14 | A |
| ATOM | 1156 | OD2 | ASP | A | 156 | 56.721 | 92.488 | 18.770 | 1.00 | 23.78 | A |
| ATOM | 1157 | C | ASP | A | 156 | 53.208 | 94.598 | 17.243 | 1.00 | 13.14 | A |
| ATOM | 1158 | O | ASP | A | 156 | 52.723 | 95.677 | 17.587 | 1.00 | 13.21 | A |
| ATOM | 1159 | N | LYS | A | 157 | 53.588 | 94.349 | 15.991 | 1.00 | 12.71 | A |
| ATOM | 1160 | CA | LYS | A | 157 | 53.422 | 95.363 | 14.955 | 1.00 | 13.09 | A |
| ATOM | 1161 | CB | LYS | A | 157 | 54.214 | 94.978 | 13.703 | 1.00 | 14.27 | A |
| ATOM | 1162 | CG | LYS | A | 157 | 54.016 | 95.968 | 12.566 | 1.00 | 15.61 | A |
| ATOM | 1163 | CD | LYS | A | 157 | 54.948 | 95.701 | 11.398 | 1.00 | 20.68 | A |
| ATOM | 1164 | CE | LYS | A | 157 | 54.727 | 96.719 | 10.290 | 1.00 | 22.88 | A |
| ATOM | 1165 | NZ | LYS | A | 157 | 55.582 | 96.451 | 9.104 | 1.00 | 25.73 | A |
| ATOM | 1166 | C | LYS | A | 157 | 51.924 | 95.507 | 14.688 | 1.00 | 12.13 | A |
| ATOM | 1167 | O | LYS | A | 157 | 51.418 | 96.610 | 14.479 | 1.00 | 12.98 | A |
| ATOM | 1168 | N | MET | A | 158 | 51.217 | 94.380 | 14.691 | 1.00 | 11.25 | A |
| ATOM | 1169 | CA | MET | A | 158 | 49.777 | 94.383 | 14.465 | 1.00 | 11.17 | A |
| ATOM | 1170 | CB | MET | A | 158 | 49.279 | 92.936 | 14.346 | 1.00 | 10.94 | A |
| ATOM | 1171 | CG | MET | A | 158 | 49.636 | 92.265 | 13.024 | 1.00 | 10.67 | A |
| ATOM | 1172 | SD | MET | A | 158 | 49.209 | 90.507 | 12.972 | 1.00 | 12.05 | A |
| ATOM | 1173 | CE | MET | A | 158 | 47.417 | 90.562 | 12.875 | 1.00 | 12.53 | A |
| ATOM | 1174 | C | MET | A | 158 | 49.084 | 95.170 | 15.570 | 1.00 | 9.74 | A |
| ATOM | 1175 | O | MET | A | 158 | 48.154 | 95.931 | 15.301 | 1.00 | 10.03 | A |
| ATOM | 1176 | N | TRP | A | 159 | 49.537 | 94.995 | 16.809 | 1.00 | 10.33 | A |
| ATOM | 1177 | CA | TRP | A | 159 | 48.938 | 95.708 | 17.933 | 1.00 | 11.27 | A |
| ATOM | 1178 | CB | TRP | A | 159 | 49.509 | 95.184 | 19.252 | 1.00 | 10.95 | A |
| ATOM | 1179 | CG | TRP | A | 159 | 49.008 | 95.972 | 20.431 | 1.00 | 12.47 | A |
| ATOM | 1180 | CD2 | TRP | A | 159 | 47.647 | 96.090 | 20.867 | 1.00 | 12.97 | A |
| ATOM | 1181 | CE2 | TRP | A | 159 | 47.638 | 96.966 | 21.976 | 1.00 | 14.72 | A |
| ATOM | 1182 | CE3 | TRP | A | 159 | 46.433 | 95.541 | 20.428 | 1.00 | 13.48 | A |
| ATOM | 1183 | CD1 | TRP | A | 159 | 49.749 | 96.757 | 21.271 | 1.00 | 15.07 | A |
| ATOM | 1184 | NE1 | TRP | A | 159 | 48.932 | 97.358 | 22.201 | 1.00 | 15.53 | A |
| ATOM | 1185 | CZ2 | TRP | A | 159 | 46.460 | 97.307 | 22.653 | 1.00 | 17.62 | A |
| ATOM | 1186 | CZ3 | TRP | A | 159 | 45.259 | 95.882 | 21.104 | 1.00 | 16.05 | A |
| ATOM | 1187 | CH2 | TRP | A | 159 | 45.285 | 96.757 | 22.203 | 1.00 | 17.88 | A |
| ATOM | 1188 | C | TRP | A | 159 | 49.138 | 97.208 | 17.785 | 1.00 | 11.68 | A |
| ATOM | 1189 | O | TRP | A | 159 | 48.205 | 97.991 | 17.958 | 1.00 | 11.37 | A |
| ATOM | 1190 | N | THR | A | 160 | 50.366 | 97.609 | 17.479 | 1.00 | 11.80 | A |
| ATOM | 1191 | CA | THR | A | 160 | 50.672 | 99.023 | 17.318 | 1.00 | 11.05 | A |
| ATOM | 1192 | CB | THR | A | 160 | 52.154 | 99.221 | 16.973 | 1.00 | 14.07 | A |
| ATOM | 1193 | OG1 | THR | A | 160 | 52.951 | 98.816 | 18.094 | 1.00 | 16.78 | A |
| ATOM | 1194 | CG2 | THR | A | 160 | 52.441 | 100.682 | 16.647 | 1.00 | 15.23 | A |
| ATOM | 1195 | C | THR | A | 160 | 49.790 | 99.621 | 16.228 | 1.00 | 11.18 | A |
| ATOM | 1196 | O | THR | A | 160 | 49.295 | 100.740 | 16.367 | 1.00 | 11.33 | A |
| ATOM | 1197 | N | TYR | A | 161 | 49.591 | 98.872 | 15.149 | 1.00 | 10.84 | A |
| ATOM | 1198 | CA | TYR | A | 161 | 48.736 | 99.325 | 14.062 | 1.00 | 11.33 | A |
| ATOM | 1199 | CB | TYR | A | 161 | 48.813 | 98.346 | 12.881 | 1.00 | 11.26 | A |
| ATOM | 1200 | CG | TYR | A | 161 | 47.562 | 98.409 | 12.034 | 1.00 | 12.15 | A |
| ATOM | 1201 | CD1 | TYR | A | 161 | 47.415 | 99.374 | 11.042 | 1.00 | 11.53 | A |
| ATOM | 1202 | CE1 | TYR | A | 161 | 46.217 | 99.517 | 10.351 | 1.00 | 12.22 | A |
| ATOM | 1203 | CD2 | TYR | A | 161 | 46.475 | 97.578 | 12.307 | 1.00 | 10.65 | A |
| ATOM | 1204 | CE2 | TYR | A | 161 | 45.275 | 97.713 | 11.625 | 1.00 | 12.74 | A |
| ATOM | 1205 | CZ | TYR | A | 161 | 45.150 | 98.686 | 10.651 | 1.00 | 12.21 | A |
| ATOM | 1206 | OH | TYR | A | 161 | 43.948 | 98.846 | 10.006 | 1.00 | 14.64 | A |
| ATOM | 1207 | C | TYR | A | 161 | 47.298 | 99.465 | 14.563 | 1.00 | 11.26 | A |
| ATOM | 1208 | O | TYR | A | 161 | 46.643 | 100.477 | 14.323 | 1.00 | 12.43 | A |
| ATOM | 1209 | N | MET | A | 162 | 46.805 | 98.433 | 15.243 | 1.00 | 11.09 | A |
| ATOM | 1210 | CA | MET | A | 162 | 45.431 | 98.433 | 15.740 | 1.00 | 12.07 | A |
| ATOM | 1211 | CB | MET | A | 162 | 45.127 | 97.092 | 16.411 | 1.00 | 13.59 | A |
| ATOM | 1212 | CG | MET | A | 162 | 44.945 | 95.934 | 15.464 | 1.00 | 12.93 | A |
| ATOM | 1213 | SD | MET | A | 162 | 44.421 | 94.492 | 16.402 | 1.00 | 18.46 | A |
| ATOM | 1214 | CE | MET | A | 162 | 45.964 | 93.644 | 16.600 | 1.00 | 21.20 | A |
| ATOM | 1215 | C | MET | A | 162 | 45.129 | 99.592 | 16.661 | 1.00 | 12.55 | A |
| ATOM | 1216 | O | MET | A | 162 | 44.095 | 100.246 | 16.530 | 1.00 | 12.30 | A |
| ATOM | 1217 | N | ARG | A | 163 | 46.031 | 99.846 | 17.598 | 1.00 | 11.90 | A |
| ATOM | 1218 | CA | ARG | A | 163 | 45.811 | 100.913 | 18.556 | 1.00 | 12.87 | A |
| ATOM | 1219 | CB | ARG | A | 163 | 46.821 | 100.766 | 19.696 | 1.00 | 16.20 | A |
| ATOM | 1220 | CG | ARG | A | 163 | 46.382 | 101.452 | 20.975 | 1.00 | 19.85 | A |
| ATOM | 1221 | CD | ARG | A | 163 | 47.426 | 101.347 | 22.070 | 1.00 | 23.43 | A |
| ATOM | 1222 | NE | ARG | A | 163 | 46.933 | 101.936 | 23.310 | 1.00 | 26.48 | A |
| ATOM | 1223 | CZ | ARG | A | 163 | 47.693 | 102.223 | 24.361 | 1.00 | 27.67 | A |
| ATOM | 1224 | NH1 | ARG | A | 163 | 48.996 | 101.978 | 24.330 | 1.00 | 27.83 | A |
| ATOM | 1225 | NH2 | ARG | A | 163 | 47.145 | 102.758 | 25.443 | 1.00 | 29.17 | A |
| ATOM | 1226 | C | ARG | A | 163 | 45.826 | 102.299 | 17.920 | 1.00 | 13.03 | A |
| ATOM | 1227 | O | ARG | A | 163 | 45.173 | 103.216 | 18.419 | 1.00 | 13.70 | A |
| ATOM | 1228 | N | SER | A | 164 | 46.550 | 102.453 | 16.816 | 1.00 | 11.77 | A |
| ATOM | 1229 | CA | SER | A | 164 | 46.645 | 103.754 | 16.157 | 1.00 | 11.84 | A |
| ATOM | 1230 | CB | SER | A | 164 | 48.124 | 104.065 | 15.902 | 1.00 | 12.55 | A |
| ATOM | 1231 | OG | SER | A | 164 | 48.682 | 103.162 | 14.961 | 1.00 | 14.03 | A |
| ATOM | 1232 | C | SER | A | 164 | 45.800 | 103.911 | 14.894 | 1.00 | 12.15 | A |
| ATOM | 1233 | O | SER | A | 164 | 45.750 | 104.995 | 14.316 | 1.00 | 13.15 | A |
| ATOM | 1234 | N | ALA | A | 165 | 45.127 | 102.847 | 14.468 | 1.00 | 12.42 | A |
| ATOM | 1235 | CA | ALA | A | 165 | 44.315 | 102.906 | 13.256 | 1.00 | 12.32 | A |
| ATOM | 1236 | CB | ALA | A | 165 | 43.963 | 101.495 | 12.803 | 1.00 | 13.52 | A |
| ATOM | 1237 | C | ALA | A | 165 | 43.040 | 103.742 | 13.380 | 1.00 | 12.82 | A |
| ATOM | 1238 | O | ALA | A | 165 | 42.374 | 103.731 | 14.413 | 1.00 | 13.31 | A |
| ATOM | 1239 | N | GLU | A | 166 | 42.710 | 104.459 | 12.307 | 1.00 | 13.69 | A |
| ATOM | 1240 | CA | GLU | A | 166 | 41.518 | 105.300 | 12.257 | 1.00 | 14.95 | A |
| ATOM | 1241 | CB | GLU | A | 166 | 41.923 | 106.767 | 12.392 | 1.00 | 14.11 | A |
| ATOM | 1242 | CG | GLU | A | 166 | 42.629 | 107.113 | 13.680 | 1.00 | 14.49 | A |
| ATOM | 1243 | CD | GLU | A | 166 | 42.760 | 108.609 | 13.864 | 1.00 | 13.93 | A |
| ATOM | 1244 | OE1 | GLU | A | 166 | 43.460 | 109.257 | 13.055 | 1.00 | 14.76 | A |
| ATOM | 1245 | OE2 | GLU | A | 166 | 42.149 | 109.138 | 14.813 | 1.00 | 14.89 | A |
| ATOM | 1246 | C | GLU | A | 166 | 40.776 | 105.033 | 10.952 | 1.00 | 15.15 | A |
| ATOM | 1247 | O | GLU | A | 166 | 41.384 | 105.028 | 9.881 | 1.00 | 17.39 | A |
| ATOM | 1248 | N | PRO | A | 167 | 39.453 | 104.808 | 11.020 | 1.00 | 15.65 | A |
| ATOM | 1249 | CD | PRO | A | 167 | 38.614 | 104.781 | 9.813 | 1.00 | 15.88 | A |
| ATOM | 1250 | CA | PRO | A | 167 | 38.617 | 104.783 | 12.226 | 1.00 | 14.51 | A |
| ATOM | 1251 | CB | PRO | A | 167 | 37.191 | 104.766 | 11.655 | 1.00 | 15.59 | A |
| ATOM | 1252 | CG | PRO | A | 167 | 37.307 | 105.477 | 10.328 | 1.00 | 19.02 | A |
| ATOM | 1253 | C | PRO | A | 167 | 38.980 | 103.575 | 13.082 | 1.00 | 13.89 | A |
| ATOM | 1254 | O | PRO | A | 167 | 39.716 | 102.697 | 12.635 | 1.00 | 13.09 | A |
| ATOM | 1255 | N | SER | A | 168 | 38.462 | 103.530 | 14.306 | 1.00 | 14.02 | A |
| ATOM | 1256 | CA | SER | A | 168 | 38.744 | 102.419 | 15.210 | 1.00 | 13.10 | A |
| ATOM | 1257 | CB | SER | A | 168 | 37.928 | 102.582 | 16.490 | 1.00 | 14.12 | A |
| ATOM | 1258 | OG | SER | A | 168 | 38.111 | 101.448 | 17.317 | 1.00 | 15.45 | A |
| ATOM | 1259 | C | SER | A | 168 | 38.482 | 101.080 | 14.538 | 1.00 | 11.80 | A |
| ATOM | 1260 | O | SER | A | 168 | 37.459 | 100.895 | 13.875 | 1.00 | 13.09 | A |
| ATOM | 1261 | N | VAL | A | 169 | 39.409 | 100.146 | 14.727 | 1.00 | 11.45 | A |
| ATOM | 1262 | CA | VAL | A | 169 | 39.291 | 98.813 | 14.147 | 1.00 | 11.66 | A |
| ATOM | 1263 | CB | VAL | A | 169 | 40.660 | 98.291 | 13.646 | 1.00 | 11.62 | A |
| ATOM | 1264 | CG1 | VAL | A | 169 | 41.163 | 99.175 | 12.515 | 1.00 | 12.91 | A |
| ATOM | 1265 | CG2 | VAL | A | 169 | 41.668 | 98.251 | 14.795 | 1.00 | 11.90 | A |
| ATOM | 1266 | C | VAL | A | 169 | 38.730 | 97.814 | 15.150 | 1.00 | 11.83 | A |
| ATOM | 1267 | O | VAL | A | 169 | 38.575 | 96.633 | 14.840 | 1.00 | 11.71 | A |
| ATOM | 1268 | N | PHE | A | 170 | 38.424 | 98.287 | 16.353 | 1.00 | 11.38 | A |
| ATOM | 1269 | CA | PHE | A | 170 | 37.881 | 97.413 | 17.384 | 1.00 | 12.11 | A |
| ATOM | 1270 | CB | PHE | A | 170 | 38.343 | 97.905 | 18.762 | 1.00 | 11.45 | A |
| ATOM | 1271 | CG | PHE | A | 170 | 39.834 | 97.882 | 18.943 | 1.00 | 12.69 | A |
| ATOM | 1272 | CD1 | PHE | A | 170 | 40.454 | 96.752 | 19.066 | 1.00 | 13.27 | A |
| ATOM | 1273 | CD2 | PHE | A | 170 | 40.499 | 98.993 | 18.954 | 1.00 | 13.73 | A |
| ATOM | 1274 | CE1 | PHE | A | 170 | 41.845 | 96.679 | 19.213 | 1.00 | 13.67 | A |
| ATOM | 1275 | CE2 | PHE | A | 170 | 41.890 | 99.030 | 19.095 | 1.00 | 14.80 | A |
| ATOM | 1276 | CZ | PHE | A | 170 | 42.574 | 97.845 | 19.227 | 1.00 | 14.22 | A |
| ATOM | 1277 | C | PHE | A | 170 | 36.368 | 97.324 | 17.266 | 1.00 | 12.92 | A |
| ATOM | 1278 | O | PHE | A | 170 | 35.707 | 98.276 | 16.843 | 1.00 | 13.95 | A |
| ATOM | 1279 | N | VAL | A | 171 | 35.828 | 96.164 | 17.627 | 1.00 | 13.05 | A |
| ATOM | 1280 | CA | VAL | A | 171 | 34.388 | 95.933 | 17.599 | 1.00 | 13.17 | A |
| ATOM | 1281 | CB | VAL | A | 171 | 34.006 | 94.876 | 16.535 | 1.00 | 13.33 | A |
| ATOM | 1282 | CG1 | VAL | A | 171 | 34.385 | 95.385 | 15.147 | 1.00 | 13.29 | A |
| ATOM | 1283 | CG2 | VAL | A | 171 | 34.696 | 93.554 | 16.830 | 1.00 | 13.56 | A |
| ATOM | 1284 | C | VAL | A | 171 | 33.941 | 95.486 | 18.990 | 1.00 | 13.90 | A |
| ATOM | 1285 | O | VAL | A | 171 | 34.747 | 94.994 | 19.778 | 1.00 | 13.56 | A |
| ATOM | 1286 | N | ALA | A | 172 | 32.658 | 95.668 | 19.291 | 1.00 | 15.24 | A |
| ATOM | 1287 | CA | ALA | A | 172 | 32.123 | 95.320 | 20.604 | 1.00 | 15.32 | A |
| ATOM | 1288 | CB | ALA | A | 172 | 30.989 | 96.277 | 20.961 | 1.00 | 16.56 | A |
| ATOM | 1289 | C | ALA | A | 172 | 31.643 | 93.883 | 20.754 | 1.00 | 14.93 | A |
| ATOM | 1290 | O | ALA | A | 172 | 31.708 | 93.318 | 21.846 | 1.00 | 15.25 | A |
| ATOM | 1291 | N | THR | A | 173 | 31.162 | 93.293 | 19.666 | 1.00 | 14.48 | A |
| ATOM | 1292 | CA | THR | A | 173 | 30.650 | 91.928 | 19.713 | 1.00 | 14.40 | A |
| ATOM | 1293 | CB | THR | A | 173 | 29.120 | 91.917 | 19.736 | 1.00 | 14.21 | A |
| ATOM | 1294 | OG1 | THR | A | 173 | 28.639 | 92.402 | 18.477 | 1.00 | 15.42 | A |
| ATOM | 1295 | CG2 | THR | A | 173 | 28.586 | 92.804 | 20.853 | 1.00 | 15.19 | A |
| ATOM | 1296 | C | THR | A | 173 | 31.077 | 91.135 | 18.489 | 1.00 | 13.03 | A |
| ATOM | 1297 | O | THR | A | 173 | 31.485 | 91.707 | 17.477 | 1.00 | 13.31 | A |
| ATOM | 1298 | N | THR | A | 174 | 30.959 | 89.815 | 18.578 | 1.00 | 12.27 | A |
| ATOM | 1299 | CA | THR | A | 174 | 31.320 | 88.952 | 17.465 | 1.00 | 13.54 | A |
| ATOM | 1300 | CB | THR | A | 174 | 31.082 | 87.475 | 17.820 | 1.00 | 12.72 | A |
| ATOM | 1301 | OG1 | THR | A | 174 | 31.902 | 87.124 | 18.941 | 1.00 | 13.63 | A |
| ATOM | 1302 | CG2 | THR | A | 174 | 31.437 | 86.572 | 16.643 | 1.00 | 12.54 | A |
| ATOM | 1303 | C | THR | A | 174 | 30.478 | 89.326 | 16.249 | 1.00 | 13.64 | A |
| ATOM | 1304 | O | THR | A | 174 | 30.994 | 89.446 | 15.136 | 1.00 | 12.34 | A |
| ATOM | 1305 | N | ALA | A | 175 | 29.182 | 89.528 | 16.470 | 1.00 | 13.64 | A |
| ATOM | 1306 | CA | ALA | A | 175 | 28.267 | 89.889 | 15.392 | 1.00 | 14.24 | A |
| ATOM | 1307 | CB | ALA | A | 175 | 26.849 | 90.026 | 15.939 | 1.00 | 15.47 | A |
| ATOM | 1308 | C | ALA | A | 175 | 28.689 | 91.183 | 14.697 | 1.00 | 14.01 | A |
| ATOM | 1309 | O | ALA | A | 175 | 28.568 | 91.308 | 13.478 | 1.00 | 14.70 | A |
| ATOM | 1310 | N | GLU | A | 176 | 29.181 | 92.143 | 15.476 | 1.00 | 13.28 | A |
| ATOM | 1311 | CA | GLU | A | 176 | 29.624 | 93.423 | 14.930 | 1.00 | 14.06 | A |
| ATOM | 1312 | CB | GLU | A | 176 | 29.903 | 94.387 | 16.085 | 1.00 | 15.36 | A |
| ATOM | 1313 | CG | GLU | A | 176 | 30.259 | 95.796 | 15.652 | 1.00 | 21.05 | A |
| ATOM | 1314 | CD | GLU | A | 176 | 30.652 | 96.674 | 16.823 | 1.00 | 23.31 | A |
| ATOM | 1315 | OE1 | GLU | A | 176 | 29.946 | 96.641 | 17.852 | 1.00 | 25.59 | A |
| ATOM | 1316 | OE2 | GLU | A | 176 | 31.660 | 97.402 | 16.713 | 1.00 | 25.39 | A |
| ATOM | 1317 | C | GLU | A | 176 | 30.841 | 93.202 | 14.036 | 1.00 | 13.19 | A |
| ATOM | 1318 | O | GLU | A | 176 | 30.954 | 93.799 | 12.965 | 1.00 | 12.97 | A |
| ATOM | 1319 | N | GLY | A | 177 | 31.757 | 92.351 | 14.491 | 1.00 | 11.87 | A |
| ATOM | 1320 | CA | GLY | A | 177 | 32.948 | 92.062 | 13.711 | 1.00 | 12.60 | A |
| ATOM | 1321 | C | GLY | A | 177 | 32.588 | 91.401 | 12.394 | 1.00 | 12.97 | A |
| ATOM | 1322 | O | GLY | A | 177 | 33.098 | 91.779 | 11.334 | 1.00 | 11.59 | A |
| ATOM | 1323 | N | VAL | A | 178 | 31.699 | 90.416 | 12.460 | 1.00 | 12.50 | A |
| ATOM | 1324 | CA | VAL | A | 178 | 31.257 | 89.697 | 11.272 | 1.00 | 12.49 | A |
| ATOM | 1325 | CB | VAL | A | 178 | 30.294 | 88.547 | 11.645 | 1.00 | 13.51 | A |
| ATOM | 1326 | CG1 | VAL | A | 178 | 29.691 | 87.941 | 10.384 | 1.00 | 12.61 | A |
| ATOM | 1327 | CG2 | VAL | A | 178 | 31.035 | 87.484 | 12.434 | 1.00 | 14.57 | A |
| ATOM | 1328 | C | VAL | A | 178 | 30.557 | 90.637 | 10.293 | 1.00 | 12.98 | A |
| ATOM | 1329 | O | VAL | A | 178 | 30.807 | 90.590 | 9.089 | 1.00 | 13.29 | A |
| ATOM | 1330 | N | ALA | A | 179 | 29.677 | 91.489 | 10.810 | 1.00 | 12.81 | A |
| ATOM | 1331 | CA | ALA | A | 179 | 28.962 | 92.436 | 9.961 | 1.00 | 13.11 | A |
| ATOM | 1332 | CB | ALA | A | 179 | 27.945 | 93.223 | 10.788 | 1.00 | 13.80 | A |
| ATOM | 1333 | C | ALA | A | 179 | 29.938 | 93.394 | 9.288 | 1.00 | 14.25 | A |
| ATOM | 1334 | O | ALA | A | 179 | 29.770 | 93.745 | 8.122 | 1.00 | 14.57 | A |
| ATOM | 1335 | N | ARG | A | 180 | 30.962 | 93.816 | 10.025 | 1.00 | 12.82 | A |
| ATOM | 1336 | CA | ARG | A | 180 | 31.946 | 94.737 | 9.472 | 1.00 | 12.12 | A |
| ATOM | 1337 | CB | ARG | A | 180 | 32.859 | 95.239 | 10.589 | 1.00 | 12.50 | A |
| ATOM | 1338 | CG | ARG | A | 180 | 33.793 | 96.343 | 10.145 | 1.00 | 13.41 | A |
| ATOM | 1339 | CD | ARG | A | 180 | 34.401 | 97.029 | 11.346 | 1.00 | 13.48 | A |
| ATOM | 1340 | NE | ARG | A | 180 | 35.338 | 98.071 | 10.948 | 1.00 | 14.00 | A |
| ATOM | 1341 | CZ | ARG | A | 180 | 36.000 | 98.838 | 11.805 | 1.00 | 14.74 | A |
| ATOM | 1342 | NH1 | ARG | A | 180 | 35.821 | 98.682 | 13.110 | 1.00 | 15.16 | A |
| ATOM | 1343 | NH2 | ARG | A | 180 | 36.855 | 99.746 | 11.355 | 1.00 | 16.18 | A |
| ATOM | 1344 | C | ARG | A | 180 | 32.692 | 94.070 | 8.326 | 1.00 | 12.59 | A |
| ATOM | 1345 | O | ARG | A | 180 | 32.947 | 94.694 | 7.298 | 1.00 | 13.27 | A |
| ATOM | 1346 | N | VAL | A | 181 | 33.048 | 92.802 | 8.501 | 1.00 | 11.72 | A |
| ATOM | 1347 | CA | VAL | A | 181 | 33.738 | 92.076 | 7.441 | 1.00 | 11.47 | A |
| ATOM | 1348 | CB | VAL | A | 181 | 34.089 | 90.636 | 7.883 | 1.00 | 11.29 | A |
| ATOM | 1349 | CG1 | VAL | A | 181 | 34.543 | 89.810 | 6.685 | 1.00 | 12.77 | A |
| ATOM | 1350 | CG2 | VAL | A | 181 | 35.180 | 90.669 | 8.944 | 1.00 | 11.42 | A |
| ATOM | 1351 | C | VAL | A | 181 | 32.832 | 92.000 | 6.209 | 1.00 | 12.09 | A |
| ATOM | 1352 | O | VAL | A | 181 | 33.257 | 92.286 | 5.088 | 1.00 | 12.61 | A |
| ATOM | 1353 | N | ARG | A | 182 | 31.574 | 91.640 | 6.433 | 1.00 | 11.83 | A |
| ATOM | 1354 | CA | ARG | A | 182 | 30.607 | 91.492 | 5.350 | 1.00 | 12.81 | A |
| ATOM | 1355 | CB | ARG | A | 182 | 29.352 | 90.805 | 5.899 | 1.00 | 13.78 | A |
| ATOM | 1356 | CG | ARG | A | 182 | 29.567 | 89.354 | 6.291 | 1.00 | 14.34 | A |
| ATOM | 1357 | CD | ARG | A | 182 | 28.296 | 88.715 | 6.820 | 1.00 | 13.02 | A |
| ATOM | 1358 | NE | ARG | A | 182 | 28.426 | 87.264 | 6.889 | 1.00 | 13.79 | A |
| ATOM | 1359 | CZ | ARG | A | 182 | 27.743 | 86.491 | 7.726 | 1.00 | 15.56 | A |
| ATOM | 1360 | NH1 | ARG | A | 182 | 26.878 | 87.028 | 8.576 | 1.00 | 15.17 | A |
| ATOM | 1361 | NH2 | ARG | A | 182 | 27.924 | 85.178 | 7.711 | 1.00 | 16.94 | A |
| ATOM | 1362 | C | ARG | A | 182 | 30.272 | 92.769 | 4.596 | 1.00 | 14.15 | A |
| ATOM | 1363 | O | ARG | A | 182 | 30.014 | 92.724 | 3.392 | 1.00 | 15.65 | A |
| ATOM | 1364 | N | ALA | A | 183 | 30.275 | 93.903 | 5.289 | 1.00 | 14.36 | A |
| ATOM | 1365 | CA | ALA | A | 183 | 29.922 | 95.168 | 4.655 | 1.00 | 15.55 | A |
| ATOM | 1366 | CB | ALA | A | 183 | 28.981 | 95.942 | 5.570 | 1.00 | 16.27 | A |
| ATOM | 1367 | C | ALA | A | 183 | 31.081 | 96.067 | 4.242 | 1.00 | 16.15 | A |
| ATOM | 1368 | O | ALA | A | 183 | 30.852 | 97.151 | 3.701 | 1.00 | 18.41 | A |
| ATOM | 1369 | N | SER | A | 184 | 32.316 | 95.628 | 4.465 | 1.00 | 15.26 | A |
| ATOM | 1370 | CA | SER | A | 184 | 33.470 | 96.462 | 4.133 | 1.00 | 15.42 | A |
| ATOM | 1371 | CB | SER | A | 184 | 34.459 | 96.412 | 5.303 | 1.00 | 15.55 | A |
| ATOM | 1372 | OG | SER | A | 184 | 33.877 | 96.983 | 6.466 | 1.00 | 16.22 | A |
| ATOM | 1373 | C | SER | A | 184 | 34.125 | 96.194 | 2.780 | 1.00 | 14.72 | A |
| ATOM | 1374 | O | SER | A | 184 | 35.223 | 96.681 | 2.517 | 1.00 | 15.02 | A |
| ATOM | 1375 | N | ALA | A | 185 | 33.451 | 95.430 | 1.925 | 1.00 | 15.34 | A |
| ATOM | 1376 | CA | ALA | A | 185 | 33.961 | 95.129 | 0.588 | 1.00 | 16.07 | A |
| ATOM | 1377 | CB | ALA | A | 185 | 33.834 | 96.364 | −0.299 | 1.00 | 16.92 | A |
| ATOM | 1378 | C | ALA | A | 185 | 35.403 | 94.628 | 0.569 | 1.00 | 16.48 | A |
| ATOM | 1379 | O | ALA | A | 185 | 36.176 | 94.967 | −0.334 | 1.00 | 17.01 | A |
| ATOM | 1380 | N | GLY | A | 186 | 35.761 | 93.824 | 1.565 | 1.00 | 15.70 | A |
| ATOM | 1381 | CA | GLY | A | 186 | 37.104 | 93.282 | 1.640 | 1.00 | 15.00 | A |
| ATOM | 1382 | C | GLY | A | 186 | 38.130 | 94.205 | 2.272 | 1.00 | 12.92 | A |
| ATOM | 1383 | O | GLY | A | 186 | 39.321 | 93.894 | 2.260 | 1.00 | 15.00 | A |
| ATOM | 1384 | N | LYS | A | 187 | 37.678 | 95.326 | 2.831 | 1.00 | 12.80 | A |
| ATOM | 1385 | CA | LYS | A | 187 | 38.588 | 96.285 | 3.452 | 1.00 | 13.16 | A |
| ATOM | 1386 | CB | LYS | A | 187 | 38.183 | 97.706 | 3.044 | 1.00 | 14.68 | A |
| ATOM | 1387 | CG | LYS | A | 187 | 38.165 | 97.942 | 1.541 | 1.00 | 16.39 | A |
| ATOM | 1388 | CD | LYS | A | 187 | 39.530 | 97.720 | 0.916 | 1.00 | 19.92 | A |
| ATOM | 1389 | CE | LYS | A | 187 | 39.476 | 97.944 | −0.590 | 1.00 | 22.39 | A |
| ATOM | 1390 | NZ | LYS | A | 187 | 40.797 | 97.715 | −1.237 | 1.00 | 24.55 | A |
| ATOM | 1391 | C | LYS | A | 187 | 38.710 | 96.122 | 4.962 | 1.00 | 13.20 | A |
| ATOM | 1392 | O | LYS | A | 187 | 39.306 | 96.960 | 5.630 | 1.00 | 12.61 | A |
| ATOM | 1393 | N | TYR | A | 188 | 38.131 | 95.059 | 5.506 | 1.00 | 11.47 | A |
| ATOM | 1394 | CA | TYR | A | 188 | 38.238 | 94.798 | 6.936 | 1.00 | 10.48 | A |
| ATOM | 1395 | CB | TYR | A | 188 | 37.025 | 95.349 | 7.697 | 1.00 | 9.76 | A |
| ATOM | 1396 | CG | TYR | A | 188 | 37.156 | 95.179 | 9.205 | 1.00 | 9.93 | A |
| ATOM | 1397 | CD1 | TYR | A | 188 | 37.983 | 96.021 | 9.956 | 1.00 | 11.85 | A |
| ATOM | 1398 | CE1 | TYR | A | 188 | 38.152 | 95.837 | 11.332 | 1.00 | 11.50 | A |
| ATOM | 1399 | CD2 | TYR | A | 188 | 36.498 | 94.144 | 9.875 | 1.00 | 9.54 | A |
| ATOM | 1400 | CE2 | TYR | A | 188 | 36.664 | 93.950 | 11.252 | 1.00 | 10.41 | A |
| ATOM | 1401 | CZ | TYR | A | 188 | 37.491 | 94.801 | 11.973 | 1.00 | 9.98 | A |
| ATOM | 1402 | OH | TYR | A | 188 | 37.657 | 94.612 | 13.324 | 1.00 | 10.94 | A |
| ATOM | 1403 | C | TYR | A | 188 | 38.390 | 93.309 | 7.155 | 1.00 | 9.90 | A |
| ATOM | 1404 | O | TYR | A | 188 | 37.642 | 92.514 | 6.587 | 1.00 | 11.93 | A |
| ATOM | 1405 | N | ALA | A | 189 | 39.365 | 92.943 | 7.977 | 1.00 | 9.93 | A |
| ATOM | 1406 | CA | ALA | A | 189 | 39.605 | 91.549 | 8.334 | 1.00 | 10.69 | A |
| ATOM | 1407 | CB | ALA | A | 189 | 41.013 | 91.124 | 7.947 | 1.00 | 11.59 | A |
| ATOM | 1408 | C | ALA | A | 189 | 39.426 | 91.504 | 9.849 | 1.00 | 9.86 | A |
| ATOM | 1409 | O | ALA | A | 189 | 39.845 | 92.421 | 10.556 | 1.00 | 11.37 | A |
| ATOM | 1410 | N | TYR | A | 190 | 38.807 | 90.441 | 10.348 | 1.00 | 9.99 | A |
| ATOM | 1411 | CA | TYR | A | 190 | 38.535 | 90.327 | 11.772 | 1.00 | 9.79 | A |
| ATOM | 1412 | CB | TYR | A | 190 | 37.011 | 90.171 | 11.928 | 1.00 | 9.92 | A |
| ATOM | 1413 | CG | TYR | A | 190 | 36.500 | 89.957 | 13.337 | 1.00 | 10.09 | A |
| ATOM | 1414 | CD1 | TYR | A | 190 | 37.043 | 90.648 | 14.415 | 1.00 | 11.70 | A |
| ATOM | 1415 | CE1 | TYR | A | 190 | 36.576 | 90.437 | 15.714 | 1.00 | 12.47 | A |
| ATOM | 1416 | CD2 | TYR | A | 190 | 35.468 | 89.052 | 13.587 | 1.00 | 11.76 | A |
| ATOM | 1417 | CE2 | TYR | A | 190 | 34.995 | 88.833 | 14.876 | 1.00 | 12.05 | A |
| ATOM | 1418 | CZ | TYR | A | 190 | 35.555 | 89.523 | 15.934 | 1.00 | 12.04 | A |
| ATOM | 1419 | OH | TYR | A | 190 | 35.124 | 89.258 | 17.215 | 1.00 | 14.89 | A |
| ATOM | 1420 | C | TYR | A | 190 | 39.351 | 89.225 | 12.439 | 1.00 | 8.97 | A |
| ATOM | 1421 | O | TYR | A | 190 | 39.390 | 88.095 | 11.957 | 1.00 | 10.33 | A |
| ATOM | 1422 | N | LEU | A | 191 | 40.020 | 89.568 | 13.537 | 1.00 | 9.20 | A |
| ATOM | 1423 | CA | LEU | A | 191 | 40.817 | 88.606 | 14.295 | 1.00 | 9.62 | A |
| ATOM | 1424 | CB | LEU | A | 191 | 42.075 | 89.294 | 14.825 | 1.00 | 10.20 | A |
| ATOM | 1425 | CG | LEU | A | 191 | 43.170 | 89.563 | 13.791 | 1.00 | 11.97 | A |
| ATOM | 1426 | CD1 | LEU | A | 191 | 44.047 | 90.708 | 14.253 | 1.00 | 15.67 | A |
| ATOM | 1427 | CD2 | LEU | A | 191 | 43.984 | 88.297 | 13.565 | 1.00 | 12.16 | A |
| ATOM | 1428 | C | LEU | A | 191 | 39.928 | 88.062 | 15.400 | 1.00 | 10.23 | A |
| ATOM | 1429 | O | LEU | A | 191 | 39.533 | 88.803 | 16.301 | 1.00 | 10.12 | A |
| ATOM | 1430 | N | LEU | A | 192 | 39.624 | 86.769 | 15.329 | 1.00 | 10.06 | A |
| ATOM | 1431 | CA | LEU | A | 192 | 38.761 | 86.128 | 16.315 | 1.00 | 9.01 | A |
| ATOM | 1432 | CB | LEU | A | 192 | 37.301 | 86.240 | 15.861 | 1.00 | 10.17 | A |
| ATOM | 1433 | CG | LEU | A | 192 | 36.844 | 85.219 | 14.813 | 1.00 | 9.95 | A |
| ATOM | 1434 | CD1 | LEU | A | 192 | 35.344 | 85.357 | 14.609 | 1.00 | 10.24 | A |
| ATOM | 1435 | CD2 | LEU | A | 192 | 37.582 | 85.425 | 13.495 | 1.00 | 10.31 | A |
| ATOM | 1436 | C | LEU | A | 192 | 39.183 | 84.682 | 16.532 | 1.00 | 8.95 | A |
| ATOM | 1437 | O | LEU | A | 192 | 40.000 | 84.136 | 15.788 | 1.00 | 8.84 | A |
| ATOM | 1438 | N | GLU | A | 193 | 38.616 | 84.042 | 17.546 | 1.00 | 9.42 | A |
| ATOM | 1439 | CA | GLU | A | 193 | 38.969 | 82.658 | 17.815 | 1.00 | 9.32 | A |
| ATOM | 1440 | CB | GLU | A | 193 | 38.484 | 82.303 | 19.226 | 1.00 | 9.15 | A |
| ATOM | 1441 | CG | GLU | A | 193 | 39.210 | 83.161 | 20.264 | 1.00 | 9.00 | A |
| ATOM | 1442 | CD | GLU | A | 193 | 38.783 | 82.924 | 21.698 | 1.00 | 7.63 | A |
| ATOM | 1443 | OE1 | GLU | A | 193 | 37.618 | 82.541 | 21.929 | 1.00 | 8.76 | A |
| ATOM | 1444 | OE2 | GLU | A | 193 | 39.619 | 83.143 | 22.596 | 1.00 | 8.73 | A |
| ATOM | 1445 | C | GLU | A | 193 | 38.477 | 81.746 | 16.686 | 1.00 | 9.08 | A |
| ATOM | 1446 | O | GLU | A | 193 | 37.391 | 81.937 | 16.132 | 1.00 | 8.87 | A |
| ATOM | 1447 | N | SER | A | 194 | 39.312 | 80.769 | 16.338 | 1.00 | 9.00 | A |
| ATOM | 1448 | CA | SER | A | 194 | 39.031 | 79.836 | 15.247 | 1.00 | 9.40 | A |
| ATOM | 1449 | CB | SER | A | 194 | 40.172 | 78.819 | 15.167 | 1.00 | 9.63 | A |
| ATOM | 1450 | OG | SER | A | 194 | 40.297 | 78.090 | 16.375 | 1.00 | 9.18 | A |
| ATOM | 1451 | C | SER | A | 194 | 37.659 | 79.183 | 15.344 | 1.00 | 9.07 | A |
| ATOM | 1452 | O | SER | A | 194 | 37.016 | 78.919 | 14.327 | 1.00 | 8.31 | A |
| ATOM | 1453 | N | THR | A | 195 | 37.226 | 78.913 | 16.569 | 1.00 | 8.83 | A |
| ATOM | 1454 | CA | THR | A | 195 | 35.928 | 78.314 | 16.827 | 1.00 | 9.21 | A |
| ATOM | 1455 | CB | THR | A | 195 | 35.771 | 78.100 | 18.328 | 1.00 | 11.82 | A |
| ATOM | 1456 | OG1 | THR | A | 195 | 36.318 | 79.236 | 19.005 | 1.00 | 10.54 | A |
| ATOM | 1457 | CG2 | THR | A | 195 | 36.518 | 76.845 | 18.773 | 1.00 | 11.99 | A |
| ATOM | 1458 | C | THR | A | 195 | 34.797 | 79.203 | 16.287 | 1.00 | 9.86 | A |
| ATOM | 1459 | O | THR | A | 195 | 33.918 | 78.723 | 15.580 | 1.00 | 9.91 | A |
| ATOM | 1460 | N | MET | A | 196 | 34.814 | 80.496 | 16.607 | 1.00 | 10.11 | A |
| ATOM | 1461 | CA | MET | A | 196 | 33.776 | 81.396 | 16.100 | 1.00 | 11.13 | A |
| ATOM | 1462 | CB | MET | A | 196 | 33.866 | 82.767 | 16.781 | 1.00 | 10.81 | A |
| ATOM | 1463 | CG | MET | A | 196 | 33.280 | 82.832 | 18.176 | 1.00 | 12.64 | A |
| ATOM | 1464 | SD | MET | A | 196 | 34.294 | 81.964 | 19.362 | 1.00 | 12.02 | A |
| ATOM | 1465 | CE | MET | A | 196 | 33.945 | 82.894 | 20.858 | 1.00 | 13.21 | A |
| ATOM | 1466 | C | MET | A | 196 | 33.895 | 81.525 | 14.589 | 1.00 | 10.05 | A |
| ATOM | 1467 | O | MET | A | 196 | 32.892 | 81.583 | 13.878 | 1.00 | 11.28 | A |
| ATOM | 1468 | N | ASN | A | 197 | 35.133 | 81.585 | 14.111 | 1.00 | 10.27 | A |
| ATOM | 1469 | CA | ASN | A | 197 | 35.415 | 81.724 | 12.685 | 1.00 | 10.27 | A |
| ATOM | 1470 | CB | ASN | A | 197 | 36.936 | 81.798 | 12.506 | 1.00 | 10.10 | A |
| ATOM | 1471 | CG | ASN | A | 197 | 37.349 | 82.493 | 11.223 | 1.00 | 11.64 | A |
| ATOM | 1472 | OD1 | ASN | A | 197 | 36.534 | 82.715 | 10.330 | 1.00 | 10.77 | A |
| ATOM | 1473 | ND2 | ASN | A | 197 | 38.628 | 82.833 | 11.125 | 1.00 | 9.60 | A |
| ATOM | 1474 | C | ASN | A | 197 | 34.777 | 80.572 | 11.902 | 1.00 | 10.52 | A |
| ATOM | 1475 | O | ASN | A | 197 | 34.086 | 80.786 | 10.901 | 1.00 | 10.37 | A |
| ATOM | 1476 | N | GLU | A | 198 | 35.022 | 79.349 | 12.367 | 1.00 | 11.10 | A |
| ATOM | 1477 | CA | GLU | A | 198 | 34.493 | 78.140 | 11.732 | 1.00 | 11.31 | A |
| ATOM | 1478 | CB | GLU | A | 198 | 35.101 | 76.914 | 12.420 | 1.00 | 13.92 | A |
| ATOM | 1479 | CG | GLU | A | 198 | 36.607 | 76.801 | 12.283 | 1.00 | 15.71 | A |
| ATOM | 1480 | CD | GLU | A | 198 | 37.231 | 75.942 | 13.370 | 1.00 | 19.06 | A |
| ATOM | 1481 | OE1 | GLU | A | 198 | 38.462 | 75.737 | 13.329 | 1.00 | 20.07 | A |
| ATOM | 1482 | OE2 | GLU | A | 198 | 36.497 | 75.477 | 14.269 | 1.00 | 19.02 | A |
| ATOM | 1483 | C | GLU | A | 198 | 32.970 | 78.125 | 11.762 | 1.00 | 10.82 | A |
| ATOM | 1484 | O | GLU | A | 198 | 32.322 | 77.698 | 10.803 | 1.00 | 12.14 | A |
| ATOM | 1485 | N | TYR | A | 199 | 32.402 | 78.583 | 12.872 | 1.00 | 11.22 | A |
| ATOM | 1486 | CA | TYR | A | 199 | 30.956 | 78.622 | 13.024 | 1.00 | 11.52 | A |
| ATOM | 1487 | CB | TYR | A | 199 | 30.615 | 79.051 | 14.449 | 1.00 | 12.35 | A |
| ATOM | 1488 | CG | TYR | A | 199 | 29.172 | 79.466 | 14.616 | 1.00 | 11.56 | A |
| ATOM | 1489 | CD1 | TYR | A | 199 | 28.144 | 78.523 | 14.590 | 1.00 | 13.00 | A |
| ATOM | 1490 | CE1 | TYR | A | 199 | 26.807 | 78.911 | 14.722 | 1.00 | 12.91 | A |
| ATOM | 1491 | CD2 | TYR | A | 199 | 28.832 | 80.809 | 14.778 | 1.00 | 12.75 | A |
| ATOM | 1492 | CE2 | TYR | A | 199 | 27.504 | 81.207 | 14.910 | 1.00 | 14.56 | A |
| ATOM | 1493 | CZ | TYR | A | 199 | 26.499 | 80.254 | 14.882 | 1.00 | 13.63 | A |
| ATOM | 1494 | OH | TYR | A | 199 | 25.189 | 80.651 | 15.018 | 1.00 | 17.35 | A |
| ATOM | 1495 | C | TYR | A | 199 | 30.343 | 79.544 | 11.979 | 1.00 | 11.53 | A |
| ATOM | 1496 | O | TYR | A | 199 | 29.429 | 79.155 | 11.254 | 1.00 | 13.28 | A |
| ATOM | 1497 | N | ILE | A | 200 | 30.854 | 80.770 | 11.912 | 1.00 | 12.00 | A |
| ATOM | 1498 | CA | ILE | A | 200 | 30.359 | 81.771 | 10.972 | 1.00 | 12.68 | A |
| ATOM | 1499 | CB | ILE | A | 200 | 31.058 | 83.135 | 11.214 | 1.00 | 13.01 | A |
| ATOM | 1500 | CG2 | ILE | A | 200 | 30.630 | 84.144 | 10.155 | 1.00 | 12.44 | A |
| ATOM | 1501 | CG1 | ILE | A | 200 | 30.715 | 83.664 | 12.614 | 1.00 | 14.61 | A |
| ATOM | 1502 | CD1 | ILE | A | 200 | 29.244 | 84.000 | 12.829 | 1.00 | 14.43 | A |
| ATOM | 1503 | C | ILE | A | 200 | 30.563 | 81.350 | 9.515 | 1.00 | 12.69 | A |
| ATOM | 1504 | O | ILE | A | 200 | 29.748 | 81.668 | 8.646 | 1.00 | 12.52 | A |
| ATOM | 1505 | N | GLU | A | 201 | 31.651 | 80.634 | 9.255 | 1.00 | 12.45 | A |
| ATOM | 1506 | CA | GLU | A | 201 | 31.964 | 80.170 | 7.910 | 1.00 | 15.01 | A |
| ATOM | 1507 | CB | GLU | A | 201 | 33.330 | 79.483 | 7.942 | 1.00 | 15.01 | A |
| ATOM | 1508 | CG | GLU | A | 201 | 33.910 | 79.134 | 6.586 | 1.00 | 20.19 | A |
| ATOM | 1509 | CD | GLU | A | 201 | 35.322 | 78.593 | 6.698 | 1.00 | 21.08 | A |
| ATOM | 1510 | OE1 | GLU | A | 201 | 35.507 | 77.530 | 7.328 | 1.00 | 24.42 | A |
| ATOM | 1511 | OE2 | GLU | A | 201 | 36.249 | 79.234 | 6.164 | 1.00 | 25.13 | A |
| ATOM | 1512 | C | GLU | A | 201 | 30.857 | 79.272 | 7.366 | 1.00 | 15.85 | A |
| ATOM | 1513 | O | GLU | A | 201 | 30.718 | 79.120 | 6.151 | 1.00 | 15.98 | A |
| ATOM | 1514 | N | GLN | A | 202 | 30.070 | 78.682 | 8.263 | 1.00 | 15.03 | A |
| ATOM | 1515 | CA | GLN | A | 202 | 28.982 | 77.792 | 7.864 | 1.00 | 17.87 | A |
| ATOM | 1516 | CB | GLN | A | 202 | 29.070 | 76.499 | 8.687 | 1.00 | 18.85 | A |
| ATOM | 1517 | CG | GLN | A | 202 | 30.394 | 75.758 | 8.569 | 1.00 | 22.01 | A |
| ATOM | 1518 | CD | GLN | A | 202 | 30.753 | 75.418 | 7.135 | 1.00 | 25.59 | A |
| ATOM | 1519 | OE1 | GLN | A | 202 | 29.948 | 74.848 | 6.399 | 1.00 | 28.46 | A |
| ATOM | 1520 | NE2 | GLN | A | 202 | 31.973 | 75.758 | 6.734 | 1.00 | 26.17 | A |
| ATOM | 1521 | C | GLN | A | 202 | 27.622 | 78.479 | 7.987 | 1.00 | 18.30 | A |
| ATOM | 1522 | O | GLN | A | 202 | 26.586 | 77.813 | 8.026 | 1.00 | 21.15 | A |
| ATOM | 1523 | N | ARG | A | 203 | 27.624 | 79.806 | 8.044 | 1.00 | 17.72 | A |
| ATOM | 1524 | CA | ARG | A | 203 | 26.384 | 80.569 | 8.167 | 1.00 | 17.74 | A |
| ATOM | 1525 | CB | ARG | A | 203 | 26.448 | 81.435 | 9.432 | 1.00 | 19.84 | A |
| ATOM | 1526 | CG | ARG | A | 203 | 26.722 | 80.670 | 10.716 | 1.00 | 20.83 | A |
| ATOM | 1527 | CD | ARG | A | 203 | 25.507 | 79.882 | 11.176 | 1.00 | 24.68 | A |
| ATOM | 1528 | NE | ARG | A | 203 | 24.412 | 80.761 | 11.576 | 1.00 | 27.02 | A |
| ATOM | 1529 | CZ | ARG | A | 203 | 23.261 | 80.336 | 12.087 | 1.00 | 28.82 | A |
| ATOM | 1530 | NH1 | ARG | A | 203 | 23.050 | 79.039 | 12.262 | 1.00 | 28.91 | A |
| ATOM | 1531 | NH2 | ARG | A | 203 | 22.322 | 81.210 | 12.426 | 1.00 | 28.42 | A |
| ATOM | 1532 | C | ARG | A | 203 | 26.159 | 81.401 | 6.912 | 1.00 | 17.54 | A |
| ATOM | 1533 | O | ARG | A | 203 | 27.105 | 81.941 | 6.337 | 1.00 | 16.78 | A |
| ATOM | 1534 | N | LYS | A | 204 | 24.904 | 81.502 | 6.487 | 1.00 | 19.44 | A |
| ATOM | 1535 | CA | LYS | A | 204 | 24.568 | 82.301 | 5.316 | 1.00 | 20.89 | A |
| ATOM | 1536 | CB | LYS | A | 204 | 23.086 | 82.103 | 4.981 | 1.00 | 22.65 | A |
| ATOM | 1537 | CG | LYS | A | 204 | 22.733 | 80.694 | 4.520 | 1.00 | 26.49 | A |
| ATOM | 1538 | CD | LYS | A | 204 | 21.223 | 80.488 | 4.438 | 1.00 | 29.93 | A |
| ATOM | 1539 | CE | LYS | A | 204 | 20.572 | 81.455 | 3.461 | 1.00 | 32.13 | A |
| ATOM | 1540 | NZ | LYS | A | 204 | 19.090 | 81.293 | 3.421 | 1.00 | 34.80 | A |
| ATOM | 1541 | C | LYS | A | 204 | 24.926 | 83.737 | 5.682 | 1.00 | 20.57 | A |
| ATOM | 1542 | O | LYS | A | 204 | 24.876 | 84.113 | 6.855 | 1.00 | 21.17 | A |
| ATOM | 1543 | N | PRO | A | 205 | 25.259 | 84.573 | 4.687 | 1.00 | 20.27 | A |
| ATOM | 1544 | CD | PRO | A | 205 | 25.404 | 86.015 | 4.951 | 1.00 | 20.25 | A |
| ATOM | 1545 | CA | PRO | A | 205 | 25.332 | 84.310 | 3.247 | 1.00 | 19.98 | A |
| ATOM | 1546 | CB | PRO | A | 205 | 25.111 | 85.700 | 2.659 | 1.00 | 20.23 | A |
| ATOM | 1547 | CG | PRO | A | 205 | 25.918 | 86.568 | 3.569 | 1.00 | 22.10 | A |
| ATOM | 1548 | C | PRO | A | 205 | 26.578 | 83.585 | 2.727 | 1.00 | 19.52 | A |
| ATOM | 1549 | O | PRO | A | 205 | 26.893 | 83.678 | 1.541 | 1.00 | 20.02 | A |
| ATOM | 1550 | N | CYS | A | 206 | 27.292 | 82.877 | 3.597 | 1.00 | 17.94 | A |
| ATOM | 1551 | CA | CYS | A | 206 | 28.474 | 82.127 | 3.171 | 1.00 | 17.10 | A |
| ATOM | 1552 | C | CYS | A | 206 | 29.475 | 83.007 | 2.435 | 1.00 | 16.19 | A |
| ATOM | 1553 | O | CYS | A | 206 | 30.006 | 82.610 | 1.396 | 1.00 | 16.94 | A |
| ATOM | 1554 | CB | CYS | A | 206 | 28.018 | 80.945 | 2.310 | 1.00 | 18.59 | A |
| ATOM | 1555 | SG | CYS | A | 206 | 26.707 | 79.952 | 3.079 | 1.00 | 20.55 | A |
| ATOM | 1556 | N | ASP | A | 207 | 29.744 | 84.189 | 2.976 | 1.00 | 15.45 | A |
| ATOM | 1557 | CA | ASP | A | 207 | 30.666 | 85.116 | 2.334 | 1.00 | 14.32 | A |
| ATOM | 1558 | CB | ASP | A | 207 | 29.907 | 86.408 | 2.009 | 1.00 | 16.26 | A |
| ATOM | 1559 | CG | ASP | A | 207 | 29.412 | 87.120 | 3.252 | 1.00 | 15.80 | A |
| ATOM | 1560 | OD1 | ASP | A | 207 | 29.376 | 86.488 | 4.330 | 1.00 | 16.40 | A |
| ATOM | 1561 | OD2 | ASP | A | 207 | 29.048 | 88.311 | 3.150 | 1.00 | 18.95 | A |
| ATOM | 1562 | C | ASP | A | 207 | 31.937 | 85.361 | 3.139 | 1.00 | 14.39 | A |
| ATOM | 1563 | O | ASP | A | 207 | 32.711 | 86.261 | 2.817 | 1.00 | 13.94 | A |
| ATOM | 1564 | N | THR | A | 208 | 32.155 | 84.573 | 4.187 | 1.00 | 14.10 | A |
| ATOM | 1565 | CA | THR | A | 208 | 33.358 | 84.734 | 4.997 | 1.00 | 13.35 | A |
| ATOM | 1566 | CB | THR | A | 208 | 33.027 | 84.977 | 6.483 | 1.00 | 13.59 | A |
| ATOM | 1567 | OG1 | THR | A | 208 | 32.375 | 83.823 | 7.026 | 1.00 | 15.09 | A |
| ATOM | 1568 | CG2 | THR | A | 208 | 32.123 | 86.193 | 6.634 | 1.00 | 14.70 | A |
| ATOM | 1569 | C | THR | A | 208 | 34.226 | 83.487 | 4.891 | 1.00 | 13.65 | A |
| ATOM | 1570 | O | THR | A | 208 | 33.753 | 82.414 | 4.506 | 1.00 | 14.29 | A |
| ATOM | 1571 | N | MET | A | 209 | 35.497 | 83.627 | 5.241 | 1.00 | 13.49 | A |
| ATOM | 1572 | CA | MET | A | 209 | 36.419 | 82.509 | 5.166 | 1.00 | 14.22 | A |
| ATOM | 1573 | CB | MET | A | 209 | 36.919 | 82.399 | 3.723 | 1.00 | 17.35 | A |
| ATOM | 1574 | CG | MET | A | 209 | 38.022 | 81.385 | 3.508 | 1.00 | 20.95 | A |
| ATOM | 1575 | SD | MET | A | 209 | 38.507 | 81.287 | 1.773 | 1.00 | 25.45 | A |
| ATOM | 1576 | CE | MET | A | 209 | 39.516 | 82.733 | 1.609 | 1.00 | 25.90 | A |
| ATOM | 1577 | C | MET | A | 209 | 37.550 | 82.654 | 6.168 | 1.00 | 13.82 | A |
| ATOM | 1578 | O | MET | A | 209 | 38.030 | 83.762 | 6.417 | 1.00 | 13.46 | A |
| ATOM | 1579 | N | LYS | A | 210 | 37.963 | 81.531 | 6.749 | 1.00 | 15.10 | A |
| ATOM | 1580 | CA | LYS | A | 210 | 39.076 | 81.515 | 7.690 | 1.00 | 15.37 | A |
| ATOM | 1581 | CB | LYS | A | 210 | 38.870 | 80.410 | 8.734 | 1.00 | 16.35 | A |
| ATOM | 1582 | CG | LYS | A | 210 | 40.045 | 80.256 | 9.702 | 1.00 | 19.49 | A |
| ATOM | 1583 | CD | LYS | A | 210 | 39.752 | 79.249 | 10.813 | 1.00 | 21.65 | A |
| ATOM | 1584 | CE | LYS | A | 210 | 39.665 | 77.824 | 10.288 | 1.00 | 23.45 | A |
| ATOM | 1585 | NZ | LYS | A | 210 | 40.975 | 77.345 | 9.768 | 1.00 | 24.20 | A |
| ATOM | 1586 | C | LYS | A | 210 | 40.316 | 81.294 | 6.834 | 1.00 | 15.15 | A |
| ATOM | 1587 | O | LYS | A | 210 | 40.356 | 80.361 | 6.032 | 1.00 | 16.64 | A |
| ATOM | 1588 | N | VAL | A | 211 | 41.323 | 82.148 | 6.990 | 1.00 | 14.55 | A |
| ATOM | 1589 | CA | VAL | A | 211 | 42.544 | 82.007 | 6.202 | 1.00 | 15.65 | A |
| ATOM | 1590 | CB | VAL | A | 211 | 42.695 | 83.150 | 5.167 | 1.00 | 15.98 | A |
| ATOM | 1591 | CG1 | VAL | A | 211 | 41.593 | 83.061 | 4.133 | 1.00 | 17.55 | A |
| ATOM | 1592 | CG2 | VAL | A | 211 | 42.666 | 84.500 | 5.867 | 1.00 | 15.82 | A |
| ATOM | 1593 | C | VAL | A | 211 | 43.806 | 81.967 | 7.047 | 1.00 | 16.70 | A |
| ATOM | 1594 | O | VAL | A | 211 | 43.872 | 82.574 | 8.117 | 1.00 | 15.53 | A |
| ATOM | 1595 | N | GLY | A | 212 | 44.809 | 81.250 | 6.547 | 1.00 | 18.12 | A |
| ATOM | 1596 | CA | GLY | A | 212 | 46.072 | 81.134 | 7.249 | 1.00 | 19.13 | A |
| ATOM | 1597 | C | GLY | A | 212 | 45.986 | 80.237 | 8.465 | 1.00 | 18.29 | A |
| ATOM | 1598 | O | GLY | A | 212 | 44.898 | 79.830 | 8.876 | 1.00 | 20.17 | A |
| ATOM | 1599 | N | GLY | A | 213 | 47.139 | 79.912 | 9.036 | 1.00 | 17.49 | A |
| ATOM | 1600 | CA | GLY | A | 213 | 47.147 | 79.076 | 10.218 | 1.00 | 16.45 | A |
| ATOM | 1601 | C | GLY | A | 213 | 46.769 | 79.928 | 11.411 | 1.00 | 14.83 | A |
| ATOM | 1602 | O | GLY | A | 213 | 46.769 | 81.161 | 11.328 | 1.00 | 17.42 | A |
| ATOM | 1603 | N | ASN | A | 214 | 46.433 | 79.288 | 12.522 | 1.00 | 13.24 | A |
| ATOM | 1604 | CA | ASN | A | 214 | 46.073 | 80.039 | 13.713 | 1.00 | 11.96 | A |
| ATOM | 1605 | CB | ASN | A | 214 | 45.423 | 79.103 | 14.735 | 1.00 | 13.42 | A |
| ATOM | 1606 | CG | ASN | A | 214 | 44.093 | 78.554 | 14.262 | 1.00 | 14.31 | A |
| ATOM | 1607 | OD1 | ASN | A | 214 | 43.445 | 79.128 | 13.386 | 1.00 | 14.64 | A |
| ATOM | 1608 | ND2 | ASN | A | 214 | 43.671 | 77.444 | 14.854 | 1.00 | 16.40 | A |
| ATOM | 1609 | C | ASN | A | 214 | 47.329 | 80.722 | 14.238 | 1.00 | 11.82 | A |
| ATOM | 1610 | O | ASN | A | 214 | 48.442 | 80.237 | 14.027 | 1.00 | 13.30 | A |
| ATOM | 1611 | N | LEU | A | 215 | 47.145 | 81.842 | 14.927 | 1.00 | 10.88 | A |
| ATOM | 1612 | CA | LEU | A | 215 | 48.267 | 82.592 | 15.478 | 1.00 | 11.07 | A |
| ATOM | 1613 | CB | LEU | A | 215 | 47.867 | 84.061 | 15.644 | 1.00 | 11.85 | A |
| ATOM | 1614 | CG | LEU | A | 215 | 47.760 | 84.915 | 14.375 | 1.00 | 12.48 | A |
| ATOM | 1615 | CD1 | LEU | A | 215 | 46.698 | 84.359 | 13.439 | 1.00 | 15.01 | A |
| ATOM | 1616 | CD2 | LEU | A | 215 | 47.430 | 86.344 | 14.768 | 1.00 | 13.40 | A |
| ATOM | 1617 | C | LEU | A | 215 | 48.784 | 81.968 | 16.768 | 1.00 | 11.18 | A |
| ATOM | 1618 | O | LEU | A | 215 | 49.931 | 82.197 | 17.151 | 1.00 | 13.06 | A |
| ATOM | 1619 | N | ASP | A | 216 | 47.935 | 81.203 | 17.451 | 1.00 | 10.77 | A |
| ATOM | 1620 | CA | ASP | A | 216 | 48.328 | 80.531 | 18.686 | 1.00 | 10.05 | A |
| ATOM | 1621 | CB | ASP | A | 216 | 48.230 | 81.498 | 19.887 | 1.00 | 10.46 | A |
| ATOM | 1622 | CG | ASP | A | 216 | 46.805 | 81.909 | 20.231 | 1.00 | 9.55 | A |
| ATOM | 1623 | OD1 | ASP | A | 216 | 45.849 | 81.513 | 19.528 | 1.00 | 9.84 | A |
| ATOM | 1624 | OD2 | ASP | A | 216 | 46.658 | 82.649 | 21.231 | 1.00 | 10.37 | A |
| ATOM | 1625 | C | ASP | A | 216 | 47.519 | 79.250 | 18.841 | 1.00 | 9.18 | A |
| ATOM | 1626 | O | ASP | A | 216 | 46.689 | 78.937 | 17.990 | 1.00 | 10.30 | A |
| ATOM | 1627 | N | SER | A | 217 | 47.782 | 78.511 | 19.916 | 1.00 | 10.24 | A |
| ATOM | 1628 | CA | SER | A | 217 | 47.103 | 77.244 | 20.179 | 1.00 | 10.49 | A |
| ATOM | 1629 | CB | SER | A | 217 | 48.017 | 76.095 | 19.747 | 1.00 | 13.97 | A |
| ATOM | 1630 | OG | SER | A | 217 | 48.283 | 76.142 | 18.362 | 1.00 | 20.86 | A |
| ATOM | 1631 | C | SER | A | 217 | 46.729 | 77.169 | 21.649 | 1.00 | 10.89 | A |
| ATOM | 1632 | O | SER | A | 217 | 47.595 | 77.133 | 22.519 | 1.00 | 12.10 | A |
| ATOM | 1633 | N | LYS | A | 218 | 45.431 | 77.124 | 21.926 | 1.00 | 11.01 | A |
| ATOM | 1634 | CA | LYS | A | 218 | 44.961 | 77.075 | 23.300 | 1.00 | 10.66 | A |
| ATOM | 1635 | CB | LYS | A | 218 | 44.917 | 78.507 | 23.845 | 1.00 | 10.42 | A |
| ATOM | 1636 | CG | LYS | A | 218 | 44.102 | 79.476 | 22.999 | 1.00 | 10.17 | A |
| ATOM | 1637 | CD | LYS | A | 218 | 44.537 | 80.910 | 23.264 | 1.00 | 9.85 | A |
| ATOM | 1638 | CE | LYS | A | 218 | 43.734 | 81.900 | 22.438 | 1.00 | 8.64 | A |
| ATOM | 1639 | NZ | LYS | A | 218 | 44.316 | 83.269 | 22.501 | 1.00 | 8.85 | A |
| ATOM | 1640 | C | LYS | A | 218 | 43.628 | 76.348 | 23.369 | 1.00 | 9.98 | A |
| ATOM | 1641 | O | LYS | A | 218 | 43.281 | 75.584 | 22.469 | 1.00 | 9.15 | A |
| ATOM | 1642 | N | GLY | A | 219 | 42.877 | 76.587 | 24.438 | 1.00 | 10.04 | A |
| ATOM | 1643 | CA | GLY | A | 219 | 41.597 | 75.924 | 24.582 | 1.00 | 9.98 | A |
| ATOM | 1644 | C | GLY | A | 219 | 40.682 | 76.612 | 25.570 | 1.00 | 9.33 | A |
| ATOM | 1645 | O | GLY | A | 219 | 41.098 | 77.525 | 26.286 | 1.00 | 9.61 | A |
| ATOM | 1646 | N | TYR | A | 220 | 39.424 | 76.182 | 25.575 | 1.00 | 8.52 | A |
| ATOM | 1647 | CA | TYR | A | 220 | 38.418 | 76.700 | 26.502 | 1.00 | 9.25 | A |
| ATOM | 1648 | CB | TYR | A | 220 | 37.034 | 76.765 | 25.852 | 1.00 | 8.84 | A |
| ATOM | 1649 | CG | TYR | A | 220 | 36.885 | 77.684 | 24.677 | 1.00 | 8.82 | A |
| ATOM | 1650 | CD1 | TYR | A | 220 | 37.475 | 78.946 | 24.664 | 1.00 | 7.89 | A |
| ATOM | 1651 | CE1 | TYR | A | 220 | 37.277 | 79.813 | 23.606 | 1.00 | 8.92 | A |
| ATOM | 1652 | CD2 | TYR | A | 220 | 36.094 | 77.312 | 23.596 | 1.00 | 9.12 | A |
| ATOM | 1653 | CE2 | TYR | A | 220 | 35.887 | 78.172 | 22.531 | 1.00 | 9.33 | A |
| ATOM | 1654 | CZ | TYR | A | 220 | 36.483 | 79.421 | 22.543 | 1.00 | 8.81 | A |
| ATOM | 1655 | OH | TYR | A | 220 | 36.288 | 80.272 | 21.483 | 1.00 | 9.44 | A |
| ATOM | 1656 | C | TYR | A | 220 | 38.386 | 75.740 | 27.670 | 1.00 | 8.73 | A |
| ATOM | 1657 | O | TYR | A | 220 | 38.493 | 74.524 | 27.484 | 1.00 | 8.20 | A |
| ATOM | 1658 | N | GLY | A | 221 | 38.221 | 76.277 | 28.870 | 1.00 | 8.26 | A |
| ATOM | 1659 | CA | GLY | A | 221 | 38.146 | 75.423 | 30.035 | 1.00 | 8.92 | A |
| ATOM | 1660 | C | GLY | A | 221 | 36.988 | 75.801 | 30.934 | 1.00 | 7.99 | A |
| ATOM | 1661 | O | GLY | A | 221 | 36.505 | 76.936 | 30.900 | 1.00 | 8.94 | A |
| ATOM | 1662 | N | ILE | A | 222 | 36.516 | 74.838 | 31.716 | 1.00 | 9.12 | A |
| ATOM | 1663 | CA | ILE | A | 222 | 35.442 | 75.092 | 32.663 | 1.00 | 9.00 | A |
| ATOM | 1664 | CB | ILE | A | 222 | 34.692 | 73.787 | 33.019 | 1.00 | 9.58 | A |
| ATOM | 1665 | CG2 | ILE | A | 222 | 33.793 | 74.015 | 34.229 | 1.00 | 9.65 | A |
| ATOM | 1666 | CG1 | ILE | A | 222 | 33.871 | 73.324 | 31.809 | 1.00 | 12.96 | A |
| ATOM | 1667 | CD1 | ILE | A | 222 | 33.042 | 72.072 | 32.047 | 1.00 | 14.76 | A |
| ATOM | 1668 | C | ILE | A | 222 | 36.147 | 75.659 | 33.893 | 1.00 | 9.66 | A |
| ATOM | 1669 | O | ILE | A | 222 | 37.166 | 75.120 | 34.334 | 1.00 | 11.79 | A |
| ATOM | 1670 | N | ALA | A | 223 | 35.622 | 76.746 | 34.445 | 1.00 | 9.36 | A |
| ATOM | 1671 | CA | ALA | A | 223 | 36.260 | 77.373 | 35.590 | 1.00 | 9.69 | A |
| ATOM | 1672 | CB | ALA | A | 223 | 36.540 | 78.843 | 35.280 | 1.00 | 9.35 | A |
| ATOM | 1673 | C | ALA | A | 223 | 35.460 | 77.258 | 36.879 | 1.00 | 9.65 | A |
| ATOM | 1674 | O | ALA | A | 223 | 34.230 | 77.289 | 36.877 | 1.00 | 9.55 | A |
| ATOM | 1675 | N | THR | A | 224 | 36.188 | 77.124 | 37.982 | 1.00 | 10.12 | A |
| ATOM | 1676 | CA | THR | A | 224 | 35.601 | 77.035 | 39.314 | 1.00 | 10.80 | A |
| ATOM | 1677 | CB | THR | A | 224 | 35.577 | 75.578 | 39.829 | 1.00 | 11.59 | A |
| ATOM | 1678 | OG1 | THR | A | 224 | 36.849 | 74.967 | 39.589 | 1.00 | 12.02 | A |
| ATOM | 1679 | CG2 | THR | A | 224 | 34.489 | 74.778 | 39.141 | 1.00 | 11.59 | A |
| ATOM | 1680 | C | THR | A | 224 | 36.473 | 77.867 | 40.251 | 1.00 | 11.60 | A |
| ATOM | 1681 | O | THR | A | 224 | 37.629 | 78.153 | 39.940 | 1.00 | 11.33 | A |
| ATOM | 1682 | N | PRO | A | 225 | 35.928 | 78.290 | 41.403 | 1.00 | 11.75 | A |
| ATOM | 1683 | CD | PRO | A | 225 | 34.573 | 78.100 | 41.944 | 1.00 | 12.51 | A |
| ATOM | 1684 | CA | PRO | A | 225 | 36.755 | 79.082 | 42.317 | 1.00 | 12.26 | A |
| ATOM | 1685 | CB | PRO | A | 225 | 35.744 | 79.535 | 43.372 | 1.00 | 14.55 | A |
| ATOM | 1686 | CG | PRO | A | 225 | 34.792 | 78.384 | 43.476 | 1.00 | 17.48 | A |
| ATOM | 1687 | C | PRO | A | 225 | 37.904 | 78.220 | 42.829 | 1.00 | 14.13 | A |
| ATOM | 1688 | O | PRO | A | 225 | 37.762 | 77.004 | 42.948 | 1.00 | 13.19 | A |
| ATOM | 1689 | N | LYS | A | 226 | 39.043 | 78.838 | 43.118 | 1.00 | 15.99 | A |
| ATOM | 1690 | CA | LYS | A | 226 | 40.187 | 78.087 | 43.617 | 1.00 | 19.79 | A |
| ATOM | 1691 | CB | LYS | A | 226 | 41.352 | 79.040 | 43.890 | 1.00 | 20.79 | A |
| ATOM | 1692 | CG | LYS | A | 226 | 42.257 | 79.278 | 42.705 | 1.00 | 22.75 | A |
| ATOM | 1693 | CD | LYS | A | 226 | 43.553 | 79.930 | 43.156 | 1.00 | 24.00 | A |
| ATOM | 1694 | CE | LYS | A | 226 | 44.558 | 80.005 | 42.025 | 1.00 | 25.83 | A |
| ATOM | 1695 | NZ | LYS | A | 226 | 45.870 | 80.514 | 42.503 | 1.00 | 28.01 | A |
| ATOM | 1696 | C | LYS | A | 226 | 39.807 | 77.277 | 44.845 | 1.00 | 21.58 | A |
| ATOM | 1697 | O | LYS | A | 226 | 39.138 | 77.778 | 45.750 | 1.00 | 21.79 | A |
| ATOM | 1698 | N | GLY | A | 227 | 40.236 | 76.019 | 44.869 | 1.00 | 22.89 | A |
| ATOM | 1699 | CA | GLY | A | 227 | 39.950 | 75.159 | 46.001 | 1.00 | 23.86 | A |
| ATOM | 1700 | C | GLY | A | 227 | 38.567 | 74.540 | 46.021 | 1.00 | 24.69 | A |
| ATOM | 1701 | O | GLY | A | 227 | 38.251 | 73.767 | 46.924 | 1.00 | 25.45 | A |
| ATOM | 1702 | N | SER | A | 228 | 37.738 | 74.871 | 45.034 | 1.00 | 23.96 | A |
| ATOM | 1703 | CA | SER | A | 228 | 36.386 | 74.326 | 44.964 | 1.00 | 24.91 | A |
| ATOM | 1704 | CB | SER | A | 228 | 35.671 | 74.896 | 43.738 | 1.00 | 24.44 | A |
| ATOM | 1705 | OG | SER | A | 228 | 34.442 | 74.224 | 43.526 | 1.00 | 27.04 | A |
| ATOM | 1706 | C | SER | A | 228 | 36.406 | 72.804 | 44.966 | 1.00 | 25.46 | A |
| ATOM | 1707 | O | SER | A | 228 | 37.220 | 72.183 | 44.284 | 1.00 | 24.95 | A |
| ATOM | 1708 | N | SER | A | 229 | 35.493 | 72.210 | 45.725 | 1.00 | 26.15 | A |
| ATOM | 1709 | CA | SER | A | 229 | 35.400 | 70.759 | 45.821 | 1.00 | 26.28 | A |
| ATOM | 1710 | CB | SER | A | 229 | 34.715 | 70.393 | 47.141 | 1.00 | 27.58 | A |
| ATOM | 1711 | OG | SER | A | 229 | 33.401 | 70.921 | 47.192 | 1.00 | 29.90 | A |
| ATOM | 1712 | C | SER | A | 229 | 34.684 | 70.183 | 44.604 | 1.00 | 25.17 | A |
| ATOM | 1713 | O | SER | A | 229 | 34.470 | 68.975 | 44.518 | 1.00 | 25.95 | A |
| ATOM | 1714 | N | LEU | A | 230 | 34.312 | 71.050 | 43.669 | 1.00 | 22.39 | A |
| ATOM | 1715 | CA | LEU | A | 230 | 33.619 | 70.621 | 42.461 | 1.00 | 21.14 | A |
| ATOM | 1716 | CB | LEU | A | 230 | 32.500 | 71.625 | 42.148 | 1.00 | 23.01 | A |
| ATOM | 1717 | CG | LEU | A | 230 | 31.110 | 71.083 | 41.797 | 1.00 | 25.72 | A |
| ATOM | 1718 | CD1 | LEU | A | 230 | 31.209 | 70.108 | 40.638 | 1.00 | 26.03 | A |
| ATOM | 1719 | CD2 | LEU | A | 230 | 30.507 | 70.401 | 43.012 | 1.00 | 26.48 | A |
| ATOM | 1720 | C | LEU | A | 230 | 34.636 | 70.494 | 41.333 | 1.00 | 19.19 | A |
| ATOM | 1721 | O | LEU | A | 230 | 34.412 | 69.776 | 40.357 | 1.00 | 18.25 | A |
| ATOM | 1722 | N | GLY | A | 231 | 35.760 | 71.189 | 41.489 | 1.00 | 17.43 | A |
| ATOM | 1723 | CA | GLY | A | 231 | 36.808 | 71.184 | 40.483 | 1.00 | 18.17 | A |
| ATOM | 1724 | C | GLY | A | 231 | 37.296 | 69.838 | 39.983 | 1.00 | 17.92 | A |
| ATOM | 1725 | O | GLY | A | 231 | 37.350 | 69.611 | 38.774 | 1.00 | 15.95 | A |
| ATOM | 1726 | N | ASN | A | 232 | 37.655 | 68.944 | 40.898 | 1.00 | 17.85 | A |
| ATOM | 1727 | CA | ASN | A | 232 | 38.158 | 67.632 | 40.508 | 1.00 | 18.27 | A |
| ATOM | 1728 | CB | ASN | A | 232 | 38.634 | 66.880 | 41.754 | 1.00 | 21.28 | A |
| ATOM | 1729 | CG | ASN | A | 232 | 39.138 | 65.484 | 41.432 | 1.00 | 23.57 | A |
| ATOM | 1730 | OD1 | ASN | A | 232 | 40.094 | 65.315 | 40.673 | 1.00 | 24.62 | A |
| ATOM | 1731 | ND2 | ASN | A | 232 | 38.491 | 64.474 | 42.005 | 1.00 | 24.98 | A |
| ATOM | 1732 | C | ASN | A | 232 | 37.137 | 66.841 | 39.699 | 1.00 | 16.30 | A |
| ATOM | 1733 | O | ASN | A | 232 | 37.456 | 66.323 | 38.628 | 1.00 | 15.12 | A |
| ATOM | 1734 | N | ALA | A | 233 | 35.914 | 66.742 | 40.214 | 1.00 | 15.63 | A |
| ATOM | 1735 | CA | ALA | A | 233 | 34.861 | 66.000 | 39.532 | 1.00 | 14.98 | A |
| ATOM | 1736 | CB | ALA | A | 233 | 33.581 | 66.015 | 40.364 | 1.00 | 16.59 | A |
| ATOM | 1737 | C | ALA | A | 233 | 34.586 | 66.559 | 38.140 | 1.00 | 13.06 | A |
| ATOM | 1738 | O | ALA | A | 233 | 34.446 | 65.810 | 37.176 | 1.00 | 13.37 | A |
| ATOM | 1739 | N | VAL | A | 234 | 34.513 | 67.881 | 38.039 | 1.00 | 12.94 | A |
| ATOM | 1740 | CA | VAL | A | 234 | 34.250 | 68.521 | 36.758 | 1.00 | 13.25 | A |
| ATOM | 1741 | CB | VAL | A | 234 | 34.108 | 70.048 | 36.928 | 1.00 | 14.43 | A |
| ATOM | 1742 | CG1 | VAL | A | 234 | 34.200 | 70.742 | 35.577 | 1.00 | 16.13 | A |
| ATOM | 1743 | CG2 | VAL | A | 234 | 32.780 | 70.362 | 37.598 | 1.00 | 16.27 | A |
| ATOM | 1744 | C | VAL | A | 234 | 35.347 | 68.210 | 35.751 | 1.00 | 11.85 | A |
| ATOM | 1745 | O | VAL | A | 234 | 35.064 | 67.942 | 34.582 | 1.00 | 11.28 | A |
| ATOM | 1746 | N | ASN | A | 235 | 36.597 | 68.240 | 36.202 | 1.00 | 12.84 | A |
| ATOM | 1747 | CA | ASN | A | 235 | 37.712 | 67.947 | 35.311 | 1.00 | 12.21 | A |
| ATOM | 1748 | CB | ASN | A | 235 | 39.034 | 68.259 | 36.013 | 1.00 | 13.94 | A |
| ATOM | 1749 | CG | ASN | A | 235 | 40.228 | 68.035 | 35.112 | 1.00 | 14.29 | A |
| ATOM | 1750 | OD1 | ASN | A | 235 | 40.335 | 68.647 | 34.050 | 1.00 | 12.99 | A |
| ATOM | 1751 | ND2 | ASN | A | 235 | 41.129 | 67.146 | 35.521 | 1.00 | 15.45 | A |
| ATOM | 1752 | C | ASN | A | 235 | 37.638 | 66.502 | 34.822 | 1.00 | 12.06 | A |
| ATOM | 1753 | O | ASN | A | 235 | 37.816 | 66.231 | 33.636 | 1.00 | 12.54 | A |
| ATOM | 1754 | N | LEU | A | 236 | 37.383 | 65.572 | 35.737 | 1.00 | 12.33 | A |
| ATOM | 1755 | CA | LEU | A | 236 | 37.289 | 64.170 | 35.356 | 1.00 | 12.19 | A |
| ATOM | 1756 | CB | LEU | A | 236 | 37.142 | 63.304 | 36.610 | 1.00 | 12.94 | A |
| ATOM | 1757 | CG | LEU | A | 236 | 38.330 | 63.274 | 37.571 | 1.00 | 14.02 | A |
| ATOM | 1758 | CD1 | LEU | A | 236 | 38.016 | 62.336 | 38.727 | 1.00 | 16.16 | A |
| ATOM | 1759 | CD2 | LEU | A | 236 | 39.582 | 62.816 | 36.838 | 1.00 | 15.03 | A |
| ATOM | 1760 | C | LEU | A | 236 | 36.144 | 63.990 | 34.369 | 1.00 | 11.11 | A |
| ATOM | 1761 | O | LEU | A | 236 | 36.234 | 63.181 | 33.450 | 1.00 | 11.50 | A |
| ATOM | 1762 | N | ALA | A | 237 | 35.064 | 64.744 | 34.555 | 1.00 | 10.73 | A |
| ATOM | 1763 | CA | ALA | A | 237 | 33.922 | 64.641 | 33.653 | 1.00 | 11.06 | A |
| ATOM | 1764 | CB | ALA | A | 237 | 32.761 | 65.476 | 34.174 | 1.00 | 11.63 | A |
| ATOM | 1765 | C | ALA | A | 237 | 34.314 | 65.092 | 32.246 | 1.00 | 8.82 | A |
| ATOM | 1766 | O | ALA | A | 237 | 33.938 | 64.466 | 31.254 | 1.00 | 10.10 | A |
| ATOM | 1767 | N | VAL | A | 238 | 35.069 | 66.181 | 32.155 | 1.00 | 9.53 | A |
| ATOM | 1768 | CA | VAL | A | 238 | 35.514 | 66.669 | 30.855 | 1.00 | 9.58 | A |
| ATOM | 1769 | CB | VAL | A | 238 | 36.331 | 67.979 | 30.992 | 1.00 | 9.67 | A |
| ATOM | 1770 | CG1 | VAL | A | 238 | 37.014 | 68.306 | 29.673 | 1.00 | 11.56 | A |
| ATOM | 1771 | CG2 | VAL | A | 238 | 35.412 | 69.124 | 31.410 | 1.00 | 10.33 | A |
| ATOM | 1772 | C | VAL | A | 238 | 36.375 | 65.602 | 30.166 | 1.00 | 9.59 | A |
| ATOM | 1773 | O | VAL | A | 238 | 36.224 | 65.344 | 28.974 | 1.00 | 10.14 | A |
| ATOM | 1774 | N | LEU | A | 239 | 37.268 | 64.975 | 30.924 | 1.00 | 10.44 | A |
| ATOM | 1775 | CA | LEU | A | 239 | 38.131 | 63.946 | 30.363 | 1.00 | 10.94 | A |
| ATOM | 1776 | CB | LEU | A | 239 | 39.161 | 63.531 | 31.417 | 1.00 | 12.73 | A |
| ATOM | 1777 | CG | LEU | A | 239 | 40.180 | 64.613 | 31.790 | 1.00 | 16.19 | A |
| ATOM | 1778 | CD1 | LEU | A | 239 | 41.084 | 64.109 | 32.887 | 1.00 | 18.37 | A |
| ATOM | 1779 | CD2 | LEU | A | 239 | 40.998 | 64.978 | 30.587 | 1.00 | 19.19 | A |
| ATOM | 1780 | C | LEU | A | 239 | 37.300 | 62.778 | 29.842 | 1.00 | 10.02 | A |
| ATOM | 1781 | O | LEU | A | 239 | 37.566 | 62.255 | 28.760 | 1.00 | 10.81 | A |
| ATOM | 1782 | N | LYS | A | 240 | 36.292 | 62.370 | 30.609 | 1.00 | 10.83 | A |
| ATOM | 1783 | CA | LYS | A | 240 | 35.445 | 61.261 | 30.196 | 1.00 | 11.07 | A |
| ATOM | 1784 | CB | LYS | A | 240 | 34.516 | 60.869 | 31.349 | 1.00 | 13.99 | A |
| ATOM | 1785 | CG | LYS | A | 240 | 33.663 | 59.642 | 31.052 | 1.00 | 19.14 | A |
| ATOM | 1786 | CD | LYS | A | 240 | 32.876 | 59.203 | 32.278 | 1.00 | 25.66 | A |
| ATOM | 1787 | CE | LYS | A | 240 | 32.113 | 57.914 | 32.011 | 1.00 | 29.89 | A |
| ATOM | 1788 | NZ | LYS | A | 240 | 31.381 | 57.433 | 33.218 | 1.00 | 33.32 | A |
| ATOM | 1789 | C | LYS | A | 240 | 34.699 | 61.620 | 28.919 | 1.00 | 10.37 | A |
| ATOM | 1790 | O | LYS | A | 240 | 34.679 | 60.841 | 27.972 | 1.00 | 11.74 | A |
| ATOM | 1791 | N | LEU | A | 241 | 34.082 | 62.800 | 28.889 | 1.00 | 10.03 | A |
| ATOM | 1792 | CA | LEU | A | 241 | 33.344 | 63.226 | 27.702 | 1.00 | 9.53 | A |
| ATOM | 1793 | CB | LEU | A | 241 | 32.672 | 64.576 | 27.979 | 1.00 | 10.84 | A |
| ATOM | 1794 | CG | LEU | A | 241 | 31.605 | 64.579 | 29.081 | 1.00 | 11.02 | A |
| ATOM | 1795 | CD1 | LEU | A | 241 | 31.140 | 66.000 | 29.344 | 1.00 | 12.25 | A |
| ATOM | 1796 | CD2 | LEU | A | 241 | 30.437 | 63.698 | 28.669 | 1.00 | 12.82 | A |
| ATOM | 1797 | C | LEU | A | 241 | 34.275 | 63.255 | 26.495 | 1.00 | 9.51 | A |
| ATOM | 1798 | O | LEU | A | 241 | 33.883 | 62.890 | 25.389 | 1.00 | 10.36 | A |
| ATOM | 1799 | N | SER | A | 242 | 35.507 | 63.705 | 26.714 | 1.00 | 9.60 | A |
| ATOM | 1800 | CA | SER | A | 242 | 36.500 | 63.760 | 25.653 | 1.00 | 10.00 | A |
| ATOM | 1801 | CB | SER | A | 242 | 37.769 | 64.418 | 26.191 | 1.00 | 10.04 | A |
| ATOM | 1802 | OG | SER | A | 242 | 38.843 | 64.252 | 25.274 | 1.00 | 11.08 | A |
| ATOM | 1803 | C | SER | A | 242 | 36.768 | 62.363 | 25.111 | 1.00 | 9.77 | A |
| ATOM | 1804 | O | SER | A | 242 | 36.660 | 62.120 | 23.911 | 1.00 | 10.50 | A |
| ATOM | 1805 | N | GLU | A | 243 | 37.122 | 61.455 | 26.017 | 1.00 | 10.78 | A |
| ATOM | 1806 | CA | GLU | A | 243 | 37.441 | 60.082 | 25.654 | 1.00 | 12.16 | A |
| ATOM | 1807 | CB | GLU | A | 243 | 38.086 | 59.396 | 26.865 | 1.00 | 12.47 | A |
| ATOM | 1808 | CG | GLU | A | 243 | 39.500 | 59.917 | 27.109 | 1.00 | 13.04 | A |
| ATOM | 1809 | CD | GLU | A | 243 | 40.207 | 59.282 | 28.288 | 1.00 | 13.31 | A |
| ATOM | 1810 | OE1 | GLU | A | 243 | 39.712 | 58.267 | 28.825 | 1.00 | 14.76 | A |
| ATOM | 1811 | OE2 | GLU | A | 243 | 41.278 | 59.801 | 28.671 | 1.00 | 13.41 | A |
| ATOM | 1812 | C | GLU | A | 243 | 36.284 | 59.307 | 25.043 | 1.00 | 11.98 | A |
| ATOM | 1813 | O | GLU | A | 243 | 36.511 | 58.373 | 24.275 | 1.00 | 13.80 | A |
| ATOM | 1814 | N | GLN | A | 244 | 35.053 | 59.692 | 25.368 | 1.00 | 12.01 | A |
| ATOM | 1815 | CA | GLN | A | 244 | 33.880 | 59.015 | 24.816 | 1.00 | 12.65 | A |
| ATOM | 1816 | CB | GLN | A | 244 | 32.699 | 59.154 | 25.782 | 1.00 | 15.70 | A |
| ATOM | 1817 | CG | GLN | A | 244 | 32.901 | 58.503 | 27.137 | 1.00 | 21.85 | A |
| ATOM | 1818 | CD | GLN | A | 244 | 31.694 | 58.672 | 28.041 | 1.00 | 26.51 | A |
| ATOM | 1819 | OE1 | GLN | A | 244 | 31.224 | 59.788 | 28.270 | 1.00 | 30.47 | A |
| ATOM | 1820 | NE2 | GLN | A | 244 | 31.188 | 57.563 | 28.565 | 1.00 | 29.00 | A |
| ATOM | 1821 | C | GLN | A | 244 | 33.542 | 59.545 | 23.431 | 1.00 | 12.25 | A |
| ATOM | 1822 | O | GLN | A | 244 | 32.700 | 58.978 | 22.740 | 1.00 | 14.55 | A |
| ATOM | 1823 | N | GLY | A | 245 | 34.193 | 60.634 | 23.035 | 1.00 | 12.61 | A |
| ATOM | 1824 | CA | GLY | A | 245 | 33.934 | 61.229 | 21.734 | 1.00 | 12.75 | A |
| ATOM | 1825 | C | GLY | A | 245 | 32.721 | 62.146 | 21.742 | 1.00 | 11.07 | A |
| ATOM | 1826 | O | GLY | A | 245 | 32.313 | 62.667 | 20.702 | 1.00 | 10.93 | A |
| ATOM | 1827 | N | LEU | A | 246 | 32.155 | 62.359 | 22.925 | 1.00 | 12.32 | A |
| ATOM | 1828 | CA | LEU | A | 246 | 30.971 | 63.196 | 23.075 | 1.00 | 12.25 | A |
| ATOM | 1829 | CB | LEU | A | 246 | 30.386 | 62.969 | 24.473 | 1.00 | 13.71 | A |
| ATOM | 1830 | CG | LEU | A | 246 | 29.713 | 61.612 | 24.706 | 1.00 | 16.08 | A |
| ATOM | 1831 | CD1 | LEU | A | 246 | 29.284 | 61.490 | 26.158 | 1.00 | 20.32 | A |
| ATOM | 1832 | CD2 | LEU | A | 246 | 28.511 | 61.472 | 23.782 | 1.00 | 18.93 | A |
| ATOM | 1833 | C | LEU | A | 246 | 31.229 | 64.665 | 22.754 | 1.00 | 11.72 | A |
| ATOM | 1834 | O | LEU | A | 246 | 30.354 | 65.346 | 22.224 | 1.00 | 11.90 | A |
| ATOM | 1835 | N | LEU | A | 247 | 32.420 | 65.158 | 23.076 | 1.00 | 10.24 | A |
| ATOM | 1836 | CA | LEU | A | 247 | 32.741 | 66.548 | 22.784 | 1.00 | 10.90 | A |
| ATOM | 1837 | CB | LEU | A | 247 | 34.016 | 66.951 | 23.532 | 1.00 | 12.45 | A |
| ATOM | 1838 | CG | LEU | A | 247 | 33.937 | 66.865 | 25.059 | 1.00 | 10.89 | A |
| ATOM | 1839 | CD1 | LEU | A | 247 | 35.259 | 67.333 | 25.661 | 1.00 | 12.02 | A |
| ATOM | 1840 | CD2 | LEU | A | 247 | 32.785 | 67.714 | 25.578 | 1.00 | 13.73 | A |
| ATOM | 1841 | C | LEU | A | 247 | 32.830 | 66.729 | 21.273 | 1.00 | 10.25 | A |
| ATOM | 1842 | O | LEU | A | 247 | 32.450 | 67.775 | 20.747 | 1.00 | 11.03 | A |
| ATOM | 1843 | N | ASP | A | 248 | 33.334 | 65.716 | 20.568 | 1.00 | 10.83 | A |
| ATOM | 1844 | CA | ASP | A | 248 | 33.431 | 65.796 | 19.113 | 1.00 | 12.99 | A |
| ATOM | 1845 | CB | ASP | A | 248 | 34.254 | 64.622 | 18.565 | 1.00 | 12.21 | A |
| ATOM | 1846 | CG | ASP | A | 248 | 35.707 | 64.672 | 18.983 | 1.00 | 13.84 | A |
| ATOM | 1847 | OD1 | ASP | A | 248 | 36.291 | 65.777 | 18.999 | 1.00 | 15.46 | A |
| ATOM | 1848 | OD2 | ASP | A | 248 | 36.271 | 63.597 | 19.278 | 1.00 | 17.21 | A |
| ATOM | 1849 | C | ASP | A | 248 | 32.024 | 65.841 | 18.526 | 1.00 | 12.51 | A |
| ATOM | 1850 | O | ASP | A | 248 | 31.750 | 66.614 | 17.610 | 1.00 | 13.15 | A |
| ATOM | 1851 | N | LYS | A | 249 | 31.136 | 65.005 | 19.058 | 1.00 | 12.00 | A |
| ATOM | 1852 | CA | LYS | A | 249 | 29.757 | 64.957 | 18.589 | 1.00 | 13.30 | A |
| ATOM | 1853 | CB | LYS | A | 249 | 29.030 | 63.802 | 19.287 | 1.00 | 15.41 | A |
| ATOM | 1854 | CG | LYS | A | 249 | 27.582 | 63.635 | 18.860 | 1.00 | 22.53 | A |
| ATOM | 1855 | CD | LYS | A | 249 | 26.910 | 62.498 | 19.611 | 1.00 | 25.97 | A |
| ATOM | 1856 | CE | LYS | A | 249 | 25.468 | 62.317 | 19.163 | 1.00 | 28.10 | A |
| ATOM | 1857 | NZ | LYS | A | 249 | 25.374 | 62.030 | 17.702 | 1.00 | 29.71 | A |
| ATOM | 1858 | C | LYS | A | 249 | 29.073 | 66.304 | 18.812 | 1.00 | 12.10 | A |
| ATOM | 1859 | O | LYS | A | 249 | 28.326 | 66.779 | 17.956 | 1.00 | 12.22 | A |
| ATOM | 1860 | N | LEU | A | 250 | 29.339 | 66.912 | 19.963 | 1.00 | 11.38 | A |
| ATOM | 1861 | CA | LEU | A | 250 | 28.747 | 68.192 | 20.320 | 1.00 | 13.81 | A |
| ATOM | 1862 | CB | LEU | A | 250 | 29.052 | 68.476 | 21.791 | 1.00 | 15.34 | A |
| ATOM | 1863 | CG | LEU | A | 250 | 28.126 | 69.460 | 22.502 | 1.00 | 19.52 | A |
| ATOM | 1864 | CD1 | LEU | A | 250 | 26.693 | 68.947 | 22.444 | 1.00 | 19.78 | A |
| ATOM | 1865 | CD2 | LEU | A | 250 | 28.575 | 69.624 | 23.942 | 1.00 | 18.91 | A |
| ATOM | 1866 | C | LEU | A | 250 | 29.207 | 69.299 | 19.380 | 1.00 | 11.31 | A |
| ATOM | 1867 | O | LEU | A | 250 | 28.409 | 70.147 | 18.983 | 1.00 | 11.72 | A |
| ATOM | 1868 | N | LYS | A | 251 | 30.489 | 69.303 | 19.015 | 1.00 | 11.59 | A |
| ATOM | 1869 | CA | LYS | A | 251 | 30.966 | 70.338 | 18.104 | 1.00 | 12.31 | A |
| ATOM | 1870 | CB | LYS | A | 251 | 32.499 | 70.357 | 18.036 | 1.00 | 13.49 | A |
| ATOM | 1871 | CG | LYS | A | 251 | 32.995 | 71.489 | 17.133 | 1.00 | 16.13 | A |
| ATOM | 1872 | CD | LYS | A | 251 | 34.501 | 71.665 | 17.107 | 1.00 | 16.95 | A |
| ATOM | 1873 | CE | LYS | A | 251 | 34.851 | 72.951 | 16.360 | 1.00 | 18.24 | A |
| ATOM | 1874 | NZ | LYS | A | 251 | 36.310 | 73.139 | 16.173 | 1.00 | 21.00 | A |
| ATOM | 1875 | C | LYS | A | 251 | 30.332 | 70.150 | 16.730 | 1.00 | 11.48 | A |
| ATOM | 1876 | O | LYS | A | 251 | 29.900 | 71.116 | 16.105 | 1.00 | 11.25 | A |
| ATOM | 1877 | N | ASN | A | 252 | 30.284 | 68.909 | 16.252 | 1.00 | 11.86 | A |
| ATOM | 1878 | CA | ASN | A | 252 | 29.687 | 68.649 | 14.947 | 1.00 | 13.33 | A |
| ATOM | 1879 | CB | ASN | A | 252 | 29.837 | 67.168 | 14.586 | 1.00 | 16.75 | A |
| ATOM | 1880 | CG | ASN | A | 252 | 31.266 | 66.787 | 14.287 | 1.00 | 20.29 | A |
| ATOM | 1881 | OD1 | ASN | A | 252 | 32.017 | 67.571 | 13.709 | 1.00 | 23.39 | A |
| ATOM | 1882 | ND2 | ASN | A | 252 | 31.648 | 65.572 | 14.663 | 1.00 | 22.44 | A |
| ATOM | 1883 | C | ASN | A | 252 | 28.240 | 69.106 | 14.939 | 1.00 | 13.81 | A |
| ATOM | 1884 | O | ASN | A | 252 | 27.777 | 69.704 | 13.975 | 1.00 | 13.29 | A |
| ATOM | 1885 | N | LYS | A | 253 | 27.529 | 68.819 | 16.023 | 1.00 | 12.19 | A |
| ATOM | 1886 | CA | LYS | A | 253 | 26.127 | 69.199 | 16.134 | 1.00 | 13.41 | A |
| ATOM | 1887 | CB | LYS | A | 253 | 25.583 | 68.695 | 17.475 | 1.00 | 14.37 | A |
| ATOM | 1888 | CG | LYS | A | 253 | 24.183 | 69.195 | 17.808 | 1.00 | 13.86 | A |
| ATOM | 1889 | CD | LYS | A | 253 | 23.730 | 68.716 | 19.185 | 1.00 | 14.28 | A |
| ATOM | 1890 | CE | LYS | A | 253 | 22.346 | 69.242 | 19.527 | 1.00 | 16.70 | A |
| ATOM | 1891 | NZ | LYS | A | 253 | 21.928 | 68.869 | 20.909 | 1.00 | 20.13 | A |
| ATOM | 1892 | C | LYS | A | 253 | 25.905 | 70.696 | 15.953 | 1.00 | 13.48 | A |
| ATOM | 1893 | O | LYS | A | 253 | 25.082 | 71.113 | 15.139 | 1.00 | 14.39 | A |
| ATOM | 1894 | N | TRP | A | 254 | 26.657 | 71.500 | 16.699 | 1.00 | 11.82 | A |
| ATOM | 1895 | CA | TRP | A | 254 | 26.484 | 72.945 | 16.660 | 1.00 | 12.08 | A |
| ATOM | 1896 | CB | TRP | A | 254 | 26.754 | 73.493 | 18.068 | 1.00 | 11.54 | A |
| ATOM | 1897 | CG | TRP | A | 254 | 25.733 | 73.030 | 19.059 | 1.00 | 10.58 | A |
| ATOM | 1898 | CD2 | TRP | A | 254 | 24.393 | 73.514 | 19.185 | 1.00 | 9.75 | A |
| ATOM | 1899 | CE2 | TRP | A | 254 | 23.766 | 72.760 | 20.201 | 1.00 | 10.91 | A |
| ATOM | 1900 | CE3 | TRP | A | 254 | 23.658 | 74.512 | 18.531 | 1.00 | 10.72 | A |
| ATOM | 1901 | CD1 | TRP | A | 254 | 25.867 | 72.028 | 19.981 | 1.00 | 10.86 | A |
| ATOM | 1902 | NE1 | TRP | A | 254 | 24.686 | 71.859 | 20.672 | 1.00 | 10.73 | A |
| ATOM | 1903 | CZ2 | TRP | A | 254 | 22.438 | 72.974 | 20.580 | 1.00 | 11.83 | A |
| ATOM | 1904 | CZ3 | TRP | A | 254 | 22.340 | 74.725 | 18.907 | 1.00 | 12.00 | A |
| ATOM | 1905 | CH2 | TRP | A | 254 | 21.745 | 73.959 | 19.923 | 1.00 | 11.75 | A |
| ATOM | 1906 | C | TRP | A | 254 | 27.238 | 73.701 | 15.572 | 1.00 | 11.48 | A |
| ATOM | 1907 | O | TRP | A | 254 | 26.907 | 74.853 | 15.287 | 1.00 | 12.01 | A |
| ATOM | 1908 | N | TRP | A | 255 | 28.226 | 73.062 | 14.957 | 1.00 | 11.38 | A |
| ATOM | 1909 | CA | TRP | A | 255 | 29.018 | 73.707 | 13.912 | 1.00 | 13.24 | A |
| ATOM | 1910 | CB | TRP | A | 255 | 30.506 | 73.489 | 14.201 | 1.00 | 13.18 | A |
| ATOM | 1911 | CG | TRP | A | 255 | 31.156 | 74.556 | 14.996 | 1.00 | 11.40 | A |
| ATOM | 1912 | CD2 | TRP | A | 255 | 31.010 | 74.782 | 16.398 | 1.00 | 10.32 | A |
| ATOM | 1913 | CE2 | TRP | A | 255 | 31.820 | 75.890 | 16.727 | 1.00 | 10.52 | A |
| ATOM | 1914 | CE3 | TRP | A | 255 | 30.271 | 74.154 | 17.409 | 1.00 | 11.07 | A |
| ATOM | 1915 | CD1 | TRP | A | 255 | 32.022 | 75.507 | 14.539 | 1.00 | 11.83 | A |
| ATOM | 1916 | NE1 | TRP | A | 255 | 32.426 | 76.312 | 15.574 | 1.00 | 10.82 | A |
| ATOM | 1917 | CZ2 | TRP | A | 255 | 31.914 | 76.387 | 18.029 | 1.00 | 9.89 | A |
| ATOM | 1918 | CZ3 | TRP | A | 255 | 30.363 | 74.647 | 18.706 | 1.00 | 10.99 | A |
| ATOM | 1919 | CH2 | TRP | A | 255 | 31.181 | 75.756 | 19.005 | 1.00 | 10.54 | A |
| ATOM | 1920 | C | TRP | A | 255 | 28.693 | 73.270 | 12.494 | 1.00 | 14.87 | A |
| ATOM | 1921 | O | TRP | A | 255 | 28.460 | 74.104 | 11.622 | 1.00 | 15.77 | A |
| ATOM | 1922 | N | TYR | A | 256 | 28.679 | 71.960 | 12.269 | 1.00 | 17.01 | A |
| ATOM | 1923 | CA | TYR | A | 256 | 28.487 | 71.426 | 10.927 | 1.00 | 18.48 | A |
| ATOM | 1924 | CB | TYR | A | 256 | 29.692 | 70.530 | 10.639 | 1.00 | 17.86 | A |
| ATOM | 1925 | CG | TYR | A | 256 | 31.002 | 71.189 | 11.015 | 1.00 | 17.74 | A |
| ATOM | 1926 | CD1 | TYR | A | 256 | 31.445 | 72.332 | 10.350 | 1.00 | 19.92 | A |
| ATOM | 1927 | CE1 | TYR | A | 256 | 32.626 | 72.971 | 10.725 | 1.00 | 19.08 | A |
| ATOM | 1928 | CD2 | TYR | A | 256 | 31.776 | 70.697 | 12.065 | 1.00 | 18.11 | A |
| ATOM | 1929 | CE2 | TYR | A | 256 | 32.959 | 71.329 | 12.447 | 1.00 | 20.70 | A |
| ATOM | 1930 | CZ | TYR | A | 256 | 33.375 | 72.466 | 11.773 | 1.00 | 19.95 | A |
| ATOM | 1931 | OH | TYR | A | 256 | 34.536 | 73.104 | 12.151 | 1.00 | 23.67 | A |
| ATOM | 1932 | C | TYR | A | 256 | 27.158 | 70.754 | 10.605 | 1.00 | 21.44 | A |
| ATOM | 1933 | O | TYR | A | 256 | 26.645 | 70.903 | 9.495 | 1.00 | 22.92 | A |
| ATOM | 1934 | N | ASP | A | 257 | 26.608 | 70.006 | 11.554 | 1.00 | 23.80 | A |
| ATOM | 1935 | CA | ASP | A | 257 | 25.342 | 69.312 | 11.337 | 1.00 | 27.48 | A |
| ATOM | 1936 | CB | ASP | A | 257 | 24.902 | 68.668 | 12.655 | 1.00 | 27.47 | A |
| ATOM | 1937 | CG | ASP | A | 257 | 25.709 | 67.427 | 12.997 | 1.00 | 28.50 | A |
| ATOM | 1938 | OD1 | ASP | A | 257 | 26.875 | 67.328 | 12.561 | 1.00 | 29.59 | A |
| ATOM | 1939 | OD2 | ASP | A | 257 | 25.179 | 66.552 | 13.713 | 1.00 | 28.83 | A |
| ATOM | 1940 | C | ASP | A | 257 | 24.292 | 70.241 | 10.733 | 1.00 | 29.42 | A |
| ATOM | 1941 | O | ASP | A | 257 | 23.423 | 69.802 | 9.978 | 1.00 | 31.16 | A |
| ATOM | 1942 | N | GLY | A | 258 | 24.380 | 71.525 | 11.062 | 1.00 | 31.16 | A |
| ATOM | 1943 | CA | GLY | A | 258 | 23.429 | 72.487 | 10.536 | 1.00 | 32.33 | A |
| ATOM | 1944 | C | GLY | A | 258 | 24.059 | 73.441 | 9.539 | 1.00 | 33.05 | A |
| ATOM | 1945 | O | GLY | A | 258 | 23.484 | 74.482 | 9.220 | 1.00 | 34.08 | A |
| ATOM | 1946 | N | GLY | A | 259 | 25.242 | 73.082 | 9.048 | 1.00 | 33.37 | A |
| ATOM | 1947 | CA | GLY | A | 259 | 25.941 | 73.919 | 8.087 | 1.00 | 32.81 | A |
| ATOM | 1948 | C | GLY | A | 259 | 25.036 | 74.455 | 6.995 | 1.00 | 33.01 | A |
| ATOM | 1949 | O | GLY | A | 259 | 24.345 | 73.692 | 6.317 | 1.00 | 32.94 | A |
| ATOM | 1950 | N | GLU | A | 260 | 25.044 | 75.773 | 6.823 | 1.00 | 32.45 | A |
| ATOM | 1951 | CA | GLU | A | 260 | 24.217 | 76.427 | 5.817 | 1.00 | 31.91 | A |
| ATOM | 1952 | CB | GLU | A | 260 | 23.638 | 77.713 | 6.414 | 1.00 | 32.09 | A |
| ATOM | 1953 | CG | GLU | A | 260 | 22.761 | 77.502 | 7.635 | 1.00 | 32.14 | A |
| ATOM | 1954 | CD | GLU | A | 260 | 22.348 | 78.810 | 8.285 | 1.00 | 32.27 | A |
| ATOM | 1955 | OE1 | GLU | A | 260 | 21.521 | 78.777 | 9.223 | 1.00 | 32.26 | A |
| ATOM | 1956 | OE2 | GLU | A | 260 | 22.853 | 79.871 | 7.862 | 1.00 | 33.19 | A |
| ATOM | 1957 | C | GLU | A | 260 | 25.002 | 76.681 | 4.537 | 1.00 | 31.22 | A |
| ATOM | 1958 | O | GLU | A | 260 | 24.459 | 77.212 | 3.569 | 1.00 | 32.04 | A |
| ATOM | 1959 | N | CYS | A | 261 | 26.275 | 76.303 | 4.532 | 1.00 | 30.64 | A |
| ATOM | 1960 | CA | CYS | A | 261 | 27.120 | 76.519 | 3.365 | 1.00 | 30.66 | A |
| ATOM | 1961 | CB | CYS | A | 261 | 28.266 | 77.457 | 3.761 | 1.00 | 27.88 | A |
| ATOM | 1962 | SG | CYS | A | 261 | 27.700 | 78.992 | 4.563 | 1.00 | 22.41 | A |
| ATOM | 1963 | C | CYS | A | 261 | 27.595 | 75.199 | 2.768 | 1.00 | 32.66 | A |
| ATOM | 1964 | OT1 | CYS | A | 261 | 28.824 | 74.998 | 2.684 | 1.00 | 34.27 | A |
| ATOM | 1965 | OT2 | CYS | A | 261 | 26.729 | 74.384 | 2.386 | 1.00 | 33.85 | A |
| ATOM | 1966 | N | KAI | D | 999 | 52.259 | 58.965 | 24.382 | 1.00 | 8.63 | D |
| ATOM | 1967 | CD | KAI | D | 999 | 53.638 | 58.501 | 23.988 | 1.00 | 10.80 | D |
| ATOM | 1968 | CD1 | KAI | D | 999 | 55.552 | 56.535 | 23.071 | 1.00 | 12.20 | D |
| ATOM | 1969 | CD2 | KAI | D | 999 | 54.558 | 54.578 | 24.118 | 1.00 | 11.04 | D |
| ATOM | 1970 | CA | KAI | D | 999 | 51.419 | 57.830 | 24.582 | 1.00 | 9.63 | D |
| ATOM | 1971 | CB | KAI | D | 999 | 52.093 | 56.616 | 23.960 | 1.00 | 10.19 | D |
| ATOM | 1972 | CB1 | KAI | D | 999 | 51.980 | 56.522 | 22.435 | 1.00 | 10.60 | D |
| ATOM | 1973 | CG | KAI | D | 999 | 53.489 | 56.931 | 24.227 | 1.00 | 10.61 | D |
| ATOM | 1974 | CG1 | KAI | D | 999 | 50.770 | 55.761 | 21.918 | 1.00 | 10.59 | D |
| ATOM | 1975 | CG2 | KAI | D | 999 | 54.632 | 56.001 | 23.768 | 1.00 | 10.26 | D |
| ATOM | 1976 | C | KAI | D | 999 | 51.075 | 57.648 | 26.071 | 1.00 | 9.43 | D |
| ATOM | 1977 | O | KAI | D | 999 | 51.316 | 58.626 | 26.818 | 1.00 | 8.26 | D |
| ATOM | 1978 | OD1 | KAI | D | 999 | 50.585 | 55.802 | 20.683 | 1.00 | 10.86 | D |
| ATOM | 1979 | OD2 | KAI | D | 999 | 50.054 | 55.152 | 22.744 | 1.00 | 12.88 | D |
| ATOM | 1980 | OT | KAI | D | 999 | 50.572 | 56.547 | 26.391 | 1.00 | 10.08 | D |
| ATOM | 1981 | C | GLY | B | 4 | 66.467 | 69.848 | 45.552 | 1.00 | 25.87 | B |
| ATOM | 1982 | O | GLY | B | 4 | 65.518 | 70.397 | 44.992 | 1.00 | 26.65 | B |
| ATOM | 1983 | N | GLY | B | 4 | 65.769 | 70.992 | 47.668 | 1.00 | 28.44 | B |
| ATOM | 1984 | CA | GLY | B | 4 | 66.664 | 69.975 | 47.051 | 1.00 | 26.72 | B |
| ATOM | 1985 | N | THR | B | 5 | 67.368 | 69.121 | 44.902 | 1.00 | 23.55 | B |
| ATOM | 1986 | CA | THR | B | 5 | 67.290 | 68.918 | 43.462 | 1.00 | 20.59 | B |
| ATOM | 1987 | CB | THR | B | 5 | 68.519 | 68.144 | 42.948 | 1.00 | 20.71 | B |
| ATOM | 1988 | OG1 | THR | B | 5 | 69.704 | 68.909 | 43.207 | 1.00 | 22.39 | B |
| ATOM | 1989 | CG2 | THR | B | 5 | 68.403 | 67.883 | 41.451 | 1.00 | 19.82 | B |
| ATOM | 1990 | C | THR | B | 5 | 66.025 | 68.147 | 43.097 | 1.00 | 18.19 | B |
| ATOM | 1991 | O | THR | B | 5 | 65.721 | 67.109 | 43.684 | 1.00 | 18.92 | B |
| ATOM | 1992 | N | VAL | B | 6 | 65.289 | 68.671 | 42.125 | 1.00 | 15.33 | B |
| ATOM | 1993 | CA | VAL | B | 6 | 64.056 | 68.045 | 41.669 | 1.00 | 14.60 | B |
| ATOM | 1994 | CB | VAL | B | 6 | 63.195 | 69.063 | 40.889 | 1.00 | 12.72 | B |
| ATOM | 1995 | CG1 | VAL | B | 6 | 61.990 | 68.376 | 40.267 | 1.00 | 13.98 | B |
| ATOM | 1996 | CG2 | VAL | B | 6 | 62.754 | 70.184 | 41.826 | 1.00 | 13.51 | B |
| ATOM | 1997 | C | VAL | B | 6 | 64.373 | 66.851 | 40.778 | 1.00 | 13.37 | B |
| ATOM | 1998 | O | VAL | B | 6 | 65.198 | 66.948 | 39.872 | 1.00 | 14.05 | B |
| ATOM | 1999 | N | VAL | B | 7 | 63.732 | 65.719 | 41.049 | 1.00 | 11.84 | B |
| ATOM | 2000 | CA | VAL | B | 7 | 63.952 | 64.526 | 40.248 | 1.00 | 11.72 | B |
| ATOM | 2001 | CB | VAL | B | 7 | 63.857 | 63.247 | 41.096 | 1.00 | 12.00 | B |
| ATOM | 2002 | CG1 | VAL | B | 7 | 63.972 | 62.018 | 40.198 | 1.00 | 13.16 | B |
| ATOM | 2003 | CG2 | VAL | B | 7 | 64.962 | 63.244 | 42.138 | 1.00 | 13.65 | B |
| ATOM | 2004 | C | VAL | B | 7 | 62.924 | 64.461 | 39.131 | 1.00 | 10.82 | B |
| ATOM | 2005 | O | VAL | B | 7 | 61.726 | 64.290 | 39.373 | 1.00 | 11.42 | B |
| ATOM | 2006 | N | VAL | B | 8 | 63.404 | 64.612 | 37.904 | 1.00 | 11.28 | B |
| ATOM | 2007 | CA | VAL | B | 8 | 62.545 | 64.571 | 36.737 | 1.00 | 11.25 | B |
| ATOM | 2008 | CB | VAL | B | 8 | 62.989 | 65.613 | 35.679 | 1.00 | 11.15 | B |
| ATOM | 2009 | CG1 | VAL | B | 8 | 62.102 | 65.517 | 34.436 | 1.00 | 12.30 | B |
| ATOM | 2010 | CG2 | VAL | B | 8 | 62.937 | 67.010 | 36.272 | 1.00 | 10.83 | B |
| ATOM | 2011 | C | VAL | B | 8 | 62.634 | 63.198 | 36.106 | 1.00 | 11.06 | B |
| ATOM | 2012 | O | VAL | B | 8 | 63.735 | 62.692 | 35.870 | 1.00 | 11.58 | B |
| ATOM | 2013 | N | THR | B | 9 | 61.489 | 62.570 | 35.871 | 1.00 | 9.47 | B |
| ATOM | 2014 | CA | THR | B | 9 | 61.508 | 61.287 | 35.196 | 1.00 | 9.39 | B |
| ATOM | 2015 | CB | THR | B | 9 | 60.576 | 60.227 | 35.847 | 1.00 | 10.31 | B |
| ATOM | 2016 | OG1 | THR | B | 9 | 60.678 | 59.000 | 35.106 | 1.00 | 11.00 | B |
| ATOM | 2017 | CG2 | THR | B | 9 | 59.121 | 60.683 | 35.852 | 1.00 | 11.65 | B |
| ATOM | 2018 | C | THR | B | 9 | 61.059 | 61.550 | 33.768 | 1.00 | 9.23 | B |
| ATOM | 2019 | O | THR | B | 9 | 60.139 | 62.340 | 33.518 | 1.00 | 9.76 | B |
| ATOM | 2020 | N | THR | B | 10 | 61.747 | 60.921 | 32.826 | 1.00 | 8.57 | B |
| ATOM | 2021 | CA | THR | B | 10 | 61.413 | 61.075 | 31.427 | 1.00 | 8.66 | B |
| ATOM | 2022 | CB | THR | B | 10 | 62.136 | 62.304 | 30.801 | 1.00 | 9.52 | B |
| ATOM | 2023 | OG1 | THR | B | 10 | 61.581 | 62.579 | 29.510 | 1.00 | 9.17 | B |
| ATOM | 2024 | CG2 | THR | B | 10 | 63.632 | 62.070 | 30.681 | 1.00 | 9.62 | B |
| ATOM | 2025 | C | THR | B | 10 | 61.766 | 59.771 | 30.729 | 1.00 | 9.19 | B |
| ATOM | 2026 | O | THR | B | 10 | 62.116 | 58.793 | 31.388 | 1.00 | 8.78 | B |
| ATOM | 2027 | N | ILE | B | 11 | 61.676 | 59.746 | 29.408 | 1.00 | 8.42 | B |
| ATOM | 2028 | CA | ILE | B | 11 | 61.925 | 58.516 | 28.674 | 1.00 | 9.57 | B |
| ATOM | 2029 | CB | ILE | B | 11 | 60.584 | 57.731 | 28.534 | 1.00 | 10.19 | B |
| ATOM | 2030 | CG2 | ILE | B | 11 | 59.605 | 58.521 | 27.674 | 1.00 | 10.71 | B |
| ATOM | 2031 | CG1 | ILE | B | 11 | 60.817 | 56.335 | 27.958 | 1.00 | 9.87 | B |
| ATOM | 2032 | CD1 | ILE | B | 11 | 59.587 | 55.440 | 28.079 | 1.00 | 12.32 | B |
| ATOM | 2033 | C | ILE | B | 11 | 62.507 | 58.851 | 27.310 | 1.00 | 10.25 | B |
| ATOM | 2034 | O | ILE | B | 11 | 62.250 | 59.928 | 26.768 | 1.00 | 10.08 | B |
| ATOM | 2035 | N | LEU | B | 12 | 63.306 | 57.937 | 26.765 | 1.00 | 10.50 | B |
| ATOM | 2036 | CA | LEU | B | 12 | 63.905 | 58.152 | 25.459 | 1.00 | 12.34 | B |
| ATOM | 2037 | CB | LEU | B | 12 | 65.150 | 57.266 | 25.313 | 1.00 | 12.17 | B |
| ATOM | 2038 | CG | LEU | B | 12 | 66.280 | 57.520 | 26.317 | 1.00 | 12.21 | B |
| ATOM | 2039 | CD1 | LEU | B | 12 | 67.424 | 56.543 | 26.062 | 1.00 | 13.28 | B |
| ATOM | 2040 | CD2 | LEU | B | 12 | 66.766 | 58.958 | 26.204 | 1.00 | 15.41 | B |
| ATOM | 2041 | C | LEU | B | 12 | 62.850 | 57.900 | 24.391 | 1.00 | 12.73 | B |
| ATOM | 2042 | O | LEU | B | 12 | 62.301 | 56.802 | 24.287 | 1.00 | 15.53 | B |
| ATOM | 2043 | N | GLU | B | 13 | 62.551 | 58.940 | 23.621 | 1.00 | 11.26 | B |
| ATOM | 2044 | CA | GLU | B | 13 | 61.581 | 58.878 | 22.529 | 1.00 | 11.65 | B |
| ATOM | 2045 | CB | GLU | B | 13 | 60.151 | 58.869 | 23.090 | 1.00 | 11.63 | B |
| ATOM | 2046 | CG | GLU | B | 13 | 59.056 | 58.898 | 22.008 | 1.00 | 12.30 | B |
| ATOM | 2047 | CD | GLU | B | 13 | 58.836 | 57.558 | 21.315 | 1.00 | 13.74 | B |
| ATOM | 2048 | OE1 | GLU | B | 13 | 58.263 | 57.556 | 20.205 | 1.00 | 15.08 | B |
| ATOM | 2049 | OE2 | GLU | B | 13 | 59.215 | 56.506 | 21.873 | 1.00 | 13.46 | B |
| ATOM | 2050 | C | GLU | B | 13 | 61.877 | 60.097 | 21.668 | 1.00 | 10.94 | B |
| ATOM | 2051 | O | GLU | B | 13 | 61.802 | 61.226 | 22.145 | 1.00 | 11.44 | B |
| ATOM | 2052 | N | SER | B | 14 | 62.221 | 59.875 | 20.403 | 1.00 | 11.40 | B |
| ATOM | 2053 | CA | SER | B | 14 | 62.540 | 60.975 | 19.496 | 1.00 | 10.45 | B |
| ATOM | 2054 | CB | SER | B | 14 | 63.303 | 60.417 | 18.292 | 1.00 | 12.41 | B |
| ATOM | 2055 | OG | SER | B | 14 | 64.464 | 59.727 | 18.699 | 1.00 | 14.94 | B |
| ATOM | 2056 | C | SER | B | 14 | 61.277 | 61.718 | 19.085 | 1.00 | 10.94 | B |
| ATOM | 2057 | O | SER | B | 14 | 60.239 | 61.105 | 18.845 | 1.00 | 12.89 | B |
| ATOM | 2058 | N | PRO | B | 15 | 61.348 | 63.054 | 18.976 | 1.00 | 11.14 | B |
| ATOM | 2059 | CD | PRO | B | 15 | 60.292 | 63.756 | 18.224 | 1.00 | 11.21 | B |
| ATOM | 2060 | CA | PRO | B | 15 | 62.488 | 63.943 | 19.200 | 1.00 | 11.12 | B |
| ATOM | 2061 | CB | PRO | B | 15 | 62.361 | 64.903 | 18.029 | 1.00 | 10.05 | B |
| ATOM | 2062 | CG | PRO | B | 15 | 60.879 | 65.212 | 18.052 | 1.00 | 10.43 | B |
| ATOM | 2063 | C | PRO | B | 15 | 62.392 | 64.599 | 20.576 | 1.00 | 11.05 | B |
| ATOM | 2064 | O | PRO | B | 15 | 62.964 | 65.665 | 20.801 | 1.00 | 11.55 | B |
| ATOM | 2065 | N | TYR | B | 16 | 61.667 | 63.958 | 21.488 | 1.00 | 10.36 | B |
| ATOM | 2066 | CA | TYR | B | 16 | 61.458 | 64.483 | 22.837 | 1.00 | 10.25 | B |
| ATOM | 2067 | CB | TYR | B | 16 | 60.265 | 63.755 | 23.461 | 1.00 | 10.31 | B |
| ATOM | 2068 | CG | TYR | B | 16 | 59.030 | 63.764 | 22.591 | 1.00 | 8.58 | B |
| ATOM | 2069 | CD1 | TYR | B | 16 | 58.156 | 64.853 | 22.586 | 1.00 | 9.19 | B |
| ATOM | 2070 | CE1 | TYR | B | 16 | 57.020 | 64.853 | 21.775 | 1.00 | 8.77 | B |
| ATOM | 2071 | CD2 | TYR | B | 16 | 58.741 | 62.682 | 21.764 | 1.00 | 9.76 | B |
| ATOM | 2072 | CE2 | TYR | B | 16 | 57.618 | 62.674 | 20.954 | 1.00 | 10.41 | B |
| ATOM | 2073 | CZ | TYR | B | 16 | 56.761 | 63.758 | 20.965 | 1.00 | 9.82 | B |
| ATOM | 2074 | OH | TYR | B | 16 | 55.641 | 63.734 | 20.171 | 1.00 | 11.39 | B |
| ATOM | 2075 | C | TYR | B | 16 | 62.705 | 64.404 | 23.702 | 1.00 | 10.28 | B |
| ATOM | 2076 | O | TYR | B | 16 | 63.099 | 65.394 | 24.315 | 1.00 | 9.88 | B |
| ATOM | 2077 | N | VAL | B | 17 | 63.303 | 63.218 | 23.773 | 1.00 | 10.38 | B |
| ATOM | 2078 | CA | VAL | B | 17 | 64.518 | 63.004 | 24.553 | 1.00 | 10.31 | B |
| ATOM | 2079 | CB | VAL | B | 17 | 64.216 | 62.510 | 25.987 | 1.00 | 10.46 | B |
| ATOM | 2080 | CG1 | VAL | B | 17 | 65.503 | 62.456 | 26.800 | 1.00 | 11.76 | B |
| ATOM | 2081 | CG2 | VAL | B | 17 | 63.207 | 63.429 | 26.655 | 1.00 | 11.78 | B |
| ATOM | 2082 | C | VAL | B | 17 | 65.357 | 61.948 | 23.850 | 1.00 | 9.86 | B |
| ATOM | 2083 | O | VAL | B | 17 | 64.873 | 60.854 | 23.551 | 1.00 | 10.88 | B |
| ATOM | 2084 | N | MET | B | 18 | 66.612 | 62.294 | 23.581 | 1.00 | 10.26 | B |
| ATOM | 2085 | CA | MET | B | 18 | 67.549 | 61.406 | 22.906 | 1.00 | 12.06 | B |
| ATOM | 2086 | CB | MET | B | 18 | 67.583 | 61.761 | 21.409 | 1.00 | 15.22 | B |
| ATOM | 2087 | CG | MET | B | 18 | 66.216 | 61.838 | 20.728 | 1.00 | 19.02 | B |
| ATOM | 2088 | SD | MET | B | 18 | 66.244 | 62.690 | 19.120 | 1.00 | 21.27 | B |
| ATOM | 2089 | CE | MET | B | 18 | 66.218 | 64.416 | 19.671 | 1.00 | 17.44 | B |
| ATOM | 2090 | C | MET | B | 18 | 68.909 | 61.592 | 23.566 | 1.00 | 11.87 | B |
| ATOM | 2091 | O | MET | B | 18 | 69.196 | 62.662 | 24.102 | 1.00 | 11.59 | B |
| ATOM | 2092 | N | MET | B | 19 | 69.744 | 60.558 | 23.553 | 1.00 | 12.57 | B |
| ATOM | 2093 | CA | MET | B | 19 | 71.082 | 60.700 | 24.109 | 1.00 | 12.32 | B |
| ATOM | 2094 | CB | MET | B | 19 | 71.641 | 59.334 | 24.535 | 1.00 | 15.07 | B |
| ATOM | 2095 | CG | MET | B | 19 | 70.992 | 58.722 | 25.765 | 1.00 | 13.93 | B |
| ATOM | 2096 | SD | MET | B | 19 | 72.099 | 57.536 | 26.585 | 1.00 | 15.99 | B |
| ATOM | 2097 | CE | MET | B | 19 | 72.981 | 58.624 | 27.712 | 1.00 | 16.50 | B |
| ATOM | 2098 | C | MET | B | 19 | 71.911 | 61.354 | 23.008 | 1.00 | 12.90 | B |
| ATOM | 2099 | O | MET | B | 19 | 71.833 | 60.944 | 21.850 | 1.00 | 14.84 | B |
| ATOM | 2100 | N | LYS | B | 20 | 72.690 | 62.372 | 23.356 | 1.00 | 12.52 | B |
| ATOM | 2101 | CA | LYS | B | 20 | 73.533 | 63.038 | 22.367 | 1.00 | 14.47 | B |
| ATOM | 2102 | CB | LYS | B | 20 | 74.250 | 64.229 | 23.005 | 1.00 | 12.95 | B |
| ATOM | 2103 | CG | LYS | B | 20 | 73.374 | 65.417 | 23.367 | 1.00 | 13.71 | B |
| ATOM | 2104 | CD | LYS | B | 20 | 74.223 | 66.480 | 24.046 | 1.00 | 15.05 | B |
| ATOM | 2105 | CE | LYS | B | 20 | 73.396 | 67.668 | 24.509 | 1.00 | 14.15 | B |
| ATOM | 2106 | NZ | LYS | B | 20 | 74.229 | 68.600 | 25.320 | 1.00 | 15.71 | B |
| ATOM | 2107 | C | LYS | B | 20 | 74.511 | 62.008 | 21.827 | 1.00 | 16.21 | B |
| ATOM | 2108 | O | LYS | B | 20 | 74.881 | 61.068 | 22.529 | 1.00 | 14.97 | B |
| ATOM | 2109 | N | ALA | B | 21 | 74.942 | 62.196 | 20.584 | 1.00 | 18.29 | B |
| ATOM | 2110 | CA | ALA | B | 21 | 75.882 | 61.275 | 19.965 | 1.00 | 19.51 | B |
| ATOM | 2111 | CB | ALA | B | 21 | 76.177 | 61.721 | 18.535 | 1.00 | 21.05 | B |
| ATOM | 2112 | C | ALA | B | 21 | 77.176 | 61.201 | 20.773 | 1.00 | 19.76 | B |
| ATOM | 2113 | O | ALA | B | 21 | 77.802 | 60.144 | 20.864 | 1.00 | 21.42 | B |
| ATOM | 2114 | N | ASN | B | 22 | 77.557 | 62.325 | 21.372 | 1.00 | 19.02 | B |
| ATOM | 2115 | CA | ASN | B | 22 | 78.781 | 62.413 | 22.163 | 1.00 | 18.97 | B |
| ATOM | 2116 | CB | ASN | B | 22 | 79.521 | 63.693 | 21.766 | 1.00 | 20.30 | B |
| ATOM | 2117 | CG | ASN | B | 22 | 78.804 | 64.946 | 22.235 | 1.00 | 21.39 | B |
| ATOM | 2118 | OD1 | ASN | B | 22 | 77.584 | 65.061 | 22.104 | 1.00 | 22.04 | B |
| ATOM | 2119 | ND2 | ASN | B | 22 | 79.560 | 65.895 | 22.780 | 1.00 | 22.05 | B |
| ATOM | 2120 | C | ASN | B | 22 | 78.479 | 62.360 | 23.658 | 1.00 | 17.27 | B |
| ATOM | 2121 | O | ASN | B | 22 | 79.224 | 62.925 | 24.462 | 1.00 | 17.70 | B |
| ATOM | 2122 | N | HIS | B | 23 | 77.400 | 61.678 | 24.033 | 1.00 | 16.09 | B |
| ATOM | 2123 | CA | HIS | B | 23 | 77.013 | 61.598 | 25.438 | 1.00 | 16.05 | B |
| ATOM | 2124 | CB | HIS | B | 23 | 75.685 | 60.827 | 25.551 | 1.00 | 15.26 | B |
| ATOM | 2125 | CG | HIS | B | 23 | 75.793 | 59.364 | 25.245 | 1.00 | 14.60 | B |
| ATOM | 2126 | CD2 | HIS | B | 23 | 75.483 | 58.662 | 24.129 | 1.00 | 15.86 | B |
| ATOM | 2127 | ND1 | HIS | B | 23 | 76.238 | 58.440 | 26.165 | 1.00 | 14.48 | B |
| ATOM | 2128 | CE1 | HIS | B | 23 | 76.195 | 57.232 | 25.632 | 1.00 | 17.13 | B |
| ATOM | 2129 | NE2 | HIS | B | 23 | 75.740 | 57.339 | 24.397 | 1.00 | 15.98 | B |
| ATOM | 2130 | C | HIS | B | 23 | 78.118 | 61.062 | 26.351 | 1.00 | 15.69 | B |
| ATOM | 2131 | O | HIS | B | 23 | 78.184 | 61.428 | 27.521 | 1.00 | 15.50 | B |
| ATOM | 2132 | N | ALA | B | 24 | 78.995 | 60.217 | 25.817 | 1.00 | 17.18 | B |
| ATOM | 2133 | CA | ALA | B | 24 | 80.079 | 59.650 | 26.616 | 1.00 | 18.81 | B |
| ATOM | 2134 | CB | ALA | B | 24 | 80.816 | 58.586 | 25.810 | 1.00 | 19.51 | B |
| ATOM | 2135 | C | ALA | B | 24 | 81.070 | 60.702 | 27.117 | 1.00 | 18.71 | B |
| ATOM | 2136 | O | ALA | B | 24 | 81.774 | 60.482 | 28.102 | 1.00 | 18.45 | B |
| ATOM | 2137 | N | MET | B | 25 | 81.126 | 61.844 | 26.439 | 1.00 | 18.56 | B |
| ATOM | 2138 | CA | MET | B | 25 | 82.037 | 62.919 | 26.822 | 1.00 | 18.29 | B |
| ATOM | 2139 | CB | MET | B | 25 | 82.622 | 63.549 | 25.560 | 1.00 | 19.57 | B |
| ATOM | 2140 | CG | MET | B | 25 | 83.465 | 62.621 | 24.725 | 1.00 | 21.10 | B |
| ATOM | 2141 | SD | MET | B | 25 | 84.120 | 63.508 | 23.313 | 1.00 | 23.59 | B |
| ATOM | 2142 | CE | MET | B | 25 | 85.415 | 64.460 | 24.100 | 1.00 | 24.46 | B |
| ATOM | 2143 | C | MET | B | 25 | 81.326 | 63.950 | 27.684 | 1.00 | 18.23 | B |
| ATOM | 2144 | O | MET | B | 25 | 81.903 | 64.985 | 28.033 | 1.00 | 18.60 | B |
| ATOM | 2145 | N | LEU | B | 26 | 80.074 | 63.666 | 28.026 | 1.00 | 18.31 | B |
| ATOM | 2146 | CA | LEU | B | 26 | 79.266 | 64.587 | 28.812 | 1.00 | 16.85 | B |
| ATOM | 2147 | CB | LEU | B | 26 | 78.088 | 65.041 | 27.947 | 1.00 | 16.92 | B |
| ATOM | 2148 | CG | LEU | B | 26 | 78.475 | 65.583 | 26.567 | 1.00 | 17.51 | B |
| ATOM | 2149 | CD1 | LEU | B | 26 | 77.230 | 65.836 | 25.739 | 1.00 | 16.45 | B |
| ATOM | 2150 | CD2 | LEU | B | 26 | 79.279 | 66.862 | 26.726 | 1.00 | 17.51 | B |
| ATOM | 2151 | C | LEU | B | 26 | 78.830 | 63.976 | 30.137 | 1.00 | 16.24 | B |
| ATOM | 2152 | O | LEU | B | 26 | 79.043 | 62.791 | 30.394 | 1.00 | 15.00 | B |
| ATOM | 2153 | N | ALA | B | 27 | 78.207 | 64.800 | 30.971 | 1.00 | 16.05 | B |
| ATOM | 2154 | CA | ALA | B | 27 | 77.743 | 64.357 | 32.278 | 1.00 | 15.70 | B |
| ATOM | 2155 | CB | ALA | B | 27 | 78.774 | 64.724 | 33.340 | 1.00 | 16.75 | B |
| ATOM | 2156 | C | ALA | B | 27 | 76.397 | 64.975 | 32.633 | 1.00 | 14.51 | B |
| ATOM | 2157 | O | ALA | B | 27 | 76.049 | 66.059 | 32.165 | 1.00 | 14.31 | B |
| ATOM | 2158 | N | GLY | B | 28 | 75.634 | 64.272 | 33.461 | 1.00 | 12.99 | B |
| ATOM | 2159 | CA | GLY | B | 28 | 74.345 | 64.784 | 33.893 | 1.00 | 12.86 | B |
| ATOM | 2160 | C | GLY | B | 28 | 73.346 | 65.108 | 32.799 | 1.00 | 10.18 | B |
| ATOM | 2161 | O | GLY | B | 28 | 73.243 | 64.402 | 31.792 | 1.00 | 10.92 | B |
| ATOM | 2162 | N | ASN | B | 29 | 72.606 | 66.194 | 32.997 | 1.00 | 10.97 | B |
| ATOM | 2163 | CA | ASN | B | 29 | 71.588 | 66.617 | 32.042 | 1.00 | 11.28 | B |
| ATOM | 2164 | CB | ASN | B | 29 | 70.871 | 67.866 | 32.575 | 1.00 | 12.52 | B |
| ATOM | 2165 | CG | ASN | B | 29 | 70.099 | 67.613 | 33.862 | 1.00 | 13.16 | B |
| ATOM | 2166 | OD1 | ASN | B | 29 | 69.615 | 68.557 | 34.496 | 1.00 | 16.07 | B |
| ATOM | 2167 | ND2 | ASN | B | 29 | 69.968 | 66.351 | 34.249 | 1.00 | 10.44 | B |
| ATOM | 2168 | C | ASN | B | 29 | 72.159 | 66.854 | 30.647 | 1.00 | 11.11 | B |
| ATOM | 2169 | O | ASN | B | 29 | 71.440 | 66.751 | 29.662 | 1.00 | 12.20 | B |
| ATOM | 2170 | N | GLU | B | 30 | 73.445 | 67.190 | 30.574 | 1.00 | 11.25 | B |
| ATOM | 2171 | CA | GLU | B | 30 | 74.116 | 67.468 | 29.302 | 1.00 | 11.59 | B |
| ATOM | 2172 | CB | GLU | B | 30 | 75.531 | 67.982 | 29.592 | 1.00 | 13.28 | B |
| ATOM | 2173 | CG | GLU | B | 30 | 75.600 | 69.370 | 30.208 | 1.00 | 18.28 | B |
| ATOM | 2174 | CD | GLU | B | 30 | 75.494 | 70.476 | 29.176 | 1.00 | 22.46 | B |
| ATOM | 2175 | OE1 | GLU | B | 30 | 75.433 | 70.163 | 27.969 | 1.00 | 23.11 | B |
| ATOM | 2176 | OE2 | GLU | B | 30 | 75.482 | 71.662 | 29.575 | 1.00 | 24.65 | B |
| ATOM | 2177 | C | GLU | B | 30 | 74.135 | 66.296 | 28.324 | 1.00 | 10.36 | B |
| ATOM | 2178 | O | GLU | B | 30 | 74.352 | 66.487 | 27.128 | 1.00 | 11.64 | B |
| ATOM | 2179 | N | ARG | B | 31 | 73.923 | 65.086 | 28.834 | 1.00 | 9.81 | B |
| ATOM | 2180 | CA | ARG | B | 31 | 73.930 | 63.887 | 28.003 | 1.00 | 10.32 | B |
| ATOM | 2181 | CB | ARG | B | 31 | 73.955 | 62.647 | 28.899 | 1.00 | 10.62 | B |
| ATOM | 2182 | CG | ARG | B | 31 | 75.192 | 62.443 | 29.743 | 1.00 | 10.62 | B |
| ATOM | 2183 | CD | ARG | B | 31 | 74.937 | 61.315 | 30.732 | 1.00 | 9.95 | B |
| ATOM | 2184 | NE | ARG | B | 31 | 73.920 | 61.704 | 31.706 | 1.00 | 10.06 | B |
| ATOM | 2185 | CZ | ARG | B | 31 | 73.348 | 60.876 | 32.574 | 1.00 | 9.21 | B |
| ATOM | 2186 | NH1 | ARG | B | 31 | 73.681 | 59.595 | 32.597 | 1.00 | 11.18 | B |
| ATOM | 2187 | NH2 | ARG | B | 31 | 72.452 | 61.338 | 33.433 | 1.00 | 10.47 | B |
| ATOM | 2188 | C | ARG | B | 31 | 72.744 | 63.814 | 27.057 | 1.00 | 10.20 | B |
| ATOM | 2189 | O | ARG | B | 31 | 72.797 | 63.101 | 26.058 | 1.00 | 11.31 | B |
| ATOM | 2190 | N | TYR | B | 32 | 71.676 | 64.538 | 27.387 | 1.00 | 9.98 | B |
| ATOM | 2191 | CA | TYR | B | 32 | 70.425 | 64.496 | 26.627 | 1.00 | 8.83 | B |
| ATOM | 2192 | CB | TYR | B | 32 | 69.291 | 64.157 | 27.603 | 1.00 | 10.20 | B |
| ATOM | 2193 | CG | TYR | B | 32 | 69.544 | 62.935 | 28.451 | 1.00 | 9.07 | B |
| ATOM | 2194 | CD1 | TYR | B | 32 | 69.397 | 61.655 | 27.923 | 1.00 | 10.68 | B |
| ATOM | 2195 | CE1 | TYR | B | 32 | 69.645 | 60.522 | 28.698 | 1.00 | 10.81 | B |
| ATOM | 2196 | CD2 | TYR | B | 32 | 69.949 | 63.059 | 29.778 | 1.00 | 10.74 | B |
| ATOM | 2197 | CE2 | TYR | B | 32 | 70.203 | 61.935 | 30.562 | 1.00 | 10.68 | B |
| ATOM | 2198 | CZ | TYR | B | 32 | 70.048 | 60.670 | 30.015 | 1.00 | 9.71 | B |
| ATOM | 2199 | OH | TYR | B | 32 | 70.304 | 59.557 | 30.786 | 1.00 | 12.33 | B |
| ATOM | 2200 | C | TYR | B | 32 | 70.086 | 65.741 | 25.825 | 1.00 | 9.96 | B |
| ATOM | 2201 | O | TYR | B | 32 | 70.530 | 66.840 | 26.137 | 1.00 | 9.85 | B |
| ATOM | 2202 | N | GLU | B | 33 | 69.267 | 65.547 | 24.794 | 1.00 | 9.25 | B |
| ATOM | 2203 | CA | GLU | B | 33 | 68.802 | 66.637 | 23.945 | 1.00 | 9.61 | B |
| ATOM | 2204 | CB | GLU | B | 33 | 69.789 | 66.877 | 22.801 | 1.00 | 12.38 | B |
| ATOM | 2205 | CG | GLU | B | 33 | 69.882 | 65.719 | 21.823 | 1.00 | 15.84 | B |
| ATOM | 2206 | CD | GLU | B | 33 | 70.830 | 65.994 | 20.670 | 1.00 | 19.62 | B |
| ATOM | 2207 | OE1 | GLU | B | 33 | 71.160 | 65.039 | 19.936 | 1.00 | 21.62 | B |
| ATOM | 2208 | OE2 | GLU | B | 33 | 71.240 | 67.161 | 20.495 | 1.00 | 22.03 | B |
| ATOM | 2209 | C | GLU | B | 33 | 67.412 | 66.291 | 23.441 | 1.00 | 10.42 | B |
| ATOM | 2210 | O | GLU | B | 33 | 67.012 | 65.118 | 23.431 | 1.00 | 10.16 | B |
| ATOM | 2211 | N | GLY | B | 34 | 66.672 | 67.314 | 23.028 | 1.00 | 9.75 | B |
| ATOM | 2212 | CA | GLY | B | 34 | 65.339 | 67.083 | 22.510 | 1.00 | 9.73 | B |
| ATOM | 2213 | C | GLY | B | 34 | 64.321 | 68.084 | 23.016 | 1.00 | 8.85 | B |
| ATOM | 2214 | O | GLY | B | 34 | 64.616 | 68.904 | 23.887 | 1.00 | 10.30 | B |
| ATOM | 2215 | N | TYR | B | 35 | 63.115 | 68.003 | 22.468 | 1.00 | 8.53 | B |
| ATOM | 2216 | CA | TYR | B | 35 | 62.032 | 68.902 | 22.838 | 1.00 | 9.68 | B |
| ATOM | 2217 | CB | TYR | B | 35 | 60.782 | 68.528 | 22.031 | 1.00 | 10.10 | B |
| ATOM | 2218 | CG | TYR | B | 35 | 59.547 | 69.312 | 22.415 | 1.00 | 9.72 | B |
| ATOM | 2219 | CD1 | TYR | B | 35 | 58.848 | 69.024 | 23.590 | 1.00 | 9.32 | B |
| ATOM | 2220 | CE1 | TYR | B | 35 | 57.721 | 69.755 | 23.954 | 1.00 | 10.16 | B |
| ATOM | 2221 | CD2 | TYR | B | 35 | 59.086 | 70.355 | 21.613 | 1.00 | 10.84 | B |
| ATOM | 2222 | CE2 | TYR | B | 35 | 57.962 | 71.094 | 21.970 | 1.00 | 11.51 | B |
| ATOM | 2223 | CZ | TYR | B | 35 | 57.287 | 70.789 | 23.140 | 1.00 | 9.88 | B |
| ATOM | 2224 | OH | TYR | B | 35 | 56.185 | 71.530 | 23.507 | 1.00 | 12.71 | B |
| ATOM | 2225 | C | TYR | B | 35 | 61.773 | 68.928 | 24.341 | 1.00 | 9.67 | B |
| ATOM | 2226 | O | TYR | B | 35 | 61.676 | 70.000 | 24.943 | 1.00 | 10.24 | B |
| ATOM | 2227 | N | CYS | B | 36 | 61.644 | 67.755 | 24.952 | 1.00 | 9.69 | B |
| ATOM | 2228 | CA | CYS | B | 36 | 61.369 | 67.698 | 26.383 | 1.00 | 9.64 | B |
| ATOM | 2229 | CB | CYS | B | 36 | 60.823 | 66.312 | 26.744 | 1.00 | 11.19 | B |
| ATOM | 2230 | SG | CYS | B | 36 | 59.098 | 66.081 | 26.239 | 1.00 | 11.34 | B |
| ATOM | 2231 | C | CYS | B | 36 | 62.553 | 68.128 | 27.232 | 1.00 | 10.65 | B |
| ATOM | 2232 | O | CYS | B | 36 | 62.377 | 68.564 | 28.366 | 1.00 | 10.55 | B |
| ATOM | 2233 | N | VAL | B | 37 | 63.760 | 67.997 | 26.695 | 1.00 | 9.52 | B |
| ATOM | 2234 | CA | VAL | B | 37 | 64.945 | 68.430 | 27.423 | 1.00 | 10.61 | B |
| ATOM | 2235 | CB | VAL | B | 37 | 66.235 | 67.947 | 26.721 | 1.00 | 9.82 | B |
| ATOM | 2236 | CG1 | VAL | B | 37 | 67.459 | 68.492 | 27.442 | 1.00 | 11.76 | B |
| ATOM | 2237 | CG2 | VAL | B | 37 | 66.273 | 66.424 | 26.714 | 1.00 | 12.23 | B |
| ATOM | 2238 | C | VAL | B | 37 | 64.908 | 69.962 | 27.465 | 1.00 | 10.09 | B |
| ATOM | 2239 | O | VAL | B | 37 | 65.147 | 70.577 | 28.507 | 1.00 | 11.08 | B |
| ATOM | 2240 | N | ASP | B | 38 | 64.592 | 70.572 | 26.326 | 1.00 | 10.59 | B |
| ATOM | 2241 | CA | ASP | B | 38 | 64.495 | 72.024 | 26.232 | 1.00 | 9.97 | B |
| ATOM | 2242 | CB | ASP | B | 38 | 64.314 | 72.438 | 24.768 | 1.00 | 11.07 | B |
| ATOM | 2243 | CG | ASP | B | 38 | 65.571 | 72.264 | 23.939 | 1.00 | 10.71 | B |
| ATOM | 2244 | OD1 | ASP | B | 38 | 65.470 | 72.430 | 22.707 | 1.00 | 12.33 | B |
| ATOM | 2245 | OD2 | ASP | B | 38 | 66.644 | 71.977 | 24.506 | 1.00 | 13.75 | B |
| ATOM | 2246 | C | ASP | B | 38 | 63.336 | 72.504 | 27.100 | 1.00 | 10.54 | B |
| ATOM | 2247 | O | ASP | B | 38 | 63.442 | 73.529 | 27.776 | 1.00 | 10.16 | B |
| ATOM | 2248 | N | LEU | B | 39 | 62.222 | 71.772 | 27.066 | 1.00 | 9.79 | B |
| ATOM | 2249 | CA | LEU | B | 39 | 61.055 | 72.147 | 27.854 | 1.00 | 10.05 | B |
| ATOM | 2250 | CB | LEU | B | 39 | 59.870 | 71.244 | 27.490 | 1.00 | 11.14 | B |
| ATOM | 2251 | CG | LEU | B | 39 | 58.603 | 71.478 | 28.324 | 1.00 | 10.08 | B |
| ATOM | 2252 | CD1 | LEU | B | 39 | 58.137 | 72.921 | 28.178 | 1.00 | 11.75 | B |
| ATOM | 2253 | CD2 | LEU | B | 39 | 57.516 | 70.503 | 27.885 | 1.00 | 11.65 | B |
| ATOM | 2254 | C | LEU | B | 39 | 61.393 | 72.098 | 29.334 | 1.00 | 9.39 | B |
| ATOM | 2255 | O | LEU | B | 39 | 61.048 | 73.010 | 30.086 | 1.00 | 10.29 | B |
| ATOM | 2256 | N | ALA | B | 40 | 62.061 | 71.030 | 29.758 | 1.00 | 9.61 | B |
| ATOM | 2257 | CA | ALA | B | 40 | 62.432 | 70.896 | 31.162 | 1.00 | 10.73 | B |
| ATOM | 2258 | CB | ALA | B | 40 | 63.196 | 69.595 | 31.380 | 1.00 | 11.25 | B |
| ATOM | 2259 | C | ALA | B | 40 | 63.270 | 72.088 | 31.622 | 1.00 | 11.73 | B |
| ATOM | 2260 | O | ALA | B | 40 | 63.046 | 72.638 | 32.701 | 1.00 | 12.59 | B |
| ATOM | 2261 | N | ALA | B | 41 | 64.230 | 72.498 | 30.801 | 1.00 | 11.86 | B |
| ATOM | 2262 | CA | ALA | B | 41 | 65.082 | 73.626 | 31.153 | 1.00 | 12.18 | B |
| ATOM | 2263 | CB | ALA | B | 41 | 66.173 | 73.809 | 30.098 | 1.00 | 13.52 | B |
| ATOM | 2264 | C | ALA | B | 41 | 64.268 | 74.909 | 31.311 | 1.00 | 12.68 | B |
| ATOM | 2265 | O | ALA | B | 41 | 64.528 | 75.704 | 32.214 | 1.00 | 13.44 | B |
| ATOM | 2266 | N | GLU | B | 42 | 63.289 | 75.111 | 30.433 | 1.00 | 12.40 | B |
| ATOM | 2267 | CA | GLU | B | 42 | 62.445 | 76.299 | 30.499 | 1.00 | 13.32 | B |
| ATOM | 2268 | CB | GLU | B | 42 | 61.657 | 76.433 | 29.192 | 1.00 | 14.71 | B |
| ATOM | 2269 | CG | GLU | B | 42 | 62.430 | 77.116 | 28.081 | 1.00 | 18.34 | B |
| ATOM | 2270 | CD | GLU | B | 42 | 62.565 | 78.614 | 28.306 | 1.00 | 20.10 | B |
| ATOM | 2271 | OE1 | GLU | B | 42 | 62.279 | 79.083 | 29.429 | 1.00 | 20.21 | B |
| ATOM | 2272 | OE2 | GLU | B | 42 | 62.962 | 79.326 | 27.361 | 1.00 | 21.49 | B |
| ATOM | 2273 | C | GLU | B | 42 | 61.552 | 76.273 | 31.730 | 1.00 | 13.78 | B |
| ATOM | 2274 | O | GLU | B | 42 | 61.370 | 77.293 | 32.392 | 1.00 | 14.79 | B |
| ATOM | 2275 | N | ILE | B | 43 | 60.978 | 75.113 | 32.030 | 1.00 | 13.34 | B |
| ATOM | 2276 | CA | ILE | B | 43 | 60.115 | 74.987 | 33.199 | 1.00 | 13.46 | B |
| ATOM | 2277 | CB | ILE | B | 43 | 59.490 | 73.572 | 33.289 | 1.00 | 13.33 | B |
| ATOM | 2278 | CG2 | ILE | B | 43 | 58.751 | 73.408 | 34.613 | 1.00 | 12.28 | B |
| ATOM | 2279 | CG1 | ILE | B | 43 | 58.541 | 73.339 | 32.110 | 1.00 | 14.86 | B |
| ATOM | 2280 | CD1 | ILE | B | 43 | 57.323 | 74.242 | 32.101 | 1.00 | 16.43 | B |
| ATOM | 2281 | C | ILE | B | 43 | 60.925 | 75.263 | 34.468 | 1.00 | 13.03 | B |
| ATOM | 2282 | O | ILE | B | 43 | 60.498 | 76.023 | 35.339 | 1.00 | 15.24 | B |
| ATOM | 2283 | N | ALA | B | 44 | 62.102 | 74.653 | 34.563 | 1.00 | 13.22 | B |
| ATOM | 2284 | CA | ALA | B | 44 | 62.966 | 74.840 | 35.725 | 1.00 | 13.12 | B |
| ATOM | 2285 | CB | ALA | B | 44 | 64.197 | 73.962 | 35.599 | 1.00 | 13.62 | B |
| ATOM | 2286 | C | ALA | B | 44 | 63.379 | 76.298 | 35.886 | 1.00 | 14.36 | B |
| ATOM | 2287 | O | ALA | B | 44 | 63.439 | 76.812 | 37.001 | 1.00 | 14.64 | B |
| ATOM | 2288 | N | LYS | B | 45 | 63.666 | 76.959 | 34.769 | 1.00 | 15.50 | B |
| ATOM | 2289 | CA | LYS | B | 45 | 64.073 | 78.358 | 34.784 | 1.00 | 18.35 | B |
| ATOM | 2290 | CB | LYS | B | 45 | 64.464 | 78.775 | 33.366 | 1.00 | 19.84 | B |
| ATOM | 2291 | CG | LYS | B | 45 | 65.067 | 80.167 | 33.270 | 1.00 | 24.18 | B |
| ATOM | 2292 | CD | LYS | B | 45 | 65.213 | 80.612 | 31.819 | 1.00 | 28.35 | B |
| ATOM | 2293 | CE | LYS | B | 45 | 65.838 | 79.523 | 30.960 | 1.00 | 30.41 | B |
| ATOM | 2294 | NZ | LYS | B | 45 | 67.050 | 78.941 | 31.597 | 1.00 | 32.84 | B |
| ATOM | 2295 | C | LYS | B | 45 | 62.962 | 79.225 | 35.360 | 1.00 | 18.22 | B |
| ATOM | 2296 | O | LYS | B | 45 | 63.206 | 80.084 | 36.208 | 1.00 | 18.79 | B |
| ATOM | 2297 | N | HIS | B | 46 | 61.738 | 79.002 | 34.893 | 1.00 | 16.85 | B |
| ATOM | 2298 | CA | HIS | B | 46 | 60.597 | 79.781 | 35.357 | 1.00 | 18.06 | B |
| ATOM | 2299 | CB | HIS | B | 46 | 59.451 | 79.650 | 34.346 | 1.00 | 16.99 | B |
| ATOM | 2300 | CG | HIS | B | 46 | 59.668 | 80.432 | 33.089 | 1.00 | 18.62 | B |
| ATOM | 2301 | CD2 | HIS | B | 46 | 59.222 | 81.655 | 32.716 | 1.00 | 19.23 | B |
| ATOM | 2302 | ND1 | HIS | B | 46 | 60.476 | 79.990 | 32.064 | 1.00 | 20.49 | B |
| ATOM | 2303 | CE1 | HIS | B | 46 | 60.520 | 80.908 | 31.115 | 1.00 | 20.66 | B |
| ATOM | 2304 | NE2 | HIS | B | 46 | 59.768 | 81.928 | 31.486 | 1.00 | 22.31 | B |
| ATOM | 2305 | C | HIS | B | 46 | 60.166 | 79.459 | 36.783 | 1.00 | 18.25 | B |
| ATOM | 2306 | O | HIS | B | 46 | 59.643 | 80.328 | 37.481 | 1.00 | 20.05 | B |
| ATOM | 2307 | N | CYS | B | 47 | 60.381 | 78.224 | 37.224 | 1.00 | 18.44 | B |
| ATOM | 2308 | CA | CYS | B | 47 | 59.993 | 77.827 | 38.575 | 1.00 | 17.80 | B |
| ATOM | 2309 | CB | CYS | B | 47 | 59.473 | 76.389 | 38.549 | 1.00 | 18.10 | B |
| ATOM | 2310 | SG | CYS | B | 47 | 57.992 | 76.181 | 37.546 | 1.00 | 16.20 | B |
| ATOM | 2311 | C | CYS | B | 47 | 61.129 | 78.027 | 39.564 | 1.00 | 18.32 | B |
| ATOM | 2312 | O | CYS | B | 47 | 60.924 | 77.976 | 40.777 | 1.00 | 19.27 | B |
| ATOM | 2313 | N | GLY | B | 48 | 62.329 | 78.237 | 39.039 | 1.00 | 17.47 | B |
| ATOM | 2314 | CA | GLY | B | 48 | 63.483 | 78.460 | 39.887 | 1.00 | 17.87 | B |
| ATOM | 2315 | C | GLY | B | 48 | 63.989 | 77.250 | 40.647 | 1.00 | 17.16 | B |
| ATOM | 2316 | O | GLY | B | 48 | 64.315 | 77.357 | 41.829 | 1.00 | 19.29 | B |
| ATOM | 2317 | N | PHE | B | 49 | 64.056 | 76.096 | 39.991 | 1.00 | 16.57 | B |
| ATOM | 2318 | CA | PHE | B | 49 | 64.563 | 74.908 | 40.662 | 1.00 | 15.09 | B |
| ATOM | 2319 | CB | PHE | B | 49 | 63.397 | 73.957 | 41.021 | 1.00 | 15.47 | B |
| ATOM | 2320 | CG | PHE | B | 49 | 62.679 | 73.346 | 39.832 | 1.00 | 14.74 | B |
| ATOM | 2321 | CD1 | PHE | B | 49 | 63.277 | 72.476 | 39.080 | 1.00 | 13.45 | B |
| ATOM | 2322 | CD2 | PHE | B | 49 | 61.443 | 73.662 | 39.599 | 1.00 | 13.69 | B |
| ATOM | 2323 | CE1 | PHE | B | 49 | 62.630 | 71.844 | 38.006 | 1.00 | 15.09 | B |
| ATOM | 2324 | CE2 | PHE | B | 49 | 60.709 | 73.086 | 38.549 | 1.00 | 15.17 | B |
| ATOM | 2325 | CZ | PHE | B | 49 | 61.319 | 72.159 | 37.741 | 1.00 | 14.08 | B |
| ATOM | 2326 | C | PHE | B | 49 | 65.657 | 74.240 | 39.847 | 1.00 | 14.16 | B |
| ATOM | 2327 | O | PHE | B | 49 | 65.747 | 74.429 | 38.634 | 1.00 | 13.98 | B |
| ATOM | 2328 | N | ALA | B | 50 | 66.512 | 73.493 | 40.540 | 1.00 | 13.90 | B |
| ATOM | 2329 | CA | ALA | B | 50 | 67.580 | 72.740 | 39.896 | 1.00 | 13.70 | B |
| ATOM | 2330 | CB | ALA | B | 50 | 68.814 | 72.695 | 40.785 | 1.00 | 16.32 | B |
| ATOM | 2331 | C | ALA | B | 50 | 66.979 | 71.350 | 39.743 | 1.00 | 13.30 | B |
| ATOM | 2332 | O | ALA | B | 50 | 66.143 | 70.945 | 40.553 | 1.00 | 13.71 | B |
| ATOM | 2333 | N | TYR | B | 51 | 67.391 | 70.614 | 38.719 | 1.00 | 13.03 | B |
| ATOM | 2334 | CA | TYR | B | 51 | 66.812 | 69.296 | 38.500 | 1.00 | 11.76 | B |
| ATOM | 2335 | CB | TYR | B | 51 | 65.573 | 69.443 | 37.617 | 1.00 | 11.44 | B |
| ATOM | 2336 | CG | TYR | B | 51 | 65.911 | 69.967 | 36.243 | 1.00 | 11.49 | B |
| ATOM | 2337 | CD1 | TYR | B | 51 | 66.193 | 71.315 | 36.048 | 1.00 | 12.38 | B |
| ATOM | 2338 | CE1 | TYR | B | 51 | 66.566 | 71.799 | 34.802 | 1.00 | 12.32 | B |
| ATOM | 2339 | CD2 | TYR | B | 51 | 66.010 | 69.106 | 35.148 | 1.00 | 12.07 | B |
| ATOM | 2340 | CE2 | TYR | B | 51 | 66.386 | 69.583 | 33.895 | 1.00 | 11.64 | B |
| ATOM | 2341 | CZ | TYR | B | 51 | 66.662 | 70.930 | 33.732 | 1.00 | 12.48 | B |
| ATOM | 2342 | OH | TYR | B | 51 | 67.034 | 71.413 | 32.504 | 1.00 | 14.13 | B |
| ATOM | 2343 | C | TYR | B | 51 | 67.784 | 68.307 | 37.898 | 1.00 | 11.11 | B |
| ATOM | 2344 | O | TYR | B | 51 | 68.827 | 68.673 | 37.353 | 1.00 | 11.36 | B |
| ATOM | 2345 | N | ALA | B | 52 | 67.402 | 67.040 | 37.983 | 1.00 | 11.04 | B |
| ATOM | 2346 | CA | ALA | B | 52 | 68.190 | 65.953 | 37.435 | 1.00 | 11.50 | B |
| ATOM | 2347 | CB | ALA | B | 52 | 68.743 | 65.097 | 38.556 | 1.00 | 14.07 | B |
| ATOM | 2348 | C | ALA | B | 52 | 67.282 | 65.120 | 36.542 | 1.00 | 12.04 | B |
| ATOM | 2349 | O | ALA | B | 52 | 66.230 | 64.655 | 36.983 | 1.00 | 11.18 | B |
| ATOM | 2350 | N | LEU | B | 53 | 67.677 | 64.952 | 35.283 | 1.00 | 11.19 | B |
| ATOM | 2351 | CA | LEU | B | 53 | 66.907 | 64.145 | 34.342 | 1.00 | 11.11 | B |
| ATOM | 2352 | CB | LEU | B | 53 | 67.237 | 64.557 | 32.902 | 1.00 | 12.06 | B |
| ATOM | 2353 | CG | LEU | B | 53 | 66.890 | 65.967 | 32.423 | 1.00 | 11.30 | B |
| ATOM | 2354 | CD1 | LEU | B | 53 | 67.398 | 66.148 | 30.995 | 1.00 | 13.51 | B |
| ATOM | 2355 | CD2 | LEU | B | 53 | 65.389 | 66.189 | 32.492 | 1.00 | 12.62 | B |
| ATOM | 2356 | C | LEU | B | 53 | 67.235 | 62.683 | 34.591 | 1.00 | 11.07 | B |
| ATOM | 2357 | O | LEU | B | 53 | 68.401 | 62.291 | 34.550 | 1.00 | 12.22 | B |
| ATOM | 2358 | N | THR | B | 54 | 66.210 | 61.881 | 34.858 | 1.00 | 10.41 | B |
| ATOM | 2359 | CA | THR | B | 54 | 66.394 | 60.452 | 35.079 | 1.00 | 10.10 | B |
| ATOM | 2360 | CB | THR | B | 54 | 66.036 | 60.030 | 36.525 | 1.00 | 11.69 | B |
| ATOM | 2361 | OG1 | THR | B | 54 | 64.630 | 60.203 | 36.752 | 1.00 | 12.12 | B |
| ATOM | 2362 | CG2 | THR | B | 54 | 66.816 | 60.862 | 37.527 | 1.00 | 12.54 | B |
| ATOM | 2363 | C | THR | B | 54 | 65.482 | 59.730 | 34.095 | 1.00 | 10.22 | B |
| ATOM | 2364 | O | THR | B | 54 | 64.430 | 60.247 | 33.721 | 1.00 | 10.58 | B |
| ATOM | 2365 | N | ILE | B | 55 | 65.898 | 58.546 | 33.661 | 1.00 | 11.35 | B |
| ATOM | 2366 | CA | ILE | B | 55 | 65.123 | 57.775 | 32.701 | 1.00 | 9.56 | B |
| ATOM | 2367 | CB | ILE | B | 55 | 66.054 | 57.099 | 31.677 | 1.00 | 10.28 | B |
| ATOM | 2368 | CG2 | ILE | B | 55 | 65.230 | 56.422 | 30.596 | 1.00 | 10.52 | B |
| ATOM | 2369 | CG1 | ILE | B | 55 | 66.997 | 58.141 | 31.066 | 1.00 | 12.28 | B |
| ATOM | 2370 | CD1 | ILE | B | 55 | 66.297 | 59.307 | 30.389 | 1.00 | 13.29 | B |
| ATOM | 2371 | C | ILE | B | 55 | 64.293 | 56.721 | 33.415 | 1.00 | 11.06 | B |
| ATOM | 2372 | O | ILE | B | 55 | 64.822 | 55.907 | 34.175 | 1.00 | 10.82 | B |
| ATOM | 2373 | N | VAL | B | 56 | 62.986 | 56.743 | 33.170 | 1.00 | 11.05 | B |
| ATOM | 2374 | CA | VAL | B | 56 | 62.066 | 55.809 | 33.807 | 1.00 | 11.37 | B |
| ATOM | 2375 | CB | VAL | B | 56 | 60.629 | 55.969 | 33.228 | 1.00 | 9.97 | B |
| ATOM | 2376 | CG1 | VAL | B | 56 | 60.615 | 55.684 | 31.744 | 1.00 | 9.92 | B |
| ATOM | 2377 | CG2 | VAL | B | 56 | 59.667 | 55.064 | 33.966 | 1.00 | 11.61 | B |
| ATOM | 2378 | C | VAL | B | 56 | 62.543 | 54.360 | 33.703 | 1.00 | 11.84 | B |
| ATOM | 2379 | O | VAL | B | 56 | 62.792 | 53.835 | 32.616 | 1.00 | 11.48 | B |
| ATOM | 2380 | N | GLY | B | 57 | 62.657 | 53.723 | 34.862 | 1.00 | 12.37 | B |
| ATOM | 2381 | CA | GLY | B | 57 | 63.146 | 52.360 | 34.937 | 1.00 | 13.63 | B |
| ATOM | 2382 | C | GLY | B | 57 | 62.508 | 51.288 | 34.080 | 1.00 | 14.12 | B |
| ATOM | 2383 | O | GLY | B | 57 | 63.221 | 50.521 | 33.430 | 1.00 | 15.65 | B |
| ATOM | 2384 | N | ASP | B | 58 | 61.178 | 51.223 | 34.068 | 1.00 | 14.76 | B |
| ATOM | 2385 | CA | ASP | B | 58 | 60.486 | 50.189 | 33.307 | 1.00 | 14.59 | B |
| ATOM | 2386 | CB | ASP | B | 58 | 59.266 | 49.713 | 34.110 | 1.00 | 14.42 | B |
| ATOM | 2387 | CG | ASP | B | 58 | 58.191 | 50.776 | 34.243 | 1.00 | 13.18 | B |
| ATOM | 2388 | OD1 | ASP | B | 58 | 57.230 | 50.545 | 35.005 | 1.00 | 16.08 | B |
| ATOM | 2389 | OD2 | ASP | B | 58 | 58.296 | 51.835 | 33.586 | 1.00 | 12.74 | B |
| ATOM | 2390 | C | ASP | B | 58 | 60.143 | 50.567 | 31.872 | 1.00 | 14.19 | B |
| ATOM | 2391 | O | ASP | B | 58 | 59.470 | 49.805 | 31.180 | 1.00 | 15.43 | B |
| ATOM | 2392 | N | GLY | B | 59 | 60.594 | 51.737 | 31.428 | 1.00 | 14.19 | B |
| ATOM | 2393 | CA | GLY | B | 59 | 60.331 | 52.162 | 30.062 | 1.00 | 11.83 | B |
| ATOM | 2394 | C | GLY | B | 59 | 58.867 | 52.283 | 29.684 | 1.00 | 12.48 | B |
| ATOM | 2395 | O | GLY | B | 59 | 58.514 | 52.155 | 28.510 | 1.00 | 12.94 | B |
| ATOM | 2396 | N | LYS | B | 60 | 58.017 | 52.551 | 30.672 | 1.00 | 11.56 | B |
| ATOM | 2397 | CA | LYS | B | 60 | 56.577 | 52.677 | 30.450 | 1.00 | 12.01 | B |
| ATOM | 2398 | CB | LYS | B | 60 | 55.846 | 51.652 | 31.326 | 1.00 | 13.09 | B |
| ATOM | 2399 | CG | LYS | B | 60 | 56.245 | 50.200 | 31.110 | 1.00 | 15.47 | B |
| ATOM | 2400 | CD | LYS | B | 60 | 55.738 | 49.675 | 29.786 | 1.00 | 18.86 | B |
| ATOM | 2401 | CE | LYS | B | 60 | 56.022 | 48.185 | 29.656 | 1.00 | 20.26 | B |
| ATOM | 2402 | NZ | LYS | B | 60 | 55.522 | 47.641 | 28.365 | 1.00 | 21.95 | B |
| ATOM | 2403 | C | LYS | B | 60 | 56.104 | 54.098 | 30.731 | 1.00 | 10.84 | B |
| ATOM | 2404 | O | LYS | B | 60 | 56.786 | 54.875 | 31.408 | 1.00 | 11.54 | B |
| ATOM | 2405 | N | TYR | B | 61 | 54.923 | 54.432 | 30.217 | 1.00 | 10.31 | B |
| ATOM | 2406 | CA | TYR | B | 61 | 54.352 | 55.754 | 30.435 | 1.00 | 10.95 | B |
| ATOM | 2407 | CB | TYR | B | 61 | 53.617 | 56.209 | 29.172 | 1.00 | 10.41 | B |
| ATOM | 2408 | CG | TYR | B | 61 | 54.567 | 56.686 | 28.106 | 1.00 | 10.06 | B |
| ATOM | 2409 | CD1 | TYR | B | 61 | 55.372 | 55.783 | 27.409 | 1.00 | 10.42 | B |
| ATOM | 2410 | CE1 | TYR | B | 61 | 56.304 | 56.224 | 26.479 | 1.00 | 10.50 | B |
| ATOM | 2411 | CD2 | TYR | B | 61 | 54.717 | 58.047 | 27.837 | 1.00 | 10.35 | B |
| ATOM | 2412 | CE2 | TYR | B | 61 | 55.648 | 58.499 | 26.906 | 1.00 | 10.24 | B |
| ATOM | 2413 | CZ | TYR | B | 61 | 56.435 | 57.581 | 26.234 | 1.00 | 11.07 | B |
| ATOM | 2414 | OH | TYR | B | 61 | 57.349 | 58.030 | 25.310 | 1.00 | 12.79 | B |
| ATOM | 2415 | C | TYR | B | 61 | 53.487 | 55.771 | 31.685 | 1.00 | 9.95 | B |
| ATOM | 2416 | O | TYR | B | 61 | 53.714 | 56.578 | 32.585 | 1.00 | 11.01 | B |
| ATOM | 2417 | N | GLY | B | 62 | 52.499 | 54.884 | 31.750 | 1.00 | 10.51 | B |
| ATOM | 2418 | CA | GLY | B | 62 | 51.653 | 54.847 | 32.930 | 1.00 | 11.84 | B |
| ATOM | 2419 | C | GLY | B | 62 | 50.340 | 54.109 | 32.768 | 1.00 | 10.79 | B |
| ATOM | 2420 | O | GLY | B | 62 | 49.416 | 54.599 | 32.116 | 1.00 | 10.82 | B |
| ATOM | 2421 | N | ALA | B | 63 | 50.264 | 52.927 | 33.370 | 1.00 | 12.75 | B |
| ATOM | 2422 | CA | ALA | B | 63 | 49.065 | 52.100 | 33.330 | 1.00 | 13.25 | B |
| ATOM | 2423 | CB | ALA | B | 63 | 49.122 | 51.145 | 32.147 | 1.00 | 13.85 | B |
| ATOM | 2424 | C | ALA | B | 63 | 49.002 | 51.321 | 34.637 | 1.00 | 14.70 | B |
| ATOM | 2425 | O | ALA | B | 63 | 50.030 | 51.085 | 35.276 | 1.00 | 14.41 | B |
| ATOM | 2426 | N | ARG | B | 64 | 47.796 | 50.926 | 35.032 | 1.00 | 15.95 | B |
| ATOM | 2427 | CA | ARG | B | 64 | 47.603 | 50.178 | 36.268 | 1.00 | 18.84 | B |
| ATOM | 2428 | CB | ARG | B | 64 | 46.555 | 50.896 | 37.129 | 1.00 | 20.04 | B |
| ATOM | 2429 | CG | ARG | B | 64 | 46.300 | 50.249 | 38.487 | 1.00 | 23.49 | B |
| ATOM | 2430 | CD | ARG | B | 64 | 44.957 | 49.522 | 38.541 | 1.00 | 27.55 | B |
| ATOM | 2431 | NE | ARG | B | 64 | 43.825 | 50.438 | 38.421 | 1.00 | 28.94 | B |
| ATOM | 2432 | CZ | ARG | B | 64 | 42.551 | 50.076 | 38.545 | 1.00 | 30.80 | B |
| ATOM | 2433 | NH1 | ARG | B | 64 | 42.238 | 48.812 | 38.797 | 1.00 | 29.54 | B |
| ATOM | 2434 | NH2 | ARG | B | 64 | 41.587 | 50.977 | 38.417 | 1.00 | 31.20 | B |
| ATOM | 2435 | C | ARG | B | 64 | 47.233 | 48.734 | 35.961 | 1.00 | 19.59 | B |
| ATOM | 2436 | O | ARG | B | 64 | 46.286 | 48.470 | 35.220 | 1.00 | 19.24 | B |
| ATOM | 2437 | N | ASP | B | 65 | 47.990 | 47.801 | 36.530 | 1.00 | 21.81 | B |
| ATOM | 2438 | CA | ASP | B | 65 | 47.723 | 46.382 | 36.327 | 1.00 | 23.78 | B |
| ATOM | 2439 | CB | ASP | B | 65 | 48.852 | 45.563 | 36.956 | 1.00 | 24.91 | B |
| ATOM | 2440 | CG | ASP | B | 65 | 48.744 | 44.084 | 36.636 | 1.00 | 26.39 | B |
| ATOM | 2441 | OD1 | ASP | B | 65 | 48.998 | 43.707 | 35.473 | 1.00 | 27.64 | B |
| ATOM | 2442 | OD2 | ASP | B | 65 | 48.397 | 43.302 | 37.544 | 1.00 | 28.63 | B |
| ATOM | 2443 | C | ASP | B | 65 | 46.356 | 46.098 | 36.941 | 1.00 | 24.92 | B |
| ATOM | 2444 | O | ASP | B | 65 | 46.096 | 46.465 | 38.086 | 1.00 | 25.04 | B |
| ATOM | 2445 | N | ALA | B | 66 | 45.483 | 45.444 | 36.181 | 1.00 | 26.95 | B |
| ATOM | 2446 | CA | ALA | B | 66 | 44.140 | 45.136 | 36.659 | 1.00 | 28.23 | B |
| ATOM | 2447 | CB | ALA | B | 66 | 43.292 | 44.605 | 35.509 | 1.00 | 28.97 | B |
| ATOM | 2448 | C | ALA | B | 66 | 44.129 | 44.140 | 37.814 | 1.00 | 28.97 | B |
| ATOM | 2449 | O | ALA | B | 66 | 43.167 | 44.083 | 38.580 | 1.00 | 29.82 | B |
| ATOM | 2450 | N | ASN | B | 67 | 45.198 | 43.363 | 37.942 | 1.00 | 28.97 | B |
| ATOM | 2451 | CA | ASN | B | 67 | 45.289 | 42.365 | 39.000 | 1.00 | 29.76 | B |
| ATOM | 2452 | CB | ASN | B | 67 | 46.029 | 41.137 | 38.458 | 1.00 | 30.71 | B |
| ATOM | 2453 | CG | ASN | B | 67 | 45.317 | 40.501 | 37.279 | 1.00 | 32.34 | B |
| ATOM | 2454 | OD1 | ASN | B | 67 | 44.160 | 40.094 | 37.385 | 1.00 | 33.09 | B |
| ATOM | 2455 | ND2 | ASN | B | 67 | 46.007 | 40.411 | 36.146 | 1.00 | 33.17 | B |
| ATOM | 2456 | C | ASN | B | 67 | 45.917 | 42.922 | 40.274 | 1.00 | 28.95 | B |
| ATOM | 2457 | O | ASN | B | 67 | 45.293 | 42.916 | 41.336 | 1.00 | 29.70 | B |
| ATOM | 2458 | N | THR | B | 68 | 47.151 | 43.405 | 40.163 | 1.00 | 27.58 | B |
| ATOM | 2459 | CA | THR | B | 68 | 47.876 | 43.945 | 41.309 | 1.00 | 25.51 | B |
| ATOM | 2460 | CB | THR | B | 68 | 49.394 | 43.894 | 41.073 | 1.00 | 25.12 | B |
| ATOM | 2461 | OG1 | THR | B | 68 | 49.745 | 44.801 | 40.019 | 1.00 | 20.95 | B |
| ATOM | 2462 | CG2 | THR | B | 68 | 49.822 | 42.489 | 40.687 | 1.00 | 24.96 | B |
| ATOM | 2463 | C | THR | B | 68 | 47.505 | 45.384 | 41.647 | 1.00 | 24.97 | B |
| ATOM | 2464 | O | THR | B | 68 | 47.760 | 45.849 | 42.758 | 1.00 | 25.29 | B |
| ATOM | 2465 | N | LYS | B | 69 | 46.909 | 46.083 | 40.687 | 1.00 | 24.16 | B |
| ATOM | 2466 | CA | LYS | B | 69 | 46.514 | 47.475 | 40.868 | 1.00 | 23.37 | B |
| ATOM | 2467 | CB | LYS | B | 69 | 45.496 | 47.579 | 42.010 | 1.00 | 26.52 | B |
| ATOM | 2468 | CG | LYS | B | 69 | 44.139 | 46.975 | 41.679 | 1.00 | 29.62 | B |
| ATOM | 2469 | CD | LYS | B | 69 | 43.086 | 47.390 | 42.694 | 1.00 | 32.54 | B |
| ATOM | 2470 | CE | LYS | B | 69 | 41.702 | 46.906 | 42.287 | 1.00 | 34.99 | B |
| ATOM | 2471 | NZ | LYS | B | 69 | 40.648 | 47.378 | 43.230 | 1.00 | 35.23 | B |
| ATOM | 2472 | C | LYS | B | 69 | 47.732 | 48.372 | 41.062 | 1.00 | 21.74 | B |
| ATOM | 2473 | O | LYS | B | 69 | 47.623 | 49.479 | 41.593 | 1.00 | 20.74 | B |
| ATOM | 2474 | N | ILE | B | 70 | 48.891 | 47.890 | 40.628 | 1.00 | 18.80 | B |
| ATOM | 2475 | CA | ILE | B | 70 | 50.126 | 48.657 | 40.735 | 1.00 | 17.97 | B |
| ATOM | 2476 | CB | ILE | B | 70 | 51.352 | 47.729 | 40.900 | 1.00 | 18.88 | B |
| ATOM | 2477 | CG2 | ILE | B | 70 | 52.640 | 48.538 | 40.806 | 1.00 | 20.23 | B |
| ATOM | 2478 | CG1 | ILE | B | 70 | 51.269 | 46.995 | 42.241 | 1.00 | 20.76 | B |
| ATOM | 2479 | CD1 | ILE | B | 70 | 52.423 | 46.045 | 42.499 | 1.00 | 22.88 | B |
| ATOM | 2480 | C | ILE | B | 70 | 50.309 | 49.492 | 39.471 | 1.00 | 15.47 | B |
| ATOM | 2481 | O | ILE | B | 70 | 50.047 | 49.019 | 38.365 | 1.00 | 15.13 | B |
| ATOM | 2482 | N | TRP | B | 71 | 50.742 | 50.737 | 39.644 | 1.00 | 13.13 | B |
| ATOM | 2483 | CA | TRP | B | 71 | 50.975 | 51.636 | 38.520 | 1.00 | 12.67 | B |
| ATOM | 2484 | CB | TRP | B | 71 | 50.674 | 53.079 | 38.937 | 1.00 | 12.72 | B |
| ATOM | 2485 | CG | TRP | B | 71 | 49.224 | 53.410 | 38.968 | 1.00 | 13.58 | B |
| ATOM | 2486 | CD2 | TRP | B | 71 | 48.438 | 53.888 | 37.873 | 1.00 | 14.46 | B |
| ATOM | 2487 | CE2 | TRP | B | 71 | 47.116 | 54.046 | 38.345 | 1.00 | 15.69 | B |
| ATOM | 2488 | CE3 | TRP | B | 71 | 48.722 | 54.198 | 36.536 | 1.00 | 13.52 | B |
| ATOM | 2489 | CD1 | TRP | B | 71 | 48.373 | 53.297 | 40.033 | 1.00 | 14.51 | B |
| ATOM | 2490 | NE1 | TRP | B | 71 | 47.106 | 53.679 | 39.665 | 1.00 | 16.14 | B |
| ATOM | 2491 | CZ2 | TRP | B | 71 | 46.077 | 54.500 | 37.526 | 1.00 | 14.84 | B |
| ATOM | 2492 | CZ3 | TRP | B | 71 | 47.687 | 54.651 | 35.721 | 1.00 | 15.20 | B |
| ATOM | 2493 | CH2 | TRP | B | 71 | 46.381 | 54.796 | 36.222 | 1.00 | 14.88 | B |
| ATOM | 2494 | C | TRP | B | 71 | 52.402 | 51.491 | 38.026 | 1.00 | 11.67 | B |
| ATOM | 2495 | O | TRP | B | 71 | 53.335 | 51.458 | 38.826 | 1.00 | 13.19 | B |
| ATOM | 2496 | N | ASN | B | 72 | 52.574 | 51.412 | 36.708 | 1.00 | 10.89 | B |
| ATOM | 2497 | CA | ASN | B | 72 | 53.909 | 51.305 | 36.136 | 1.00 | 10.92 | B |
| ATOM | 2498 | CB | ASN | B | 72 | 53.971 | 50.121 | 35.155 | 1.00 | 11.32 | B |
| ATOM | 2499 | CG | ASN | B | 72 | 53.110 | 50.331 | 33.920 | 1.00 | 11.78 | B |
| ATOM | 2500 | OD1 | ASN | B | 72 | 52.720 | 51.456 | 33.596 | 1.00 | 12.64 | B |
| ATOM | 2501 | ND2 | ASN | B | 72 | 52.826 | 49.245 | 33.213 | 1.00 | 15.85 | B |
| ATOM | 2502 | C | ASN | B | 72 | 54.284 | 52.625 | 35.471 | 1.00 | 9.96 | B |
| ATOM | 2503 | O | ASN | B | 72 | 53.560 | 53.616 | 35.562 | 1.00 | 10.53 | B |
| ATOM | 2504 | N | GLY | B | 73 | 55.440 | 52.619 | 34.819 | 1.00 | 10.42 | B |
| ATOM | 2505 | CA | GLY | B | 73 | 55.915 | 53.781 | 34.089 | 1.00 | 9.99 | B |
| ATOM | 2506 | C | GLY | B | 73 | 56.175 | 55.046 | 34.877 | 1.00 | 9.20 | B |
| ATOM | 2507 | O | GLY | B | 73 | 56.366 | 55.017 | 36.093 | 1.00 | 9.61 | B |
| ATOM | 2508 | N | MET | B | 74 | 56.195 | 56.170 | 34.161 | 1.00 | 9.33 | B |
| ATOM | 2509 | CA | MET | B | 74 | 56.438 | 57.468 | 34.775 | 1.00 | 9.66 | B |
| ATOM | 2510 | CB | MET | B | 74 | 56.612 | 58.526 | 33.683 | 1.00 | 9.48 | B |
| ATOM | 2511 | CG | MET | B | 74 | 57.870 | 58.331 | 32.857 | 1.00 | 10.36 | B |
| ATOM | 2512 | SD | MET | B | 74 | 58.035 | 59.534 | 31.542 | 1.00 | 9.40 | B |
| ATOM | 2513 | CE | MET | B | 74 | 56.941 | 58.818 | 30.315 | 1.00 | 10.99 | B |
| ATOM | 2514 | C | MET | B | 74 | 55.345 | 57.817 | 35.767 | 1.00 | 8.74 | B |
| ATOM | 2515 | O | MET | B | 74 | 55.615 | 58.424 | 36.802 | 1.00 | 9.06 | B |
| ATOM | 2516 | N | VAL | B | 75 | 54.107 | 57.448 | 35.454 | 1.00 | 9.57 | B |
| ATOM | 2517 | CA | VAL | B | 75 | 53.012 | 57.722 | 36.372 | 1.00 | 10.23 | B |
| ATOM | 2518 | CB | VAL | B | 75 | 51.650 | 57.289 | 35.774 | 1.00 | 10.32 | B |
| ATOM | 2519 | CG1 | VAL | B | 75 | 50.555 | 57.392 | 36.832 | 1.00 | 11.55 | B |
| ATOM | 2520 | CG2 | VAL | B | 75 | 51.305 | 58.187 | 34.582 | 1.00 | 11.56 | B |
| ATOM | 2521 | C | VAL | B | 75 | 53.298 | 56.973 | 37.675 | 1.00 | 9.02 | B |
| ATOM | 2522 | O | VAL | B | 75 | 53.161 | 57.532 | 38.761 | 1.00 | 9.52 | B |
| ATOM | 2523 | N | GLY | B | 76 | 53.719 | 55.716 | 37.564 | 1.00 | 9.20 | B |
| ATOM | 2524 | CA | GLY | B | 76 | 54.035 | 54.948 | 38.756 | 1.00 | 10.49 | B |
| ATOM | 2525 | C | GLY | B | 76 | 55.137 | 55.590 | 39.585 | 1.00 | 10.06 | B |
| ATOM | 2526 | O | GLY | B | 76 | 55.057 | 55.627 | 40.812 | 1.00 | 10.24 | B |
| ATOM | 2527 | N | GLU | B | 77 | 56.172 | 56.106 | 38.927 | 1.00 | 10.03 | B |
| ATOM | 2528 | CA | GLU | B | 77 | 57.262 | 56.739 | 39.656 | 1.00 | 11.26 | B |
| ATOM | 2529 | CB | GLU | B | 77 | 58.387 | 57.115 | 38.688 | 1.00 | 12.58 | B |
| ATOM | 2530 | CG | GLU | B | 77 | 59.025 | 55.921 | 37.999 | 1.00 | 14.21 | B |
| ATOM | 2531 | CD | GLU | B | 77 | 60.416 | 56.222 | 37.479 | 1.00 | 16.09 | B |
| ATOM | 2532 | OE1 | GLU | B | 77 | 60.720 | 57.408 | 37.234 | 1.00 | 19.00 | B |
| ATOM | 2533 | OE2 | GLU | B | 77 | 61.204 | 55.270 | 37.307 | 1.00 | 16.26 | B |
| ATOM | 2534 | C | GLU | B | 77 | 56.742 | 57.918 | 40.466 | 1.00 | 10.75 | B |
| ATOM | 2535 | O | GLU | B | 77 | 57.215 | 58.169 | 41.569 | 1.00 | 11.53 | B |
| ATOM | 2536 | N | LEU | B | 78 | 55.770 | 58.644 | 39.924 | 1.00 | 9.82 | B |
| ATOM | 2537 | CA | LEU | B | 78 | 55.192 | 59.781 | 40.639 | 1.00 | 9.75 | B |
| ATOM | 2538 | CB | LEU | B | 78 | 54.402 | 60.650 | 39.664 | 1.00 | 10.18 | B |
| ATOM | 2539 | CG | LEU | B | 78 | 55.183 | 61.318 | 38.532 | 1.00 | 11.06 | B |
| ATOM | 2540 | CD1 | LEU | B | 78 | 54.201 | 62.002 | 37.607 | 1.00 | 12.43 | B |
| ATOM | 2541 | CD2 | LEU | B | 78 | 56.190 | 62.320 | 39.088 | 1.00 | 10.65 | B |
| ATOM | 2542 | C | LEU | B | 78 | 54.323 | 59.283 | 41.787 | 1.00 | 10.67 | B |
| ATOM | 2543 | O | LEU | B | 78 | 54.421 | 59.768 | 42.913 | 1.00 | 11.04 | B |
| ATOM | 2544 | N | VAL | B | 79 | 53.465 | 58.319 | 41.485 | 1.00 | 10.25 | B |
| ATOM | 2545 | CA | VAL | B | 79 | 52.557 | 57.754 | 42.470 | 1.00 | 11.27 | B |
| ATOM | 2546 | CB | VAL | B | 79 | 51.688 | 56.649 | 41.821 | 1.00 | 11.30 | B |
| ATOM | 2547 | CG1 | VAL | B | 79 | 50.928 | 55.865 | 42.888 | 1.00 | 12.72 | B |
| ATOM | 2548 | CG2 | VAL | B | 79 | 50.715 | 57.282 | 40.832 | 1.00 | 13.10 | B |
| ATOM | 2549 | C | VAL | B | 79 | 53.288 | 57.184 | 43.683 | 1.00 | 11.56 | B |
| ATOM | 2550 | O | VAL | B | 79 | 52.852 | 57.375 | 44.822 | 1.00 | 11.77 | B |
| ATOM | 2551 | N | TYR | B | 80 | 54.408 | 56.508 | 43.444 | 1.00 | 10.46 | B |
| ATOM | 2552 | CA | TYR | B | 80 | 55.167 | 55.895 | 44.532 | 1.00 | 11.28 | B |
| ATOM | 2553 | CB | TYR | B | 80 | 55.659 | 54.517 | 44.069 | 1.00 | 11.09 | B |
| ATOM | 2554 | CG | TYR | B | 80 | 54.513 | 53.621 | 43.670 | 1.00 | 10.97 | B |
| ATOM | 2555 | CD1 | TYR | B | 80 | 53.496 | 53.321 | 44.575 | 1.00 | 12.84 | B |
| ATOM | 2556 | CE1 | TYR | B | 80 | 52.384 | 52.571 | 44.191 | 1.00 | 12.75 | B |
| ATOM | 2557 | CD2 | TYR | B | 80 | 54.397 | 53.138 | 42.368 | 1.00 | 11.00 | B |
| ATOM | 2558 | CE2 | TYR | B | 80 | 53.291 | 52.388 | 41.973 | 1.00 | 12.20 | B |
| ATOM | 2559 | CZ | TYR | B | 80 | 52.286 | 52.112 | 42.887 | 1.00 | 11.32 | B |
| ATOM | 2560 | OH | TYR | B | 80 | 51.168 | 51.408 | 42.495 | 1.00 | 12.36 | B |
| ATOM | 2561 | C | TYR | B | 80 | 56.269 | 56.779 | 45.104 | 1.00 | 11.47 | B |
| ATOM | 2562 | O | TYR | B | 80 | 57.025 | 56.341 | 45.975 | 1.00 | 12.62 | B |
| ATOM | 2563 | N | GLY | B | 81 | 56.354 | 58.016 | 44.618 | 1.00 | 11.77 | B |
| ATOM | 2564 | CA | GLY | B | 81 | 57.343 | 58.963 | 45.114 | 1.00 | 13.08 | B |
| ATOM | 2565 | C | GLY | B | 81 | 58.785 | 58.763 | 44.684 | 1.00 | 14.28 | B |
| ATOM | 2566 | O | GLY | B | 81 | 59.702 | 59.201 | 45.381 | 1.00 | 16.40 | B |
| ATOM | 2567 | N | LYS | B | 82 | 58.995 | 58.121 | 43.540 | 1.00 | 12.68 | B |
| ATOM | 2568 | CA | LYS | B | 82 | 60.344 | 57.867 | 43.036 | 1.00 | 13.74 | B |
| ATOM | 2569 | CB | LYS | B | 82 | 60.338 | 56.557 | 42.236 | 1.00 | 14.63 | B |
| ATOM | 2570 | CG | LYS | B | 82 | 59.824 | 55.346 | 43.003 | 1.00 | 18.22 | B |
| ATOM | 2571 | CD | LYS | B | 82 | 60.734 | 54.978 | 44.159 | 1.00 | 21.14 | B |
| ATOM | 2572 | CE | LYS | B | 82 | 60.223 | 53.738 | 44.882 | 1.00 | 23.62 | B |
| ATOM | 2573 | NZ | LYS | B | 82 | 61.113 | 53.339 | 46.009 | 1.00 | 25.94 | B |
| ATOM | 2574 | C | LYS | B | 82 | 60.849 | 59.056 | 42.224 | 1.00 | 12.88 | B |
| ATOM | 2575 | O | LYS | B | 82 | 62.050 | 59.190 | 41.982 | 1.00 | 14.84 | B |
| ATOM | 2576 | N | ALA | B | 83 | 59.924 | 59.907 | 41.794 | 1.00 | 12.38 | B |
| ATOM | 2577 | CA | ALA | B | 83 | 60.263 | 61.096 | 41.016 | 1.00 | 11.64 | B |
| ATOM | 2578 | CB | ALA | B | 83 | 60.123 | 60.805 | 39.527 | 1.00 | 12.37 | B |
| ATOM | 2579 | C | ALA | B | 83 | 59.327 | 62.232 | 41.421 | 1.00 | 10.70 | B |
| ATOM | 2580 | O | ALA | B | 83 | 58.227 | 61.985 | 41.920 | 1.00 | 11.07 | B |
| ATOM | 2581 | N | ASP | B | 84 | 59.761 | 63.470 | 41.198 | 1.00 | 10.03 | B |
| ATOM | 2582 | CA | ASP | B | 84 | 58.968 | 64.643 | 41.560 | 1.00 | 9.72 | B |
| ATOM | 2583 | CB | ASP | B | 84 | 59.898 | 65.733 | 42.102 | 1.00 | 12.11 | B |
| ATOM | 2584 | CG | ASP | B | 84 | 60.716 | 65.271 | 43.284 | 1.00 | 14.44 | B |
| ATOM | 2585 | OD1 | ASP | B | 84 | 60.120 | 64.803 | 44.275 | 1.00 | 17.95 | B |
| ATOM | 2586 | OD2 | ASP | B | 84 | 61.958 | 65.384 | 43.221 | 1.00 | 15.95 | B |
| ATOM | 2587 | C | ASP | B | 84 | 58.118 | 65.171 | 40.418 | 1.00 | 10.31 | B |
| ATOM | 2588 | O | ASP | B | 84 | 57.076 | 65.793 | 40.643 | 1.00 | 10.64 | B |
| ATOM | 2589 | N | ILE | B | 85 | 58.563 | 64.936 | 39.191 | 1.00 | 9.52 | B |
| ATOM | 2590 | CA | ILE | B | 85 | 57.849 | 65.443 | 38.030 | 1.00 | 9.74 | B |
| ATOM | 2591 | CB | ILE | B | 85 | 58.257 | 66.925 | 37.781 | 1.00 | 11.00 | B |
| ATOM | 2592 | CG2 | ILE | B | 85 | 59.734 | 66.996 | 37.445 | 1.00 | 13.21 | B |
| ATOM | 2593 | CG1 | ILE | B | 85 | 57.403 | 67.561 | 36.683 | 1.00 | 11.45 | B |
| ATOM | 2594 | CD1 | ILE | B | 85 | 57.490 | 69.082 | 36.677 | 1.00 | 14.00 | B |
| ATOM | 2595 | C | ILE | B | 85 | 58.191 | 64.599 | 36.812 | 1.00 | 9.21 | B |
| ATOM | 2596 | O | ILE | B | 85 | 59.292 | 64.060 | 36.719 | 1.00 | 9.90 | B |
| ATOM | 2597 | N | ALA | B | 86 | 57.234 | 64.468 | 35.900 | 1.00 | 8.46 | B |
| ATOM | 2598 | CA | ALA | B | 86 | 57.446 | 63.718 | 34.664 | 1.00 | 8.54 | B |
| ATOM | 2599 | CB | ALA | B | 86 | 56.422 | 62.582 | 34.529 | 1.00 | 8.59 | B |
| ATOM | 2600 | C | ALA | B | 86 | 57.291 | 64.702 | 33.515 | 1.00 | 8.28 | B |
| ATOM | 2601 | O | ALA | B | 86 | 56.272 | 65.388 | 33.414 | 1.00 | 9.05 | B |
| ATOM | 2602 | N | ILE | B | 87 | 58.318 | 64.795 | 32.676 | 1.00 | 8.75 | B |
| ATOM | 2603 | CA | ILE | B | 87 | 58.300 | 65.686 | 31.520 | 1.00 | 9.37 | B |
| ATOM | 2604 | CB | ILE | B | 87 | 59.341 | 66.823 | 31.675 | 1.00 | 9.87 | B |
| ATOM | 2605 | CG2 | ILE | B | 87 | 59.286 | 67.753 | 30.470 | 1.00 | 11.56 | B |
| ATOM | 2606 | CG1 | ILE | B | 87 | 59.052 | 67.609 | 32.960 | 1.00 | 9.11 | B |
| ATOM | 2607 | CD1 | ILE | B | 87 | 60.022 | 68.736 | 33.238 | 1.00 | 11.51 | B |
| ATOM | 2608 | C | ILE | B | 87 | 58.612 | 64.810 | 30.311 | 1.00 | 9.80 | B |
| ATOM | 2609 | O | ILE | B | 87 | 59.768 | 64.458 | 30.053 | 1.00 | 9.23 | B |
| ATOM | 2610 | N | ALA | B | 88 | 57.563 | 64.462 | 29.572 | 1.00 | 9.10 | B |
| ATOM | 2611 | CA | ALA | B | 88 | 57.681 | 63.569 | 28.427 | 1.00 | 9.13 | B |
| ATOM | 2612 | CB | ALA | B | 88 | 57.847 | 62.140 | 28.934 | 1.00 | 11.65 | B |
| ATOM | 2613 | C | ALA | B | 88 | 56.408 | 63.667 | 27.594 | 1.00 | 8.94 | B |
| ATOM | 2614 | O | ALA | B | 88 | 55.499 | 64.424 | 27.930 | 1.00 | 8.74 | B |
| ATOM | 2615 | N | PRO | B | 89 | 56.336 | 62.920 | 26.480 | 1.00 | 8.93 | B |
| ATOM | 2616 | CD | PRO | B | 89 | 57.448 | 62.251 | 25.779 | 1.00 | 9.53 | B |
| ATOM | 2617 | CA | PRO | B | 89 | 55.127 | 62.960 | 25.649 | 1.00 | 9.58 | B |
| ATOM | 2618 | CB | PRO | B | 89 | 55.642 | 62.481 | 24.287 | 1.00 | 10.07 | B |
| ATOM | 2619 | CG | PRO | B | 89 | 56.712 | 61.472 | 24.635 | 1.00 | 9.31 | B |
| ATOM | 2620 | C | PRO | B | 89 | 54.080 | 62.072 | 26.339 | 1.00 | 8.71 | B |
| ATOM | 2621 | O | PRO | B | 89 | 53.659 | 61.038 | 25.812 | 1.00 | 9.89 | B |
| ATOM | 2622 | N | LEU | B | 90 | 53.682 | 62.494 | 27.536 | 1.00 | 8.48 | B |
| ATOM | 2623 | CA | LEU | B | 90 | 52.721 | 61.763 | 28.357 | 1.00 | 8.00 | B |
| ATOM | 2624 | CB | LEU | B | 90 | 53.138 | 61.902 | 29.822 | 1.00 | 8.24 | B |
| ATOM | 2625 | CG | LEU | B | 90 | 52.294 | 61.143 | 30.850 | 1.00 | 7.30 | B |
| ATOM | 2626 | CD1 | LEU | B | 90 | 52.467 | 59.638 | 30.677 | 1.00 | 8.93 | B |
| ATOM | 2627 | CD2 | LEU | B | 90 | 52.718 | 61.562 | 32.243 | 1.00 | 10.45 | B |
| ATOM | 2628 | C | LEU | B | 90 | 51.311 | 62.259 | 28.092 | 1.00 | 7.45 | B |
| ATOM | 2629 | O | LEU | B | 90 | 50.937 | 63.364 | 28.495 | 1.00 | 8.57 | B |
| ATOM | 2630 | N | THR | B | 91 | 50.519 | 61.427 | 27.433 | 1.00 | 7.15 | B |
| ATOM | 2631 | CA | THR | B | 91 | 49.161 | 61.794 | 27.089 | 1.00 | 8.89 | B |
| ATOM | 2632 | CB | THR | B | 91 | 48.597 | 60.775 | 26.103 | 1.00 | 9.01 | B |
| ATOM | 2633 | OG1 | THR | B | 91 | 49.485 | 60.701 | 24.981 | 1.00 | 9.51 | B |
| ATOM | 2634 | CG2 | THR | B | 91 | 47.210 | 61.182 | 25.620 | 1.00 | 10.88 | B |
| ATOM | 2635 | C | THR | B | 91 | 48.250 | 61.931 | 28.298 | 1.00 | 9.07 | B |
| ATOM | 2636 | O | THR | B | 91 | 48.247 | 61.096 | 29.204 | 1.00 | 8.09 | B |
| ATOM | 2637 | N | ILE | B | 92 | 47.489 | 63.016 | 28.312 | 1.00 | 9.29 | B |
| ATOM | 2638 | CA | ILE | B | 92 | 46.552 | 63.276 | 29.392 | 1.00 | 8.36 | B |
| ATOM | 2639 | CB | ILE | B | 92 | 46.109 | 64.758 | 29.386 | 1.00 | 8.94 | B |
| ATOM | 2640 | CG2 | ILE | B | 92 | 44.990 | 64.984 | 30.399 | 1.00 | 9.48 | B |
| ATOM | 2641 | CG1 | ILE | B | 92 | 47.314 | 65.654 | 29.682 | 1.00 | 9.04 | B |
| ATOM | 2642 | CD1 | ILE | B | 92 | 47.030 | 67.131 | 29.538 | 1.00 | 11.48 | B |
| ATOM | 2643 | C | ILE | B | 92 | 45.344 | 62.371 | 29.186 | 1.00 | 10.25 | B |
| ATOM | 2644 | O | ILE | B | 92 | 44.636 | 62.484 | 28.183 | 1.00 | 10.43 | B |
| ATOM | 2645 | N | THR | B | 93 | 45.136 | 61.447 | 30.118 | 1.00 | 9.09 | B |
| ATOM | 2646 | CA | THR | B | 93 | 44.001 | 60.532 | 30.039 | 1.00 | 10.64 | B |
| ATOM | 2647 | CB | THR | B | 93 | 44.434 | 59.090 | 29.707 | 1.00 | 10.99 | B |
| ATOM | 2648 | OG1 | THR | B | 93 | 45.168 | 58.543 | 30.810 | 1.00 | 12.28 | B |
| ATOM | 2649 | CG2 | THR | B | 93 | 45.305 | 59.065 | 28.457 | 1.00 | 11.50 | B |
| ATOM | 2650 | C | THR | B | 93 | 43.288 | 60.513 | 31.382 | 1.00 | 9.94 | B |
| ATOM | 2651 | O | THR | B | 93 | 43.864 | 60.876 | 32.407 | 1.00 | 10.71 | B |
| ATOM | 2652 | N | LEU | B | 94 | 42.034 | 60.075 | 31.368 | 1.00 | 10.44 | B |
| ATOM | 2653 | CA | LEU | B | 94 | 41.226 | 60.019 | 32.576 | 1.00 | 11.79 | B |
| ATOM | 2654 | CB | LEU | B | 94 | 39.814 | 59.549 | 32.215 | 1.00 | 12.07 | B |
| ATOM | 2655 | CG | LEU | B | 94 | 38.891 | 59.243 | 33.400 | 1.00 | 12.92 | B |
| ATOM | 2656 | CD1 | LEU | B | 94 | 38.623 | 60.506 | 34.198 | 1.00 | 14.90 | B |
| ATOM | 2657 | CD2 | LEU | B | 94 | 37.591 | 58.650 | 32.881 | 1.00 | 16.53 | B |
| ATOM | 2658 | C | LEU | B | 94 | 41.848 | 59.168 | 33.674 | 1.00 | 10.90 | B |
| ATOM | 2659 | O | LEU | B | 94 | 41.979 | 59.617 | 34.809 | 1.00 | 11.19 | B |
| ATOM | 2660 | N | VAL | B | 95 | 42.230 | 57.939 | 33.344 | 1.00 | 11.42 | B |
| ATOM | 2661 | CA | VAL | B | 95 | 42.791 | 57.059 | 34.360 | 1.00 | 12.28 | B |
| ATOM | 2662 | CB | VAL | B | 95 | 43.048 | 55.630 | 33.813 | 1.00 | 12.96 | B |
| ATOM | 2663 | CG1 | VAL | B | 95 | 41.729 | 54.989 | 33.409 | 1.00 | 16.92 | B |
| ATOM | 2664 | CG2 | VAL | B | 95 | 44.007 | 55.673 | 32.640 | 1.00 | 14.82 | B |
| ATOM | 2665 | C | VAL | B | 95 | 44.064 | 57.613 | 34.984 | 1.00 | 11.38 | B |
| ATOM | 2666 | O | VAL | B | 95 | 44.313 | 57.404 | 36.168 | 1.00 | 12.07 | B |
| ATOM | 2667 | N | ARG | B | 96 | 44.868 | 58.326 | 34.199 | 1.00 | 10.76 | B |
| ATOM | 2668 | CA | ARG | B | 96 | 46.097 | 58.910 | 34.730 | 1.00 | 10.29 | B |
| ATOM | 2669 | CB | ARG | B | 96 | 47.046 | 59.248 | 33.572 | 1.00 | 9.54 | B |
| ATOM | 2670 | CG | ARG | B | 96 | 47.619 | 58.041 | 32.856 | 1.00 | 10.00 | B |
| ATOM | 2671 | CD | ARG | B | 96 | 48.438 | 58.469 | 31.655 | 1.00 | 9.38 | B |
| ATOM | 2672 | NE | ARG | B | 96 | 49.028 | 57.323 | 30.974 | 1.00 | 9.28 | B |
| ATOM | 2673 | CZ | ARG | B | 96 | 49.552 | 57.376 | 29.757 | 1.00 | 8.53 | B |
| ATOM | 2674 | NH1 | ARG | B | 96 | 49.556 | 58.519 | 29.087 | 1.00 | 9.89 | B |
| ATOM | 2675 | NH2 | ARG | B | 96 | 50.077 | 56.290 | 29.210 | 1.00 | 9.10 | B |
| ATOM | 2676 | C | ARG | B | 96 | 45.761 | 60.121 | 35.598 | 1.00 | 9.41 | B |
| ATOM | 2677 | O | ARG | B | 96 | 46.335 | 60.308 | 36.668 | 1.00 | 10.66 | B |
| ATOM | 2678 | N | GLU | B | 97 | 44.825 | 60.939 | 35.124 | 1.00 | 9.82 | B |
| ATOM | 2679 | CA | GLU | B | 97 | 44.404 | 62.141 | 35.842 | 1.00 | 10.05 | B |
| ATOM | 2680 | CB | GLU | B | 97 | 43.397 | 62.910 | 34.981 | 1.00 | 10.93 | B |
| ATOM | 2681 | CG | GLU | B | 97 | 42.881 | 64.202 | 35.611 | 1.00 | 12.01 | B |
| ATOM | 2682 | CD | GLU | B | 97 | 43.933 | 65.292 | 35.683 | 1.00 | 11.95 | B |
| ATOM | 2683 | OE1 | GLU | B | 97 | 44.845 | 65.194 | 36.530 | 1.00 | 12.88 | B |
| ATOM | 2684 | OE2 | GLU | B | 97 | 43.846 | 66.250 | 34.885 | 1.00 | 14.79 | B |
| ATOM | 2685 | C | GLU | B | 97 | 43.841 | 61.791 | 37.213 | 1.00 | 11.65 | B |
| ATOM | 2686 | O | GLU | B | 97 | 43.883 | 62.602 | 38.138 | 1.00 | 11.62 | B |
| ATOM | 2687 | N | GLU | B | 98 | 43.305 | 60.583 | 37.335 | 1.00 | 10.80 | B |
| ATOM | 2688 | CA | GLU | B | 98 | 42.726 | 60.137 | 38.594 | 1.00 | 12.25 | B |
| ATOM | 2689 | CB | GLU | B | 98 | 41.868 | 58.892 | 38.334 | 1.00 | 13.12 | B |
| ATOM | 2690 | CG | GLU | B | 98 | 40.541 | 59.198 | 37.643 | 1.00 | 14.07 | B |
| ATOM | 2691 | CD | GLU | B | 98 | 39.787 | 57.953 | 37.208 | 1.00 | 15.79 | B |
| ATOM | 2692 | OE1 | GLU | B | 98 | 38.618 | 58.090 | 36.789 | 1.00 | 18.87 | B |
| ATOM | 2693 | OE2 | GLU | B | 98 | 40.356 | 56.841 | 37.269 | 1.00 | 17.43 | B |
| ATOM | 2694 | C | GLU | B | 98 | 43.775 | 59.913 | 39.676 | 1.00 | 12.93 | B |
| ATOM | 2695 | O | GLU | B | 98 | 43.468 | 60.027 | 40.866 | 1.00 | 13.27 | B |
| ATOM | 2696 | N | VAL | B | 99 | 45.012 | 59.617 | 39.282 | 1.00 | 11.95 | B |
| ATOM | 2697 | CA | VAL | B | 99 | 46.048 | 59.346 | 40.276 | 1.00 | 11.57 | B |
| ATOM | 2698 | CB | VAL | B | 99 | 46.627 | 57.925 | 40.091 | 1.00 | 12.29 | B |
| ATOM | 2699 | CG1 | VAL | B | 99 | 45.506 | 56.901 | 40.222 | 1.00 | 14.41 | B |
| ATOM | 2700 | CG2 | VAL | B | 99 | 47.318 | 57.800 | 38.736 | 1.00 | 11.21 | B |
| ATOM | 2701 | C | VAL | B | 99 | 47.198 | 60.345 | 40.404 | 1.00 | 11.85 | B |
| ATOM | 2702 | O | VAL | B | 99 | 47.912 | 60.334 | 41.402 | 1.00 | 11.93 | B |
| ATOM | 2703 | N | ILE | B | 100 | 47.394 | 61.195 | 39.401 | 1.00 | 10.79 | B |
| ATOM | 2704 | CA | ILE | B | 100 | 48.441 | 62.213 | 39.469 | 1.00 | 9.94 | B |
| ATOM | 2705 | CB | ILE | B | 100 | 49.712 | 61.820 | 38.640 | 1.00 | 9.74 | B |
| ATOM | 2706 | CG2 | ILE | B | 100 | 50.484 | 60.712 | 39.362 | 1.00 | 11.41 | B |
| ATOM | 2707 | CG1 | ILE | B | 100 | 49.336 | 61.392 | 37.217 | 1.00 | 10.34 | B |
| ATOM | 2708 | CD1 | ILE | B | 100 | 48.894 | 62.519 | 36.298 | 1.00 | 12.72 | B |
| ATOM | 2709 | C | ILE | B | 100 | 47.877 | 63.535 | 38.964 | 1.00 | 10.66 | B |
| ATOM | 2710 | O | ILE | B | 100 | 46.751 | 63.575 | 38.466 | 1.00 | 10.93 | B |
| ATOM | 2711 | N | ASP | B | 101 | 48.638 | 64.616 | 39.124 | 1.00 | 10.24 | B |
| ATOM | 2712 | CA | ASP | B | 101 | 48.210 | 65.934 | 38.650 | 1.00 | 11.76 | B |
| ATOM | 2713 | CB | ASP | B | 101 | 48.604 | 67.031 | 39.655 | 1.00 | 12.35 | B |
| ATOM | 2714 | CG | ASP | B | 101 | 47.795 | 66.994 | 40.940 | 1.00 | 11.56 | B |
| ATOM | 2715 | OD1 | ASP | B | 101 | 46.712 | 66.373 | 40.967 | 1.00 | 13.87 | B |
| ATOM | 2716 | OD2 | ASP | B | 101 | 48.255 | 67.614 | 41.925 | 1.00 | 14.17 | B |
| ATOM | 2717 | C | ASP | B | 101 | 48.845 | 66.195 | 37.285 | 1.00 | 10.88 | B |
| ATOM | 2718 | O | ASP | B | 101 | 50.017 | 65.872 | 37.068 | 1.00 | 13.39 | B |
| ATOM | 2719 | N | PHE | B | 102 | 48.070 | 66.775 | 36.371 | 1.00 | 10.57 | B |
| ATOM | 2720 | CA | PHE | B | 102 | 48.546 | 67.128 | 35.034 | 1.00 | 10.95 | B |
| ATOM | 2721 | CB | PHE | B | 102 | 47.659 | 66.498 | 33.956 | 1.00 | 10.59 | B |
| ATOM | 2722 | CG | PHE | B | 102 | 48.068 | 65.123 | 33.520 | 1.00 | 10.43 | B |
| ATOM | 2723 | CD1 | PHE | B | 102 | 49.236 | 64.916 | 32.991 | 1.00 | 10.86 | B |
| ATOM | 2724 | CD2 | PHE | B | 102 | 47.214 | 64.157 | 33.603 | 1.00 | 11.54 | B |
| ATOM | 2725 | CE1 | PHE | B | 102 | 49.609 | 63.664 | 32.488 | 1.00 | 10.96 | B |
| ATOM | 2726 | CE2 | PHE | B | 102 | 47.490 | 62.875 | 33.132 | 1.00 | 12.28 | B |
| ATOM | 2727 | CZ | PHE | B | 102 | 48.712 | 62.626 | 32.562 | 1.00 | 10.89 | B |
| ATOM | 2728 | C | PHE | B | 102 | 48.503 | 68.638 | 34.881 | 1.00 | 11.31 | B |
| ATOM | 2729 | O | PHE | B | 102 | 47.583 | 69.294 | 35.373 | 1.00 | 11.38 | B |
| ATOM | 2730 | N | SER | B | 103 | 49.488 | 69.185 | 34.182 | 1.00 | 10.15 | B |
| ATOM | 2731 | CA | SER | B | 103 | 49.504 | 70.606 | 33.882 | 1.00 | 10.78 | B |
| ATOM | 2732 | CB | SER | B | 103 | 50.916 | 71.025 | 33.479 | 1.00 | 10.95 | B |
| ATOM | 2733 | OG | SER | B | 103 | 51.238 | 70.473 | 32.201 | 1.00 | 11.20 | B |
| ATOM | 2734 | C | SER | B | 103 | 48.559 | 70.713 | 32.694 | 1.00 | 11.47 | B |
| ATOM | 2735 | O | SER | B | 103 | 48.115 | 69.701 | 32.145 | 1.00 | 11.65 | B |
| ATOM | 2736 | N | LYS | B | 104 | 48.244 | 71.941 | 32.298 | 1.00 | 12.25 | B |
| ATOM | 2737 | CA | LYS | B | 104 | 47.439 | 72.141 | 31.111 | 1.00 | 12.94 | B |
| ATOM | 2738 | CB | LYS | B | 104 | 47.125 | 73.633 | 30.960 | 1.00 | 14.44 | B |
| ATOM | 2739 | CG | LYS | B | 104 | 46.200 | 74.190 | 32.033 | 1.00 | 16.62 | B |
| ATOM | 2740 | CD | LYS | B | 104 | 44.876 | 73.439 | 32.048 | 1.00 | 15.36 | B |
| ATOM | 2741 | CE | LYS | B | 104 | 43.868 | 74.061 | 33.019 | 1.00 | 15.44 | B |
| ATOM | 2742 | NZ | LYS | B | 104 | 44.262 | 73.945 | 34.457 | 1.00 | 15.24 | B |
| ATOM | 2743 | C | LYS | B | 104 | 48.357 | 71.583 | 30.015 | 1.00 | 11.94 | B |
| ATOM | 2744 | O | LYS | B | 104 | 49.576 | 71.535 | 30.192 | 1.00 | 11.20 | B |
| ATOM | 2745 | N | PRO | B | 105 | 47.796 | 71.169 | 28.872 | 1.00 | 11.15 | B |
| ATOM | 2746 | CD | PRO | B | 105 | 46.369 | 71.191 | 28.518 | 1.00 | 13.19 | B |
| ATOM | 2747 | CA | PRO | B | 105 | 48.604 | 70.611 | 27.777 | 1.00 | 11.83 | B |
| ATOM | 2748 | CB | PRO | B | 105 | 47.554 | 70.259 | 26.718 | 1.00 | 14.56 | B |
| ATOM | 2749 | CG | PRO | B | 105 | 46.275 | 70.014 | 27.488 | 1.00 | 16.32 | B |
| ATOM | 2750 | C | PRO | B | 105 | 49.698 | 71.534 | 27.261 | 1.00 | 9.71 | B |
| ATOM | 2751 | O | PRO | B | 105 | 49.480 | 72.734 | 27.112 | 1.00 | 10.01 | B |
| ATOM | 2752 | N | PHE | B | 106 | 50.872 | 70.969 | 26.978 | 1.00 | 9.28 | B |
| ATOM | 2753 | CA | PHE | B | 106 | 51.961 | 71.764 | 26.423 | 1.00 | 9.25 | B |
| ATOM | 2754 | CB | PHE | B | 106 | 53.271 | 71.499 | 27.189 | 1.00 | 8.75 | B |
| ATOM | 2755 | CG | PHE | B | 106 | 53.781 | 70.080 | 27.104 | 1.00 | 8.48 | B |
| ATOM | 2756 | CD1 | PHE | B | 106 | 54.389 | 69.661 | 26.034 | 1.00 | 8.53 | B |
| ATOM | 2757 | CD2 | PHE | B | 106 | 53.661 | 69.290 | 28.130 | 1.00 | 8.74 | B |
| ATOM | 2758 | CE1 | PHE | B | 106 | 54.941 | 68.365 | 25.944 | 1.00 | 8.18 | B |
| ATOM | 2759 | CE2 | PHE | B | 106 | 54.176 | 67.980 | 28.138 | 1.00 | 9.15 | B |
| ATOM | 2760 | CZ | PHE | B | 106 | 54.830 | 67.513 | 27.021 | 1.00 | 8.81 | B |
| ATOM | 2761 | C | PHE | B | 106 | 52.060 | 71.513 | 24.920 | 1.00 | 8.84 | B |
| ATOM | 2762 | O | PHE | B | 106 | 52.751 | 72.238 | 24.207 | 1.00 | 10.20 | B |
| ATOM | 2763 | N | MET | B | 107 | 51.349 | 70.499 | 24.437 | 1.00 | 8.41 | B |
| ATOM | 2764 | CA | MET | B | 107 | 51.357 | 70.156 | 23.019 | 1.00 | 9.63 | B |
| ATOM | 2765 | CB | MET | B | 107 | 52.612 | 69.329 | 22.702 | 1.00 | 12.06 | B |
| ATOM | 2766 | CG | MET | B | 107 | 52.735 | 68.894 | 21.240 | 1.00 | 13.02 | B |
| ATOM | 2767 | SD | MET | B | 107 | 54.220 | 67.912 | 20.886 | 1.00 | 14.69 | B |
| ATOM | 2768 | CE | MET | B | 107 | 55.453 | 69.181 | 20.827 | 1.00 | 16.10 | B |
| ATOM | 2769 | C | MET | B | 107 | 50.074 | 69.407 | 22.689 | 1.00 | 9.16 | B |
| ATOM | 2770 | O | MET | B | 107 | 49.612 | 68.580 | 23.477 | 1.00 | 8.38 | B |
| ATOM | 2771 | N | SER | B | 108 | 49.509 | 69.688 | 21.522 | 1.00 | 9.03 | B |
| ATOM | 2772 | CA | SER | B | 108 | 48.289 | 69.020 | 21.085 | 1.00 | 11.10 | B |
| ATOM | 2773 | CB | SER | B | 108 | 47.425 | 70.011 | 20.314 | 1.00 | 13.63 | B |
| ATOM | 2774 | OG | SER | B | 108 | 48.083 | 70.392 | 19.120 | 1.00 | 14.86 | B |
| ATOM | 2775 | C | SER | B | 108 | 48.640 | 67.805 | 20.234 | 1.00 | 10.23 | B |
| ATOM | 2776 | O | SER | B | 108 | 49.702 | 67.744 | 19.614 | 1.00 | 10.94 | B |
| ATOM | 2777 | N | LEU | B | 109 | 47.739 | 66.832 | 20.215 | 1.00 | 11.38 | B |
| ATOM | 2778 | CA | LEU | B | 109 | 47.932 | 65.645 | 19.397 | 1.00 | 10.10 | B |
| ATOM | 2779 | CB | LEU | B | 109 | 49.071 | 64.789 | 19.953 | 1.00 | 11.95 | B |
| ATOM | 2780 | CG | LEU | B | 109 | 48.706 | 63.945 | 21.173 | 1.00 | 15.12 | B |
| ATOM | 2781 | CD1 | LEU | B | 109 | 49.597 | 62.739 | 21.219 | 1.00 | 19.71 | B |
| ATOM | 2782 | CD2 | LEU | B | 109 | 48.824 | 64.759 | 22.437 | 1.00 | 14.94 | B |
| ATOM | 2783 | C | LEU | B | 109 | 46.631 | 64.872 | 19.325 | 1.00 | 9.73 | B |
| ATOM | 2784 | O | LEU | B | 109 | 45.696 | 65.114 | 20.088 | 1.00 | 10.33 | B |
| ATOM | 2785 | N | GLY | B | 110 | 46.593 | 63.932 | 18.393 | 1.00 | 9.90 | B |
| ATOM | 2786 | CA | GLY | B | 110 | 45.441 | 63.075 | 18.220 | 1.00 | 9.38 | B |
| ATOM | 2787 | C | GLY | B | 110 | 45.945 | 61.804 | 17.577 | 1.00 | 9.48 | B |
| ATOM | 2788 | O | GLY | B | 110 | 46.981 | 61.824 | 16.912 | 1.00 | 9.14 | B |
| ATOM | 2789 | N | ILE | B | 111 | 45.246 | 60.694 | 17.787 | 1.00 | 9.65 | B |
| ATOM | 2790 | CA | ILE | B | 111 | 45.655 | 59.443 | 17.166 | 1.00 | 9.35 | B |
| ATOM | 2791 | CB | ILE | B | 111 | 44.767 | 58.279 | 17.643 | 1.00 | 9.94 | B |
| ATOM | 2792 | CG2 | ILE | B | 111 | 45.057 | 57.020 | 16.831 | 1.00 | 10.66 | B |
| ATOM | 2793 | CG1 | ILE | B | 111 | 45.028 | 58.033 | 19.130 | 1.00 | 10.22 | B |
| ATOM | 2794 | CD1 | ILE | B | 111 | 44.019 | 57.120 | 19.792 | 1.00 | 10.21 | B |
| ATOM | 2795 | C | ILE | B | 111 | 45.512 | 59.671 | 15.666 | 1.00 | 9.48 | B |
| ATOM | 2796 | O | ILE | B | 111 | 44.561 | 60.313 | 15.212 | 1.00 | 9.88 | B |
| ATOM | 2797 | N | SER | B | 112 | 46.475 | 59.164 | 14.905 | 1.00 | 9.02 | B |
| ATOM | 2798 | CA | SER | B | 112 | 46.490 | 59.360 | 13.462 | 1.00 | 9.29 | B |
| ATOM | 2799 | CB | SER | B | 112 | 47.377 | 60.566 | 13.145 | 1.00 | 9.53 | B |
| ATOM | 2800 | OG | SER | B | 112 | 48.730 | 60.281 | 13.466 | 1.00 | 10.83 | B |
| ATOM | 2801 | C | SER | B | 112 | 46.965 | 58.115 | 12.745 | 1.00 | 9.10 | B |
| ATOM | 2802 | O | SER | B | 112 | 47.467 | 57.177 | 13.360 | 1.00 | 9.87 | B |
| ATOM | 2803 | N | ILE | B | 113 | 46.831 | 58.143 | 11.425 | 1.00 | 9.61 | B |
| ATOM | 2804 | CA | ILE | B | 113 | 47.237 | 57.036 | 10.573 | 1.00 | 10.86 | B |
| ATOM | 2805 | CB | ILE | B | 113 | 46.128 | 56.682 | 9.556 | 1.00 | 10.47 | B |
| ATOM | 2806 | CG2 | ILE | B | 113 | 46.583 | 55.521 | 8.678 | 1.00 | 12.50 | B |
| ATOM | 2807 | CG1 | ILE | B | 113 | 44.829 | 56.346 | 10.290 | 1.00 | 10.99 | B |
| ATOM | 2808 | CD1 | ILE | B | 113 | 43.621 | 56.260 | 9.385 | 1.00 | 11.97 | B |
| ATOM | 2809 | C | ILE | B | 113 | 48.500 | 57.360 | 9.779 | 1.00 | 9.78 | B |
| ATOM | 2810 | O | ILE | B | 113 | 48.582 | 58.403 | 9.126 | 1.00 | 10.59 | B |
| ATOM | 2811 | N | MET | B | 114 | 49.483 | 56.468 | 9.852 | 1.00 | 10.31 | B |
| ATOM | 2812 | CA | MET | B | 114 | 50.712 | 56.611 | 9.079 | 1.00 | 10.19 | B |
| ATOM | 2813 | CB | MET | B | 114 | 51.952 | 56.472 | 9.968 | 1.00 | 10.87 | B |
| ATOM | 2814 | CG | MET | B | 114 | 53.248 | 56.420 | 9.141 | 1.00 | 10.87 | B |
| ATOM | 2815 | SD | MET | B | 114 | 54.716 | 55.935 | 10.065 | 1.00 | 12.20 | B |
| ATOM | 2816 | CE | MET | B | 114 | 54.960 | 57.401 | 11.082 | 1.00 | 13.18 | B |
| ATOM | 2817 | C | MET | B | 114 | 50.679 | 55.522 | 8.021 | 1.00 | 11.79 | B |
| ATOM | 2818 | O | MET | B | 114 | 50.500 | 54.345 | 8.341 | 1.00 | 10.71 | B |
| ATOM | 2819 | N | ILE | B | 115 | 50.848 | 55.916 | 6.764 | 1.00 | 11.46 | B |
| ATOM | 2820 | CA | ILE | B | 115 | 50.869 | 54.964 | 5.664 | 1.00 | 11.48 | B |
| ATOM | 2821 | CB | ILE | B | 115 | 49.708 | 55.182 | 4.658 | 1.00 | 12.30 | B |
| ATOM | 2822 | CG2 | ILE | B | 115 | 48.376 | 54.889 | 5.317 | 1.00 | 12.90 | B |
| ATOM | 2823 | CG1 | ILE | B | 115 | 49.768 | 56.606 | 4.090 | 1.00 | 13.44 | B |
| ATOM | 2824 | CD1 | ILE | B | 115 | 48.838 | 56.840 | 2.906 | 1.00 | 17.65 | B |
| ATOM | 2825 | C | ILE | B | 115 | 52.150 | 55.147 | 4.873 | 1.00 | 11.42 | B |
| ATOM | 2826 | O | ILE | B | 115 | 52.814 | 56.183 | 4.966 | 1.00 | 12.08 | B |
| ATOM | 2827 | N | LYS | B | 116 | 52.502 | 54.125 | 4.104 | 1.00 | 13.24 | B |
| ATOM | 2828 | CA | LYS | B | 116 | 53.655 | 54.213 | 3.227 | 1.00 | 10.86 | B |
| ATOM | 2829 | CB | LYS | B | 116 | 54.161 | 52.812 | 2.879 | 1.00 | 12.45 | B |
| ATOM | 2830 | CG | LYS | B | 116 | 55.410 | 52.821 | 2.009 | 1.00 | 13.34 | B |
| ATOM | 2831 | CD | LYS | B | 116 | 55.911 | 51.416 | 1.723 | 1.00 | 16.54 | B |
| ATOM | 2832 | CE | LYS | B | 116 | 57.216 | 51.446 | 0.938 | 1.00 | 18.02 | B |
| ATOM | 2833 | NZ | LYS | B | 116 | 57.032 | 51.944 | −0.453 | 1.00 | 19.21 | B |
| ATOM | 2834 | C | LYS | B | 116 | 53.041 | 54.945 | 2.040 | 1.00 | 11.09 | B |
| ATOM | 2835 | O | LYS | B | 116 | 51.917 | 54.638 | 1.644 | 1.00 | 11.68 | B |
| ATOM | 2836 | N | LYS | B | 117 | 53.755 | 55.914 | 1.482 | 1.00 | 11.23 | B |
| ATOM | 2837 | CA | LYS | B | 117 | 53.224 | 56.665 | 0.349 | 1.00 | 11.74 | B |
| ATOM | 2838 | CB | LYS | B | 117 | 54.313 | 57.586 | −0.209 | 1.00 | 12.60 | B |
| ATOM | 2839 | CG | LYS | B | 117 | 54.610 | 58.789 | 0.672 | 1.00 | 13.79 | B |
| ATOM | 2840 | CD | LYS | B | 117 | 55.636 | 59.715 | 0.031 | 1.00 | 13.40 | B |
| ATOM | 2841 | CE | LYS | B | 117 | 55.903 | 60.935 | 0.904 | 1.00 | 15.47 | B |
| ATOM | 2842 | NZ | LYS | B | 117 | 56.912 | 61.846 | 0.294 | 1.00 | 15.56 | B |
| ATOM | 2843 | C | LYS | B | 117 | 52.633 | 55.741 | −0.705 | 1.00 | 11.47 | B |
| ATOM | 2844 | O | LYS | B | 117 | 53.233 | 54.725 | −1.067 | 1.00 | 11.50 | B |
| ATOM | 2845 | N | GLY | B | 118 | 51.447 | 56.095 | −1.187 | 1.00 | 12.24 | B |
| ATOM | 2846 | CA | GLY | B | 118 | 50.801 | 55.294 | −2.212 | 1.00 | 13.17 | B |
| ATOM | 2847 | C | GLY | B | 118 | 49.736 | 54.336 | −1.709 | 1.00 | 14.01 | B |
| ATOM | 2848 | O | GLY | B | 118 | 48.956 | 53.802 | −2.499 | 1.00 | 15.21 | B |
| ATOM | 2849 | N | THR | B | 119 | 49.698 | 54.108 | −0.400 | 1.00 | 13.30 | B |
| ATOM | 2850 | CA | THR | B | 119 | 48.702 | 53.210 | 0.176 | 1.00 | 13.36 | B |
| ATOM | 2851 | CB | THR | B | 119 | 48.958 | 52.999 | 1.676 | 1.00 | 13.62 | B |
| ATOM | 2852 | OG1 | THR | B | 119 | 50.257 | 52.426 | 1.854 | 1.00 | 13.79 | B |
| ATOM | 2853 | CG2 | THR | B | 119 | 47.920 | 52.059 | 2.272 | 1.00 | 14.41 | B |
| ATOM | 2854 | C | THR | B | 119 | 47.308 | 53.801 | −0.022 | 1.00 | 13.25 | B |
| ATOM | 2855 | O | THR | B | 119 | 47.061 | 54.957 | 0.315 | 1.00 | 13.63 | B |
| ATOM | 2856 | N | PRO | B | 120 | 46.376 | 53.008 | −0.578 | 1.00 | 15.18 | B |
| ATOM | 2857 | CD | PRO | B | 120 | 46.588 | 51.676 | −1.171 | 1.00 | 14.18 | B |
| ATOM | 2858 | CA | PRO | B | 120 | 45.008 | 53.472 | −0.820 | 1.00 | 15.67 | B |
| ATOM | 2859 | CB | PRO | B | 120 | 44.527 | 52.508 | −1.904 | 1.00 | 16.05 | B |
| ATOM | 2860 | CG | PRO | B | 120 | 45.117 | 51.200 | −1.471 | 1.00 | 16.26 | B |
| ATOM | 2861 | C | PRO | B | 120 | 44.141 | 53.538 | 0.435 | 1.00 | 15.82 | B |
| ATOM | 2862 | O | PRO | B | 120 | 43.130 | 52.843 | 0.541 | 1.00 | 17.43 | B |
| ATOM | 2863 | N | ILE | B | 121 | 44.544 | 54.391 | 1.372 | 1.00 | 15.76 | B |
| ATOM | 2864 | CA | ILE | B | 121 | 43.828 | 54.587 | 2.632 | 1.00 | 14.92 | B |
| ATOM | 2865 | CB | ILE | B | 121 | 44.529 | 53.848 | 3.796 | 1.00 | 14.97 | B |
| ATOM | 2866 | CG2 | ILE | B | 121 | 43.962 | 54.318 | 5.131 | 1.00 | 12.68 | B |
| ATOM | 2867 | CG1 | ILE | B | 121 | 44.370 | 52.334 | 3.634 | 1.00 | 14.33 | B |
| ATOM | 2868 | CD1 | ILE | B | 121 | 42.945 | 51.832 | 3.803 | 1.00 | 15.40 | B |
| ATOM | 2869 | C | ILE | B | 121 | 43.800 | 56.082 | 2.945 | 1.00 | 15.76 | B |
| ATOM | 2870 | O | ILE | B | 121 | 44.832 | 56.750 | 2.885 | 1.00 | 16.49 | B |
| ATOM | 2871 | N | GLU | B | 122 | 42.625 | 56.604 | 3.284 | 1.00 | 18.35 | B |
| ATOM | 2872 | CA | GLU | B | 122 | 42.489 | 58.025 | 3.581 | 1.00 | 20.17 | B |
| ATOM | 2873 | CB | GLU | B | 122 | 41.780 | 58.710 | 2.406 | 1.00 | 23.24 | B |
| ATOM | 2874 | CG | GLU | B | 122 | 42.475 | 58.553 | 1.066 | 1.00 | 29.97 | B |
| ATOM | 2875 | CD | GLU | B | 122 | 41.732 | 59.255 | −0.054 | 1.00 | 34.16 | B |
| ATOM | 2876 | OE1 | GLU | B | 122 | 40.546 | 58.930 | −0.277 | 1.00 | 37.06 | B |
| ATOM | 2877 | OE2 | GLU | B | 122 | 42.332 | 60.131 | −0.712 | 1.00 | 37.63 | B |
| ATOM | 2878 | C | GLU | B | 122 | 41.787 | 58.303 | 4.905 | 1.00 | 19.62 | B |
| ATOM | 2879 | O | GLU | B | 122 | 41.633 | 59.463 | 5.287 | 1.00 | 20.59 | B |
| ATOM | 2880 | N | SER | B | 123 | 41.362 | 57.257 | 5.609 | 1.00 | 17.88 | B |
| ATOM | 2881 | CA | SER | B | 123 | 40.658 | 57.453 | 6.874 | 1.00 | 16.36 | B |
| ATOM | 2882 | CB | SER | B | 123 | 39.255 | 57.981 | 6.575 | 1.00 | 16.51 | B |
| ATOM | 2883 | OG | SER | B | 123 | 38.469 | 56.965 | 5.970 | 1.00 | 19.10 | B |
| ATOM | 2884 | C | SER | B | 123 | 40.604 | 56.182 | 7.707 | 1.00 | 14.92 | B |
| ATOM | 2885 | O | SER | B | 123 | 40.928 | 55.096 | 7.230 | 1.00 | 13.22 | B |
| ATOM | 2886 | N | ALA | B | 124 | 40.183 | 56.330 | 8.960 | 1.00 | 14.58 | B |
| ATOM | 2887 | CA | ALA | B | 124 | 40.047 | 55.186 | 9.852 | 1.00 | 14.74 | B |
| ATOM | 2888 | CB | ALA | B | 124 | 39.681 | 55.653 | 11.253 | 1.00 | 14.54 | B |
| ATOM | 2889 | C | ALA | B | 124 | 38.948 | 54.287 | 9.289 | 1.00 | 14.66 | B |
| ATOM | 2890 | O | ALA | B | 124 | 39.046 | 53.063 | 9.342 | 1.00 | 15.39 | B |
| ATOM | 2891 | N | ALA | B | 125 | 37.900 | 54.903 | 8.749 | 1.00 | 14.87 | B |
| ATOM | 2892 | CA | ALA | B | 125 | 36.803 | 54.146 | 8.160 | 1.00 | 14.63 | B |
| ATOM | 2893 | CB | ALA | B | 125 | 35.747 | 55.092 | 7.603 | 1.00 | 16.21 | B |
| ATOM | 2894 | C | ALA | B | 125 | 37.353 | 53.249 | 7.053 | 1.00 | 14.66 | B |
| ATOM | 2895 | O | ALA | B | 125 | 36.984 | 52.077 | 6.951 | 1.00 | 15.81 | B |
| ATOM | 2896 | N | ASP | B | 126 | 38.239 | 53.798 | 6.224 | 1.00 | 15.21 | B |
| ATOM | 2897 | CA | ASP | B | 126 | 38.841 | 53.022 | 5.142 | 1.00 | 14.84 | B |
| ATOM | 2898 | CB | ASP | B | 126 | 39.836 | 53.890 | 4.359 | 1.00 | 16.41 | B |
| ATOM | 2899 | CG | ASP | B | 126 | 39.157 | 54.897 | 3.449 | 1.00 | 19.30 | B |
| ATOM | 2900 | OD1 | ASP | B | 126 | 37.951 | 54.740 | 3.157 | 1.00 | 19.38 | B |
| ATOM | 2901 | OD2 | ASP | B | 126 | 39.845 | 55.841 | 3.012 | 1.00 | 20.31 | B |
| ATOM | 2902 | C | ASP | B | 126 | 39.502 | 51.767 | 5.707 | 1.00 | 14.18 | B |
| ATOM | 2903 | O | ASP | B | 126 | 39.317 | 50.667 | 5.188 | 1.00 | 16.38 | B |
| ATOM | 2904 | N | LEU | B | 127 | 40.283 | 51.939 | 6.768 | 1.00 | 14.10 | B |
| ATOM | 2905 | CA | LEU | B | 127 | 40.971 | 50.816 | 7.392 | 1.00 | 14.07 | B |
| ATOM | 2906 | CB | LEU | B | 127 | 41.920 | 51.336 | 8.478 | 1.00 | 13.27 | B |
| ATOM | 2907 | CG | LEU | B | 127 | 43.217 | 52.037 | 8.074 | 1.00 | 13.45 | B |
| ATOM | 2908 | CD1 | LEU | B | 127 | 43.886 | 52.575 | 9.329 | 1.00 | 13.91 | B |
| ATOM | 2909 | CD2 | LEU | B | 127 | 44.141 | 51.071 | 7.345 | 1.00 | 12.03 | B |
| ATOM | 2910 | C | LEU | B | 127 | 40.001 | 49.782 | 7.944 | 1.00 | 14.19 | B |
| ATOM | 2911 | O | LEU | B | 127 | 40.156 | 48.588 | 7.704 | 1.00 | 14.70 | B |
| ATOM | 2912 | N | SER | B | 128 | 39.005 | 50.250 | 8.689 | 1.00 | 15.92 | B |
| ATOM | 2913 | CA | SER | B | 128 | 38.028 | 49.369 | 9.320 | 1.00 | 16.44 | B |
| ATOM | 2914 | CB | SER | B | 128 | 37.075 | 50.218 | 10.169 | 1.00 | 16.30 | B |
| ATOM | 2915 | OG | SER | B | 128 | 36.163 | 50.936 | 9.353 | 1.00 | 17.54 | B |
| ATOM | 2916 | C | SER | B | 128 | 37.275 | 48.459 | 8.359 | 1.00 | 17.64 | B |
| ATOM | 2917 | O | SER | B | 128 | 36.909 | 47.340 | 8.723 | 1.00 | 17.84 | B |
| ATOM | 2918 | N | LYS | B | 129 | 37.045 | 48.933 | 7.140 | 1.00 | 18.64 | B |
| ATOM | 2919 | CA | LYS | B | 129 | 36.291 | 48.172 | 6.147 | 1.00 | 21.45 | B |
| ATOM | 2920 | CB | LYS | B | 129 | 35.591 | 49.161 | 5.210 | 1.00 | 23.01 | B |
| ATOM | 2921 | CG | LYS | B | 129 | 34.696 | 50.170 | 5.905 | 1.00 | 26.51 | B |
| ATOM | 2922 | CD | LYS | B | 129 | 34.148 | 51.180 | 4.909 | 1.00 | 30.10 | B |
| ATOM | 2923 | CE | LYS | B | 129 | 33.342 | 52.266 | 5.602 | 1.00 | 32.04 | B |
| ATOM | 2924 | NZ | LYS | B | 129 | 32.858 | 53.291 | 4.635 | 1.00 | 32.54 | B |
| ATOM | 2925 | C | LYS | B | 129 | 37.096 | 47.135 | 5.374 | 1.00 | 22.41 | B |
| ATOM | 2926 | O | LYS | B | 129 | 36.567 | 46.508 | 4.456 | 1.00 | 24.11 | B |
| ATOM | 2927 | N | GLN | B | 130 | 38.361 | 46.938 | 5.731 | 1.00 | 21.03 | B |
| ATOM | 2928 | CA | GLN | B | 130 | 39.176 | 45.976 | 5.000 | 1.00 | 18.74 | B |
| ATOM | 2929 | CB | GLN | B | 130 | 39.796 | 46.699 | 3.801 | 1.00 | 18.95 | B |
| ATOM | 2930 | CG | GLN | B | 130 | 40.709 | 47.855 | 4.180 | 1.00 | 17.18 | B |
| ATOM | 2931 | CD | GLN | B | 130 | 42.095 | 47.392 | 4.572 | 1.00 | 16.68 | B |
| ATOM | 2932 | OE1 | GLN | B | 130 | 42.830 | 46.846 | 3.753 | 1.00 | 15.68 | B |
| ATOM | 2933 | NE2 | GLN | B | 130 | 42.459 | 47.605 | 5.831 | 1.00 | 14.42 | B |
| ATOM | 2934 | C | GLN | B | 130 | 40.203 | 45.264 | 5.873 | 1.00 | 17.60 | B |
| ATOM | 2935 | O | GLN | B | 130 | 40.355 | 45.572 | 7.057 | 1.00 | 18.74 | B |
| ATOM | 2936 | N | THR | B | 131 | 40.908 | 44.309 | 5.269 | 1.00 | 18.69 | B |
| ATOM | 2937 | CA | THR | B | 131 | 41.926 | 43.533 | 5.968 | 1.00 | 18.81 | B |
| ATOM | 2938 | CB | THR | B | 131 | 41.385 | 42.152 | 6.393 | 1.00 | 19.46 | B |
| ATOM | 2939 | OG1 | THR | B | 131 | 40.921 | 41.444 | 5.237 | 1.00 | 19.99 | B |
| ATOM | 2940 | CG2 | THR | B | 131 | 40.239 | 42.310 | 7.384 | 1.00 | 18.92 | B |
| ATOM | 2941 | C | THR | B | 131 | 43.178 | 43.317 | 5.118 | 1.00 | 19.13 | B |
| ATOM | 2942 | O | THR | B | 131 | 44.192 | 42.835 | 5.619 | 1.00 | 19.35 | B |
| ATOM | 2943 | N | GLU | B | 132 | 43.101 | 43.667 | 3.836 | 1.00 | 19.48 | B |
| ATOM | 2944 | CA | GLU | B | 132 | 44.236 | 43.516 | 2.923 | 1.00 | 19.54 | B |
| ATOM | 2945 | CB | GLU | B | 132 | 43.817 | 43.982 | 1.524 | 1.00 | 21.53 | B |
| ATOM | 2946 | CG | GLU | B | 132 | 44.962 | 44.077 | 0.523 | 1.00 | 29.35 | B |
| ATOM | 2947 | CD | GLU | B | 132 | 45.722 | 42.774 | 0.370 | 1.00 | 33.86 | B |
| ATOM | 2948 | OE1 | GLU | B | 132 | 45.088 | 41.748 | 0.047 | 1.00 | 36.49 | B |
| ATOM | 2949 | OE2 | GLU | B | 132 | 46.956 | 42.776 | 0.570 | 1.00 | 37.14 | B |
| ATOM | 2950 | C | GLU | B | 132 | 45.430 | 44.285 | 3.476 | 1.00 | 17.61 | B |
| ATOM | 2951 | O | GLU | B | 132 | 46.560 | 43.791 | 3.494 | 1.00 | 17.21 | B |
| ATOM | 2952 | N | ILE | B | 133 | 45.166 | 45.508 | 3.913 | 1.00 | 15.23 | B |
| ATOM | 2953 | CA | ILE | B | 133 | 46.191 | 46.355 | 4.493 | 1.00 | 14.15 | B |
| ATOM | 2954 | CB | ILE | B | 133 | 45.970 | 47.829 | 4.103 | 1.00 | 14.40 | B |
| ATOM | 2955 | CG2 | ILE | B | 133 | 47.019 | 48.710 | 4.762 | 1.00 | 14.60 | B |
| ATOM | 2956 | CG1 | ILE | B | 133 | 46.020 | 47.970 | 2.580 | 1.00 | 16.04 | B |
| ATOM | 2957 | CD1 | ILE | B | 133 | 45.624 | 49.339 | 2.071 | 1.00 | 16.35 | B |
| ATOM | 2958 | C | ILE | B | 133 | 46.052 | 46.193 | 6.002 | 1.00 | 12.86 | B |
| ATOM | 2959 | O | ILE | B | 133 | 45.075 | 46.649 | 6.596 | 1.00 | 14.16 | B |
| ATOM | 2960 | N | ALA | B | 134 | 47.014 | 45.515 | 6.614 | 1.00 | 11.59 | B |
| ATOM | 2961 | CA | ALA | B | 134 | 46.981 | 45.294 | 8.055 | 1.00 | 12.05 | B |
| ATOM | 2962 | CB | ALA | B | 134 | 48.019 | 44.256 | 8.447 | 1.00 | 11.73 | B |
| ATOM | 2963 | C | ALA | B | 134 | 47.262 | 46.608 | 8.762 | 1.00 | 11.57 | B |
| ATOM | 2964 | O | ALA | B | 134 | 47.892 | 47.500 | 8.195 | 1.00 | 11.94 | B |
| ATOM | 2965 | N | TYR | B | 135 | 46.799 | 46.734 | 10.000 | 1.00 | 11.30 | B |
| ATOM | 2966 | CA | TYR | B | 135 | 47.032 | 47.959 | 10.750 | 1.00 | 10.32 | B |
| ATOM | 2967 | CB | TYR | B | 135 | 45.987 | 49.012 | 10.353 | 1.00 | 10.24 | B |
| ATOM | 2968 | CG | TYR | B | 135 | 44.547 | 48.604 | 10.580 | 1.00 | 10.46 | B |
| ATOM | 2969 | CD1 | TYR | B | 135 | 43.901 | 48.892 | 11.782 | 1.00 | 12.70 | B |
| ATOM | 2970 | CE1 | TYR | B | 135 | 42.569 | 48.538 | 11.985 | 1.00 | 11.96 | B |
| ATOM | 2971 | CD2 | TYR | B | 135 | 43.825 | 47.947 | 9.585 | 1.00 | 11.79 | B |
| ATOM | 2972 | CE2 | TYR | B | 135 | 42.493 | 47.588 | 9.779 | 1.00 | 11.82 | B |
| ATOM | 2973 | CZ | TYR | B | 135 | 41.873 | 47.889 | 10.979 | 1.00 | 11.81 | B |
| ATOM | 2974 | OH | TYR | B | 135 | 40.556 | 47.545 | 11.174 | 1.00 | 11.82 | B |
| ATOM | 2975 | C | TYR | B | 135 | 47.071 | 47.660 | 12.236 | 1.00 | 10.77 | B |
| ATOM | 2976 | O | TYR | B | 135 | 46.371 | 46.769 | 12.711 | 1.00 | 10.80 | B |
| ATOM | 2977 | N | GLY | B | 136 | 47.894 | 48.407 | 12.966 | 1.00 | 10.64 | B |
| ATOM | 2978 | CA | GLY | B | 136 | 48.011 | 48.187 | 14.393 | 1.00 | 10.44 | B |
| ATOM | 2979 | C | GLY | B | 136 | 48.539 | 49.378 | 15.170 | 1.00 | 11.38 | B |
| ATOM | 2980 | O | GLY | B | 136 | 48.744 | 50.469 | 14.623 | 1.00 | 10.73 | B |
| ATOM | 2981 | N | THR | B | 137 | 48.771 | 49.150 | 16.457 | 1.00 | 11.87 | B |
| ATOM | 2982 | CA | THR | B | 137 | 49.256 | 50.173 | 17.376 | 1.00 | 12.05 | B |
| ATOM | 2983 | CB | THR | B | 137 | 48.162 | 50.568 | 18.389 | 1.00 | 14.38 | B |
| ATOM | 2984 | OG1 | THR | B | 137 | 47.931 | 49.465 | 19.279 | 1.00 | 14.95 | B |
| ATOM | 2985 | CG2 | THR | B | 137 | 46.855 | 50.908 | 17.685 | 1.00 | 14.15 | B |
| ATOM | 2986 | C | THR | B | 137 | 50.414 | 49.623 | 18.201 | 1.00 | 12.31 | B |
| ATOM | 2987 | O | THR | B | 137 | 50.849 | 48.483 | 18.015 | 1.00 | 11.64 | B |
| ATOM | 2988 | N | LEU | B | 138 | 50.911 | 50.446 | 19.118 | 1.00 | 12.97 | B |
| ATOM | 2989 | CA | LEU | B | 138 | 51.967 | 50.018 | 20.019 | 1.00 | 13.12 | B |
| ATOM | 2990 | CB | LEU | B | 138 | 52.521 | 51.230 | 20.768 | 1.00 | 13.27 | B |
| ATOM | 2991 | CG | LEU | B | 138 | 53.491 | 52.127 | 20.004 | 1.00 | 14.13 | B |
| ATOM | 2992 | CD1 | LEU | B | 138 | 53.699 | 53.428 | 20.771 | 1.00 | 14.57 | B |
| ATOM | 2993 | CD2 | LEU | B | 138 | 54.806 | 51.390 | 19.798 | 1.00 | 15.37 | B |
| ATOM | 2994 | C | LEU | B | 138 | 51.309 | 48.994 | 20.938 | 1.00 | 15.45 | B |
| ATOM | 2995 | O | LEU | B | 138 | 50.087 | 49.008 | 21.116 | 1.00 | 15.27 | B |
| ATOM | 2996 | N | ASP | B | 139 | 52.115 | 48.113 | 21.523 | 1.00 | 16.69 | B |
| ATOM | 2997 | CA | ASP | B | 139 | 51.596 | 47.074 | 22.410 | 1.00 | 20.52 | B |
| ATOM | 2998 | CB | ASP | B | 139 | 52.621 | 45.943 | 22.503 | 1.00 | 24.45 | B |
| ATOM | 2999 | CG | ASP | B | 139 | 53.920 | 46.390 | 23.137 | 1.00 | 30.93 | B |
| ATOM | 3000 | OD1 | ASP | B | 139 | 54.543 | 47.337 | 22.611 | 1.00 | 35.31 | B |
| ATOM | 3001 | OD2 | ASP | B | 139 | 54.318 | 45.794 | 24.160 | 1.00 | 36.08 | B |
| ATOM | 3002 | C | ASP | B | 139 | 51.200 | 47.629 | 23.773 | 1.00 | 20.31 | B |
| ATOM | 3003 | O | ASP | B | 139 | 50.639 | 46.912 | 24.604 | 1.00 | 20.48 | B |
| ATOM | 3004 | N | SER | B | 140 | 51.505 | 48.900 | 24.008 | 1.00 | 18.89 | B |
| ATOM | 3005 | CA | SER | B | 140 | 51.163 | 49.553 | 25.266 | 1.00 | 18.19 | B |
| ATOM | 3006 | CB | SER | B | 140 | 52.215 | 49.208 | 26.325 | 1.00 | 20.09 | B |
| ATOM | 3007 | OG | SER | B | 140 | 53.475 | 49.758 | 25.990 | 1.00 | 24.92 | B |
| ATOM | 3008 | C | SER | B | 140 | 51.048 | 51.046 | 24.996 | 1.00 | 17.29 | B |
| ATOM | 3009 | O | SER | B | 140 | 51.628 | 51.551 | 24.038 | 1.00 | 18.70 | B |
| ATOM | 3010 | N | GLY | B | 141 | 50.299 | 51.752 | 25.835 | 1.00 | 15.40 | B |
| ATOM | 3011 | CA | GLY | B | 141 | 50.142 | 53.180 | 25.638 | 1.00 | 13.91 | B |
| ATOM | 3012 | C | GLY | B | 141 | 48.694 | 53.598 | 25.478 | 1.00 | 13.36 | B |
| ATOM | 3013 | O | GLY | B | 141 | 47.800 | 52.758 | 25.365 | 1.00 | 13.55 | B |
| ATOM | 3014 | N | SER | B | 142 | 48.468 | 54.905 | 25.437 | 1.00 | 10.87 | B |
| ATOM | 3015 | CA | SER | B | 142 | 47.123 | 55.452 | 25.328 | 1.00 | 10.24 | B |
| ATOM | 3016 | CB | SER | B | 142 | 47.177 | 56.953 | 25.615 | 1.00 | 12.05 | B |
| ATOM | 3017 | OG | SER | B | 142 | 47.796 | 57.203 | 26.862 | 1.00 | 18.50 | B |
| ATOM | 3018 | C | SER | B | 142 | 46.408 | 55.141 | 24.021 | 1.00 | 10.17 | B |
| ATOM | 3019 | O | SER | B | 142 | 45.182 | 55.122 | 23.980 | 1.00 | 10.97 | B |
| ATOM | 3020 | N | THR | B | 143 | 47.152 | 54.911 | 22.946 | 1.00 | 9.32 | B |
| ATOM | 3021 | CA | THR | B | 143 | 46.499 | 54.606 | 21.684 | 1.00 | 9.97 | B |
| ATOM | 3022 | CB | THR | B | 143 | 47.488 | 54.685 | 20.518 | 1.00 | 9.07 | B |
| ATOM | 3023 | OG1 | THR | B | 143 | 47.987 | 56.024 | 20.423 | 1.00 | 10.01 | B |
| ATOM | 3024 | CG2 | THR | B | 143 | 46.807 | 54.318 | 19.213 | 1.00 | 10.12 | B |
| ATOM | 3025 | C | THR | B | 143 | 45.858 | 53.222 | 21.743 | 1.00 | 10.20 | B |
| ATOM | 3026 | O | THR | B | 143 | 44.717 | 53.046 | 21.324 | 1.00 | 11.59 | B |
| ATOM | 3027 | N | LYS | B | 144 | 46.589 | 52.244 | 22.268 | 1.00 | 10.97 | B |
| ATOM | 3028 | CA | LYS | B | 144 | 46.046 | 50.899 | 22.382 | 1.00 | 12.59 | B |
| ATOM | 3029 | CB | LYS | B | 144 | 47.135 | 49.953 | 22.892 | 1.00 | 14.35 | B |
| ATOM | 3030 | CG | LYS | B | 144 | 46.688 | 48.500 | 22.991 | 1.00 | 17.17 | B |
| ATOM | 3031 | CD | LYS | B | 144 | 47.786 | 47.635 | 23.588 | 1.00 | 21.73 | B |
| ATOM | 3032 | CE | LYS | B | 144 | 47.327 | 46.200 | 23.777 | 1.00 | 23.63 | B |
| ATOM | 3033 | NZ | LYS | B | 144 | 48.380 | 45.367 | 24.418 | 1.00 | 25.69 | B |
| ATOM | 3034 | C | LYS | B | 144 | 44.824 | 50.955 | 23.293 | 1.00 | 12.47 | B |
| ATOM | 3035 | O | LYS | B | 144 | 43.800 | 50.339 | 23.005 | 1.00 | 13.35 | B |
| ATOM | 3036 | N | GLU | B | 145 | 44.934 | 51.703 | 24.388 | 1.00 | 13.84 | B |
| ATOM | 3037 | CA | GLU | B | 145 | 43.829 | 51.833 | 25.334 | 1.00 | 13.52 | B |
| ATOM | 3038 | CB | GLU | B | 145 | 44.314 | 52.574 | 26.584 | 1.00 | 17.01 | B |
| ATOM | 3039 | CG | GLU | B | 145 | 45.335 | 51.804 | 27.403 | 1.00 | 22.59 | B |
| ATOM | 3040 | CD | GLU | B | 145 | 44.878 | 50.393 | 27.716 | 1.00 | 26.96 | B |
| ATOM | 3041 | OE1 | GLU | B | 145 | 43.740 | 50.229 | 28.203 | 1.00 | 28.77 | B |
| ATOM | 3042 | OE2 | GLU | B | 145 | 45.661 | 49.447 | 27.480 | 1.00 | 30.33 | B |
| ATOM | 3043 | C | GLU | B | 145 | 42.624 | 52.498 | 24.683 | 1.00 | 12.56 | B |
| ATOM | 3044 | O | GLU | B | 145 | 41.481 | 52.136 | 24.969 | 1.00 | 12.58 | B |
| ATOM | 3045 | N | PHE | B | 146 | 42.877 | 53.474 | 23.815 | 1.00 | 12.47 | B |
| ATOM | 3046 | CA | PHE | B | 146 | 41.799 | 54.171 | 23.124 | 1.00 | 12.35 | B |
| ATOM | 3047 | CB | PHE | B | 146 | 42.381 | 55.212 | 22.160 | 1.00 | 11.86 | B |
| ATOM | 3048 | CG | PHE | B | 146 | 41.356 | 55.817 | 21.234 | 1.00 | 12.49 | B |
| ATOM | 3049 | CD1 | PHE | B | 146 | 40.678 | 56.851 | 21.611 | 1.00 | 13.03 | B |
| ATOM | 3050 | CD2 | PHE | B | 146 | 41.148 | 55.295 | 20.067 | 1.00 | 13.25 | B |
| ATOM | 3051 | CE1 | PHE | B | 146 | 39.709 | 57.446 | 20.797 | 1.00 | 14.59 | B |
| ATOM | 3052 | CE2 | PHE | B | 146 | 40.198 | 55.816 | 19.175 | 1.00 | 14.19 | B |
| ATOM | 3053 | CZ | PHE | B | 146 | 39.466 | 56.917 | 19.554 | 1.00 | 14.18 | B |
| ATOM | 3054 | C | PHE | B | 146 | 40.944 | 53.148 | 22.399 | 1.00 | 11.89 | B |
| ATOM | 3055 | O | PHE | B | 146 | 39.721 | 53.133 | 22.533 | 1.00 | 13.17 | B |
| ATOM | 3056 | N | PHE | B | 147 | 41.602 | 52.292 | 21.626 | 1.00 | 10.83 | B |
| ATOM | 3057 | CA | PHE | B | 147 | 40.898 | 51.277 | 20.861 | 1.00 | 12.64 | B |
| ATOM | 3058 | CB | PHE | B | 147 | 41.865 | 50.681 | 19.831 | 1.00 | 10.75 | B |
| ATOM | 3059 | CG | PHE | B | 147 | 42.100 | 51.573 | 18.643 | 1.00 | 10.57 | B |
| ATOM | 3060 | CD1 | PHE | B | 147 | 41.169 | 51.718 | 17.754 | 1.00 | 10.12 | B |
| ATOM | 3061 | CD2 | PHE | B | 147 | 43.223 | 52.209 | 18.518 | 1.00 | 9.16 | B |
| ATOM | 3062 | CE1 | PHE | B | 147 | 41.320 | 52.549 | 16.641 | 1.00 | 11.01 | B |
| ATOM | 3063 | CE2 | PHE | B | 147 | 43.473 | 53.069 | 17.438 | 1.00 | 10.93 | B |
| ATOM | 3064 | CZ | PHE | B | 147 | 42.500 | 53.239 | 16.483 | 1.00 | 10.78 | B |
| ATOM | 3065 | C | PHE | B | 147 | 40.233 | 50.239 | 21.752 | 1.00 | 13.03 | B |
| ATOM | 3066 | O | PHE | B | 147 | 39.075 | 49.893 | 21.538 | 1.00 | 13.65 | B |
| ATOM | 3067 | N | ARG | B | 148 | 40.952 | 49.761 | 22.760 | 1.00 | 13.08 | B |
| ATOM | 3068 | CA | ARG | B | 148 | 40.400 | 48.755 | 23.658 | 1.00 | 14.29 | B |
| ATOM | 3069 | CB | ARG | B | 148 | 41.491 | 48.312 | 24.641 | 1.00 | 14.69 | B |
| ATOM | 3070 | CG | ARG | B | 148 | 41.017 | 47.284 | 25.665 | 1.00 | 18.17 | B |
| ATOM | 3071 | CD | ARG | B | 148 | 42.129 | 46.894 | 26.629 | 1.00 | 22.36 | B |
| ATOM | 3072 | NE | ARG | B | 148 | 43.229 | 46.208 | 25.957 | 1.00 | 25.91 | B |
| ATOM | 3073 | CZ | ARG | B | 148 | 44.326 | 45.772 | 26.570 | 1.00 | 27.70 | B |
| ATOM | 3074 | NH1 | ARG | B | 148 | 44.475 | 45.949 | 27.876 | 1.00 | 27.74 | B |
| ATOM | 3075 | NH2 | ARG | B | 148 | 45.275 | 45.154 | 25.878 | 1.00 | 26.51 | B |
| ATOM | 3076 | C | ARG | B | 148 | 39.126 | 49.217 | 24.366 | 1.00 | 15.76 | B |
| ATOM | 3077 | O | ARG | B | 148 | 38.210 | 48.419 | 24.566 | 1.00 | 16.77 | B |
| ATOM | 3078 | N | ARG | B | 149 | 39.052 | 50.494 | 24.736 | 1.00 | 15.90 | B |
| ATOM | 3079 | CA | ARG | B | 149 | 37.880 | 50.992 | 25.458 | 1.00 | 17.70 | B |
| ATOM | 3080 | CB | ARG | B | 149 | 38.350 | 51.867 | 26.625 | 1.00 | 20.41 | B |
| ATOM | 3081 | CG | ARG | B | 149 | 39.513 | 51.316 | 27.425 | 1.00 | 25.96 | B |
| ATOM | 3082 | CD | ARG | B | 149 | 39.611 | 52.011 | 28.777 | 1.00 | 30.05 | B |
| ATOM | 3083 | NE | ARG | B | 149 | 39.203 | 53.414 | 28.720 | 1.00 | 33.23 | B |
| ATOM | 3084 | CZ | ARG | B | 149 | 39.840 | 54.363 | 28.039 | 1.00 | 34.57 | B |
| ATOM | 3085 | NH1 | ARG | B | 149 | 40.933 | 54.072 | 27.344 | 1.00 | 35.30 | B |
| ATOM | 3086 | NH2 | ARG | B | 149 | 39.379 | 55.608 | 28.052 | 1.00 | 34.31 | B |
| ATOM | 3087 | C | ARG | B | 149 | 36.837 | 51.717 | 24.614 | 1.00 | 16.79 | B |
| ATOM | 3088 | O | ARG | B | 149 | 35.791 | 52.110 | 25.135 | 1.00 | 17.75 | B |
| ATOM | 3089 | N | SER | B | 150 | 37.093 | 51.898 | 23.325 | 1.00 | 16.03 | B |
| ATOM | 3090 | CA | SER | B | 150 | 36.143 | 52.625 | 22.491 | 1.00 | 16.26 | B |
| ATOM | 3091 | CB | SER | B | 150 | 36.792 | 52.953 | 21.145 | 1.00 | 16.56 | B |
| ATOM | 3092 | OG | SER | B | 150 | 35.916 | 53.744 | 20.358 | 1.00 | 16.88 | B |
| ATOM | 3093 | C | SER | B | 150 | 34.797 | 51.932 | 22.318 | 1.00 | 17.00 | B |
| ATOM | 3094 | O | SER | B | 150 | 34.717 | 50.705 | 22.226 | 1.00 | 17.43 | B |
| ATOM | 3095 | N | ALA | B | 151 | 33.744 | 52.746 | 22.270 | 1.00 | 16.82 | B |
| ATOM | 3096 | CA | ALA | B | 151 | 32.379 | 52.272 | 22.073 | 1.00 | 17.70 | B |
| ATOM | 3097 | CB | ALA | B | 151 | 31.475 | 52.764 | 23.209 | 1.00 | 18.36 | B |
| ATOM | 3098 | C | ALA | B | 151 | 31.890 | 52.816 | 20.730 | 1.00 | 18.27 | B |
| ATOM | 3099 | O | ALA | B | 151 | 30.736 | 52.618 | 20.345 | 1.00 | 20.02 | B |
| ATOM | 3100 | N | ILE | B | 152 | 32.780 | 53.518 | 20.029 | 1.00 | 17.82 | B |
| ATOM | 3101 | CA | ILE | B | 152 | 32.472 | 54.083 | 18.716 | 1.00 | 17.05 | B |
| ATOM | 3102 | CB | ILE | B | 152 | 33.450 | 55.229 | 18.363 | 1.00 | 17.75 | B |
| ATOM | 3103 | CG2 | ILE | B | 152 | 33.186 | 55.730 | 16.949 | 1.00 | 17.27 | B |
| ATOM | 3104 | CG1 | ILE | B | 152 | 33.301 | 56.367 | 19.379 | 1.00 | 19.71 | B |
| ATOM | 3105 | CD1 | ILE | B | 152 | 34.204 | 57.559 | 19.120 | 1.00 | 23.70 | B |
| ATOM | 3106 | C | ILE | B | 152 | 32.599 | 52.960 | 17.683 | 1.00 | 16.26 | B |
| ATOM | 3107 | O | ILE | B | 152 | 33.642 | 52.317 | 17.582 | 1.00 | 16.10 | B |
| ATOM | 3108 | N | ALA | B | 153 | 31.537 | 52.732 | 16.918 | 1.00 | 15.92 | B |
| ATOM | 3109 | CA | ALA | B | 153 | 31.502 | 51.659 | 15.924 | 1.00 | 15.10 | B |
| ATOM | 3110 | CB | ALA | B | 153 | 30.302 | 51.848 | 15.004 | 1.00 | 15.55 | B |
| ATOM | 3111 | C | ALA | B | 153 | 32.763 | 51.445 | 15.088 | 1.00 | 13.13 | B |
| ATOM | 3112 | O | ALA | B | 153 | 33.301 | 50.334 | 15.037 | 1.00 | 12.87 | B |
| ATOM | 3113 | N | VAL | B | 154 | 33.237 | 52.491 | 14.422 | 1.00 | 13.30 | B |
| ATOM | 3114 | CA | VAL | B | 154 | 34.427 | 52.346 | 13.593 | 1.00 | 13.40 | B |
| ATOM | 3115 | CB | VAL | B | 154 | 34.762 | 53.660 | 12.843 | 1.00 | 14.79 | B |
| ATOM | 3116 | CG1 | VAL | B | 154 | 36.114 | 53.540 | 12.150 | 1.00 | 15.68 | B |
| ATOM | 3117 | CG2 | VAL | B | 154 | 33.682 | 53.955 | 11.818 | 1.00 | 16.59 | B |
| ATOM | 3118 | C | VAL | B | 154 | 35.646 | 51.914 | 14.402 | 1.00 | 11.56 | B |
| ATOM | 3119 | O | VAL | B | 154 | 36.395 | 51.027 | 13.984 | 1.00 | 12.09 | B |
| ATOM | 3120 | N | PHE | B | 155 | 35.831 | 52.519 | 15.572 | 1.00 | 11.82 | B |
| ATOM | 3121 | CA | PHE | B | 155 | 36.986 | 52.203 | 16.403 | 1.00 | 12.53 | B |
| ATOM | 3122 | CB | PHE | B | 155 | 37.251 | 53.361 | 17.377 | 1.00 | 12.92 | B |
| ATOM | 3123 | CG | PHE | B | 155 | 37.352 | 54.714 | 16.714 | 1.00 | 13.23 | B |
| ATOM | 3124 | CD1 | PHE | B | 155 | 37.914 | 54.839 | 15.552 | 1.00 | 13.45 | B |
| ATOM | 3125 | CD2 | PHE | B | 155 | 36.884 | 55.756 | 17.320 | 1.00 | 13.49 | B |
| ATOM | 3126 | CE1 | PHE | B | 155 | 38.038 | 56.083 | 14.914 | 1.00 | 13.54 | B |
| ATOM | 3127 | CE2 | PHE | B | 155 | 36.959 | 57.038 | 16.770 | 1.00 | 14.37 | B |
| ATOM | 3128 | CZ | PHE | B | 155 | 37.547 | 57.201 | 15.543 | 1.00 | 13.87 | B |
| ATOM | 3129 | C | PHE | B | 155 | 36.894 | 50.850 | 17.102 | 1.00 | 13.04 | B |
| ATOM | 3130 | O | PHE | B | 155 | 37.905 | 50.151 | 17.219 | 1.00 | 12.48 | B |
| ATOM | 3131 | N | ASP | B | 156 | 35.712 | 50.467 | 17.586 | 1.00 | 13.73 | B |
| ATOM | 3132 | CA | ASP | B | 156 | 35.623 | 49.170 | 18.249 | 1.00 | 14.18 | B |
| ATOM | 3133 | CB | ASP | B | 156 | 34.350 | 49.070 | 19.118 | 1.00 | 17.34 | B |
| ATOM | 3134 | CG | ASP | B | 156 | 33.076 | 49.316 | 18.349 | 1.00 | 20.27 | B |
| ATOM | 3135 | OD1 | ASP | B | 156 | 32.889 | 48.711 | 17.276 | 1.00 | 21.62 | B |
| ATOM | 3136 | OD2 | ASP | B | 156 | 32.243 | 50.109 | 18.841 | 1.00 | 22.52 | B |
| ATOM | 3137 | C | ASP | B | 156 | 35.788 | 48.054 | 17.221 | 1.00 | 13.30 | B |
| ATOM | 3138 | O | ASP | B | 156 | 36.299 | 46.983 | 17.549 | 1.00 | 13.86 | B |
| ATOM | 3139 | N | LYS | B | 157 | 35.382 | 48.299 | 15.978 | 1.00 | 12.32 | B |
| ATOM | 3140 | CA | LYS | B | 157 | 35.564 | 47.294 | 14.938 | 1.00 | 13.15 | B |
| ATOM | 3141 | CB | LYS | B | 157 | 34.775 | 47.679 | 13.684 | 1.00 | 14.71 | B |
| ATOM | 3142 | CG | LYS | B | 157 | 34.974 | 46.697 | 12.541 | 1.00 | 16.66 | B |
| ATOM | 3143 | CD | LYS | B | 157 | 33.986 | 46.924 | 11.409 | 1.00 | 20.89 | B |
| ATOM | 3144 | CE | LYS | B | 157 | 34.159 | 45.872 | 10.321 | 1.00 | 22.42 | B |
| ATOM | 3145 | NZ | LYS | B | 157 | 33.183 | 46.041 | 9.211 | 1.00 | 26.26 | B |
| ATOM | 3146 | C | LYS | B | 157 | 37.067 | 47.166 | 14.681 | 1.00 | 11.75 | B |
| ATOM | 3147 | O | LYS | B | 157 | 37.583 | 46.070 | 14.477 | 1.00 | 12.82 | B |
| ATOM | 3148 | N | MET | B | 158 | 37.769 | 48.298 | 14.692 | 1.00 | 11.60 | B |
| ATOM | 3149 | CA | MET | B | 158 | 39.209 | 48.291 | 14.475 | 1.00 | 11.34 | B |
| ATOM | 3150 | CB | MET | B | 158 | 39.707 | 49.734 | 14.344 | 1.00 | 10.92 | B |
| ATOM | 3151 | CG | MET | B | 158 | 39.355 | 50.389 | 13.013 | 1.00 | 10.61 | B |
| ATOM | 3152 | SD | MET | B | 158 | 39.778 | 52.145 | 12.967 | 1.00 | 11.68 | B |
| ATOM | 3153 | CE | MET | B | 158 | 41.562 | 52.071 | 12.823 | 1.00 | 11.70 | B |
| ATOM | 3154 | C | MET | B | 158 | 39.907 | 47.506 | 15.579 | 1.00 | 11.19 | B |
| ATOM | 3155 | O | MET | B | 158 | 40.836 | 46.749 | 15.309 | 1.00 | 10.94 | B |
| ATOM | 3156 | N | TRP | B | 159 | 39.455 | 47.675 | 16.817 | 1.00 | 11.18 | B |
| ATOM | 3157 | CA | TRP | B | 159 | 40.056 | 46.966 | 17.943 | 1.00 | 11.28 | B |
| ATOM | 3158 | CB | TRP | B | 159 | 39.478 | 47.492 | 19.259 | 1.00 | 10.97 | B |
| ATOM | 3159 | CG | TRP | B | 159 | 39.968 | 46.698 | 20.436 | 1.00 | 12.16 | B |
| ATOM | 3160 | CD2 | TRP | B | 159 | 41.328 | 46.562 | 20.866 | 1.00 | 13.67 | B |
| ATOM | 3161 | CE2 | TRP | B | 159 | 41.332 | 45.679 | 21.968 | 1.00 | 14.30 | B |
| ATOM | 3162 | CE3 | TRP | B | 159 | 42.545 | 47.101 | 20.425 | 1.00 | 14.40 | B |
| ATOM | 3163 | CD1 | TRP | B | 159 | 39.221 | 45.915 | 21.274 | 1.00 | 13.44 | B |
| ATOM | 3164 | NE1 | TRP | B | 159 | 40.035 | 45.300 | 22.197 | 1.00 | 14.53 | B |
| ATOM | 3165 | CZ2 | TRP | B | 159 | 42.508 | 45.320 | 22.637 | 1.00 | 16.70 | B |
| ATOM | 3166 | CZ3 | TRP | B | 159 | 43.716 | 46.744 | 21.092 | 1.00 | 15.71 | B |
| ATOM | 3167 | CH2 | TRP | B | 159 | 43.687 | 45.861 | 22.185 | 1.00 | 16.50 | B |
| ATOM | 3168 | C | TRP | B | 159 | 39.865 | 45.465 | 17.806 | 1.00 | 11.68 | B |
| ATOM | 3169 | O | TRP | B | 159 | 40.800 | 44.688 | 17.998 | 1.00 | 11.26 | B |
| ATOM | 3170 | N | THR | B | 160 | 38.642 | 45.062 | 17.487 | 1.00 | 12.22 | B |
| ATOM | 3171 | CA | THR | B | 160 | 38.339 | 43.646 | 17.334 | 1.00 | 11.26 | B |
| ATOM | 3172 | CB | THR | B | 160 | 36.858 | 43.441 | 16.993 | 1.00 | 13.83 | B |
| ATOM | 3173 | OG1 | THR | B | 160 | 36.063 | 43.862 | 18.110 | 1.00 | 16.23 | B |
| ATOM | 3174 | CG2 | THR | B | 160 | 36.571 | 41.978 | 16.688 | 1.00 | 15.16 | B |
| ATOM | 3175 | C | THR | B | 160 | 39.223 | 43.048 | 16.246 | 1.00 | 10.15 | B |
| ATOM | 3176 | O | THR | B | 160 | 39.730 | 41.937 | 16.394 | 1.00 | 10.71 | B |
| ATOM | 3177 | N | TYR | B | 161 | 39.408 | 43.790 | 15.157 | 1.00 | 11.18 | B |
| ATOM | 3178 | CA | TYR | B | 161 | 40.261 | 43.332 | 14.067 | 1.00 | 11.24 | B |
| ATOM | 3179 | CB | TYR | B | 161 | 40.176 | 44.301 | 12.880 | 1.00 | 10.21 | B |
| ATOM | 3180 | CG | TYR | B | 161 | 41.426 | 44.244 | 12.029 | 1.00 | 11.28 | B |
| ATOM | 3181 | CD1 | TYR | B | 161 | 41.574 | 43.286 | 11.027 | 1.00 | 11.96 | B |
| ATOM | 3182 | CE1 | TYR | B | 161 | 42.774 | 43.152 | 10.330 | 1.00 | 12.51 | B |
| ATOM | 3183 | CD2 | TYR | B | 161 | 42.511 | 45.076 | 12.306 | 1.00 | 10.43 | B |
| ATOM | 3184 | CE2 | TYR | B | 161 | 43.710 | 44.950 | 11.619 | 1.00 | 12.75 | B |
| ATOM | 3185 | CZ | TYR | B | 161 | 43.839 | 43.984 | 10.635 | 1.00 | 12.24 | B |
| ATOM | 3186 | OH | TYR | B | 161 | 45.046 | 43.836 | 9.993 | 1.00 | 15.00 | B |
| ATOM | 3187 | C | TYR | B | 161 | 41.696 | 43.195 | 14.567 | 1.00 | 11.23 | B |
| ATOM | 3188 | O | TYR | B | 161 | 42.354 | 42.180 | 14.334 | 1.00 | 11.92 | B |
| ATOM | 3189 | N | MET | B | 162 | 42.182 | 44.229 | 15.249 | 1.00 | 11.29 | B |
| ATOM | 3190 | CA | MET | B | 162 | 43.552 | 44.231 | 15.754 | 1.00 | 11.30 | B |
| ATOM | 3191 | CB | MET | B | 162 | 43.849 | 45.563 | 16.442 | 1.00 | 13.62 | B |
| ATOM | 3192 | CG | MET | B | 162 | 44.043 | 46.742 | 15.531 | 1.00 | 12.58 | B |
| ATOM | 3193 | SD | MET | B | 162 | 44.548 | 48.154 | 16.533 | 1.00 | 18.19 | B |
| ATOM | 3194 | CE | MET | B | 162 | 43.054 | 49.101 | 16.546 | 1.00 | 20.27 | B |
| ATOM | 3195 | C | MET | B | 162 | 43.851 | 43.072 | 16.679 | 1.00 | 12.13 | B |
| ATOM | 3196 | O | MET | B | 162 | 44.889 | 42.423 | 16.560 | 1.00 | 12.09 | B |
| ATOM | 3197 | N | ARG | B | 163 | 42.943 | 42.820 | 17.614 | 1.00 | 11.82 | B |
| ATOM | 3198 | CA | ARG | B | 163 | 43.153 | 41.759 | 18.582 | 1.00 | 13.21 | B |
| ATOM | 3199 | CB | ARG | B | 163 | 42.128 | 41.911 | 19.710 | 1.00 | 16.98 | B |
| ATOM | 3200 | CG | ARG | B | 163 | 42.556 | 41.238 | 20.999 | 1.00 | 20.62 | B |
| ATOM | 3201 | CD | ARG | B | 163 | 41.505 | 41.347 | 22.087 | 1.00 | 24.44 | B |
| ATOM | 3202 | NE | ARG | B | 163 | 42.003 | 40.782 | 23.337 | 1.00 | 27.50 | B |
| ATOM | 3203 | CZ | ARG | B | 163 | 41.245 | 40.491 | 24.387 | 1.00 | 28.64 | B |
| ATOM | 3204 | NH1 | ARG | B | 163 | 39.938 | 40.711 | 24.348 | 1.00 | 28.24 | B |
| ATOM | 3205 | NH2 | ARG | B | 163 | 41.797 | 39.977 | 25.478 | 1.00 | 29.63 | B |
| ATOM | 3206 | C | ARG | B | 163 | 43.142 | 40.376 | 17.947 | 1.00 | 12.18 | B |
| ATOM | 3207 | O | ARG | B | 163 | 43.786 | 39.459 | 18.454 | 1.00 | 12.88 | B |
| ATOM | 3208 | N | SER | B | 164 | 42.435 | 40.226 | 16.831 | 1.00 | 10.89 | B |
| ATOM | 3209 | CA | SER | B | 164 | 42.333 | 38.925 | 16.173 | 1.00 | 11.71 | B |
| ATOM | 3210 | CB | SER | B | 164 | 40.853 | 38.625 | 15.918 | 1.00 | 12.69 | B |
| ATOM | 3211 | OG | SER | B | 164 | 40.312 | 39.503 | 14.945 | 1.00 | 14.22 | B |
| ATOM | 3212 | C | SER | B | 164 | 43.176 | 38.762 | 14.907 | 1.00 | 11.81 | B |
| ATOM | 3213 | O | SER | B | 164 | 43.220 | 37.674 | 14.330 | 1.00 | 12.97 | B |
| ATOM | 3214 | N | ALA | B | 165 | 43.849 | 39.824 | 14.478 | 1.00 | 12.37 | B |
| ATOM | 3215 | CA | ALA | B | 165 | 44.662 | 39.768 | 13.267 | 1.00 | 11.61 | B |
| ATOM | 3216 | CB | ALA | B | 165 | 45.026 | 41.184 | 12.826 | 1.00 | 12.88 | B |
| ATOM | 3217 | C | ALA | B | 165 | 45.931 | 38.923 | 13.391 | 1.00 | 12.82 | B |
| ATOM | 3218 | O | ALA | B | 165 | 46.588 | 38.921 | 14.428 | 1.00 | 12.59 | B |
| ATOM | 3219 | N | GLU | B | 166 | 46.267 | 38.213 | 12.314 | 1.00 | 13.40 | B |
| ATOM | 3220 | CA | GLU | B | 166 | 47.460 | 37.370 | 12.265 | 1.00 | 14.47 | B |
| ATOM | 3221 | CB | GLU | B | 166 | 47.061 | 35.898 | 12.407 | 1.00 | 13.75 | B |
| ATOM | 3222 | CG | GLU | B | 166 | 46.338 | 35.546 | 13.692 | 1.00 | 13.66 | B |
| ATOM | 3223 | CD | GLU | B | 166 | 46.229 | 34.045 | 13.885 | 1.00 | 12.43 | B |
| ATOM | 3224 | OE1 | GLU | B | 166 | 45.542 | 33.383 | 13.078 | 1.00 | 14.72 | B |
| ATOM | 3225 | OE2 | GLU | B | 166 | 46.844 | 33.530 | 14.838 | 1.00 | 15.12 | B |
| ATOM | 3226 | C | GLU | B | 166 | 48.198 | 37.628 | 10.955 | 1.00 | 15.00 | B |
| ATOM | 3227 | O | GLU | B | 166 | 47.583 | 37.638 | 9.887 | 1.00 | 17.00 | B |
| ATOM | 3228 | N | PRO | B | 167 | 49.523 | 37.850 | 11.017 | 1.00 | 14.83 | B |
| ATOM | 3229 | CD | PRO | B | 167 | 50.360 | 37.853 | 9.807 | 1.00 | 16.26 | B |
| ATOM | 3230 | CA | PRO | B | 167 | 50.364 | 37.879 | 12.220 | 1.00 | 13.45 | B |
| ATOM | 3231 | CB | PRO | B | 167 | 51.789 | 37.889 | 11.643 | 1.00 | 14.40 | B |
| ATOM | 3232 | CG | PRO | B | 167 | 51.666 | 37.159 | 10.327 | 1.00 | 18.88 | B |
| ATOM | 3233 | C | PRO | B | 167 | 50.008 | 39.094 | 13.071 | 1.00 | 13.13 | B |
| ATOM | 3234 | O | PRO | B | 167 | 49.294 | 39.990 | 12.614 | 1.00 | 13.35 | B |
| ATOM | 3235 | N | SER | B | 168 | 50.511 | 39.129 | 14.301 | 1.00 | 13.08 | B |
| ATOM | 3236 | CA | SER | B | 168 | 50.230 | 40.240 | 15.203 | 1.00 | 12.23 | B |
| ATOM | 3237 | CB | SER | B | 168 | 51.048 | 40.081 | 16.481 | 1.00 | 14.03 | B |
| ATOM | 3238 | OG | SER | B | 168 | 50.885 | 41.230 | 17.293 | 1.00 | 14.91 | B |
| ATOM | 3239 | C | SER | B | 168 | 50.487 | 41.578 | 14.528 | 1.00 | 11.20 | B |
| ATOM | 3240 | O | SER | B | 168 | 51.514 | 41.770 | 13.873 | 1.00 | 12.75 | B |
| ATOM | 3241 | N | VAL | B | 169 | 49.554 | 42.506 | 14.708 | 1.00 | 10.70 | B |
| ATOM | 3242 | CA | VAL | B | 169 | 49.676 | 43.839 | 14.128 | 1.00 | 11.80 | B |
| ATOM | 3243 | CB | VAL | B | 169 | 48.309 | 44.362 | 13.619 | 1.00 | 12.79 | B |
| ATOM | 3244 | CG1 | VAL | B | 169 | 47.828 | 43.496 | 12.459 | 1.00 | 13.33 | B |
| ATOM | 3245 | CG2 | VAL | B | 169 | 47.289 | 44.371 | 14.755 | 1.00 | 12.38 | B |
| ATOM | 3246 | C | VAL | B | 169 | 50.244 | 44.841 | 15.126 | 1.00 | 11.83 | B |
| ATOM | 3247 | O | VAL | B | 169 | 50.406 | 46.020 | 14.809 | 1.00 | 12.00 | B |
| ATOM | 3248 | N | PHE | B | 170 | 50.558 | 44.370 | 16.328 | 1.00 | 10.85 | B |
| ATOM | 3249 | CA | PHE | B | 170 | 51.108 | 45.237 | 17.363 | 1.00 | 12.02 | B |
| ATOM | 3250 | CB | PHE | B | 170 | 50.653 | 44.728 | 18.737 | 1.00 | 11.90 | B |
| ATOM | 3251 | CG | PHE | B | 170 | 49.163 | 44.770 | 18.935 | 1.00 | 12.25 | B |
| ATOM | 3252 | CD1 | PHE | B | 170 | 48.561 | 45.905 | 19.094 | 1.00 | 12.64 | B |
| ATOM | 3253 | CD2 | PHE | B | 170 | 48.480 | 43.669 | 18.920 | 1.00 | 12.85 | B |
| ATOM | 3254 | CE1 | PHE | B | 170 | 47.169 | 45.996 | 19.251 | 1.00 | 12.78 | B |
| ATOM | 3255 | CE2 | PHE | B | 170 | 47.088 | 43.652 | 19.068 | 1.00 | 14.94 | B |
| ATOM | 3256 | CZ | PHE | B | 170 | 46.423 | 44.843 | 19.236 | 1.00 | 14.35 | B |
| ATOM | 3257 | C | PHE | B | 170 | 52.623 | 45.335 | 17.238 | 1.00 | 12.05 | B |
| ATOM | 3258 | O | PHE | B | 170 | 53.282 | 44.396 | 16.787 | 1.00 | 14.89 | B |
| ATOM | 3259 | N | VAL | B | 171 | 53.168 | 46.482 | 17.633 | 1.00 | 12.21 | B |
| ATOM | 3260 | CA | VAL | B | 171 | 54.610 | 46.715 | 17.597 | 1.00 | 13.01 | B |
| ATOM | 3261 | CB | VAL | B | 171 | 54.996 | 47.764 | 16.522 | 1.00 | 13.51 | B |
| ATOM | 3262 | CG1 | VAL | B | 171 | 54.606 | 47.252 | 15.143 | 1.00 | 14.10 | B |
| ATOM | 3263 | CG2 | VAL | B | 171 | 54.317 | 49.093 | 16.808 | 1.00 | 14.18 | B |
| ATOM | 3264 | C | VAL | B | 171 | 55.065 | 47.178 | 18.979 | 1.00 | 13.79 | B |
| ATOM | 3265 | O | VAL | B | 171 | 54.265 | 47.699 | 19.754 | 1.00 | 13.72 | B |
| ATOM | 3266 | N | ALA | B | 172 | 56.345 | 46.986 | 19.283 | 1.00 | 15.08 | B |
| ATOM | 3267 | CA | ALA | B | 172 | 56.881 | 47.343 | 20.592 | 1.00 | 14.94 | B |
| ATOM | 3268 | CB | ALA | B | 172 | 58.007 | 46.384 | 20.960 | 1.00 | 16.72 | B |
| ATOM | 3269 | C | ALA | B | 172 | 57.366 | 48.776 | 20.744 | 1.00 | 15.27 | B |
| ATOM | 3270 | O | ALA | B | 172 | 57.327 | 49.332 | 21.842 | 1.00 | 14.95 | B |
| ATOM | 3271 | N | THR | B | 173 | 57.826 | 49.373 | 19.651 | 1.00 | 14.44 | B |
| ATOM | 3272 | CA | THR | B | 173 | 58.341 | 50.736 | 19.698 | 1.00 | 14.47 | B |
| ATOM | 3273 | CB | THR | B | 173 | 59.871 | 50.743 | 19.713 | 1.00 | 14.68 | B |
| ATOM | 3274 | OG1 | THR | B | 173 | 60.345 | 50.242 | 18.457 | 1.00 | 15.72 | B |
| ATOM | 3275 | CG2 | THR | B | 173 | 60.404 | 49.868 | 20.835 | 1.00 | 14.59 | B |
| ATOM | 3276 | C | THR | B | 173 | 57.909 | 51.529 | 18.476 | 1.00 | 13.61 | B |
| ATOM | 3277 | O | THR | B | 173 | 57.500 | 50.960 | 17.463 | 1.00 | 13.63 | B |
| ATOM | 3278 | N | THR | B | 174 | 58.023 | 52.849 | 18.570 | 1.00 | 12.92 | B |
| ATOM | 3279 | CA | THR | B | 174 | 57.667 | 53.716 | 17.461 | 1.00 | 13.32 | B |
| ATOM | 3280 | CB | THR | B | 174 | 57.910 | 55.192 | 17.821 | 1.00 | 12.58 | B |
| ATOM | 3281 | OG1 | THR | B | 174 | 57.062 | 55.556 | 18.916 | 1.00 | 13.66 | B |
| ATOM | 3282 | CG2 | THR | B | 174 | 57.601 | 56.095 | 16.632 | 1.00 | 12.16 | B |
| ATOM | 3283 | C | THR | B | 174 | 58.507 | 53.342 | 16.244 | 1.00 | 13.99 | B |
| ATOM | 3284 | O | THR | B | 174 | 57.984 | 53.199 | 15.140 | 1.00 | 12.99 | B |
| ATOM | 3285 | N | ALA | B | 175 | 59.808 | 53.165 | 16.456 | 1.00 | 13.92 | B |
| ATOM | 3286 | CA | ALA | B | 175 | 60.710 | 52.803 | 15.368 | 1.00 | 15.41 | B |
| ATOM | 3287 | CB | ALA | B | 175 | 62.135 | 52.681 | 15.894 | 1.00 | 15.77 | B |
| ATOM | 3288 | C | ALA | B | 175 | 60.284 | 51.499 | 14.696 | 1.00 | 14.93 | B |
| ATOM | 3289 | O | ALA | B | 175 | 60.398 | 51.354 | 13.481 | 1.00 | 15.03 | B |
| ATOM | 3290 | N | GLU | B | 176 | 59.789 | 50.552 | 15.488 | 1.00 | 13.48 | B |
| ATOM | 3291 | CA | GLU | B | 176 | 59.345 | 49.267 | 14.953 | 1.00 | 14.93 | B |
| ATOM | 3292 | CB | GLU | B | 176 | 59.061 | 48.314 | 16.117 | 1.00 | 16.39 | B |
| ATOM | 3293 | CG | GLU | B | 176 | 58.774 | 46.884 | 15.701 | 1.00 | 21.24 | B |
| ATOM | 3294 | CD | GLU | B | 176 | 58.344 | 46.020 | 16.870 | 1.00 | 23.16 | B |
| ATOM | 3295 | OE1 | GLU | B | 176 | 58.988 | 46.099 | 17.938 | 1.00 | 26.24 | B |
| ATOM | 3296 | OE2 | GLU | B | 176 | 57.368 | 45.256 | 16.721 | 1.00 | 25.11 | B |
| ATOM | 3297 | C | GLU | B | 176 | 58.131 | 49.476 | 14.051 | 1.00 | 13.37 | B |
| ATOM | 3298 | O | GLU | B | 176 | 58.021 | 48.867 | 12.984 | 1.00 | 13.90 | B |
| ATOM | 3299 | N | GLY | B | 177 | 57.210 | 50.327 | 14.494 | 1.00 | 11.77 | B |
| ATOM | 3300 | CA | GLY | B | 177 | 56.023 | 50.604 | 13.707 | 1.00 | 11.87 | B |
| ATOM | 3301 | C | GLY | B | 177 | 56.386 | 51.275 | 12.397 | 1.00 | 11.77 | B |
| ATOM | 3302 | O | GLY | B | 177 | 55.859 | 50.920 | 11.342 | 1.00 | 11.59 | B |
| ATOM | 3303 | N | VAL | B | 178 | 57.298 | 52.240 | 12.467 | 1.00 | 12.02 | B |
| ATOM | 3304 | CA | VAL | B | 178 | 57.734 | 52.967 | 11.280 | 1.00 | 11.85 | B |
| ATOM | 3305 | CB | VAL | B | 178 | 58.676 | 54.134 | 11.661 | 1.00 | 13.33 | B |
| ATOM | 3306 | CG1 | VAL | B | 178 | 59.278 | 54.754 | 10.408 | 1.00 | 12.69 | B |
| ATOM | 3307 | CG2 | VAL | B | 178 | 57.908 | 55.184 | 12.449 | 1.00 | 14.50 | B |
| ATOM | 3308 | C | VAL | B | 178 | 58.448 | 52.028 | 10.308 | 1.00 | 11.37 | B |
| ATOM | 3309 | O | VAL | B | 178 | 58.199 | 52.068 | 9.104 | 1.00 | 11.74 | B |
| ATOM | 3310 | N | ALA | B | 179 | 59.329 | 51.179 | 10.831 | 1.00 | 11.93 | B |
| ATOM | 3311 | CA | ALA | B | 179 | 60.050 | 50.231 | 9.987 | 1.00 | 12.51 | B |
| ATOM | 3312 | CB | ALA | B | 179 | 61.048 | 49.435 | 10.824 | 1.00 | 13.53 | B |
| ATOM | 3313 | C | ALA | B | 179 | 59.070 | 49.283 | 9.304 | 1.00 | 13.12 | B |
| ATOM | 3314 | O | ALA | B | 179 | 59.230 | 48.952 | 8.129 | 1.00 | 13.52 | B |
| ATOM | 3315 | N | ARG | B | 180 | 58.047 | 48.850 | 10.038 | 1.00 | 12.34 | B |
| ATOM | 3316 | CA | ARG | B | 180 | 57.060 | 47.936 | 9.477 | 1.00 | 11.65 | B |
| ATOM | 3317 | CB | ARG | B | 180 | 56.143 | 47.429 | 10.590 | 1.00 | 12.34 | B |
| ATOM | 3318 | CG | ARG | B | 180 | 55.216 | 46.324 | 10.142 | 1.00 | 12.89 | B |
| ATOM | 3319 | CD | ARG | B | 180 | 54.600 | 45.637 | 11.340 | 1.00 | 13.42 | B |
| ATOM | 3320 | NE | ARG | B | 180 | 53.642 | 44.618 | 10.932 | 1.00 | 14.57 | B |
| ATOM | 3321 | CZ | ARG | B | 180 | 52.972 | 43.849 | 11.781 | 1.00 | 13.41 | B |
| ATOM | 3322 | NH1 | ARG | B | 180 | 53.167 | 43.981 | 13.086 | 1.00 | 14.58 | B |
| ATOM | 3323 | NH2 | ARG | B | 180 | 52.092 | 42.967 | 11.326 | 1.00 | 15.45 | B |
| ATOM | 3324 | C | ARG | B | 180 | 56.313 | 48.602 | 8.333 | 1.00 | 11.18 | B |
| ATOM | 3325 | O | ARG | B | 180 | 56.050 | 47.981 | 7.305 | 1.00 | 12.37 | B |
| ATOM | 3326 | N | VAL | B | 181 | 55.962 | 49.870 | 8.510 | 1.00 | 10.42 | B |
| ATOM | 3327 | CA | VAL | B | 181 | 55.274 | 50.597 | 7.450 | 1.00 | 11.21 | B |
| ATOM | 3328 | CB | VAL | B | 181 | 54.931 | 52.040 | 7.895 | 1.00 | 11.89 | B |
| ATOM | 3329 | CG1 | VAL | B | 181 | 54.495 | 52.876 | 6.696 | 1.00 | 12.20 | B |
| ATOM | 3330 | CG2 | VAL | B | 181 | 53.826 | 52.009 | 8.946 | 1.00 | 12.54 | B |
| ATOM | 3331 | C | VAL | B | 181 | 56.176 | 50.663 | 6.211 | 1.00 | 11.72 | B |
| ATOM | 3332 | O | VAL | B | 181 | 55.755 | 50.346 | 5.098 | 1.00 | 11.91 | B |
| ATOM | 3333 | N | ARG | B | 182 | 57.427 | 51.051 | 6.426 | 1.00 | 12.96 | B |
| ATOM | 3334 | CA | ARG | B | 182 | 58.391 | 51.198 | 5.342 | 1.00 | 13.16 | B |
| ATOM | 3335 | CB | ARG | B | 182 | 59.645 | 51.882 | 5.889 | 1.00 | 14.59 | B |
| ATOM | 3336 | CG | ARG | B | 182 | 59.443 | 53.341 | 6.247 | 1.00 | 13.75 | B |
| ATOM | 3337 | CD | ARG | B | 182 | 60.707 | 53.964 | 6.811 | 1.00 | 14.39 | B |
| ATOM | 3338 | NE | ARG | B | 182 | 60.590 | 55.415 | 6.901 | 1.00 | 14.11 | B |
| ATOM | 3339 | CZ | ARG | B | 182 | 61.274 | 56.170 | 7.756 | 1.00 | 14.96 | B |
| ATOM | 3340 | NH1 | ARG | B | 182 | 62.126 | 55.613 | 8.604 | 1.00 | 14.64 | B |
| ATOM | 3341 | NH2 | ARG | B | 182 | 61.105 | 57.484 | 7.762 | 1.00 | 16.39 | B |
| ATOM | 3342 | C | ARG | B | 182 | 58.728 | 49.920 | 4.593 | 1.00 | 13.56 | B |
| ATOM | 3343 | O | ARG | B | 182 | 59.000 | 49.966 | 3.394 | 1.00 | 15.12 | B |
| ATOM | 3344 | N | ALA | B | 183 | 58.710 | 48.786 | 5.285 | 1.00 | 13.23 | B |
| ATOM | 3345 | CA | ALA | B | 183 | 59.071 | 47.524 | 4.650 | 1.00 | 14.60 | B |
| ATOM | 3346 | CB | ALA | B | 183 | 60.033 | 46.766 | 5.559 | 1.00 | 15.58 | B |
| ATOM | 3347 | C | ALA | B | 183 | 57.923 | 46.609 | 4.246 | 1.00 | 15.34 | B |
| ATOM | 3348 | O | ALA | B | 183 | 58.162 | 45.524 | 3.718 | 1.00 | 17.94 | B |
| ATOM | 3349 | N | SER | B | 184 | 56.684 | 47.038 | 4.461 | 1.00 | 15.42 | B |
| ATOM | 3350 | CA | SER | B | 184 | 55.542 | 46.187 | 4.134 | 1.00 | 15.22 | B |
| ATOM | 3351 | CB | SER | B | 184 | 54.556 | 46.222 | 5.307 | 1.00 | 15.25 | B |
| ATOM | 3352 | OG | SER | B | 184 | 55.158 | 45.671 | 6.468 | 1.00 | 15.24 | B |
| ATOM | 3353 | C | SER | B | 184 | 54.886 | 46.457 | 2.785 | 1.00 | 14.80 | B |
| ATOM | 3354 | O | SER | B | 184 | 53.788 | 45.972 | 2.520 | 1.00 | 14.65 | B |
| ATOM | 3355 | N | ALA | B | 185 | 55.561 | 47.225 | 1.935 | 1.00 | 15.93 | B |
| ATOM | 3356 | CA | ALA | B | 185 | 55.062 | 47.533 | 0.597 | 1.00 | 16.07 | B |
| ATOM | 3357 | CB | ALA | B | 185 | 55.203 | 46.307 | −0.302 | 1.00 | 16.51 | B |
| ATOM | 3358 | C | ALA | B | 185 | 53.619 | 48.022 | 0.579 | 1.00 | 16.96 | B |
| ATOM | 3359 | O | ALA | B | 185 | 52.847 | 47.673 | −0.320 | 1.00 | 18.07 | B |
| ATOM | 3360 | N | GLY | B | 186 | 53.262 | 48.831 | 1.572 | 1.00 | 16.20 | B |
| ATOM | 3361 | CA | GLY | B | 186 | 51.918 | 49.370 | 1.645 | 1.00 | 15.49 | B |
| ATOM | 3362 | C | GLY | B | 186 | 50.886 | 48.450 | 2.269 | 1.00 | 13.47 | B |
| ATOM | 3363 | O | GLY | B | 186 | 49.697 | 48.771 | 2.260 | 1.00 | 15.31 | B |
| ATOM | 3364 | N | LYS | B | 187 | 51.325 | 47.317 | 2.814 | 1.00 | 13.66 | B |
| ATOM | 3365 | CA | LYS | B | 187 | 50.403 | 46.366 | 3.428 | 1.00 | 13.11 | B |
| ATOM | 3366 | CB | LYS | B | 187 | 50.801 | 44.941 | 3.025 | 1.00 | 15.45 | B |
| ATOM | 3367 | CG | LYS | B | 187 | 50.827 | 44.701 | 1.525 | 1.00 | 16.91 | B |
| ATOM | 3368 | CD | LYS | B | 187 | 49.471 | 44.942 | 0.885 | 1.00 | 19.00 | B |
| ATOM | 3369 | CE | LYS | B | 187 | 49.525 | 44.665 | −0.611 | 1.00 | 21.36 | B |
| ATOM | 3370 | NZ | LYS | B | 187 | 48.215 | 44.906 | −1.274 | 1.00 | 22.80 | B |
| ATOM | 3371 | C | LYS | B | 187 | 50.281 | 46.538 | 4.939 | 1.00 | 12.81 | B |
| ATOM | 3372 | O | LYS | B | 187 | 49.689 | 45.698 | 5.608 | 1.00 | 12.65 | B |
| ATOM | 3373 | N | TYR | B | 188 | 50.853 | 47.609 | 5.483 | 1.00 | 12.17 | B |
| ATOM | 3374 | CA | TYR | B | 188 | 50.750 | 47.878 | 6.914 | 1.00 | 11.57 | B |
| ATOM | 3375 | CB | TYR | B | 188 | 51.961 | 47.319 | 7.676 | 1.00 | 11.09 | B |
| ATOM | 3376 | CG | TYR | B | 188 | 51.841 | 47.487 | 9.186 | 1.00 | 10.63 | B |
| ATOM | 3377 | CD1 | TYR | B | 188 | 51.028 | 46.636 | 9.943 | 1.00 | 10.72 | B |
| ATOM | 3378 | CE1 | TYR | B | 188 | 50.867 | 46.818 | 11.321 | 1.00 | 11.82 | B |
| ATOM | 3379 | CD2 | TYR | B | 188 | 52.495 | 48.527 | 9.852 | 1.00 | 10.42 | B |
| ATOM | 3380 | CE2 | TYR | B | 188 | 52.337 | 48.718 | 11.233 | 1.00 | 10.53 | B |
| ATOM | 3381 | CZ | TYR | B | 188 | 51.523 | 47.860 | 11.958 | 1.00 | 9.62 | B |
| ATOM | 3382 | OH | TYR | B | 188 | 51.377 | 48.051 | 13.316 | 1.00 | 10.96 | B |
| ATOM | 3383 | C | TYR | B | 188 | 50.600 | 49.371 | 7.141 | 1.00 | 10.17 | B |
| ATOM | 3384 | O | TYR | B | 188 | 51.349 | 50.169 | 6.577 | 1.00 | 12.88 | B |
| ATOM | 3385 | N | ALA | B | 189 | 49.623 | 49.737 | 7.962 | 1.00 | 10.30 | B |
| ATOM | 3386 | CA | ALA | B | 189 | 49.384 | 51.130 | 8.327 | 1.00 | 11.13 | B |
| ATOM | 3387 | CB | ALA | B | 189 | 47.984 | 51.562 | 7.928 | 1.00 | 12.49 | B |
| ATOM | 3388 | C | ALA | B | 189 | 49.550 | 51.164 | 9.844 | 1.00 | 10.34 | B |
| ATOM | 3389 | O | ALA | B | 189 | 49.112 | 50.245 | 10.545 | 1.00 | 11.00 | B |
| ATOM | 3390 | N | TYR | B | 190 | 50.175 | 52.219 | 10.352 | 1.00 | 9.97 | B |
| ATOM | 3391 | CA | TYR | B | 190 | 50.445 | 52.334 | 11.777 | 1.00 | 9.16 | B |
| ATOM | 3392 | CB | TYR | B | 190 | 51.970 | 52.483 | 11.932 | 1.00 | 10.28 | B |
| ATOM | 3393 | CG | TYR | B | 190 | 52.483 | 52.705 | 13.336 | 1.00 | 9.88 | B |
| ATOM | 3394 | CD1 | TYR | B | 190 | 51.959 | 51.999 | 14.415 | 1.00 | 10.57 | B |
| ATOM | 3395 | CE1 | TYR | B | 190 | 52.433 | 52.209 | 15.712 | 1.00 | 10.96 | B |
| ATOM | 3396 | CD2 | TYR | B | 190 | 53.503 | 53.625 | 13.581 | 1.00 | 11.85 | B |
| ATOM | 3397 | CE2 | TYR | B | 190 | 53.984 | 53.842 | 14.867 | 1.00 | 12.34 | B |
| ATOM | 3398 | CZ | TYR | B | 190 | 53.443 | 53.137 | 15.927 | 1.00 | 12.44 | B |
| ATOM | 3399 | OH | TYR | B | 190 | 53.887 | 53.392 | 17.206 | 1.00 | 15.74 | B |
| ATOM | 3400 | C | TYR | B | 190 | 49.635 | 53.443 | 12.442 | 1.00 | 9.62 | B |
| ATOM | 3401 | O | TYR | B | 190 | 49.608 | 54.576 | 11.963 | 1.00 | 10.03 | B |
| ATOM | 3402 | N | LEU | B | 191 | 48.965 | 53.105 | 13.543 | 1.00 | 9.49 | B |
| ATOM | 3403 | CA | LEU | B | 191 | 48.167 | 54.070 | 14.296 | 1.00 | 9.49 | B |
| ATOM | 3404 | CB | LEU | B | 191 | 46.900 | 53.382 | 14.815 | 1.00 | 10.49 | B |
| ATOM | 3405 | CG | LEU | B | 191 | 45.817 | 53.104 | 13.771 | 1.00 | 13.15 | B |
| ATOM | 3406 | CD1 | LEU | B | 191 | 44.943 | 51.951 | 14.230 | 1.00 | 16.03 | B |
| ATOM | 3407 | CD2 | LEU | B | 191 | 44.996 | 54.368 | 13.532 | 1.00 | 13.37 | B |
| ATOM | 3408 | C | LEU | B | 191 | 49.049 | 54.615 | 15.405 | 1.00 | 9.27 | B |
| ATOM | 3409 | O | LEU | B | 191 | 49.453 | 53.873 | 16.303 | 1.00 | 9.67 | B |
| ATOM | 3410 | N | LEU | B | 192 | 49.345 | 55.911 | 15.339 | 1.00 | 9.51 | B |
| ATOM | 3411 | CA | LEU | B | 192 | 50.213 | 56.544 | 16.326 | 1.00 | 8.61 | B |
| ATOM | 3412 | CB | LEU | B | 192 | 51.672 | 56.423 | 15.866 | 1.00 | 9.70 | B |
| ATOM | 3413 | CG | LEU | B | 192 | 52.141 | 57.440 | 14.815 | 1.00 | 10.24 | B |
| ATOM | 3414 | CD1 | LEU | B | 192 | 53.640 | 57.295 | 14.607 | 1.00 | 10.76 | B |
| ATOM | 3415 | CD2 | LEU | B | 192 | 51.398 | 57.241 | 13.497 | 1.00 | 10.44 | B |
| ATOM | 3416 | C | LEU | B | 192 | 49.792 | 57.994 | 16.532 | 1.00 | 8.70 | B |
| ATOM | 3417 | O | LEU | B | 192 | 48.979 | 58.535 | 15.779 | 1.00 | 8.53 | B |
| ATOM | 3418 | N | GLU | B | 193 | 50.364 | 58.633 | 17.543 | 1.00 | 9.07 | B |
| ATOM | 3419 | CA | GLU | B | 193 | 50.020 | 60.018 | 17.817 | 1.00 | 8.43 | B |
| ATOM | 3420 | CB | GLU | B | 193 | 50.518 | 60.365 | 19.221 | 1.00 | 8.06 | B |
| ATOM | 3421 | CG | GLU | B | 193 | 49.851 | 59.448 | 20.244 | 1.00 | 8.82 | B |
| ATOM | 3422 | CD | GLU | B | 193 | 50.226 | 59.724 | 21.680 | 1.00 | 8.07 | B |
| ATOM | 3423 | OE1 | GLU | B | 193 | 51.381 | 60.107 | 21.946 | 1.00 | 8.83 | B |
| ATOM | 3424 | OE2 | GLU | B | 193 | 49.350 | 59.529 | 22.546 | 1.00 | 9.84 | B |
| ATOM | 3425 | C | GLU | B | 193 | 50.519 | 60.919 | 16.688 | 1.00 | 8.17 | B |
| ATOM | 3426 | O | GLU | B | 193 | 51.614 | 60.729 | 16.151 | 1.00 | 8.73 | B |
| ATOM | 3427 | N | SER | B | 194 | 49.687 | 61.893 | 16.328 | 1.00 | 8.44 | B |
| ATOM | 3428 | CA | SER | B | 194 | 49.968 | 62.821 | 15.232 | 1.00 | 9.11 | B |
| ATOM | 3429 | CB | SER | B | 194 | 48.824 | 63.834 | 15.147 | 1.00 | 9.75 | B |
| ATOM | 3430 | OG | SER | B | 194 | 48.684 | 64.552 | 16.361 | 1.00 | 9.27 | B |
| ATOM | 3431 | C | SER | B | 194 | 51.334 | 63.477 | 15.333 | 1.00 | 8.86 | B |
| ATOM | 3432 | O | SER | B | 194 | 51.987 | 63.743 | 14.318 | 1.00 | 9.10 | B |
| ATOM | 3433 | N | THR | B | 195 | 51.754 | 63.753 | 16.560 | 1.00 | 8.85 | B |
| ATOM | 3434 | CA | THR | B | 195 | 53.055 | 64.353 | 16.816 | 1.00 | 9.23 | B |
| ATOM | 3435 | CB | THR | B | 195 | 53.224 | 64.564 | 18.314 | 1.00 | 10.50 | B |
| ATOM | 3436 | OG1 | THR | B | 195 | 52.689 | 63.420 | 18.990 | 1.00 | 10.30 | B |
| ATOM | 3437 | CG2 | THR | B | 195 | 52.474 | 65.813 | 18.766 | 1.00 | 11.47 | B |
| ATOM | 3438 | C | THR | B | 195 | 54.185 | 63.468 | 16.278 | 1.00 | 10.40 | B |
| ATOM | 3439 | O | THR | B | 195 | 55.065 | 63.953 | 15.577 | 1.00 | 9.85 | B |
| ATOM | 3440 | N | MET | B | 196 | 54.174 | 62.176 | 16.604 | 1.00 | 10.06 | B |
| ATOM | 3441 | CA | MET | B | 196 | 55.215 | 61.276 | 16.101 | 1.00 | 10.48 | B |
| ATOM | 3442 | CB | MET | B | 196 | 55.136 | 59.909 | 16.796 | 1.00 | 10.02 | B |
| ATOM | 3443 | CG | MET | B | 196 | 55.727 | 59.866 | 18.184 | 1.00 | 11.31 | B |
| ATOM | 3444 | SD | MET | B | 196 | 54.694 | 60.705 | 19.367 | 1.00 | 11.81 | B |
| ATOM | 3445 | CE | MET | B | 196 | 55.074 | 59.782 | 20.855 | 1.00 | 13.94 | B |
| ATOM | 3446 | C | MET | B | 196 | 55.084 | 61.135 | 14.595 | 1.00 | 9.86 | B |
| ATOM | 3447 | O | MET | B | 196 | 56.087 | 61.070 | 13.881 | 1.00 | 10.95 | B |
| ATOM | 3448 | N | ASN | B | 197 | 53.844 | 61.075 | 14.119 | 1.00 | 9.10 | B |
| ATOM | 3449 | CA | ASN | B | 197 | 53.567 | 60.924 | 12.694 | 1.00 | 9.87 | B |
| ATOM | 3450 | CB | ASN | B | 197 | 52.046 | 60.840 | 12.500 | 1.00 | 9.27 | B |
| ATOM | 3451 | CG | ASN | B | 197 | 51.647 | 60.155 | 11.201 | 1.00 | 12.08 | B |
| ATOM | 3452 | OD1 | ASN | B | 197 | 52.465 | 59.953 | 10.304 | 1.00 | 11.55 | B |
| ATOM | 3453 | ND2 | ASN | B | 197 | 50.375 | 59.802 | 11.095 | 1.00 | 9.98 | B |
| ATOM | 3454 | C | ASN | B | 197 | 54.209 | 62.081 | 11.927 | 1.00 | 10.21 | B |
| ATOM | 3455 | O | ASN | B | 197 | 54.915 | 61.869 | 10.934 | 1.00 | 10.46 | B |
| ATOM | 3456 | N | GLU | B | 198 | 53.960 | 63.303 | 12.393 | 1.00 | 10.61 | B |
| ATOM | 3457 | CA | GLU | B | 198 | 54.495 | 64.515 | 11.763 | 1.00 | 10.80 | B |
| ATOM | 3458 | CB | GLU | B | 198 | 53.880 | 65.740 | 12.447 | 1.00 | 13.24 | B |
| ATOM | 3459 | CG | GLU | B | 198 | 52.385 | 65.875 | 12.258 | 1.00 | 15.36 | B |
| ATOM | 3460 | CD | GLU | B | 198 | 51.732 | 66.735 | 13.327 | 1.00 | 18.47 | B |
| ATOM | 3461 | OE1 | GLU | B | 198 | 50.503 | 66.941 | 13.247 | 1.00 | 21.12 | B |
| ATOM | 3462 | OE2 | GLU | B | 198 | 52.440 | 67.200 | 14.249 | 1.00 | 19.03 | B |
| ATOM | 3463 | C | GLU | B | 198 | 56.016 | 64.538 | 11.787 | 1.00 | 10.81 | B |
| ATOM | 3464 | O | GLU | B | 198 | 56.656 | 64.981 | 10.828 | 1.00 | 12.73 | B |
| ATOM | 3465 | N | TYR | B | 199 | 56.596 | 64.078 | 12.889 | 1.00 | 11.33 | B |
| ATOM | 3466 | CA | TYR | B | 199 | 58.044 | 64.048 | 13.032 | 1.00 | 12.01 | B |
| ATOM | 3467 | CB | TYR | B | 199 | 58.391 | 63.624 | 14.457 | 1.00 | 12.78 | B |
| ATOM | 3468 | CG | TYR | B | 199 | 59.826 | 63.191 | 14.619 | 1.00 | 12.16 | B |
| ATOM | 3469 | CD1 | TYR | B | 199 | 60.868 | 64.117 | 14.563 | 1.00 | 14.08 | B |
| ATOM | 3470 | CE1 | TYR | B | 199 | 62.199 | 63.710 | 14.694 | 1.00 | 14.94 | B |
| ATOM | 3471 | CD2 | TYR | B | 199 | 60.145 | 61.848 | 14.812 | 1.00 | 13.76 | B |
| ATOM | 3472 | CE2 | TYR | B | 199 | 61.467 | 61.432 | 14.943 | 1.00 | 15.28 | B |
| ATOM | 3473 | CZ | TYR | B | 199 | 62.486 | 62.367 | 14.883 | 1.00 | 15.02 | B |
| ATOM | 3474 | OH | TYR | B | 199 | 63.790 | 61.949 | 15.007 | 1.00 | 17.71 | B |
| ATOM | 3475 | C | TYR | B | 199 | 58.657 | 63.129 | 11.980 | 1.00 | 11.96 | B |
| ATOM | 3476 | O | TYR | B | 199 | 59.571 | 63.519 | 11.254 | 1.00 | 12.45 | B |
| ATOM | 3477 | N | ILE | B | 200 | 58.148 | 61.905 | 11.910 | 1.00 | 11.56 | B |
| ATOM | 3478 | CA | ILE | B | 200 | 58.638 | 60.909 | 10.962 | 1.00 | 11.82 | B |
| ATOM | 3479 | CB | ILE | B | 200 | 57.940 | 59.547 | 11.205 | 1.00 | 12.48 | B |
| ATOM | 3480 | CG2 | ILE | B | 200 | 58.368 | 58.541 | 10.147 | 1.00 | 12.77 | B |
| ATOM | 3481 | CG1 | ILE | B | 200 | 58.273 | 59.021 | 12.609 | 1.00 | 13.25 | B |
| ATOM | 3482 | CD1 | ILE | B | 200 | 59.733 | 58.644 | 12.819 | 1.00 | 13.40 | B |
| ATOM | 3483 | C | ILE | B | 200 | 58.428 | 61.329 | 9.502 | 1.00 | 12.47 | B |
| ATOM | 3484 | O | ILE | B | 200 | 59.240 | 61.005 | 8.632 | 1.00 | 13.01 | B |
| ATOM | 3485 | N | GLU | B | 201 | 57.344 | 62.051 | 9.240 | 1.00 | 11.53 | B |
| ATOM | 3486 | CA | GLU | B | 201 | 57.036 | 62.508 | 7.890 | 1.00 | 14.28 | B |
| ATOM | 3487 | CB | GLU | B | 201 | 55.673 | 63.197 | 7.905 | 1.00 | 14.24 | B |
| ATOM | 3488 | CG | GLU | B | 201 | 55.102 | 63.502 | 6.535 | 1.00 | 19.66 | B |
| ATOM | 3489 | CD | GLU | B | 201 | 53.701 | 64.075 | 6.616 | 1.00 | 21.29 | B |
| ATOM | 3490 | OE1 | GLU | B | 201 | 53.544 | 65.193 | 7.151 | 1.00 | 25.24 | B |
| ATOM | 3491 | OE2 | GLU | B | 201 | 52.756 | 63.406 | 6.152 | 1.00 | 25.00 | B |
| ATOM | 3492 | C | GLU | B | 201 | 58.144 | 63.403 | 7.348 | 1.00 | 15.97 | B |
| ATOM | 3493 | O | GLU | B | 201 | 58.273 | 63.570 | 6.133 | 1.00 | 16.88 | B |
| ATOM | 3494 | N | GLN | B | 202 | 58.939 | 63.980 | 8.245 | 1.00 | 15.22 | B |
| ATOM | 3495 | CA | GLN | B | 202 | 60.029 | 64.864 | 7.844 | 1.00 | 18.83 | B |
| ATOM | 3496 | CB | GLN | B | 202 | 59.961 | 66.150 | 8.677 | 1.00 | 19.32 | B |
| ATOM | 3497 | CG | GLN | B | 202 | 58.627 | 66.882 | 8.602 | 1.00 | 24.25 | B |
| ATOM | 3498 | CD | GLN | B | 202 | 58.205 | 67.193 | 7.178 | 1.00 | 27.58 | B |
| ATOM | 3499 | OE1 | GLN | B | 202 | 58.957 | 67.795 | 6.411 | 1.00 | 31.06 | B |
| ATOM | 3500 | NE2 | GLN | B | 202 | 56.992 | 66.787 | 6.819 | 1.00 | 29.66 | B |
| ATOM | 3501 | C | GLN | B | 202 | 61.379 | 64.164 | 7.952 | 1.00 | 18.85 | B |
| ATOM | 3502 | O | GLN | B | 202 | 62.423 | 64.816 | 7.925 | 1.00 | 21.64 | B |
| ATOM | 3503 | N | ARG | B | 203 | 61.361 | 62.841 | 8.070 | 1.00 | 19.16 | B |
| ATOM | 3504 | CA | ARG | B | 203 | 62.594 | 62.069 | 8.183 | 1.00 | 19.65 | B |
| ATOM | 3505 | CB | ARG | B | 203 | 62.526 | 61.193 | 9.441 | 1.00 | 20.67 | B |
| ATOM | 3506 | CG | ARG | B | 203 | 62.292 | 61.956 | 10.734 | 1.00 | 22.05 | B |
| ATOM | 3507 | CD | ARG | B | 203 | 63.531 | 62.718 | 11.174 | 1.00 | 24.79 | B |
| ATOM | 3508 | NE | ARG | B | 203 | 64.605 | 61.819 | 11.589 | 1.00 | 25.67 | B |
| ATOM | 3509 | CZ | ARG | B | 203 | 65.771 | 62.226 | 12.079 | 1.00 | 26.71 | B |
| ATOM | 3510 | NH1 | ARG | B | 203 | 66.018 | 63.522 | 12.215 | 1.00 | 26.28 | B |
| ATOM | 3511 | NH2 | ARG | B | 203 | 66.691 | 61.338 | 12.435 | 1.00 | 26.10 | B |
| ATOM | 3512 | C | ARG | B | 203 | 62.822 | 61.249 | 6.917 | 1.00 | 19.60 | B |
| ATOM | 3513 | O | ARG | B | 203 | 61.880 | 60.713 | 6.335 | 1.00 | 17.46 | B |
| ATOM | 3514 | N | LYS | B | 204 | 64.077 | 61.156 | 6.491 | 1.00 | 20.33 | B |
| ATOM | 3515 | CA | LYS | B | 204 | 64.415 | 60.370 | 5.312 | 1.00 | 21.37 | B |
| ATOM | 3516 | CB | LYS | B | 204 | 65.896 | 60.578 | 4.975 | 1.00 | 21.77 | B |
| ATOM | 3517 | CG | LYS | B | 204 | 66.243 | 61.991 | 4.525 | 1.00 | 25.46 | B |
| ATOM | 3518 | CD | LYS | B | 204 | 67.752 | 62.222 | 4.512 | 1.00 | 28.33 | B |
| ATOM | 3519 | CE | LYS | B | 204 | 68.468 | 61.239 | 3.600 | 1.00 | 29.88 | B |
| ATOM | 3520 | NZ | LYS | B | 204 | 69.950 | 61.384 | 3.686 | 1.00 | 31.44 | B |
| ATOM | 3521 | C | LYS | B | 204 | 64.068 | 58.928 | 5.664 | 1.00 | 21.04 | B |
| ATOM | 3522 | O | LYS | B | 204 | 64.127 | 58.541 | 6.831 | 1.00 | 22.46 | B |
| ATOM | 3523 | N | PRO | B | 205 | 63.735 | 58.101 | 4.662 | 1.00 | 20.47 | B |
| ATOM | 3524 | CD | PRO | B | 205 | 63.604 | 56.655 | 4.915 | 1.00 | 20.59 | B |
| ATOM | 3525 | CA | PRO | B | 205 | 63.652 | 58.378 | 3.225 | 1.00 | 20.46 | B |
| ATOM | 3526 | CB | PRO | B | 205 | 63.880 | 56.996 | 2.620 | 1.00 | 21.06 | B |
| ATOM | 3527 | CG | PRO | B | 205 | 63.090 | 56.109 | 3.530 | 1.00 | 22.08 | B |
| ATOM | 3528 | C | PRO | B | 205 | 62.396 | 59.099 | 2.725 | 1.00 | 19.52 | B |
| ATOM | 3529 | O | PRO | B | 205 | 62.064 | 59.011 | 1.543 | 1.00 | 19.08 | B |
| ATOM | 3530 | N | CYS | B | 206 | 61.690 | 59.799 | 3.608 | 1.00 | 17.99 | B |
| ATOM | 3531 | CA | CYS | B | 206 | 60.504 | 60.544 | 3.190 | 1.00 | 16.87 | B |
| ATOM | 3532 | C | CYS | B | 206 | 59.509 | 59.659 | 2.453 | 1.00 | 15.84 | B |
| ATOM | 3533 | O | CYS | B | 206 | 58.973 | 60.055 | 1.418 | 1.00 | 15.47 | B |
| ATOM | 3534 | CB | CYS | B | 206 | 60.955 | 61.721 | 2.326 | 1.00 | 19.04 | B |
| ATOM | 3535 | SG | CYS | B | 206 | 62.275 | 62.714 | 3.076 | 1.00 | 20.10 | B |
| ATOM | 3536 | N | ASP | B | 207 | 59.245 | 58.472 | 2.988 | 1.00 | 15.12 | B |
| ATOM | 3537 | CA | ASP | B | 207 | 58.332 | 57.545 | 2.336 | 1.00 | 14.66 | B |
| ATOM | 3538 | CB | ASP | B | 207 | 59.096 | 56.256 | 2.019 | 1.00 | 15.55 | B |
| ATOM | 3539 | CG | ASP | B | 207 | 59.615 | 55.567 | 3.266 | 1.00 | 15.30 | B |
| ATOM | 3540 | OD1 | ASP | B | 207 | 59.661 | 56.220 | 4.330 | 1.00 | 17.08 | B |
| ATOM | 3541 | OD2 | ASP | B | 207 | 59.989 | 54.377 | 3.182 | 1.00 | 18.19 | B |
| ATOM | 3542 | C | ASP | B | 207 | 57.061 | 57.298 | 3.135 | 1.00 | 14.35 | B |
| ATOM | 3543 | O | ASP | B | 207 | 56.287 | 56.400 | 2.811 | 1.00 | 13.73 | B |
| ATOM | 3544 | N | THR | B | 208 | 56.845 | 58.088 | 4.181 | 1.00 | 14.62 | B |
| ATOM | 3545 | CA | THR | B | 208 | 55.645 | 57.925 | 4.992 | 1.00 | 13.43 | B |
| ATOM | 3546 | CB | THR | B | 208 | 55.978 | 57.704 | 6.485 | 1.00 | 13.85 | B |
| ATOM | 3547 | OG1 | THR | B | 208 | 56.642 | 58.860 | 7.008 | 1.00 | 15.61 | B |
| ATOM | 3548 | CG2 | THR | B | 208 | 56.869 | 56.483 | 6.657 | 1.00 | 15.09 | B |
| ATOM | 3549 | C | THR | B | 208 | 54.765 | 59.159 | 4.871 | 1.00 | 13.60 | B |
| ATOM | 3550 | O | THR | B | 208 | 55.221 | 60.228 | 4.458 | 1.00 | 14.47 | B |
| ATOM | 3551 | N | MET | B | 209 | 53.498 | 59.009 | 5.234 | 1.00 | 13.97 | B |
| ATOM | 3552 | CA | MET | B | 209 | 52.569 | 60.118 | 5.156 | 1.00 | 14.24 | B |
| ATOM | 3553 | CB | MET | B | 209 | 52.053 | 60.224 | 3.720 | 1.00 | 15.73 | B |
| ATOM | 3554 | CG | MET | B | 209 | 50.998 | 61.291 | 3.526 | 1.00 | 18.55 | B |
| ATOM | 3555 | SD | MET | B | 209 | 50.531 | 61.480 | 1.800 | 1.00 | 24.13 | B |
| ATOM | 3556 | CE | MET | B | 209 | 49.421 | 60.119 | 1.585 | 1.00 | 24.81 | B |
| ATOM | 3557 | C | MET | B | 209 | 51.445 | 59.977 | 6.168 | 1.00 | 13.47 | B |
| ATOM | 3558 | O | MET | B | 209 | 50.973 | 58.873 | 6.439 | 1.00 | 13.39 | B |
| ATOM | 3559 | N | LYS | B | 210 | 51.029 | 61.107 | 6.728 | 1.00 | 14.58 | B |
| ATOM | 3560 | CA | LYS | B | 210 | 49.925 | 61.135 | 7.677 | 1.00 | 15.13 | B |
| ATOM | 3561 | CB | LYS | B | 210 | 50.163 | 62.239 | 8.713 | 1.00 | 15.35 | B |
| ATOM | 3562 | CG | LYS | B | 210 | 48.994 | 62.449 | 9.660 | 1.00 | 17.22 | B |
| ATOM | 3563 | CD | LYS | B | 210 | 49.267 | 63.588 | 10.630 | 1.00 | 20.64 | B |
| ATOM | 3564 | CE | LYS | B | 210 | 48.098 | 63.805 | 11.570 | 1.00 | 21.70 | B |
| ATOM | 3565 | NZ | LYS | B | 210 | 48.312 | 64.990 | 12.443 | 1.00 | 25.64 | B |
| ATOM | 3566 | C | LYS | B | 210 | 48.679 | 61.367 | 6.832 | 1.00 | 15.45 | B |
| ATOM | 3567 | O | LYS | B | 210 | 48.629 | 62.316 | 6.047 | 1.00 | 16.38 | B |
| ATOM | 3568 | N | VAL | B | 211 | 47.676 | 60.508 | 6.984 | 1.00 | 14.47 | B |
| ATOM | 3569 | CA | VAL | B | 211 | 46.447 | 60.646 | 6.209 | 1.00 | 16.16 | B |
| ATOM | 3570 | CB | VAL | B | 211 | 46.308 | 59.517 | 5.161 | 1.00 | 15.70 | B |
| ATOM | 3571 | CG1 | VAL | B | 211 | 47.420 | 59.623 | 4.134 | 1.00 | 18.13 | B |
| ATOM | 3572 | CG2 | VAL | B | 211 | 46.347 | 58.158 | 5.847 | 1.00 | 16.74 | B |
| ATOM | 3573 | C | VAL | B | 211 | 45.191 | 60.655 | 7.068 | 1.00 | 17.02 | B |
| ATOM | 3574 | O | VAL | B | 211 | 45.155 | 60.059 | 8.147 | 1.00 | 15.85 | B |
| ATOM | 3575 | N | GLY | B | 212 | 44.160 | 61.332 | 6.569 | 1.00 | 18.91 | B |
| ATOM | 3576 | CA | GLY | B | 212 | 42.902 | 61.420 | 7.285 | 1.00 | 19.63 | B |
| ATOM | 3577 | C | GLY | B | 212 | 42.989 | 62.341 | 8.483 | 1.00 | 18.48 | B |
| ATOM | 3578 | O | GLY | B | 212 | 44.077 | 62.764 | 8.877 | 1.00 | 20.50 | B |
| ATOM | 3579 | N | GLY | B | 213 | 41.839 | 62.670 | 9.059 | 1.00 | 18.28 | B |
| ATOM | 3580 | CA | GLY | B | 213 | 41.837 | 63.530 | 10.224 | 1.00 | 17.13 | B |
| ATOM | 3581 | C | GLY | B | 213 | 42.213 | 62.700 | 11.433 | 1.00 | 15.25 | B |
| ATOM | 3582 | O | GLY | B | 213 | 42.205 | 61.465 | 11.373 | 1.00 | 17.51 | B |
| ATOM | 3583 | N | ASN | B | 214 | 42.560 | 63.362 | 12.530 | 1.00 | 14.08 | B |
| ATOM | 3584 | CA | ASN | B | 214 | 42.914 | 62.635 | 13.738 | 1.00 | 12.59 | B |
| ATOM | 3585 | CB | ASN | B | 214 | 43.539 | 63.594 | 14.754 | 1.00 | 13.59 | B |
| ATOM | 3586 | CG | ASN | B | 214 | 44.882 | 64.128 | 14.304 | 1.00 | 14.04 | B |
| ATOM | 3587 | OD1 | ASN | B | 214 | 45.520 | 63.570 | 13.410 | 1.00 | 15.39 | B |
| ATOM | 3588 | ND2 | ASN | B | 214 | 45.328 | 65.205 | 14.938 | 1.00 | 15.44 | B |
| ATOM | 3589 | C | ASN | B | 214 | 41.662 | 61.945 | 14.260 | 1.00 | 12.51 | B |
| ATOM | 3590 | O | ASN | B | 214 | 40.545 | 62.434 | 14.070 | 1.00 | 13.98 | B |
| ATOM | 3591 | N | LEU | B | 215 | 41.853 | 60.811 | 14.922 | 1.00 | 11.43 | B |
| ATOM | 3592 | CA | LEU | B | 215 | 40.736 | 60.052 | 15.471 | 1.00 | 10.90 | B |
| ATOM | 3593 | CB | LEU | B | 215 | 41.146 | 58.586 | 15.635 | 1.00 | 11.99 | B |
| ATOM | 3594 | CG | LEU | B | 215 | 41.241 | 57.742 | 14.361 | 1.00 | 12.59 | B |
| ATOM | 3595 | CD1 | LEU | B | 215 | 42.292 | 58.306 | 13.416 | 1.00 | 16.18 | B |
| ATOM | 3596 | CD2 | LEU | B | 215 | 41.581 | 56.316 | 14.744 | 1.00 | 13.71 | B |
| ATOM | 3597 | C | LEU | B | 215 | 40.223 | 60.670 | 16.765 | 1.00 | 10.91 | B |
| ATOM | 3598 | O | LEU | B | 215 | 39.087 | 60.410 | 17.165 | 1.00 | 12.75 | B |
| ATOM | 3599 | N | ASP | B | 216 | 41.066 | 61.453 | 17.437 | 1.00 | 10.37 | B |
| ATOM | 3600 | CA | ASP | B | 216 | 40.678 | 62.123 | 18.673 | 1.00 | 9.90 | B |
| ATOM | 3601 | CB | ASP | B | 216 | 40.772 | 61.151 | 19.873 | 1.00 | 9.05 | B |
| ATOM | 3602 | CG | ASP | B | 216 | 42.194 | 60.733 | 20.221 | 1.00 | 9.75 | B |
| ATOM | 3603 | OD1 | ASP | B | 216 | 43.159 | 61.145 | 19.546 | 1.00 | 9.43 | B |
| ATOM | 3604 | OD2 | ASP | B | 216 | 42.342 | 59.975 | 21.208 | 1.00 | 9.63 | B |
| ATOM | 3605 | C | ASP | B | 216 | 41.484 | 63.405 | 18.826 | 1.00 | 9.20 | B |
| ATOM | 3606 | O | ASP | B | 216 | 42.306 | 63.728 | 17.968 | 1.00 | 10.98 | B |
| ATOM | 3607 | N | SER | B | 217 | 41.222 | 64.143 | 19.903 | 1.00 | 9.88 | B |
| ATOM | 3608 | CA | SER | B | 217 | 41.899 | 65.409 | 20.174 | 1.00 | 9.92 | B |
| ATOM | 3609 | CB | SER | B | 217 | 40.987 | 66.560 | 19.746 | 1.00 | 12.87 | B |
| ATOM | 3610 | OG | SER | B | 217 | 40.715 | 66.507 | 18.361 | 1.00 | 20.77 | B |
| ATOM | 3611 | C | SER | B | 217 | 42.271 | 65.483 | 21.645 | 1.00 | 10.98 | B |
| ATOM | 3612 | O | SER | B | 217 | 41.398 | 65.520 | 22.511 | 1.00 | 12.32 | B |
| ATOM | 3613 | N | LYS | B | 218 | 43.567 | 65.524 | 21.928 | 1.00 | 11.11 | B |
| ATOM | 3614 | CA | LYS | B | 218 | 44.037 | 65.574 | 23.302 | 1.00 | 9.81 | B |
| ATOM | 3615 | CB | LYS | B | 218 | 44.082 | 64.143 | 23.854 | 1.00 | 11.05 | B |
| ATOM | 3616 | CG | LYS | B | 218 | 44.895 | 63.171 | 23.013 | 1.00 | 11.12 | B |
| ATOM | 3617 | CD | LYS | B | 218 | 44.435 | 61.740 | 23.254 | 1.00 | 9.73 | B |
| ATOM | 3618 | CE | LYS | B | 218 | 45.266 | 60.748 | 22.453 | 1.00 | 9.17 | B |
| ATOM | 3619 | NZ | LYS | B | 218 | 44.647 | 59.391 | 22.476 | 1.00 | 8.65 | B |
| ATOM | 3620 | C | LYS | B | 218 | 45.366 | 66.306 | 23.376 | 1.00 | 9.32 | B |
| ATOM | 3621 | O | LYS | B | 218 | 45.705 | 67.084 | 22.486 | 1.00 | 8.84 | B |
| ATOM | 3622 | N | GLY | B | 219 | 46.116 | 66.069 | 24.445 | 1.00 | 9.27 | B |
| ATOM | 3623 | CA | GLY | B | 219 | 47.398 | 66.735 | 24.585 | 1.00 | 8.98 | B |
| ATOM | 3624 | C | GLY | B | 219 | 48.316 | 66.051 | 25.573 | 1.00 | 8.88 | B |
| ATOM | 3625 | O | GLY | B | 219 | 47.897 | 65.140 | 26.292 | 1.00 | 9.06 | B |
| ATOM | 3626 | N | TYR | B | 220 | 49.578 | 66.473 | 25.574 | 1.00 | 7.83 | B |
| ATOM | 3627 | CA | TYR | B | 220 | 50.574 | 65.962 | 26.507 | 1.00 | 8.61 | B |
| ATOM | 3628 | CB | TYR | B | 220 | 51.961 | 65.899 | 25.871 | 1.00 | 8.83 | B |
| ATOM | 3629 | CG | TYR | B | 220 | 52.107 | 64.983 | 24.697 | 1.00 | 9.25 | B |
| ATOM | 3630 | CD1 | TYR | B | 220 | 51.520 | 63.719 | 24.690 | 1.00 | 9.06 | B |
| ATOM | 3631 | CE1 | TYR | B | 220 | 51.708 | 62.850 | 23.625 | 1.00 | 8.63 | B |
| ATOM | 3632 | CD2 | TYR | B | 220 | 52.883 | 65.359 | 23.607 | 1.00 | 8.80 | B |
| ATOM | 3633 | CE2 | TYR | B | 220 | 53.079 | 64.501 | 22.540 | 1.00 | 8.78 | B |
| ATOM | 3634 | CZ | TYR | B | 220 | 52.491 | 63.251 | 22.554 | 1.00 | 8.54 | B |
| ATOM | 3635 | OH | TYR | B | 220 | 52.691 | 62.403 | 21.491 | 1.00 | 9.33 | B |
| ATOM | 3636 | C | TYR | B | 220 | 50.597 | 66.928 | 27.670 | 1.00 | 7.92 | B |
| ATOM | 3637 | O | TYR | B | 220 | 50.487 | 68.145 | 27.478 | 1.00 | 8.32 | B |
| ATOM | 3638 | N | GLY | B | 221 | 50.770 | 66.391 | 28.869 | 1.00 | 8.36 | B |
| ATOM | 3639 | CA | GLY | B | 221 | 50.840 | 67.239 | 30.039 | 1.00 | 9.29 | B |
| ATOM | 3640 | C | GLY | B | 221 | 52.001 | 66.867 | 30.936 | 1.00 | 8.00 | B |
| ATOM | 3641 | O | GLY | B | 221 | 52.469 | 65.727 | 30.916 | 1.00 | 8.77 | B |
| ATOM | 3642 | N | ILE | B | 222 | 52.495 | 67.840 | 31.693 | 1.00 | 8.57 | B |
| ATOM | 3643 | CA | ILE | B | 222 | 53.565 | 67.590 | 32.646 | 1.00 | 8.46 | B |
| ATOM | 3644 | CB | ILE | B | 222 | 54.310 | 68.895 | 33.010 | 1.00 | 8.80 | B |
| ATOM | 3645 | CG2 | ILE | B | 222 | 55.246 | 68.652 | 34.190 | 1.00 | 11.10 | B |
| ATOM | 3646 | CG1 | ILE | B | 222 | 55.082 | 69.402 | 31.785 | 1.00 | 12.64 | B |
| ATOM | 3647 | CD1 | ILE | B | 222 | 55.892 | 70.668 | 32.024 | 1.00 | 14.32 | B |
| ATOM | 3648 | C | ILE | B | 222 | 52.848 | 67.016 | 33.872 | 1.00 | 8.77 | B |
| ATOM | 3649 | O | ILE | B | 222 | 51.804 | 67.531 | 34.285 | 1.00 | 10.76 | B |
| ATOM | 3650 | N | ALA | B | 223 | 53.390 | 65.951 | 34.450 | 1.00 | 9.70 | B |
| ATOM | 3651 | CA | ALA | B | 223 | 52.745 | 65.321 | 35.596 | 1.00 | 9.37 | B |
| ATOM | 3652 | CB | ALA | B | 223 | 52.454 | 63.852 | 35.284 | 1.00 | 9.67 | B |
| ATOM | 3653 | C | ALA | B | 223 | 53.536 | 65.428 | 36.889 | 1.00 | 9.44 | B |
| ATOM | 3654 | O | ALA | B | 223 | 54.764 | 65.406 | 36.890 | 1.00 | 8.84 | B |
| ATOM | 3655 | N | THR | B | 224 | 52.802 | 65.546 | 37.990 | 1.00 | 9.86 | B |
| ATOM | 3656 | CA | THR | B | 224 | 53.384 | 65.630 | 39.322 | 1.00 | 10.14 | B |
| ATOM | 3657 | CB | THR | B | 224 | 53.408 | 67.088 | 39.846 | 1.00 | 10.69 | B |
| ATOM | 3658 | OG1 | THR | B | 224 | 52.133 | 67.698 | 39.623 | 1.00 | 12.13 | B |
| ATOM | 3659 | CG2 | THR | B | 224 | 54.491 | 67.895 | 39.153 | 1.00 | 10.75 | B |
| ATOM | 3660 | C | THR | B | 224 | 52.507 | 64.796 | 40.252 | 1.00 | 10.82 | B |
| ATOM | 3661 | O | THR | B | 224 | 51.354 | 64.513 | 39.937 | 1.00 | 11.22 | B |
| ATOM | 3662 | N | PRO | B | 225 | 53.049 | 64.370 | 41.403 | 1.00 | 11.54 | B |
| ATOM | 3663 | CD | PRO | B | 225 | 54.399 | 64.568 | 41.953 | 1.00 | 12.42 | B |
| ATOM | 3664 | CA | PRO | B | 225 | 52.216 | 63.576 | 42.312 | 1.00 | 12.84 | B |
| ATOM | 3665 | CB | PRO | B | 225 | 53.217 | 63.129 | 43.380 | 1.00 | 13.64 | B |
| ATOM | 3666 | CG | PRO | B | 225 | 54.171 | 64.288 | 43.486 | 1.00 | 17.80 | B |
| ATOM | 3667 | C | PRO | B | 225 | 51.063 | 64.440 | 42.812 | 1.00 | 14.09 | B |
| ATOM | 3668 | O | PRO | B | 225 | 51.195 | 65.658 | 42.906 | 1.00 | 14.20 | B |
| ATOM | 3669 | N | LYS | B | 226 | 49.931 | 63.816 | 43.119 | 1.00 | 15.70 | B |
| ATOM | 3670 | CA | LYS | B | 226 | 48.783 | 64.565 | 43.612 | 1.00 | 19.44 | B |
| ATOM | 3671 | CB | LYS | B | 226 | 47.619 | 63.607 | 43.874 | 1.00 | 20.33 | B |
| ATOM | 3672 | CG | LYS | B | 226 | 46.650 | 63.471 | 42.723 | 1.00 | 22.92 | B |
| ATOM | 3673 | CD | LYS | B | 226 | 45.386 | 62.769 | 43.184 | 1.00 | 24.41 | B |
| ATOM | 3674 | CE | LYS | B | 226 | 44.326 | 62.765 | 42.102 | 1.00 | 25.95 | B |
| ATOM | 3675 | NZ | LYS | B | 226 | 43.062 | 62.157 | 42.594 | 1.00 | 27.19 | B |
| ATOM | 3676 | C | LYS | B | 226 | 49.154 | 65.371 | 44.845 | 1.00 | 20.40 | B |
| ATOM | 3677 | O | LYS | B | 226 | 49.795 | 64.861 | 45.765 | 1.00 | 20.12 | B |
| ATOM | 3678 | N | GLY | B | 227 | 48.751 | 66.637 | 44.856 | 1.00 | 21.76 | B |
| ATOM | 3679 | CA | GLY | B | 227 | 49.034 | 67.493 | 45.992 | 1.00 | 22.87 | B |
| ATOM | 3680 | C | GLY | B | 227 | 50.411 | 68.126 | 45.994 | 1.00 | 23.69 | B |
| ATOM | 3681 | O | GLY | B | 227 | 50.720 | 68.932 | 46.871 | 1.00 | 24.90 | B |
| ATOM | 3682 | N | SER | B | 228 | 51.243 | 67.767 | 45.020 | 1.00 | 23.21 | B |
| ATOM | 3683 | CA | SER | B | 228 | 52.591 | 68.318 | 44.926 | 1.00 | 24.09 | B |
| ATOM | 3684 | CB | SER | B | 228 | 53.279 | 67.758 | 43.678 | 1.00 | 23.78 | B |
| ATOM | 3685 | OG | SER | B | 228 | 54.504 | 68.431 | 43.442 | 1.00 | 24.30 | B |
| ATOM | 3686 | C | SER | B | 228 | 52.570 | 69.839 | 44.942 | 1.00 | 24.66 | B |
| ATOM | 3687 | O | SER | B | 228 | 51.767 | 70.466 | 44.254 | 1.00 | 24.48 | B |
| ATOM | 3688 | N | SER | B | 229 | 53.470 | 70.428 | 45.721 | 1.00 | 25.30 | B |
| ATOM | 3689 | CA | SER | B | 229 | 53.561 | 71.879 | 45.828 | 1.00 | 25.59 | B |
| ATOM | 3690 | CB | SER | B | 229 | 54.252 | 72.236 | 47.146 | 1.00 | 25.94 | B |
| ATOM | 3691 | OG | SER | B | 229 | 55.577 | 71.737 | 47.170 | 1.00 | 28.01 | B |
| ATOM | 3692 | C | SER | B | 229 | 54.278 | 72.456 | 44.613 | 1.00 | 24.69 | B |
| ATOM | 3693 | O | SER | B | 229 | 54.496 | 73.664 | 44.528 | 1.00 | 25.48 | B |
| ATOM | 3694 | N | LEU | B | 230 | 54.651 | 71.587 | 43.678 | 1.00 | 22.34 | B |
| ATOM | 3695 | CA | LEU | B | 230 | 55.346 | 72.009 | 42.468 | 1.00 | 21.30 | B |
| ATOM | 3696 | CB | LEU | B | 230 | 56.461 | 71.002 | 42.157 | 1.00 | 23.11 | B |
| ATOM | 3697 | CG | LEU | B | 230 | 57.896 | 71.524 | 42.050 | 1.00 | 25.25 | B |
| ATOM | 3698 | CD1 | LEU | B | 230 | 57.978 | 72.580 | 40.962 | 1.00 | 24.39 | B |
| ATOM | 3699 | CD2 | LEU | B | 230 | 58.338 | 72.097 | 43.387 | 1.00 | 27.73 | B |
| ATOM | 3700 | C | LEU | B | 230 | 54.340 | 72.143 | 41.333 | 1.00 | 19.83 | B |
| ATOM | 3701 | O | LEU | B | 230 | 54.577 | 72.858 | 40.359 | 1.00 | 19.58 | B |
| ATOM | 3702 | N | GLY | B | 231 | 53.212 | 71.455 | 41.479 | 1.00 | 18.69 | B |
| ATOM | 3703 | CA | GLY | B | 231 | 52.177 | 71.473 | 40.461 | 1.00 | 18.22 | B |
| ATOM | 3704 | C | GLY | B | 231 | 51.704 | 72.832 | 39.981 | 1.00 | 17.36 | B |
| ATOM | 3705 | O | GLY | B | 231 | 51.672 | 73.084 | 38.778 | 1.00 | 16.54 | B |
| ATOM | 3706 | N | ASN | B | 232 | 51.332 | 73.711 | 40.907 | 1.00 | 17.32 | B |
| ATOM | 3707 | CA | ASN | B | 232 | 50.844 | 75.033 | 40.534 | 1.00 | 18.12 | B |
| ATOM | 3708 | CB | ASN | B | 232 | 50.409 | 75.789 | 41.792 | 1.00 | 19.85 | B |
| ATOM | 3709 | CG | ASN | B | 232 | 49.897 | 77.181 | 41.479 | 1.00 | 21.52 | B |
| ATOM | 3710 | OD1 | ASN | B | 232 | 48.916 | 77.345 | 40.751 | 1.00 | 22.55 | B |
| ATOM | 3711 | ND2 | ASN | B | 232 | 50.561 | 78.194 | 42.025 | 1.00 | 22.41 | B |
| ATOM | 3712 | C | ASN | B | 232 | 51.861 | 75.814 | 39.710 | 1.00 | 16.03 | B |
| ATOM | 3713 | O | ASN | B | 232 | 51.540 | 76.321 | 38.635 | 1.00 | 14.82 | B |
| ATOM | 3714 | N | ALA | B | 233 | 53.083 | 75.917 | 40.223 | 1.00 | 16.42 | B |
| ATOM | 3715 | CA | ALA | B | 233 | 54.139 | 76.650 | 39.536 | 1.00 | 15.32 | B |
| ATOM | 3716 | CB | ALA | B | 233 | 55.423 | 76.610 | 40.355 | 1.00 | 16.83 | B |
| ATOM | 3717 | C | ALA | B | 233 | 54.391 | 76.089 | 38.140 | 1.00 | 13.41 | B |
| ATOM | 3718 | O | ALA | B | 233 | 54.495 | 76.838 | 37.171 | 1.00 | 13.67 | B |
| ATOM | 3719 | N | VAL | B | 234 | 54.489 | 74.769 | 38.043 | 1.00 | 13.56 | B |
| ATOM | 3720 | CA | VAL | B | 234 | 54.732 | 74.124 | 36.760 | 1.00 | 13.50 | B |
| ATOM | 3721 | CB | VAL | B | 234 | 54.857 | 72.597 | 36.933 | 1.00 | 15.11 | B |
| ATOM | 3722 | CG1 | VAL | B | 234 | 54.762 | 71.899 | 35.585 | 1.00 | 15.97 | B |
| ATOM | 3723 | CG2 | VAL | B | 234 | 56.181 | 72.272 | 37.602 | 1.00 | 15.87 | B |
| ATOM | 3724 | C | VAL | B | 234 | 53.635 | 74.445 | 35.753 | 1.00 | 13.01 | B |
| ATOM | 3725 | O | VAL | B | 234 | 53.917 | 74.715 | 34.586 | 1.00 | 12.10 | B |
| ATOM | 3726 | N | ASN | B | 235 | 52.382 | 74.427 | 36.199 | 1.00 | 12.91 | B |
| ATOM | 3727 | CA | ASN | B | 235 | 51.275 | 74.729 | 35.303 | 1.00 | 12.22 | B |
| ATOM | 3728 | CB | ASN | B | 235 | 49.948 | 74.416 | 35.998 | 1.00 | 12.94 | B |
| ATOM | 3729 | CG | ASN | B | 235 | 48.758 | 74.628 | 35.086 | 1.00 | 12.76 | B |
| ATOM | 3730 | OD1 | ASN | B | 235 | 48.660 | 74.013 | 34.025 | 1.00 | 12.94 | B |
| ATOM | 3731 | ND2 | ASN | B | 235 | 47.849 | 75.512 | 35.488 | 1.00 | 13.11 | B |
| ATOM | 3732 | C | ASN | B | 235 | 51.356 | 76.175 | 34.820 | 1.00 | 12.14 | B |
| ATOM | 3733 | O | ASN | B | 235 | 51.184 | 76.449 | 33.634 | 1.00 | 12.71 | B |
| ATOM | 3734 | N | LEU | B | 236 | 51.607 | 77.102 | 35.741 | 1.00 | 12.49 | B |
| ATOM | 3735 | CA | LEU | B | 236 | 51.708 | 78.505 | 35.364 | 1.00 | 11.62 | B |
| ATOM | 3736 | CB | LEU | B | 236 | 51.878 | 79.369 | 36.618 | 1.00 | 13.06 | B |
| ATOM | 3737 | CG | LEU | B | 236 | 50.698 | 79.361 | 37.594 | 1.00 | 14.67 | B |
| ATOM | 3738 | CD1 | LEU | B | 236 | 50.983 | 80.306 | 38.747 | 1.00 | 16.14 | B |
| ATOM | 3739 | CD2 | LEU | B | 236 | 49.426 | 79.778 | 36.872 | 1.00 | 16.50 | B |
| ATOM | 3740 | C | LEU | B | 236 | 52.841 | 78.678 | 34.367 | 1.00 | 11.05 | B |
| ATOM | 3741 | O | LEU | B | 236 | 52.729 | 79.461 | 33.433 | 1.00 | 10.82 | B |
| ATOM | 3742 | N | ALA | B | 237 | 53.931 | 77.942 | 34.563 | 1.00 | 10.62 | B |
| ATOM | 3743 | CA | ALA | B | 237 | 55.071 | 78.039 | 33.658 | 1.00 | 11.05 | B |
| ATOM | 3744 | CB | ALA | B | 237 | 56.226 | 77.198 | 34.174 | 1.00 | 11.84 | B |
| ATOM | 3745 | C | ALA | B | 237 | 54.670 | 77.585 | 32.257 | 1.00 | 8.78 | B |
| ATOM | 3746 | O | ALA | B | 237 | 55.036 | 78.210 | 31.261 | 1.00 | 10.25 | B |
| ATOM | 3747 | N | VAL | B | 238 | 53.916 | 76.493 | 32.175 | 1.00 | 8.92 | B |
| ATOM | 3748 | CA | VAL | B | 238 | 53.468 | 75.998 | 30.882 | 1.00 | 9.79 | B |
| ATOM | 3749 | CB | VAL | B | 238 | 52.646 | 74.697 | 31.038 | 1.00 | 8.65 | B |
| ATOM | 3750 | CG1 | VAL | B | 238 | 51.961 | 74.350 | 29.726 | 1.00 | 11.53 | B |
| ATOM | 3751 | CG2 | VAL | B | 238 | 53.563 | 73.557 | 31.469 | 1.00 | 10.18 | B |
| ATOM | 3752 | C | VAL | B | 238 | 52.615 | 77.059 | 30.188 | 1.00 | 9.93 | B |
| ATOM | 3753 | O | VAL | B | 238 | 52.772 | 77.318 | 28.992 | 1.00 | 9.81 | B |
| ATOM | 3754 | N | LEU | B | 239 | 51.713 | 77.684 | 30.940 | 1.00 | 10.23 | B |
| ATOM | 3755 | CA | LEU | B | 239 | 50.861 | 78.704 | 30.351 | 1.00 | 9.82 | B |
| ATOM | 3756 | CB | LEU | B | 239 | 49.767 | 79.092 | 31.349 | 1.00 | 12.39 | B |
| ATOM | 3757 | CG | LEU | B | 239 | 48.681 | 78.022 | 31.517 | 1.00 | 13.67 | B |
| ATOM | 3758 | CD1 | LEU | B | 239 | 47.624 | 78.512 | 32.492 | 1.00 | 14.95 | B |
| ATOM | 3759 | CD2 | LEU | B | 239 | 48.046 | 77.710 | 30.169 | 1.00 | 16.99 | B |
| ATOM | 3760 | C | LEU | B | 239 | 51.683 | 79.887 | 29.853 | 1.00 | 9.15 | B |
| ATOM | 3761 | O | LEU | B | 239 | 51.403 | 80.427 | 28.786 | 1.00 | 9.90 | B |
| ATOM | 3762 | N | LYS | B | 240 | 52.700 | 80.287 | 30.611 | 1.00 | 11.16 | B |
| ATOM | 3763 | CA | LYS | B | 240 | 53.535 | 81.405 | 30.184 | 1.00 | 10.48 | B |
| ATOM | 3764 | CB | LYS | B | 240 | 54.473 | 81.823 | 31.317 | 1.00 | 12.71 | B |
| ATOM | 3765 | CG | LYS | B | 240 | 55.399 | 82.972 | 30.928 | 1.00 | 14.68 | B |
| ATOM | 3766 | CD | LYS | B | 240 | 56.197 | 83.484 | 32.115 | 1.00 | 20.09 | B |
| ATOM | 3767 | CE | LYS | B | 240 | 55.297 | 84.164 | 33.136 | 1.00 | 22.68 | B |
| ATOM | 3768 | NZ | LYS | B | 240 | 56.076 | 84.727 | 34.276 | 1.00 | 24.21 | B |
| ATOM | 3769 | C | LYS | B | 240 | 54.279 | 81.045 | 28.905 | 1.00 | 10.44 | B |
| ATOM | 3770 | O | LYS | B | 240 | 54.281 | 81.819 | 27.949 | 1.00 | 11.84 | B |
| ATOM | 3771 | N | LEU | B | 241 | 54.909 | 79.871 | 28.884 | 1.00 | 10.42 | B |
| ATOM | 3772 | CA | LEU | B | 241 | 55.649 | 79.440 | 27.698 | 1.00 | 10.58 | B |
| ATOM | 3773 | CB | LEU | B | 241 | 56.324 | 78.091 | 27.976 | 1.00 | 10.58 | B |
| ATOM | 3774 | CG | LEU | B | 241 | 57.391 | 78.072 | 29.078 | 1.00 | 10.95 | B |
| ATOM | 3775 | CD1 | LEU | B | 241 | 57.857 | 76.646 | 29.303 | 1.00 | 11.46 | B |
| ATOM | 3776 | CD2 | LEU | B | 241 | 58.559 | 78.971 | 28.691 | 1.00 | 12.35 | B |
| ATOM | 3777 | C | LEU | B | 241 | 54.717 | 79.409 | 26.493 | 1.00 | 10.01 | B |
| ATOM | 3778 | O | LEU | B | 241 | 55.105 | 79.781 | 25.387 | 1.00 | 11.26 | B |
| ATOM | 3779 | N | SER | B | 242 | 53.485 | 78.958 | 26.712 | 1.00 | 9.61 | B |
| ATOM | 3780 | CA | SER | B | 242 | 52.489 | 78.907 | 25.652 | 1.00 | 10.02 | B |
| ATOM | 3781 | CB | SER | B | 242 | 51.216 | 78.249 | 26.186 | 1.00 | 9.83 | B |
| ATOM | 3782 | OG | SER | B | 242 | 50.149 | 78.410 | 25.262 | 1.00 | 11.46 | B |
| ATOM | 3783 | C | SER | B | 242 | 52.217 | 80.307 | 25.121 | 1.00 | 9.68 | B |
| ATOM | 3784 | O | SER | B | 242 | 52.314 | 80.558 | 23.921 | 1.00 | 10.65 | B |
| ATOM | 3785 | N | GLU | B | 243 | 51.870 | 81.212 | 26.032 | 1.00 | 10.68 | B |
| ATOM | 3786 | CA | GLU | B | 243 | 51.541 | 82.582 | 25.666 | 1.00 | 12.37 | B |
| ATOM | 3787 | CB | GLU | B | 243 | 50.899 | 83.274 | 26.876 | 1.00 | 12.07 | B |
| ATOM | 3788 | CG | GLU | B | 243 | 49.549 | 82.660 | 27.240 | 1.00 | 12.41 | B |
| ATOM | 3789 | CD | GLU | B | 243 | 48.811 | 83.390 | 28.345 | 1.00 | 12.64 | B |
| ATOM | 3790 | OE1 | GLU | B | 243 | 49.314 | 84.415 | 28.855 | 1.00 | 14.32 | B |
| ATOM | 3791 | OE2 | GLU | B | 243 | 47.710 | 82.925 | 28.704 | 1.00 | 13.44 | B |
| ATOM | 3792 | C | GLU | B | 243 | 52.694 | 83.364 | 25.054 | 1.00 | 11.58 | B |
| ATOM | 3793 | O | GLU | B | 243 | 52.460 | 84.308 | 24.295 | 1.00 | 13.14 | B |
| ATOM | 3794 | N | GLN | B | 244 | 53.927 | 82.974 | 25.367 | 1.00 | 12.52 | B |
| ATOM | 3795 | CA | GLN | B | 244 | 55.107 | 83.650 | 24.830 | 1.00 | 12.54 | B |
| ATOM | 3796 | CB | GLN | B | 244 | 56.275 | 83.506 | 25.811 | 1.00 | 15.35 | B |
| ATOM | 3797 | CG | GLN | B | 244 | 56.078 | 84.191 | 27.149 | 1.00 | 19.99 | B |
| ATOM | 3798 | CD | GLN | B | 244 | 57.242 | 83.953 | 28.095 | 1.00 | 24.17 | B |
| ATOM | 3799 | OE1 | GLN | B | 244 | 57.579 | 82.809 | 28.409 | 1.00 | 28.41 | B |
| ATOM | 3800 | NE2 | GLN | B | 244 | 57.860 | 85.032 | 28.555 | 1.00 | 26.83 | B |
| ATOM | 3801 | C | GLN | B | 244 | 55.457 | 83.116 | 23.450 | 1.00 | 12.24 | B |
| ATOM | 3802 | O | GLN | B | 244 | 56.320 | 83.672 | 22.769 | 1.00 | 13.81 | B |
| ATOM | 3803 | N | GLY | B | 245 | 54.795 | 82.038 | 23.043 | 1.00 | 12.05 | B |
| ATOM | 3804 | CA | GLY | B | 245 | 55.061 | 81.440 | 21.745 | 1.00 | 12.13 | B |
| ATOM | 3805 | C | GLY | B | 245 | 56.273 | 80.523 | 21.751 | 1.00 | 10.48 | B |
| ATOM | 3806 | O | GLY | B | 245 | 56.667 | 79.992 | 20.710 | 1.00 | 11.40 | B |
| ATOM | 3807 | N | LEU | B | 246 | 56.850 | 80.316 | 22.929 | 1.00 | 10.40 | B |
| ATOM | 3808 | CA | LEU | B | 246 | 58.035 | 79.473 | 23.072 | 1.00 | 11.35 | B |
| ATOM | 3809 | CB | LEU | B | 246 | 58.634 | 79.705 | 24.461 | 1.00 | 12.45 | B |
| ATOM | 3810 | CG | LEU | B | 246 | 59.258 | 81.086 | 24.690 | 1.00 | 15.77 | B |
| ATOM | 3811 | CD1 | LEU | B | 246 | 59.700 | 81.221 | 26.139 | 1.00 | 19.66 | B |
| ATOM | 3812 | CD2 | LEU | B | 246 | 60.440 | 81.273 | 23.750 | 1.00 | 17.98 | B |
| ATOM | 3813 | C | LEU | B | 246 | 57.770 | 77.999 | 22.771 | 1.00 | 10.58 | B |
| ATOM | 3814 | O | LEU | B | 246 | 58.653 | 77.292 | 22.283 | 1.00 | 11.68 | B |
| ATOM | 3815 | N | LEU | B | 247 | 56.566 | 77.521 | 23.063 | 1.00 | 10.70 | B |
| ATOM | 3816 | CA | LEU | B | 247 | 56.247 | 76.127 | 22.787 | 1.00 | 10.56 | B |
| ATOM | 3817 | CB | LEU | B | 247 | 54.971 | 75.736 | 23.539 | 1.00 | 12.11 | B |
| ATOM | 3818 | CG | LEU | B | 247 | 55.059 | 75.852 | 25.063 | 1.00 | 11.27 | B |
| ATOM | 3819 | CD1 | LEU | B | 247 | 53.732 | 75.415 | 25.671 | 1.00 | 11.53 | B |
| ATOM | 3820 | CD2 | LEU | B | 247 | 56.198 | 74.997 | 25.601 | 1.00 | 13.61 | B |
| ATOM | 3821 | C | LEU | B | 247 | 56.161 | 75.934 | 21.277 | 1.00 | 10.81 | B |
| ATOM | 3822 | O | LEU | B | 247 | 56.552 | 74.891 | 20.760 | 1.00 | 10.76 | B |
| ATOM | 3823 | N | ASP | B | 248 | 55.654 | 76.938 | 20.565 | 1.00 | 10.50 | B |
| ATOM | 3824 | CA | ASP | B | 248 | 55.564 | 76.843 | 19.112 | 1.00 | 12.17 | B |
| ATOM | 3825 | CB | ASP | B | 248 | 54.736 | 78.007 | 18.552 | 1.00 | 12.81 | B |
| ATOM | 3826 | CG | ASP | B | 248 | 53.288 | 77.971 | 18.998 | 1.00 | 14.09 | B |
| ATOM | 3827 | OD1 | ASP | B | 248 | 52.693 | 76.873 | 19.027 | 1.00 | 16.17 | B |
| ATOM | 3828 | OD2 | ASP | B | 248 | 52.737 | 79.050 | 19.302 | 1.00 | 17.55 | B |
| ATOM | 3829 | C | ASP | B | 248 | 56.979 | 76.806 | 18.537 | 1.00 | 12.32 | B |
| ATOM | 3830 | O | ASP | B | 248 | 57.263 | 76.046 | 17.608 | 1.00 | 13.88 | B |
| ATOM | 3831 | N | LYS | B | 249 | 57.862 | 77.631 | 19.091 | 1.00 | 12.62 | B |
| ATOM | 3832 | CA | LYS | B | 249 | 59.243 | 77.686 | 18.628 | 1.00 | 13.25 | B |
| ATOM | 3833 | CB | LYS | B | 249 | 59.971 | 78.828 | 19.343 | 1.00 | 15.24 | B |
| ATOM | 3834 | CG | LYS | B | 249 | 61.402 | 79.036 | 18.878 | 1.00 | 21.15 | B |
| ATOM | 3835 | CD | LYS | B | 249 | 62.062 | 80.194 | 19.609 | 1.00 | 23.46 | B |
| ATOM | 3836 | CE | LYS | B | 249 | 63.487 | 80.412 | 19.123 | 1.00 | 25.24 | B |
| ATOM | 3837 | NZ | LYS | B | 249 | 63.529 | 80.703 | 17.659 | 1.00 | 27.50 | B |
| ATOM | 3838 | C | LYS | B | 249 | 59.922 | 76.337 | 18.830 | 1.00 | 11.89 | B |
| ATOM | 3839 | O | LYS | B | 249 | 60.638 | 75.860 | 17.948 | 1.00 | 11.35 | B |
| ATOM | 3840 | N | LEU | B | 250 | 59.696 | 75.727 | 19.990 | 1.00 | 11.05 | B |
| ATOM | 3841 | CA | LEU | B | 250 | 60.292 | 74.434 | 20.296 | 1.00 | 12.56 | B |
| ATOM | 3842 | CB | LEU | B | 250 | 60.072 | 74.106 | 21.776 | 1.00 | 14.40 | B |
| ATOM | 3843 | CG | LEU | B | 250 | 60.913 | 74.904 | 22.777 | 1.00 | 15.38 | B |
| ATOM | 3844 | CD1 | LEU | B | 250 | 60.628 | 74.408 | 24.188 | 1.00 | 18.75 | B |
| ATOM | 3845 | CD2 | LEU | B | 250 | 62.391 | 74.745 | 22.451 | 1.00 | 19.61 | B |
| ATOM | 3846 | C | LEU | B | 250 | 59.791 | 73.343 | 19.358 | 1.00 | 10.20 | B |
| ATOM | 3847 | O | LEU | B | 250 | 60.569 | 72.483 | 18.944 | 1.00 | 11.44 | B |
| ATOM | 3848 | N | LYS | B | 251 | 58.504 | 73.360 | 19.009 | 1.00 | 10.95 | B |
| ATOM | 3849 | CA | LYS | B | 251 | 58.017 | 72.330 | 18.097 | 1.00 | 12.43 | B |
| ATOM | 3850 | CB | LYS | B | 251 | 56.485 | 72.312 | 18.023 | 1.00 | 13.29 | B |
| ATOM | 3851 | CG | LYS | B | 251 | 55.996 | 71.181 | 17.113 | 1.00 | 16.55 | B |
| ATOM | 3852 | CD | LYS | B | 251 | 54.490 | 71.004 | 17.085 | 1.00 | 17.86 | B |
| ATOM | 3853 | CE | LYS | B | 251 | 54.135 | 69.736 | 16.308 | 1.00 | 19.60 | B |
| ATOM | 3854 | NZ | LYS | B | 251 | 52.675 | 69.544 | 16.140 | 1.00 | 20.85 | B |
| ATOM | 3855 | C | LYS | B | 251 | 58.654 | 72.517 | 16.726 | 1.00 | 11.79 | B |
| ATOM | 3856 | O | LYS | B | 251 | 59.086 | 71.549 | 16.105 | 1.00 | 11.81 | B |
| ATOM | 3857 | N | ASN | B | 252 | 58.713 | 73.759 | 16.250 | 1.00 | 11.81 | B |
| ATOM | 3858 | CA | ASN | B | 252 | 59.316 | 74.021 | 14.946 | 1.00 | 13.39 | B |
| ATOM | 3859 | CB | ASN | B | 252 | 59.170 | 75.502 | 14.585 | 1.00 | 16.26 | B |
| ATOM | 3860 | CG | ASN | B | 252 | 57.746 | 75.882 | 14.262 | 1.00 | 19.99 | B |
| ATOM | 3861 | OD1 | ASN | B | 252 | 57.000 | 75.092 | 13.684 | 1.00 | 23.83 | B |
| ATOM | 3862 | ND2 | ASN | B | 252 | 57.363 | 77.103 | 14.614 | 1.00 | 22.55 | B |
| ATOM | 3863 | C | ASN | B | 252 | 60.764 | 73.564 | 14.941 | 1.00 | 13.53 | B |
| ATOM | 3864 | O | ASN | B | 252 | 61.230 | 72.966 | 13.974 | 1.00 | 13.33 | B |
| ATOM | 3865 | N | LYS | B | 253 | 61.471 | 73.844 | 16.029 | 1.00 | 12.13 | B |
| ATOM | 3866 | CA | LYS | B | 253 | 62.872 | 73.461 | 16.147 | 1.00 | 13.00 | B |
| ATOM | 3867 | CB | LYS | B | 253 | 63.413 | 73.951 | 17.495 | 1.00 | 14.25 | B |
| ATOM | 3868 | CG | LYS | B | 253 | 64.812 | 73.439 | 17.823 | 1.00 | 13.83 | B |
| ATOM | 3869 | CD | LYS | B | 253 | 65.278 | 73.913 | 19.192 | 1.00 | 14.51 | B |
| ATOM | 3870 | CE | LYS | B | 253 | 66.663 | 73.377 | 19.522 | 1.00 | 17.96 | B |
| ATOM | 3871 | NZ | LYS | B | 253 | 67.083 | 73.685 | 20.922 | 1.00 | 20.12 | B |
| ATOM | 3872 | C | LYS | B | 253 | 63.098 | 71.966 | 15.954 | 1.00 | 12.93 | B |
| ATOM | 3873 | O | LYS | B | 253 | 63.931 | 71.555 | 15.139 | 1.00 | 13.54 | B |
| ATOM | 3874 | N | TRP | B | 254 | 62.350 | 71.154 | 16.696 | 1.00 | 11.94 | B |
| ATOM | 3875 | CA | TRP | B | 254 | 62.516 | 69.709 | 16.654 | 1.00 | 12.21 | B |
| ATOM | 3876 | CB | TRP | B | 254 | 62.240 | 69.165 | 18.063 | 1.00 | 12.24 | B |
| ATOM | 3877 | CG | TRP | B | 254 | 63.252 | 69.634 | 19.062 | 1.00 | 10.70 | B |
| ATOM | 3878 | CD2 | TRP | B | 254 | 64.591 | 69.148 | 19.204 | 1.00 | 9.88 | B |
| ATOM | 3879 | CE2 | TRP | B | 254 | 65.213 | 69.920 | 20.210 | 1.00 | 10.80 | B |
| ATOM | 3880 | CE3 | TRP | B | 254 | 65.327 | 68.137 | 18.573 | 1.00 | 10.93 | B |
| ATOM | 3881 | CD1 | TRP | B | 254 | 63.116 | 70.653 | 19.965 | 1.00 | 10.24 | B |
| ATOM | 3882 | NE1 | TRP | B | 254 | 64.292 | 70.831 | 20.659 | 1.00 | 10.60 | B |
| ATOM | 3883 | CZ2 | TRP | B | 254 | 66.540 | 69.711 | 20.600 | 1.00 | 10.82 | B |
| ATOM | 3884 | CZ3 | TRP | B | 254 | 66.647 | 67.929 | 18.960 | 1.00 | 11.90 | B |
| ATOM | 3885 | CH2 | TRP | B | 254 | 67.238 | 68.713 | 19.965 | 1.00 | 10.73 | B |
| ATOM | 3886 | C | TRP | B | 254 | 61.752 | 68.957 | 15.571 | 1.00 | 10.95 | B |
| ATOM | 3887 | O | TRP | B | 254 | 62.073 | 67.801 | 15.287 | 1.00 | 12.40 | B |
| ATOM | 3888 | N | TRP | B | 255 | 60.765 | 69.598 | 14.957 | 1.00 | 11.38 | B |
| ATOM | 3889 | CA | TRP | B | 255 | 59.962 | 68.952 | 13.923 | 1.00 | 13.10 | B |
| ATOM | 3890 | CB | TRP | B | 255 | 58.475 | 69.182 | 14.224 | 1.00 | 12.80 | B |
| ATOM | 3891 | CG | TRP | B | 255 | 57.813 | 68.115 | 15.019 | 1.00 | 10.68 | B |
| ATOM | 3892 | CD2 | TRP | B | 255 | 57.957 | 67.882 | 16.420 | 1.00 | 9.63 | B |
| ATOM | 3893 | CE2 | TRP | B | 255 | 57.149 | 66.771 | 16.742 | 1.00 | 10.00 | B |
| ATOM | 3894 | CE3 | TRP | B | 255 | 58.693 | 68.505 | 17.437 | 1.00 | 10.25 | B |
| ATOM | 3895 | CD1 | TRP | B | 255 | 56.947 | 67.167 | 14.556 | 1.00 | 11.48 | B |
| ATOM | 3896 | NE1 | TRP | B | 255 | 56.543 | 66.355 | 15.586 | 1.00 | 10.11 | B |
| ATOM | 3897 | CZ2 | TRP | B | 255 | 57.054 | 66.265 | 18.041 | 1.00 | 10.61 | B |
| ATOM | 3898 | CZ3 | TRP | B | 255 | 58.598 | 68.002 | 18.732 | 1.00 | 10.77 | B |
| ATOM | 3899 | CH2 | TRP | B | 255 | 57.783 | 66.890 | 19.021 | 1.00 | 11.15 | B |
| ATOM | 3900 | C | TRP | B | 255 | 60.289 | 69.392 | 12.505 | 1.00 | 14.72 | B |
| ATOM | 3901 | O | TRP | B | 255 | 60.491 | 68.560 | 11.620 | 1.00 | 15.12 | B |
| ATOM | 3902 | N | TYR | B | 256 | 60.349 | 70.702 | 12.295 | 1.00 | 17.10 | B |
| ATOM | 3903 | CA | TYR | B | 256 | 60.544 | 71.233 | 10.954 | 1.00 | 19.22 | B |
| ATOM | 3904 | CB | TYR | B | 256 | 59.340 | 72.129 | 10.666 | 1.00 | 19.05 | B |
| ATOM | 3905 | CG | TYR | B | 256 | 58.030 | 71.468 | 11.035 | 1.00 | 19.92 | B |
| ATOM | 3906 | CD1 | TYR | B | 256 | 57.599 | 70.317 | 10.378 | 1.00 | 22.52 | B |
| ATOM | 3907 | CE1 | TYR | B | 256 | 56.415 | 69.677 | 10.746 | 1.00 | 21.25 | B |
| ATOM | 3908 | CD2 | TYR | B | 256 | 57.242 | 71.968 | 12.071 | 1.00 | 20.19 | B |
| ATOM | 3909 | CE2 | TYR | B | 256 | 56.059 | 71.336 | 12.446 | 1.00 | 22.92 | B |
| ATOM | 3910 | CZ | TYR | B | 256 | 55.653 | 70.191 | 11.779 | 1.00 | 22.06 | B |
| ATOM | 3911 | OH | TYR | B | 256 | 54.491 | 69.554 | 12.153 | 1.00 | 24.67 | B |
| ATOM | 3912 | C | TYR | B | 256 | 61.871 | 71.894 | 10.609 | 1.00 | 21.40 | B |
| ATOM | 3913 | O | TYR | B | 256 | 62.350 | 71.749 | 9.482 | 1.00 | 22.61 | B |
| ATOM | 3914 | N | ASP | B | 257 | 62.464 | 72.623 | 11.547 | 1.00 | 23.95 | B |
| ATOM | 3915 | CA | ASP | B | 257 | 63.732 | 73.290 | 11.266 | 1.00 | 27.44 | B |
| ATOM | 3916 | CB | ASP | B | 257 | 64.165 | 74.112 | 12.487 | 1.00 | 27.78 | B |
| ATOM | 3917 | CG | ASP | B | 257 | 63.211 | 75.254 | 12.789 | 1.00 | 29.85 | B |
| ATOM | 3918 | OD1 | ASP | B | 257 | 62.565 | 75.759 | 11.846 | 1.00 | 32.11 | B |
| ATOM | 3919 | OD2 | ASP | B | 257 | 63.118 | 75.659 | 13.966 | 1.00 | 31.30 | B |
| ATOM | 3920 | C | ASP | B | 257 | 64.784 | 72.285 | 10.812 | 1.00 | 29.29 | B |
| ATOM | 3921 | O | ASP | B | 257 | 65.688 | 72.622 | 10.047 | 1.00 | 30.78 | B |
| ATOM | 3922 | N | GLY | B | 258 | 64.659 | 71.050 | 11.283 | 1.00 | 31.43 | B |
| ATOM | 3923 | CA | GLY | B | 258 | 65.605 | 70.017 | 10.904 | 1.00 | 32.34 | B |
| ATOM | 3924 | C | GLY | B | 258 | 65.005 | 69.063 | 9.891 | 1.00 | 33.02 | B |
| ATOM | 3925 | O | GLY | B | 258 | 65.565 | 68.000 | 9.619 | 1.00 | 33.65 | B |
| ATOM | 3926 | N | GLY | B | 259 | 63.860 | 69.445 | 9.332 | 1.00 | 33.63 | B |
| ATOM | 3927 | CA | GLY | B | 259 | 63.195 | 68.610 | 8.348 | 1.00 | 33.47 | B |
| ATOM | 3928 | C | GLY | B | 259 | 64.149 | 68.089 | 7.291 | 1.00 | 34.04 | B |
| ATOM | 3929 | O | GLY | B | 259 | 65.116 | 68.761 | 6.933 | 1.00 | 34.51 | B |
| ATOM | 3930 | N | GLU | B | 260 | 63.878 | 66.888 | 6.792 | 1.00 | 33.23 | B |
| ATOM | 3931 | CA | GLU | B | 260 | 64.722 | 66.272 | 5.775 | 1.00 | 32.24 | B |
| ATOM | 3932 | CB | GLU | B | 260 | 65.348 | 65.000 | 6.355 | 1.00 | 31.96 | B |
| ATOM | 3933 | CG | GLU | B | 260 | 66.243 | 65.236 | 7.560 | 1.00 | 32.09 | B |
| ATOM | 3934 | CD | GLU | B | 260 | 66.683 | 63.943 | 8.220 | 1.00 | 32.53 | B |
| ATOM | 3935 | OE1 | GLU | B | 260 | 67.511 | 64.000 | 9.154 | 1.00 | 33.55 | B |
| ATOM | 3936 | OE2 | GLU | B | 260 | 66.196 | 62.868 | 7.808 | 1.00 | 32.24 | B |
| ATOM | 3937 | C | GLU | B | 260 | 63.916 | 66.009 | 4.512 | 1.00 | 31.67 | B |
| ATOM | 3938 | O | GLU | B | 260 | 64.439 | 65.473 | 3.534 | 1.00 | 32.39 | B |
| ATOM | 3939 | N | CYS | B | 261 | 62.643 | 66.388 | 4.534 | 1.00 | 31.23 | B |
| ATOM | 3940 | CA | CYS | B | 261 | 61.765 | 66.175 | 3.391 | 1.00 | 31.10 | B |
| ATOM | 3941 | CB | CYS | B | 261 | 60.671 | 65.181 | 3.789 | 1.00 | 28.33 | B |
| ATOM | 3942 | SG | CYS | B | 261 | 61.295 | 63.662 | 4.576 | 1.00 | 22.63 | B |
| ATOM | 3943 | C | CYS | B | 261 | 61.212 | 67.499 | 2.882 | 1.00 | 33.06 | B |
| ATOM | 3944 | OT1 | CYS | B | 261 | 61.395 | 67.786 | 1.682 | 1.00 | 34.09 | B |
| ATOM | 3945 | OT2 | CYS | B | 261 | 60.596 | 68.229 | 3.687 | 1.00 | 34.77 | B |
| ATOM | 3946 | N | KAI | C | 999 | 36.732 | 83.699 | 24.381 | 1.00 | 8.97 | C |
| ATOM | 3947 | CD | KAI | C | 999 | 35.356 | 84.162 | 23.978 | 1.00 | 11.13 | C |
| ATOM | 3948 | CD1 | KAI | C | 999 | 33.448 | 86.124 | 23.049 | 1.00 | 11.15 | C |
| ATOM | 3949 | CD2 | KAI | C | 999 | 34.433 | 88.083 | 24.099 | 1.00 | 10.37 | C |
| ATOM | 3950 | CA | KAI | C | 999 | 37.571 | 84.834 | 24.580 | 1.00 | 9.63 | C |
| ATOM | 3951 | CB | KAI | C | 999 | 36.895 | 86.049 | 23.967 | 1.00 | 9.40 | C |
| ATOM | 3952 | CB1 | KAI | C | 999 | 37.007 | 86.149 | 22.439 | 1.00 | 9.89 | C |
| ATOM | 3953 | CG | KAI | C | 999 | 35.496 | 85.730 | 24.227 | 1.00 | 10.71 | C |
| ATOM | 3954 | CG1 | KAI | C | 999 | 38.226 | 86.898 | 21.921 | 1.00 | 11.01 | C |
| ATOM | 3955 | CG2 | KAI | C | 999 | 34.361 | 86.658 | 23.754 | 1.00 | 9.12 | C |
| ATOM | 3956 | C | KAI | C | 999 | 37.920 | 85.004 | 26.067 | 1.00 | 9.43 | C |
| ATOM | 3957 | O | KAI | C | 999 | 37.684 | 84.018 | 26.801 | 1.00 | 9.25 | C |
| ATOM | 3958 | OD1 | KAI | C | 999 | 38.397 | 86.868 | 20.684 | 1.00 | 11.02 | C |
| ATOM | 3959 | OD2 | KAI | C | 999 | 38.955 | 87.487 | 22.751 | 1.00 | 11.20 | C |
| ATOM | 3960 | OT | KAI | C | 999 | 38.417 | 86.102 | 26.400 | 1.00 | 9.41 | C |
| ATOM | 3961 | N1 | LY3 | E | 888 | 50.824 | 75.300 | 24.218 | 0.50 | 14.69 | E |
| ATOM | 3962 | C2 | LY3 | E | 888 | 49.837 | 74.767 | 24.101 | 0.50 | 12.99 | E |
| ATOM | 3963 | C3 | LY3 | E | 888 | 48.551 | 74.064 | 23.955 | 0.50 | 14.06 | E |
| ATOM | 3964 | C4 | LY3 | E | 888 | 47.446 | 74.389 | 24.833 | 0.50 | 11.93 | E |
| ATOM | 3965 | C5 | LY3 | E | 888 | 46.201 | 73.657 | 24.724 | 0.50 | 11.26 | E |
| ATOM | 3966 | C6 | LY3 | E | 888 | 46.065 | 72.599 | 23.727 | 0.50 | 9.88 | E |
| ATOM | 3967 | C7 | LY3 | E | 888 | 47.174 | 72.297 | 22.862 | 0.50 | 12.86 | E |
| ATOM | 3968 | C8 | LY3 | E | 888 | 48.410 | 73.022 | 22.968 | 0.50 | 13.14 | E |
| ATOM | 3969 | C9 | LY3 | E | 888 | 44.688 | 71.853 | 23.704 | 0.50 | 9.90 | E |
| ATOM | 3970 | C10 | LY3 | E | 888 | 43.409 | 72.534 | 23.750 | 0.50 | 8.29 | E |
| ATOM | 3971 | C11 | LY3 | E | 888 | 42.177 | 71.761 | 23.749 | 0.50 | 6.63 | E |
| ATOM | 3972 | C12 | LY3 | E | 888 | 42.220 | 70.314 | 23.697 | 0.50 | 8.27 | E |
| ATOM | 3973 | C13 | LY3 | E | 888 | 43.501 | 69.642 | 23.639 | 0.50 | 9.35 | E |
| ATOM | 3974 | C14 | LY3 | E | 888 | 44.721 | 70.401 | 23.646 | 0.50 | 9.41 | E |
| ATOM | 3975 | C15 | LY3 | E | 888 | 40.937 | 69.538 | 23.732 | 0.50 | 6.94 | E |
| ATOM | 3976 | C16 | LY3 | E | 888 | 40.519 | 69.153 | 22.327 | 0.50 | 9.61 | E |
| ATOM | 3977 | C17 | LY3 | E | 888 | 41.080 | 68.279 | 24.574 | 0.50 | 9.87 | E |
| ATOM | 3978 | N18 | LY3 | E | 888 | 41.562 | 68.515 | 25.961 | 0.50 | 9.17 | E |
| ATOM | 3979 | S19 | LY3 | E | 888 | 42.499 | 67.367 | 26.745 | 0.50 | 8.97 | E |
| ATOM | 3980 | O20 | LY3 | E | 888 | 43.612 | 67.035 | 25.887 | 0.50 | 10.09 | E |
| ATOM | 3981 | O21 | LY3 | E | 888 | 42.912 | 68.015 | 27.994 | 0.50 | 10.41 | E |
| ATOM | 3982 | C22 | LY3 | E | 888 | 41.536 | 66.117 | 26.870 | 0.50 | 9.66 | E |
| ATOM | 3983 | C23 | LY3 | E | 888 | 42.386 | 65.070 | 27.545 | 0.50 | 14.10 | E |
| ATOM | 3984 | C24 | LY3 | E | 888 | 40.409 | 66.529 | 27.730 | 0.50 | 11.22 | E |
| ATOM | 3985 | OH2 | WAT | W | 1 | 47.102 | 58.092 | 22.068 | 1.00 | 9.42 | W |
| ATOM | 3986 | OH2 | WAT | W | 2 | 41.892 | 84.589 | 22.087 | 1.00 | 9.65 | W |
| ATOM | 3987 | OH2 | WAT | W | 3 | 36.678 | 85.311 | 19.219 | 1.00 | 9.95 | W |
| ATOM | 3988 | OH2 | WAT | W | 4 | 52.289 | 57.357 | 19.194 | 1.00 | 10.29 | W |
| ATOM | 3989 | OH2 | WAT | W | 5 | 46.379 | 81.533 | 26.710 | 1.00 | 10.86 | W |
| ATOM | 3990 | OH2 | WAT | W | 6 | 33.845 | 77.899 | 30.965 | 1.00 | 11.49 | W |
| ATOM | 3991 | OH2 | WAT | W | 7 | 50.870 | 71.891 | 20.044 | 1.00 | 11.56 | W |
| ATOM | 3992 | OH2 | WAT | W | 8 | 55.114 | 64.760 | 30.992 | 1.00 | 11.41 | W |
| ATOM | 3993 | OH2 | WAT | W | 9 | 55.092 | 72.606 | 21.327 | 1.00 | 12.00 | W |
| ATOM | 3994 | OH2 | WAT | W | 10 | 48.599 | 82.653 | 23.121 | 1.00 | 12.64 | W |
| ATOM | 3995 | OH2 | WAT | W | 11 | 38.122 | 70.791 | 20.060 | 1.00 | 12.16 | W |
| ATOM | 3996 | OH2 | WAT | W | 12 | 40.419 | 60.055 | 23.138 | 1.00 | 12.75 | W |
| ATOM | 3997 | OH2 | WAT | W | 13 | 42.595 | 61.145 | 26.695 | 1.00 | 11.43 | W |
| ATOM | 3998 | OH2 | WAT | W | 14 | 37.945 | 60.776 | 21.876 | 1.00 | 11.88 | W |
| ATOM | 3999 | OH2 | WAT | W | 15 | 68.135 | 69.797 | 23.658 | 1.00 | 12.94 | W |
| ATOM | 4000 | OH2 | WAT | W | 16 | 33.881 | 70.066 | 21.295 | 1.00 | 12.65 | W |
| ATOM | 4001 | OH2 | WAT | W | 17 | 39.517 | 90.174 | 22.369 | 1.00 | 11.67 | W |
| ATOM | 4002 | OH2 | WAT | W | 18 | 20.860 | 72.869 | 23.649 | 1.00 | 14.37 | W |
| ATOM | 4003 | OH2 | WAT | W | 19 | 40.816 | 62.182 | 24.797 | 1.00 | 12.94 | W |
| ATOM | 4004 | OH2 | WAT | W | 20 | 50.159 | 61.009 | 42.996 | 1.00 | 14.37 | W |
| ATOM | 4005 | OH2 | WAT | W | 21 | 49.483 | 52.474 | 22.361 | 1.00 | 12.62 | W |
| ATOM | 4006 | OH2 | WAT | W | 22 | 41.787 | 101.520 | 16.042 | 1.00 | 12.71 | W |
| ATOM | 4007 | OH2 | WAT | W | 23 | 66.949 | 69.772 | 30.438 | 1.00 | 13.34 | W |
| ATOM | 4008 | OH2 | WAT | W | 24 | 47.186 | 41.155 | 16.033 | 1.00 | 12.63 | W |
| ATOM | 4009 | OH2 | WAT | W | 25 | 22.033 | 72.872 | 30.430 | 1.00 | 13.42 | W |
| ATOM | 4010 | OH2 | WAT | W | 26 | 38.836 | 81.698 | 42.991 | 1.00 | 14.92 | W |
| ATOM | 4011 | OH2 | WAT | W | 27 | 38.981 | 40.210 | 18.711 | 1.00 | 13.28 | W |
| ATOM | 4012 | OH2 | WAT | W | 28 | 45.202 | 83.684 | 25.166 | 1.00 | 13.63 | W |
| ATOM | 4013 | OH2 | WAT | W | 29 | 50.995 | 81.871 | 21.899 | 1.00 | 12.75 | W |
| ATOM | 4014 | OH2 | WAT | W | 30 | 51.886 | 68.752 | 36.988 | 1.00 | 14.14 | W |
| ATOM | 4015 | OH2 | WAT | W | 31 | 53.804 | 78.573 | 22.322 | 1.00 | 13.21 | W |
| ATOM | 4016 | OH2 | WAT | W | 32 | 37.114 | 73.904 | 36.993 | 1.00 | 13.79 | W |
| ATOM | 4017 | OH2 | WAT | W | 33 | 50.186 | 77.511 | 22.712 | 1.00 | 13.81 | W |
| ATOM | 4018 | OH2 | WAT | W | 34 | 49.758 | 96.872 | 9.527 | 1.00 | 14.63 | W |
| ATOM | 4019 | OH2 | WAT | W | 35 | 39.170 | 45.814 | 9.527 | 1.00 | 15.33 | W |
| ATOM | 4020 | OH2 | WAT | W | 36 | 35.210 | 64.071 | 22.298 | 1.00 | 13.53 | W |
| ATOM | 4021 | OH2 | WAT | W | 37 | 43.727 | 58.990 | 25.122 | 1.00 | 14.75 | W |
| ATOM | 4022 | OH2 | WAT | W | 38 | 38.820 | 65.158 | 22.768 | 1.00 | 13.75 | W |
| ATOM | 4023 | OH2 | WAT | W | 39 | 70.814 | 63.704 | 34.074 | 1.00 | 13.03 | W |
| ATOM | 4024 | OH2 | WAT | W | 40 | 46.180 | 87.108 | 37.618 | 1.00 | 15.72 | W |
| ATOM | 4025 | OH2 | WAT | W | 41 | 64.124 | 55.179 | 27.857 | 1.00 | 13.51 | W |
| ATOM | 4026 | OH2 | WAT | W | 42 | 48.163 | 80.493 | 24.804 | 1.00 | 13.14 | W |
| ATOM | 4027 | OH2 | WAT | W | 43 | 24.858 | 87.455 | 27.843 | 1.00 | 13.24 | W |
| ATOM | 4028 | OH2 | WAT | W | 44 | 42.829 | 55.548 | 37.593 | 1.00 | 15.05 | W |
| ATOM | 4029 | OH2 | WAT | W | 45 | 36.635 | 48.648 | 21.536 | 1.00 | 14.77 | W |
| ATOM | 4030 | OH2 | WAT | W | 46 | 43.414 | 105.324 | 17.763 | 1.00 | 15.06 | W |
| ATOM | 4031 | OH2 | WAT | W | 47 | 41.508 | 56.892 | 30.417 | 1.00 | 14.57 | W |
| ATOM | 4032 | OH2 | WAT | W | 48 | 49.989 | 102.506 | 18.712 | 1.00 | 14.52 | W |
| ATOM | 4033 | OH2 | WAT | W | 49 | 25.983 | 88.936 | 29.899 | 1.00 | 14.36 | W |
| ATOM | 4034 | OH2 | WAT | W | 50 | 54.492 | 56.093 | 18.081 | 1.00 | 12.38 | W |
| ATOM | 4035 | OH2 | WAT | W | 51 | 38.480 | 63.295 | 20.839 | 1.00 | 13.21 | W |
| ATOM | 4036 | OH2 | WAT | W | 52 | 34.506 | 86.536 | 18.104 | 1.00 | 13.07 | W |
| ATOM | 4037 | OH2 | WAT | W | 53 | 62.976 | 53.766 | 29.878 | 1.00 | 15.14 | W |
| ATOM | 4038 | OH2 | WAT | W | 54 | 52.374 | 94.064 | 21.522 | 1.00 | 15.10 | W |
| ATOM | 4039 | OH2 | WAT | W | 55 | 32.437 | 75.719 | 43.086 | 1.00 | 18.07 | W |
| ATOM | 4040 | OH2 | WAT | W | 56 | 45.572 | 37.362 | 17.727 | 1.00 | 14.20 | W |
| ATOM | 4041 | OH2 | WAT | W | 57 | 18.811 | 85.476 | 29.350 | 1.00 | 15.28 | W |
| ATOM | 4042 | OH2 | WAT | W | 58 | 44.689 | 74.110 | 20.571 | 1.00 | 13.59 | W |
| ATOM | 4043 | OH2 | WAT | W | 59 | 70.181 | 57.203 | 29.405 | 1.00 | 15.34 | W |
| ATOM | 4044 | OH2 | WAT | W | 60 | 50.535 | 79.365 | 20.810 | 1.00 | 14.25 | W |
| ATOM | 4045 | OH2 | WAT | W | 61 | 59.364 | 59.220 | 6.177 | 1.00 | 13.93 | W |
| ATOM | 4046 | OH2 | WAT | W | 62 | 18.152 | 78.966 | 34.116 | 1.00 | 13.75 | W |
| ATOM | 4047 | OH2 | WAT | W | 63 | 47.480 | 85.774 | 30.437 | 1.00 | 15.69 | W |
| ATOM | 4048 | OH2 | WAT | W | 64 | 52.509 | 73.385 | 21.803 | 1.00 | 12.40 | W |
| ATOM | 4049 | OH2 | WAT | W | 65 | 36.499 | 69.305 | 21.796 | 1.00 | 13.29 | W |
| ATOM | 4050 | OH2 | WAT | W | 66 | 56.541 | 66.956 | 43.061 | 1.00 | 19.69 | W |
| ATOM | 4051 | OH2 | WAT | W | 67 | 44.592 | 65.330 | 39.631 | 1.00 | 15.23 | W |
| ATOM | 4052 | OH2 | WAT | W | 68 | 43.422 | 66.065 | 16.840 | 1.00 | 16.31 | W |
| ATOM | 4053 | OH2 | WAT | W | 69 | 45.595 | 76.658 | 16.838 | 1.00 | 15.43 | W |
| ATOM | 4054 | OH2 | WAT | W | 70 | 52.117 | 76.180 | 21.515 | 1.00 | 15.25 | W |
| ATOM | 4055 | OH2 | WAT | W | 71 | 34.389 | 82.628 | 8.611 | 1.00 | 15.39 | W |
| ATOM | 4056 | OH2 | WAT | W | 72 | 43.502 | 56.313 | 25.895 | 1.00 | 13.93 | W |
| ATOM | 4057 | OH2 | WAT | W | 73 | 44.383 | 77.298 | 39.674 | 1.00 | 15.09 | W |
| ATOM | 4058 | OH2 | WAT | W | 74 | 31.402 | 80.991 | 4.150 | 1.00 | 16.58 | W |
| ATOM | 4059 | OH2 | WAT | W | 75 | 45.484 | 86.340 | 25.866 | 1.00 | 13.07 | W |
| ATOM | 4060 | OH2 | WAT | W | 76 | 29.658 | 83.471 | 6.193 | 1.00 | 16.11 | W |
| ATOM | 4061 | OH2 | WAT | W | 77 | 50.842 | 53.536 | 18.587 | 1.00 | 16.64 | W |
| ATOM | 4062 | OH2 | WAT | W | 78 | 52.363 | 98.944 | 13.085 | 1.00 | 16.61 | W |
| ATOM | 4063 | OH2 | WAT | W | 79 | 38.066 | 89.045 | 18.567 | 1.00 | 16.20 | W |
| ATOM | 4064 | OH2 | WAT | W | 80 | 44.290 | 68.562 | 20.537 | 1.00 | 13.98 | W |
| ATOM | 4065 | OH2 | WAT | W | 81 | 54.651 | 59.977 | 8.635 | 1.00 | 16.03 | W |
| ATOM | 4066 | OH2 | WAT | W | 82 | 36.867 | 66.495 | 21.576 | 1.00 | 15.68 | W |
| ATOM | 4067 | OH2 | WAT | W | 83 | 23.603 | 84.009 | 21.786 | 1.00 | 15.33 | W |
| ATOM | 4068 | OH2 | WAT | W | 84 | 54.729 | 50.430 | −1.548 | 1.00 | 19.38 | W |
| ATOM | 4069 | OH2 | WAT | W | 85 | 57.530 | 61.744 | 4.120 | 1.00 | 16.87 | W |
| ATOM | 4070 | OH2 | WAT | W | 86 | 41.088 | 105.022 | 16.290 | 1.00 | 15.69 | W |
| ATOM | 4071 | OH2 | WAT | W | 87 | 38.319 | 71.451 | 36.984 | 1.00 | 17.14 | W |
| ATOM | 4072 | OH2 | WAT | W | 88 | 31.517 | 89.824 | 37.467 | 1.00 | 16.58 | W |
| ATOM | 4073 | OH2 | WAT | W | 89 | 50.641 | 71.222 | 36.997 | 1.00 | 18.14 | W |
| ATOM | 4074 | OH2 | WAT | W | 90 | 55.999 | 54.399 | −1.005 | 1.00 | 14.41 | W |
| ATOM | 4075 | OH2 | WAT | W | 91 | 48.673 | 35.111 | 15.911 | 1.00 | 17.63 | W |
| ATOM | 4076 | OH2 | WAT | W | 92 | 36.357 | 90.765 | −0.686 | 1.00 | 15.56 | W |
| ATOM | 4077 | OH2 | WAT | W | 93 | 47.926 | 37.673 | 16.359 | 1.00 | 15.70 | W |
| ATOM | 4078 | OH2 | WAT | W | 94 | 61.370 | 53.955 | 18.871 | 1.00 | 17.39 | W |
| ATOM | 4079 | OH2 | WAT | W | 95 | 48.563 | 49.341 | −0.111 | 1.00 | 19.51 | W |
| ATOM | 4080 | OH2 | WAT | W | 96 | 57.430 | 52.827 | 37.437 | 1.00 | 15.87 | W |
| ATOM | 4081 | OH2 | WAT | W | 97 | 32.940 | 88.241 | −0.945 | 1.00 | 16.26 | W |
| ATOM | 4082 | OH2 | WAT | W | 98 | 65.341 | 58.661 | 21.797 | 1.00 | 17.64 | W |
| ATOM | 4083 | OH2 | WAT | W | 99 | 59.363 | 46.482 | 12.477 | 1.00 | 20.25 | W |
| ATOM | 4084 | OH2 | WAT | W | 100 | 59.009 | 55.962 | 24.448 | 1.00 | 17.12 | W |
| ATOM | 4085 | OH2 | WAT | W | 101 | 27.659 | 88.655 | 18.900 | 1.00 | 19.39 | W |
| ATOM | 4086 | OH2 | WAT | W | 102 | 19.552 | 70.631 | 37.180 | 1.00 | 19.27 | W |
| ATOM | 4087 | OH2 | WAT | W | 103 | 62.732 | 52.896 | 9.518 | 1.00 | 18.07 | W |
| ATOM | 4088 | OH2 | WAT | W | 104 | 45.507 | 67.690 | 37.224 | 1.00 | 17.53 | W |
| ATOM | 4089 | OH2 | WAT | W | 105 | 38.378 | 83.608 | 45.067 | 1.00 | 17.12 | W |
| ATOM | 4090 | OH2 | WAT | W | 106 | 50.624 | 59.091 | 45.021 | 1.00 | 17.16 | W |
| ATOM | 4091 | OH2 | WAT | W | 107 | 36.693 | 43.685 | 13.087 | 1.00 | 17.34 | W |
| ATOM | 4092 | OH2 | WAT | W | 108 | 30.022 | 86.690 | 24.483 | 1.00 | 16.79 | W |
| ATOM | 4093 | OH2 | WAT | W | 109 | 26.705 | 89.770 | 12.172 | 1.00 | 19.72 | W |
| ATOM | 4094 | OH2 | WAT | W | 110 | 78.751 | 67.589 | 30.438 | 1.00 | 16.39 | W |
| ATOM | 4095 | OH2 | WAT | W | 111 | 43.442 | 74.935 | 37.288 | 1.00 | 18.43 | W |
| ATOM | 4096 | OH2 | WAT | W | 112 | 62.328 | 52.870 | 12.127 | 1.00 | 18.43 | W |
| ATOM | 4097 | OH2 | WAT | W | 113 | 52.635 | 51.855 | −0.691 | 1.00 | 15.44 | W |
| ATOM | 4098 | OH2 | WAT | W | 114 | 40.454 | 93.344 | −0.107 | 1.00 | 20.15 | W |
| ATOM | 4099 | OH2 | WAT | W | 115 | 40.297 | 107.578 | 15.942 | 1.00 | 17.59 | W |
| ATOM | 4100 | OH2 | WAT | W | 116 | 53.414 | 49.405 | 4.296 | 1.00 | 16.45 | W |
| ATOM | 4101 | OH2 | WAT | W | 117 | 69.415 | 72.026 | 37.172 | 1.00 | 18.90 | W |
| ATOM | 4102 | OH2 | WAT | W | 118 | 38.129 | 90.784 | 4.520 | 1.00 | 17.56 | W |
| ATOM | 4103 | OH2 | WAT | W | 119 | 38.146 | 55.292 | 23.299 | 1.00 | 16.51 | W |
| ATOM | 4104 | OH2 | WAT | W | 120 | 18.017 | 73.683 | 27.997 | 1.00 | 17.00 | W |
| ATOM | 4105 | OH2 | WAT | W | 121 | 53.528 | 43.974 | 7.984 | 1.00 | 16.88 | W |
| ATOM | 4106 | OH2 | WAT | W | 122 | 70.985 | 68.949 | 27.995 | 1.00 | 17.79 | W |
| ATOM | 4107 | OH2 | WAT | W | 123 | 35.576 | 93.322 | 4.318 | 1.00 | 16.95 | W |
| ATOM | 4108 | OH2 | WAT | W | 124 | 43.104 | 86.584 | 29.818 | 1.00 | 19.05 | W |
| ATOM | 4109 | OH2 | WAT | W | 125 | 48.853 | 41.039 | 10.115 | 1.00 | 18.07 | W |
| ATOM | 4110 | OH2 | WAT | W | 126 | 29.628 | 96.265 | 12.429 | 1.00 | 20.43 | W |
| ATOM | 4111 | OH2 | WAT | W | 127 | 37.667 | 89.314 | 29.451 | 1.00 | 18.21 | W |
| ATOM | 4112 | OH2 | WAT | W | 128 | 10.197 | 75.072 | 30.468 | 1.00 | 17.84 | W |
| ATOM | 4113 | OH2 | WAT | W | 129 | 27.695 | 76.838 | 11.844 | 1.00 | 17.47 | W |
| ATOM | 4114 | OH2 | WAT | W | 130 | 51.352 | 53.281 | 29.447 | 1.00 | 17.26 | W |
| ATOM | 4115 | OH2 | WAT | W | 131 | 46.024 | 56.163 | 29.682 | 1.00 | 18.32 | W |
| ATOM | 4116 | OH2 | WAT | W | 132 | 50.807 | 87.407 | 23.278 | 1.00 | 17.70 | W |
| ATOM | 4117 | OH2 | WAT | W | 133 | 29.565 | 83.957 | 17.933 | 1.00 | 19.40 | W |
| ATOM | 4118 | OH2 | WAT | W | 134 | 40.142 | 101.601 | 10.131 | 1.00 | 17.69 | W |
| ATOM | 4119 | OH2 | WAT | W | 135 | 53.886 | 52.579 | 28.038 | 1.00 | 16.20 | W |
| ATOM | 4120 | OH2 | WAT | W | 136 | 30.858 | 94.458 | 1.501 | 1.00 | 19.15 | W |
| ATOM | 4121 | OH2 | WAT | W | 137 | 48.663 | 73.041 | 18.714 | 1.00 | 17.83 | W |
| ATOM | 4122 | OH2 | WAT | W | 138 | 59.527 | 58.698 | 17.871 | 1.00 | 19.95 | W |
| ATOM | 4123 | OH2 | WAT | W | 139 | 58.111 | 48.183 | 1.475 | 1.00 | 18.47 | W |
| ATOM | 4124 | OH2 | WAT | W | 140 | 26.262 | 89.710 | 9.524 | 1.00 | 18.76 | W |
| ATOM | 4125 | OH2 | WAT | W | 141 | 61.241 | 65.789 | 11.822 | 1.00 | 18.51 | W |
| ATOM | 4126 | OH2 | WAT | W | 142 | 35.455 | 98.631 | 8.012 | 1.00 | 18.57 | W |
| ATOM | 4127 | OH2 | WAT | W | 143 | 35.097 | 90.126 | 28.102 | 1.00 | 15.69 | W |
| ATOM | 4128 | OH2 | WAT | W | 144 | 40.256 | 69.610 | 18.629 | 1.00 | 18.94 | W |
| ATOM | 4129 | OH2 | WAT | W | 145 | 24.207 | 86.822 | 36.953 | 1.00 | 21.47 | W |
| ATOM | 4130 | OH2 | WAT | W | 146 | 50.943 | 51.838 | 4.464 | 1.00 | 18.39 | W |
| ATOM | 4131 | OH2 | WAT | W | 147 | 31.576 | 56.567 | 23.098 | 1.00 | 19.24 | W |
| ATOM | 4132 | OH2 | WAT | W | 148 | 64.776 | 55.837 | 37.034 | 1.00 | 20.18 | W |
| ATOM | 4133 | OH2 | WAT | W | 149 | 41.752 | 88.655 | 30.593 | 1.00 | 19.15 | W |
| ATOM | 4134 | OH2 | WAT | W | 150 | 28.943 | 90.064 | 36.740 | 1.00 | 18.17 | W |
| ATOM | 4135 | OH2 | WAT | W | 151 | 53.126 | 74.689 | 17.462 | 1.00 | 21.71 | W |
| ATOM | 4136 | OH2 | WAT | W | 152 | 57.481 | 86.100 | 23.079 | 1.00 | 19.09 | W |
| ATOM | 4137 | OH2 | WAT | W | 153 | 35.919 | 68.039 | 17.377 | 1.00 | 22.31 | W |
| ATOM | 4138 | OH2 | WAT | W | 154 | 51.862 | 84.976 | 9.501 | 1.00 | 19.42 | W |
| ATOM | 4139 | OH2 | WAT | W | 155 | 34.813 | 82.161 | 45.754 | 1.00 | 19.06 | W |
| ATOM | 4140 | OH2 | WAT | W | 156 | 60.087 | 52.558 | 36.734 | 1.00 | 18.62 | W |
| ATOM | 4141 | OH2 | WAT | W | 157 | 41.492 | 74.254 | 17.305 | 1.00 | 22.53 | W |
| ATOM | 4142 | OH2 | WAT | W | 158 | 51.400 | 69.620 | 18.407 | 1.00 | 19.54 | W |
| ATOM | 4143 | OH2 | WAT | W | 159 | 43.474 | 90.722 | 33.704 | 1.00 | 22.13 | W |
| ATOM | 4144 | OH2 | WAT | W | 160 | 55.691 | 62.546 | −2.287 | 1.00 | 19.40 | W |
| ATOM | 4145 | OH2 | WAT | W | 161 | 33.174 | 80.145 | −2.336 | 1.00 | 17.53 | W |
| ATOM | 4146 | OH2 | WAT | W | 162 | 54.074 | 60.472 | 45.738 | 1.00 | 20.40 | W |
| ATOM | 4147 | OH2 | WAT | W | 163 | 45.519 | 51.887 | 33.616 | 1.00 | 24.73 | W |
| ATOM | 4148 | OH2 | WAT | W | 164 | 32.388 | 61.060 | 18.277 | 1.00 | 17.09 | W |
| ATOM | 4149 | OH2 | WAT | W | 165 | 56.542 | 81.596 | 18.358 | 1.00 | 18.68 | W |
| ATOM | 4150 | OH2 | WAT | W | 166 | 50.758 | 68.823 | 41.601 | 1.00 | 18.56 | W |
| ATOM | 4151 | OH2 | WAT | W | 167 | 10.404 | 82.696 | 30.006 | 1.00 | 17.07 | W |
| ATOM | 4152 | OH2 | WAT | W | 168 | 39.208 | 57.693 | 24.249 | 1.00 | 15.79 | W |
| ATOM | 4153 | OH2 | WAT | W | 169 | 47.497 | 68.384 | 17.369 | 1.00 | 22.69 | W |
| ATOM | 4154 | OH2 | WAT | W | 170 | 20.230 | 68.989 | 32.778 | 1.00 | 19.32 | W |
| ATOM | 4155 | OH2 | WAT | W | 171 | 38.240 | 73.855 | 41.614 | 1.00 | 19.18 | W |
| ATOM | 4156 | OH2 | WAT | W | 172 | 52.104 | 36.869 | 15.182 | 1.00 | 23.40 | W |
| ATOM | 4157 | OH2 | WAT | W | 173 | 47.160 | 53.954 | 30.617 | 1.00 | 20.86 | W |
| ATOM | 4158 | OH2 | WAT | W | 174 | 29.888 | 88.727 | 21.062 | 1.00 | 19.28 | W |
| ATOM | 4159 | OH2 | WAT | W | 175 | 32.681 | 90.508 | 26.429 | 1.00 | 23.03 | W |
| ATOM | 4160 | OH2 | WAT | W | 176 | 57.504 | 94.320 | 14.819 | 1.00 | 19.43 | W |
| ATOM | 4161 | OH2 | WAT | W | 177 | 31.443 | 48.305 | 14.849 | 1.00 | 18.59 | W |
| ATOM | 4162 | OH2 | WAT | W | 178 | 57.549 | 87.754 | 14.045 | 1.00 | 20.94 | W |
| ATOM | 4163 | OH2 | WAT | W | 179 | 45.086 | 60.385 | 10.733 | 1.00 | 23.96 | W |
| ATOM | 4164 | OH2 | WAT | W | 180 | 68.770 | 73.686 | 32.727 | 1.00 | 18.69 | W |
| ATOM | 4165 | OH2 | WAT | W | 181 | 31.593 | 54.992 | 13.997 | 1.00 | 21.30 | W |
| ATOM | 4166 | OH2 | WAT | W | 182 | 37.645 | 73.062 | 18.423 | 1.00 | 20.42 | W |
| ATOM | 4167 | OH2 | WAT | W | 183 | 76.394 | 58.932 | 28.770 | 1.00 | 18.62 | W |
| ATOM | 4168 | OH2 | WAT | W | 184 | 37.029 | 105.750 | 15.195 | 1.00 | 25.05 | W |
| ATOM | 4169 | OH2 | WAT | W | 185 | 49.799 | 84.965 | 24.229 | 1.00 | 16.64 | W |
| ATOM | 4170 | OH2 | WAT | W | 186 | 29.729 | 49.663 | 19.033 | 1.00 | 24.99 | W |
| ATOM | 4171 | OH2 | WAT | W | 187 | 78.577 | 60.024 | 29.973 | 1.00 | 18.33 | W |
| ATOM | 4172 | OH2 | WAT | W | 188 | 73.129 | 67.863 | 35.211 | 1.00 | 19.22 | W |
| ATOM | 4173 | OH2 | WAT | W | 189 | 56.199 | 52.141 | 26.472 | 1.00 | 21.85 | W |
| ATOM | 4174 | OH2 | WAT | W | 190 | 51.216 | 48.189 | 35.676 | 1.00 | 22.68 | W |
| ATOM | 4175 | OH2 | WAT | W | 191 | 38.496 | 37.885 | 13.551 | 1.00 | 20.85 | W |
| ATOM | 4176 | OH2 | WAT | W | 192 | 42.144 | 83.131 | 43.841 | 1.00 | 22.20 | W |
| ATOM | 4177 | OH2 | WAT | W | 193 | 37.800 | 99.161 | 6.541 | 1.00 | 20.77 | W |
| ATOM | 4178 | OH2 | WAT | W | 194 | 79.202 | 59.134 | 23.199 | 1.00 | 22.25 | W |
| ATOM | 4179 | OH2 | WAT | W | 195 | 43.464 | 84.043 | 0.966 | 1.00 | 22.99 | W |
| ATOM | 4180 | OH2 | WAT | W | 196 | 9.774 | 83.521 | 23.123 | 1.00 | 21.99 | W |
| ATOM | 4181 | OH2 | WAT | W | 197 | 40.774 | 56.321 | 25.940 | 1.00 | 24.08 | W |
| ATOM | 4182 | OH2 | WAT | W | 198 | 36.174 | 56.191 | 21.678 | 1.00 | 20.43 | W |
| ATOM | 4183 | OH2 | WAT | W | 199 | 37.100 | 57.779 | 9.382 | 1.00 | 21.73 | W |
| ATOM | 4184 | OH2 | WAT | W | 200 | 67.040 | 54.065 | 35.139 | 1.00 | 19.42 | W |
| ATOM | 4185 | OH2 | WAT | W | 201 | 54.990 | 87.263 | 23.288 | 1.00 | 18.23 | W |
| ATOM | 4186 | OH2 | WAT | W | 202 | 29.566 | 81.588 | −1.127 | 1.00 | 24.32 | W |
| ATOM | 4187 | OH2 | WAT | W | 203 | 50.482 | 104.877 | 13.543 | 1.00 | 21.51 | W |
| ATOM | 4188 | OH2 | WAT | W | 204 | 33.809 | 75.981 | 8.987 | 1.00 | 21.22 | W |
| ATOM | 4189 | OH2 | WAT | W | 205 | 58.446 | 82.817 | 21.143 | 1.00 | 24.77 | W |
| ATOM | 4190 | OH2 | WAT | W | 206 | 33.990 | 55.358 | 23.381 | 1.00 | 19.17 | W |
| ATOM | 4191 | OH2 | WAT | W | 207 | 59.100 | 53.954 | 21.096 | 1.00 | 21.68 | W |
| ATOM | 4192 | OH2 | WAT | W | 208 | 43.863 | 75.725 | 43.193 | 1.00 | 25.02 | W |
| ATOM | 4193 | OH2 | WAT | W | 209 | 59.257 | 92.897 | 18.903 | 1.00 | 24.44 | W |
| ATOM | 4194 | OH2 | WAT | W | 210 | 15.872 | 74.869 | 35.220 | 1.00 | 18.53 | W |
| ATOM | 4195 | OH2 | WAT | W | 211 | 37.990 | 100.485 | 8.868 | 1.00 | 19.08 | W |
| ATOM | 4196 | OH2 | WAT | W | 212 | 44.528 | 104.608 | 10.069 | 1.00 | 28.45 | W |
| ATOM | 4197 | OH2 | WAT | W | 213 | 22.020 | 67.110 | 33.698 | 1.00 | 22.25 | W |
| ATOM | 4198 | OH2 | WAT | W | 214 | 45.524 | 58.541 | 0.865 | 1.00 | 24.20 | W |
| ATOM | 4199 | OH2 | WAT | W | 215 | 46.880 | 59.443 | 43.760 | 1.00 | 21.51 | W |
| ATOM | 4200 | OH2 | WAT | W | 216 | 44.398 | 37.868 | 10.230 | 1.00 | 29.45 | W |
| ATOM | 4201 | OH2 | WAT | W | 217 | 42.403 | 47.455 | 1.037 | 1.00 | 22.17 | W |
| ATOM | 4202 | OH2 | WAT | W | 218 | 21.877 | 88.573 | 35.113 | 1.00 | 20.88 | W |
| ATOM | 4203 | OH2 | WAT | W | 219 | 55.162 | 66.703 | 8.963 | 1.00 | 22.51 | W |
| ATOM | 4204 | OH2 | WAT | W | 220 | 52.791 | 86.448 | 21.601 | 1.00 | 21.18 | W |
| ATOM | 4205 | OH2 | WAT | W | 221 | 51.147 | 43.483 | 6.559 | 1.00 | 20.91 | W |
| ATOM | 4206 | OH2 | WAT | W | 222 | 43.808 | 82.212 | 10.720 | 1.00 | 26.33 | W |
| ATOM | 4207 | OH2 | WAT | W | 223 | 12.617 | 83.802 | 28.751 | 1.00 | 20.30 | W |
| ATOM | 4208 | OH2 | WAT | W | 224 | 6.078 | 75.280 | 26.850 | 1.00 | 22.38 | W |
| ATOM | 4209 | OH2 | WAT | W | 225 | 47.881 | 57.392 | −0.792 | 1.00 | 22.09 | W |
| ATOM | 4210 | OH2 | WAT | W | 226 | 50.980 | 42.104 | 8.831 | 1.00 | 19.78 | W |
| ATOM | 4211 | OH2 | WAT | W | 227 | 41.154 | 85.258 | −0.774 | 1.00 | 22.59 | W |
| ATOM | 4212 | OH2 | WAT | W | 228 | 25.986 | 84.606 | 36.424 | 1.00 | 19.93 | W |
| ATOM | 4213 | OH2 | WAT | W | 229 | 37.760 | 94.424 | 35.734 | 1.00 | 24.94 | W |
| ATOM | 4214 | OH2 | WAT | W | 230 | 53.942 | 75.516 | 42.827 | 1.00 | 21.96 | W |
| ATOM | 4215 | OH2 | WAT | W | 231 | 62.956 | 58.032 | 36.430 | 1.00 | 20.68 | W |
| ATOM | 4216 | OH2 | WAT | W | 232 | 48.172 | 76.513 | 38.212 | 1.00 | 24.35 | W |
| ATOM | 4217 | OH2 | WAT | W | 233 | 47.611 | 102.405 | 12.413 | 1.00 | 18.05 | W |
| ATOM | 4218 | OH2 | WAT | W | 234 | 66.970 | 75.519 | 33.669 | 1.00 | 22.17 | W |
| ATOM | 4219 | OH2 | WAT | W | 235 | 42.530 | 53.001 | 37.024 | 1.00 | 25.29 | W |
| ATOM | 4220 | OH2 | WAT | W | 236 | 46.593 | 95.318 | 0.982 | 1.00 | 22.70 | W |
| ATOM | 4221 | OH2 | WAT | W | 237 | 59.434 | 61.102 | −1.153 | 1.00 | 23.03 | W |
| ATOM | 4222 | OH2 | WAT | W | 238 | 41.428 | 40.251 | 12.333 | 1.00 | 19.70 | W |
| ATOM | 4223 | OH2 | WAT | W | 239 | 30.566 | 59.844 | 21.226 | 1.00 | 24.21 | W |
| ATOM | 4224 | OH2 | WAT | W | 240 | 40.963 | 66.286 | 38.250 | 1.00 | 25.23 | W |
| ATOM | 4225 | OH2 | WAT | W | 241 | 35.100 | 102.269 | 13.651 | 1.00 | 23.54 | W |
| ATOM | 4226 | OH2 | WAT | W | 242 | 46.138 | 72.116 | 35.284 | 1.00 | 22.17 | W |
| ATOM | 4227 | OH2 | WAT | W | 243 | 59.052 | 47.058 | 31.892 | 1.00 | 23.41 | W |
| ATOM | 4228 | OH2 | WAT | W | 244 | 27.083 | 65.207 | 15.974 | 1.00 | 24.57 | W |
| ATOM | 4229 | OH2 | WAT | W | 245 | 49.228 | 51.393 | 44.483 | 1.00 | 25.93 | W |
| ATOM | 4230 | OH2 | WAT | W | 246 | 53.883 | 40.364 | 13.609 | 1.00 | 20.63 | W |
| ATOM | 4231 | OH2 | WAT | W | 247 | 19.324 | 82.747 | 33.510 | 1.00 | 19.13 | W |
| ATOM | 4232 | OH2 | WAT | W | 248 | 26.043 | 85.533 | 19.642 | 1.00 | 23.45 | W |
| ATOM | 4233 | OH2 | WAT | W | 249 | 35.021 | 67.121 | 42.825 | 1.00 | 22.49 | W |
| ATOM | 4234 | OH2 | WAT | W | 250 | 46.735 | 76.516 | 12.252 | 1.00 | 24.05 | W |
| ATOM | 4235 | OH2 | WAT | W | 251 | 82.969 | 67.303 | 26.717 | 1.00 | 22.57 | W |
| ATOM | 4236 | OH2 | WAT | W | 252 | 45.209 | 67.018 | 43.177 | 1.00 | 27.94 | W |
| ATOM | 4237 | OH2 | WAT | W | 253 | 55.602 | 79.331 | 37.534 | 1.00 | 24.03 | W |
| ATOM | 4238 | OH2 | WAT | W | 254 | 69.676 | 59.919 | 33.683 | 1.00 | 20.99 | W |
| ATOM | 4239 | OH2 | WAT | W | 255 | 64.125 | 59.002 | 14.884 | 1.00 | 25.33 | W |
| ATOM | 4240 | OH2 | WAT | W | 256 | 42.314 | 66.230 | 12.402 | 1.00 | 23.88 | W |
| ATOM | 4241 | OH2 | WAT | W | 257 | 33.279 | 63.349 | 37.448 | 1.00 | 22.10 | W |
| ATOM | 4242 | OH2 | WAT | W | 258 | 39.649 | 91.675 | 44.503 | 1.00 | 27.13 | W |
| ATOM | 4243 | OH2 | WAT | W | 259 | 50.183 | 77.624 | 17.514 | 1.00 | 28.33 | W |
| ATOM | 4244 | OH2 | WAT | W | 260 | 61.881 | 77.494 | 15.876 | 1.00 | 23.81 | W |
| ATOM | 4245 | OH2 | WAT | W | 261 | 62.925 | 57.128 | 19.612 | 1.00 | 21.49 | W |
| ATOM | 4246 | OH2 | WAT | W | 262 | 78.761 | 69.710 | 28.537 | 1.00 | 21.54 | W |
| ATOM | 4247 | OH2 | WAT | W | 263 | 43.443 | 78.531 | 26.450 | 1.00 | 51.57 | W |
| ATOM | 4248 | OH2 | WAT | W | 264 | 64.436 | 66.462 | 15.185 | 1.00 | 24.50 | W |
| ATOM | 4249 | OH2 | WAT | W | 265 | 29.863 | 95.634 | 31.799 | 1.00 | 22.57 | W |
| ATOM | 4250 | OH2 | WAT | W | 266 | 38.733 | 65.028 | 17.513 | 1.00 | 22.79 | W |
| ATOM | 4251 | OH2 | WAT | W | 267 | 42.862 | 70.588 | 35.156 | 1.00 | 23.17 | W |
| ATOM | 4252 | OH2 | WAT | W | 268 | 18.772 | 72.838 | 25.393 | 1.00 | 22.19 | W |
| ATOM | 4253 | OH2 | WAT | W | 269 | 73.723 | 64.597 | 19.000 | 1.00 | 31.96 | W |
| ATOM | 4254 | OH2 | WAT | W | 270 | 62.723 | 57.662 | 10.545 | 1.00 | 24.92 | W |
| ATOM | 4255 | OH2 | WAT | W | 271 | 57.874 | 62.395 | 44.609 | 1.00 | 21.51 | W |
| ATOM | 4256 | OH2 | WAT | W | 272 | 48.768 | 50.303 | 27.819 | 1.00 | 19.31 | W |
| ATOM | 4257 | OH2 | WAT | W | 273 | 29.763 | 86.426 | 47.578 | 1.00 | 28.69 | W |
| ATOM | 4258 | OH2 | WAT | W | 274 | 31.100 | 59.737 | 35.193 | 1.00 | 31.03 | W |
| ATOM | 4259 | OH2 | WAT | W | 275 | 57.280 | 52.145 | 23.039 | 1.00 | 31.70 | W |
| ATOM | 4260 | OH2 | WAT | W | 276 | 39.141 | 75.601 | 16.301 | 1.00 | 19.15 | W |
| ATOM | 4261 | OH2 | WAT | W | 277 | 40.242 | 92.360 | 27.881 | 1.00 | 21.57 | W |
| ATOM | 4262 | OH2 | WAT | W | 278 | 45.848 | 39.924 | 20.297 | 1.00 | 19.61 | W |
| ATOM | 4263 | OH2 | WAT | W | 279 | 37.239 | 92.961 | 30.038 | 1.00 | 35.26 | W |
| ATOM | 4264 | OH2 | WAT | W | 280 | 34.443 | 91.256 | 24.199 | 1.00 | 23.25 | W |
| ATOM | 4265 | OH2 | WAT | W | 281 | 32.844 | 98.633 | 13.982 | 1.00 | 25.62 | W |
| ATOM | 4266 | OH2 | WAT | W | 282 | 27.691 | 84.905 | −0.900 | 1.00 | 31.39 | W |
| ATOM | 4267 | OH2 | WAT | W | 283 | 49.770 | 67.058 | 16.244 | 1.00 | 19.14 | W |
| ATOM | 4268 | OH2 | WAT | W | 284 | 7.549 | 84.030 | 30.735 | 1.00 | 23.79 | W |
| ATOM | 4269 | OH2 | WAT | W | 285 | 41.424 | 76.017 | 13.321 | 1.00 | 26.91 | W |
| ATOM | 4270 | OH2 | WAT | W | 286 | 26.035 | 92.828 | 18.463 | 1.00 | 27.45 | W |
| ATOM | 4271 | OH2 | WAT | W | 287 | 32.937 | 94.119 | 24.137 | 1.00 | 48.00 | W |
| ATOM | 4272 | OH2 | WAT | W | 288 | 26.166 | 88.282 | 25.556 | 1.00 | 20.51 | W |
| ATOM | 4273 | OH2 | WAT | W | 289 | 63.983 | 65.811 | 11.880 | 1.00 | 27.50 | W |
| ATOM | 4274 | OH2 | WAT | W | 290 | 22.109 | 58.615 | 30.718 | 1.00 | 23.84 | W |
| ATOM | 4275 | OH2 | WAT | W | 291 | 19.238 | 68.372 | 19.647 | 1.00 | 28.58 | W |
| ATOM | 4276 | OH2 | WAT | W | 292 | 56.100 | 44.047 | 14.035 | 1.00 | 24.23 | W |
| ATOM | 4277 | OH2 | WAT | W | 293 | 54.648 | 51.202 | 24.011 | 1.00 | 23.26 | W |
| ATOM | 4278 | OH2 | WAT | W | 294 | 31.678 | 90.398 | 23.056 | 1.00 | 26.00 | W |
| ATOM | 4279 | OH2 | WAT | W | 295 | 53.694 | 43.318 | 2.052 | 1.00 | 22.76 | W |
| ATOM | 4280 | OH2 | WAT | W | 296 | 26.404 | 85.036 | 10.533 | 1.00 | 25.60 | W |
| ATOM | 4281 | OH2 | WAT | W | 297 | 57.999 | 82.967 | 35.181 | 1.00 | 32.74 | W |
| ATOM | 4282 | OH2 | WAT | W | 298 | 24.844 | 83.608 | 14.725 | 1.00 | 32.65 | W |
| ATOM | 4283 | OH2 | WAT | W | 299 | 66.137 | 73.414 | 43.426 | 1.00 | 23.33 | W |
| ATOM | 4284 | OH2 | WAT | W | 300 | 60.389 | 52.123 | 26.401 | 1.00 | 27.82 | W |
| ATOM | 4285 | OH2 | WAT | W | 301 | 45.077 | 83.036 | 43.695 | 1.00 | 28.17 | W |
| ATOM | 4286 | OH2 | WAT | W | 302 | 63.568 | 69.731 | 13.225 | 1.00 | 40.57 | W |
| ATOM | 4287 | OH2 | WAT | W | 303 | 15.309 | 78.322 | 19.060 | 1.00 | 29.40 | W |
| ATOM | 4288 | OH2 | WAT | W | 304 | 6.803 | 75.844 | 23.827 | 1.00 | 27.70 | W |
| ATOM | 4289 | OH2 | WAT | W | 305 | 24.548 | 76.310 | 15.211 | 1.00 | 27.04 | W |
| ATOM | 4290 | OH2 | WAT | W | 306 | 21.315 | 69.478 | 26.567 | 1.00 | 25.89 | W |
| ATOM | 4291 | OH2 | WAT | W | 307 | 55.614 | 92.278 | 9.486 | 1.00 | 28.03 | W |
| ATOM | 4292 | OH2 | WAT | W | 308 | 33.401 | 50.305 | 9.283 | 1.00 | 26.50 | W |
| ATOM | 4293 | OH2 | WAT | W | 309 | 44.860 | 41.748 | 8.296 | 1.00 | 22.14 | W |
| ATOM | 4294 | OH2 | WAT | W | 310 | 36.981 | 98.022 | 38.919 | 1.00 | 27.75 | W |
| ATOM | 4295 | OH2 | WAT | W | 311 | 36.049 | 71.473 | 13.872 | 1.00 | 40.12 | W |
| ATOM | 4296 | OH2 | WAT | W | 312 | 62.799 | 54.411 | 25.492 | 1.00 | 20.27 | W |
| ATOM | 4297 | OH2 | WAT | W | 313 | 68.900 | 66.841 | 46.634 | 1.00 | 40.74 | W |
| ATOM | 4298 | OH2 | WAT | W | 314 | 52.771 | 46.326 | −2.783 | 1.00 | 27.19 | W |
| ATOM | 4299 | OH2 | WAT | W | 315 | 25.076 | 76.838 | 11.881 | 1.00 | 29.09 | W |
| ATOM | 4300 | OH2 | WAT | W | 316 | 37.345 | 55.941 | 35.837 | 1.00 | 31.26 | W |
| END | | | | | | | | | | | |

What is claimed:

1. A pharmaceutical composition comprising a pharmaceutically acceptable excipient and a compound or a pharmaceutically acceptable salt thereof having the structure of formula 1:
R¹—R²R³R⁴—R⁵
where R¹ and R⁵ are the same and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl;
where R² and R⁴ are preferably, but not necessarily, the same, and are ring structures, optionally substituted, which may be aryl, cycloalkyl, or heterocyclyl; and
where R³ may be a bond between R² and R⁴ which either is shared by or joins the two rings without being shared in the rings themselves.

2. The pharmaceutical composition of claim 1 wherein R², R³, R⁴ is a naphthyl group.

3. The pharmaceutical composition of claim 1 wherein R², R³, R⁴ is a bi-aryl group.

4. A pharmaceutical composition comprising a pharmaceutically acceptable excipient and a compound or a pharmaceutically acceptable salt thereof having the structure of formula 2: 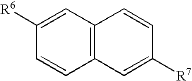
where R⁶ and R⁷ are the same or different and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl.

5. The pharmaceutical composition of claim 4 wherein R⁶ and R⁷ are the same and are both alkylaminosulfonyl groups.

6. The pharmaceutical composition of claim 4 wherein R⁶ and R⁷ are the same and are both alkylaminosulfonylalkyl groups.

7. The pharmaceutical composition of claim 6 wherein the compound has the structure of formula 3:

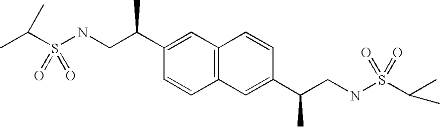

8. A pharmaceutical composition comprising a pharmaceutically acceptable excipient and a compound or a pharmaceutically acceptable salt thereof having the structure of formula 4: 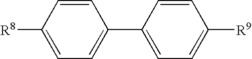
where R⁸ and R⁹ are the same and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl.

9. The pharmaceutical composition of claim 8 wherein R⁸ and R⁹ are both alkylaminosulfonyl groups.

10. The pharmaceutical composition of claim 8 wherein R⁸ and R⁹ are both alkylaminosulfonylalkyl groups.

11. The pharmaceutical composition of claim 10 wherein the compound has the structure of formula 5:

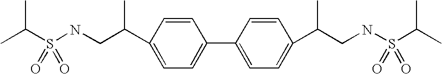

12. The pharmaceutical composition of claim 8 wherein the compound has the structure of formula 6:

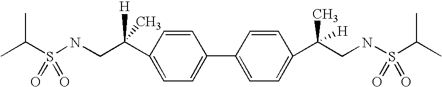

13. The pharmaceutical composition of claim 4 wherein the compound has the structure of formula 7: 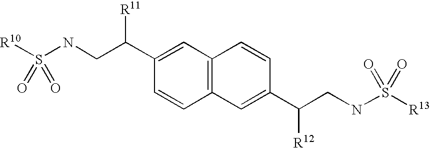
where R¹⁰ and R¹³ are the same, or, if they are not, then R¹¹ and R¹² are the same, and each of R¹⁰, R¹¹, R¹², and R¹³ may be H, methyl, ethyl, propyl, isopropyl or butyl.

14. A method of treating a disorder selected from the group consisting of anxiety and depression, comprising administering an effective amount of a pharmaceutical composition of claim 1.

15. A method of treating a disorder selected from the group consisting of anxiety and depression, comprising administering an effective amount of a pharmaceutical composition of claim 2.

16. A method of treating a disorder selected from the group consisting of anxiety and depression, comprising administering an effective amount of a pharmaceutical composition of claim 3.

17. A method of treating a disorder selected from the group consisting of anxiety and depression, comprising administering an effective amount of a pharmaceutical composition of claim 4.

18. A method of treating a disorder selected from the group consisting of anxiety and depression, comprising administering an effective amount of a pharmaceutical composition of claim 5.

19. A method of treating a disorder selected from the group consisting of anxiety and depression, comprising administering an effective amount of a pharmaceutical composition of claim 6.

20. A method of treating a disorder selected from the group consisting of anxiety and depression, comprising administering an effective amount of a pharmaceutical composition of claim 7.

21. A method of treating a disorder selected from the group consisting of anxiety and depression, comprising administering an effective amount of a pharmaceutical composition of claim 8.

22. A method of treating a disorder selected from the group consisting of anxiety and depression, comprising administering an effective amount of a pharmaceutical composition of claim 9.

23. A method of treating a disorder selected from the group consisting of anxiety and depression, comprising administering an effective amount of a pharmaceutical composition of claim 10.

24. A method of treating a disorder selected from the group consisting of anxiety and depression, comprising administering an effective amount of a pharmaceutical composition of claim 11.

25. A method of treating a disorder selected from the group consisting of anxiety and depression, comprising administering an effective amount of a pharmaceutical composition of claim 12.

26. A method of treating a disorder selected from the group consisting of anxiety and depression, comprising administering an effective amount of a pharmaceutical composition of claim 13.

27. Use of a compound of claim 1 for preparing a pharmaceutical for use in treating anxiety or depression.

28. The compound N,N′-(2R,2′R)-2,2′-(biphenyl-4,4′-diyl)bis(propane-2,1-diyl)dipropane-2-sulfonamide) or a pharmaceutically acceptable salt thereof.

29. The compound N,N′-(2S,2′S)-2,2′-(biphenyl-4,4′-diyl)bis(2-fluoropropane-2,1-diyl)dipropane-2-sulfonamide) or a pharmaceutically acceptable salt thereof.

30. The compound N,N′-(2S,2′S)-2,2′-(biphenyl-4,4′-diyl)bis(2-hydroxypropane-2,1-diyl)dipropane-2-sulfonamide or a pharmaceutically acceptable salt thereof.

31. A pharmaceutical composition in unit dosage form comprising a compound or pharmaceutically acceptable salt thereof of claim 28, 29 or 30 in combination with a pharmaceutically acceptable carrier.

32. A method of modulating the GluR2 receptor comprising administering to a human an effective amount of a pharmaceutical composition of claim 31.
